(19) United States
(12) Patent Application Publication
(10) Pub. No.: US 2026/0013513 A1
(43) Pub. Date: Jan. 15, 2026

(54) PESTICIDAL ACTX PEPTIDE VARIANTS

(71) Applicant: Vestaron Corporation, Kalamazoo, MI (US)

(72) Inventors: Alexandra M. Haase, Martin, MI (US); Nicole Marie Dubs, Kalamazoo, MI (US); Lilly Ann Ruell, Kalamazoo, MI (US)

(73) Assignee: Vestaron Corporation, Kalamazoo, MI (US)

(21) Appl. No.: 18/866,408

(22) PCT Filed: May 17, 2023
(86) PCT No.: PCT/US2023/067118

§ 371 Date: Nov. 15, 2024

Related U.S. Application Data
(60) Provisional application No. 63/343,399, filed on May 18, 2022.

(51) Int. Cl. A01N 63/50; A01P 7/00; A01P 7/04; C07K 14/435; C12P 21/02

(52) U.S. Cl. CPC A01N 63/50; A01P 7/00; A01P 7/04; C07K 14/43518; C12P 21/02

(57) ABSTRACT

An engineered, non-naturally occurring pesticidal Kappa Peptide Variant (KPV), or an agriculturally acceptable salt thereof, comprising, consisting essentially of, or consists of, an amino sequence that is at least 90%, 95%, 96%, 97%, 97%, 98%, 99%, or 100% identical to a sequence set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 35, is described, along with methods of making and using the same. The present disclosure is also related to and describes novel pesticidal compositions, formulations, and methods of using the same, that are useful for the control of plant pests.

### CROSS REFERENCE TO RELATED APPLICATIONS

This application is a National Stage Application under 35 U.S.C. § 371 of PCT Application No. PCT/US2023/067118, filed on May 17, 2023, and claims the benefit of, and priority to, earlier filed U.S. Provisional Application Ser. No. 63/343,399, filed on May 18, 2022, the entire contents of each of the aforementioned applications are incorporated by reference herein in their entireties.

### SEQUENCE LISTING

This application incorporates in its entirety the Sequence Listing XML entitled “277702-541259” (340,324 bytes), which was created on Sep. 5, 2025, and filed electronically herewith.

### TECHNICAL FIELD

Engineered, non-naturally occurring pesticidal peptides called Kappa Peptide Variants (KPVs), and agriculturally acceptable salts thereof, agricultural compositions and methods containing the same, for the control and/or eradication of pests, are described and claimed.

### BACKGROUND

Deleterious insects represent a worldwide threat to human health and food security. Insects pose a threat to human health because they are a vector for disease. One of the most notorious insect-vectors of disease is the mosquito. Mosquitoes in the genus Anopheles are the principal vectors of Zika virus, Chikungunya virus, and malaria—a disease caused by protozoa in the genus Trypanosoma. Another mosquito, Aedes aegypti, is the main vector of the viruses that cause Yellow fever and Dengue. And, Aedes spp. mosquitos are also the vectors for the viruses responsible for various types of encephalitis. Wuchereria bancrofti and Brugia malayi, parasitic roundworms that cause filariasis, are usually spread by mosquitoes in the genera Culex, Mansonia, and Anopheles.

Similar to the mosquito, other members of the Diptera order have likewise plagued humankind since time immemorial. In addition to producing painful bites, Horseflies and deerflies transmit the bacterial pathogens of tularemia (Pasteurella tularensis) and anthrax (Bacillus anthracis), as well as a parasitic roundworm (Loa loa) that causes loiasis in tropical Africa.

Blowflies (Chrysomya megacephala) and houseflies (Musca domestica) will in one moment take off from carrion and dung, and in the next moment alight in our homes and on our food-spreading dysentery, typhoid fever, cholera, poliomyelitis, yaws, leprosy, and tuberculosis in their wake.

Eye gnats in the genus Hippelates can carry the spirochaete pathogen that causes yaws (Treponema pertenue), and may also spread conjunctivitis (pinkeye). Tsetse flies in the genus Glossina transmit the protozoan pathogens that cause African sleeping sickness (Trypanosoma gambiense and T. rhodesiense). Sand flies in the genus Phlebotomus are vectors of a bacterium (Bartonella bacilliformis) that causes Carrion's disease (Oroyo fever) in South America. In parts of Asia and North Africa, they spread a viral agent that causes sand fly fever (Pappataci fever) as well as protozoan pathogens (Leishmania spp.) that cause Leishmaniasis.

Human food security is also threatened by insects. Insect pests indiscriminately target food crops earmarked for commercial purposes and personal use alike; indeed, the damage caused by insect pests can run the gamut from mere inconvenience to financial ruin in the former, to extremes such as malnutrition or starvation in the latter. Insect pests also cause stress and disease in domesticated animals. And, insect pests once limited by geographical and climate boundaries have expanded their range due to global travel and climate change.

Accordingly, there is a need for alternative, environmentally friendly pesticidal agents and compositions to protect economically important commodity and staple calorie crops from the threats posed by destructive insects.

### SUMMARY

The present disclosure describes novel engineered, non-naturally occurring pesticidal Kappa Peptide Variants (KPVs), and agriculturally acceptable salts thereof.

In addition, the present disclosure describes a Kappa Peptide Variant (KPV) having pesticidal (for example, insecticidal) activity against one or more pests, said KPV comprising an amino acid sequence that is at least 85%, 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In addition, the present disclosure describes a KPV comprises, consists essentially of, or consists of an amino acid sequence that is at least 85%, 90%, 95%, 96%, 97%, 97%, 98%, 99%, or 100% identical to the amino acid sequence according to any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; or an agriculturally acceptable salt thereof.

In addition, the present disclosure describes a combination comprising two or more Kappa Peptide Variants (KPVs) having pesticidal activity against one or more pest species, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that each of the KPVs does not comprise the amino acid sequence of SEQ ID NOs: 1 or 253.

In addition, the present disclosure describes a composition comprising one or more KPVs and one or more agriculturally acceptable excipients; wherein the KPV comprises an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In addition, the present disclosure describes a polynucleotide operable to encode an Kappa Peptide Variant (KPV), said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary polynucleotide sequence thereof, with the proviso that the polynucleotide does not encode a KPV comprising or consisting of the amino acid sequence of SEQ ID NOs: 1 or 253.

In addition, the present disclosure describes vector comprising a polynucleotide operable to encode an Kappa Peptide Variant (KPV), said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary polynucleotide sequence thereof, with the proviso that the polynucleotide does not encode a KPV comprising or consisting of the amino acid sequence of SEQ ID NOs: 1 or 253.

In addition, the present disclosure describes a polynucleotide operable to hybridize under stringent hybridization conditions with a polynucleotide segment encoding a Kappa Peptide Variant (KPV), said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary polynucleotide sequence thereof, with the proviso that the polynucleotide does not encode a KPV comprising or consisting of the amino acid sequence of SEQ ID NOs: 1 or 253.

In addition, the present disclosure describes a yeast strain comprising: a first expression cassette comprising a polynucleotide operable to encode a KPV, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof; with the proviso that the KPV does not comprise the amino acid sequence of SEQ ID NOs: 1 or 253.

In addition, the present disclosure describes a method of producing a KPV, the method comprising: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NO: 1 or 253; (b) introducing the vector into a host cell; and (c) growing the host cell in a growth medium under conditions operable to enable expression of the KPV and secretion into the growth medium.

In addition, the present disclosure describes a method of combating, controlling, or inhibiting a pest comprising, applying a pesticidally effective amount of an Kappa Peptide Variant (KPV) as described herein; a combination comprising two or more KPVs as described herein; a composition comprising a KPV and an agriculturally acceptable excipient, as described herein; and/or a composition comprising a combination of two or more KPVs and an agriculturally acceptable excipient, as described herein; to: the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof.

### BRIEF DESCRIPTION OF THE DRAWINGS

FIG. 1 shows a sequence logo demonstrating amino acid sequence conservation in all known Kappa-like sequences.

FIG. 2 depicts a box and whiskers graph showing the results for the deep-well expression comparison of Kappa Peptide Variants (KPVs) KPV2, KPV3, and KPV4, relative to WT Kappa.

FIG. 3 depicts a graph showing the impact each mutation has on the expression of a given KPV relative to WT Kappa. The X-axis (top) shows the position of each amino acid; the X-axis (bottom) shows the WT kappa sequence. The Y-axis shows possible amino acid substitutions. The intersection of the X- and Y-axis shows the level of expression, as indicated by a number that represents the fold increase relative to WT Kappa.

FIG. 4 depicts a graph showing the impact each mutation has on the activity of a given KPV relative to WT Kappa. The X-axis (top) shows the position of each amino acid; the X-axis (bottom) shows the WT kappa sequence. The Y-axis shows possible amino acid substitutions. The intersection of the X- and Y-axis shows the level of activity, as indicated by number that represent the fold increase relative to WT Kappa. Here, activity was assessed based on the % KD of flies dosed with 10 ng/μL of a given KPV, relative to the % KD of flies dosed with 10 ng/μL of WT Kappa. Here, the number in each box indicates the level of activity relative to WT Kappa.

FIG. 5 depicts a graph showing the site-specific impact each mutation has on the expression and activity of a given KPV relative to WT Kappa. The X-axis (top) shows the position of each amino acid; the X-axis (bottom) shows the WT kappa sequence. The Y-axis shows possible amino acid substitutions. The intersection of the X- and Y-axis shows the effect each site-specific mutation has on activity and expression relative to WT Kappa. Here, activity was assessed based on the % KD of flies dosed with 10 ng/μL of a given KPV, relative to the % KD of flies dosed with 10 ng/μL of WT Kappa. Here, the number in each box indicates the level of activity relative to WT Kappa.

FIG. 6 shows a graph illustrating the effect multiple mutations have on expression, as compared to a single point mutation. Each axis of the graph shows the effect a single mutation has on the expression of a KPV relative to WT Kappa. The points along the graph where the X- and Y-axis intersect, shows the expression of a KPV having both of the mutations (i.e., the mutation corresponding to the X—and the Y-axis), relative to WT Kappa. As shown here, the influence of each individual mutation is not always predictive of expression for a KPV with both of those mutations. For example, each of the single point mutations, C3Y and Y31N, have result in no expression for a KPV having each of those individual mutations; however, when C3Y and Y31N are both present in a KPV, it results in an improvement in expression.

FIG. 7 shows a graph depicting the yield-per-gene-copy number for WT Kappa, KPV2, KPV3, and KPV4.

FIG. 8 shows the results of the pre-incubation peak (blue) and the peaks post-incubation at 54° C. and pH 3.5, for 14 days (line with peaks labeled “4-1.431” and “5—Peak-1.466”) in WT Kappa. As shown here, a new peak was observed that was 225 Da less which corresponds to the removal of the C-terminus residues EP.

FIG. 9 shows the results of KPV2 after incubation in water and acidic conditions (pH 3.0) at 54° C. for 7 days. Here, the pre-incubation peak (outside line indicated with “7—Peak-1.901”) and post-incubation peak (inside line) of KPV2 incubated at 54° C. and pH 3.0 for 7 days reveals that no new peaks formed.

FIG. 10 shows the results of KPV2 after incubation in Tris buffer (pH 7.2) at 54° C. for 7 days. Here, the pre-incubation peak at day 0 (line labeled T0), and post-incubation peak at day 7 (line labeled T7) are shown. Two new peaks appear indicating less stability in this condition.

FIG. 11 shows the results of KVP2 fermentation beer after incubation at 55° C. for 6 days. Here, the pre-incubation peak (line labeled “9—Peak-1.873”; T0) and post-incubation peak (T6) are shown. No new peaks formed.

FIG. 12 depicts the results of a housefly injection assay where activity of WT Kappa and KVP2 were evaluated. Here, WT Kappa or KVP2 was injected intrathoracically into houseflies, and the dose of a given peptide required for LD50 was then determined at 18-hours post-injection.

FIG. 13 depicts the results of a lepidopteran diet assay where activity of WT Kappa and concentrated KVP2 fermentation beer were evaluated. Here, WT Kappa or concentrated KVP2 fermentation beer was provided to neonate corn earworms (CEW) in their diet, and LD50 was assessed after 4 days.

### DETAILED DESCRIPTION

### Definitions

“5′-end” and “3′-end” refers to the directionality, i.e., the end-to-end orientation of a nucleotide polymer (e.g., DNA). The 5′-end of a polynucleotide is the end of the polynucleotide that has the fifth carbon.

“Affect” refers to how a something influences another thing, e.g., how a peptide, polypeptide, protein, drug, or chemical influences a pest, e.g., a deleterious insect pest.

“Agent” refers to one or more chemical substances, molecules, nucleotides, polynucleotides, peptides, polypeptides, proteins, poisons, insecticides, pesticides, organic compounds, inorganic compounds, prokaryote organisms and agents produced therefrom, or eukaryote organisms and agents produced therefrom.

“Agriculturally-acceptable carrier” covers all adjuvants, inert components, dispersants, surfactants, tackifiers, binders, etc. that are ordinarily used in pesticide formulation technology; these are well known to those skilled in pesticide formulation.

“Agriculturally acceptable salt” is synonymous with pharmaceutically acceptable salt, and as used herein refers to a compound that is modified by making acid or base salts thereof.

“Alignment” refers to a method of comparing two or more sequences (e.g., nucleotide, polynucleotide, amino acid, peptide, polypeptide or protein sequences) for the purpose of determining their relationship to each other. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5: 113, 2004); Mafft, Kalign, ProbCons, and T-Coffee (see Notredame et al., T-Coffee: A novel method for multiple sequence alignments, Journal of Molecular Biology 302: 205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In some embodiments, an alignment will introduce “phase shifts” and/or “gaps” into one or both of the sequences being compared in order to maximize the similarity between the two sequences, and scoring refers to the process of quantitatively expressing the relatedness of the aligned sequences.

“Alpha-MF signal” or “αMF secretion signal” refers to a protein that directs nascent recombinant polypeptides to the secretory pathway.

“Applying” or “application” or “apply” or “administering” or “administration” or “administer” means to dispense and/or otherwise provide, and refers to any method of application or route of administration. For example, applying can refer to, e.g., application of a of an engineered, non-naturally occurring pesticidal KPV, or an agriculturally acceptable salt thereof, or an pesticidal composition comprising an engineered, non-naturally occurring pesticidal KPV, or an agriculturally acceptable salt thereof, and one or more excipients, e.g., a sprayable composition, a foam; a burning formulation; a fabric treatment; a surface-treatment; a dispersant; or a microencapsulation. By “co-application” or “co-administer” it is meant that a combination or composition described herein is applied or administered at the same time, just prior to, or just after the application of: an engineered, non-naturally occurring pesticidal KPV, or an agriculturally acceptable salt thereof comprising an amino acid sequence set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; and optionally, one or more excipients. By “co-application” or “co-administer” it is meant that two or more components are applied or administered at the same time; or a one or more components are applied or administered just prior to, or just after the application the other one or more components. For example, in some embodiments, a first KPV and a second KPV, wherein the first and second KPV can be the same or different, can be applied or administered separately, sequentially, simultaneously, concurrently or chronologically staggered.

“Ameliorate” or “amelioration” includes the arrest, prevention, decrease, or improvement in one or more the symptoms, signs, and features of the disease being treated, both temporary and long-term.

“Binary vector” or “binary expression vector” means an expression vector which can replicate itself in both E. coli strains and Agrobacterium strains. Also, the vector contains a region of DNA (often referred to as t-DNA) bracketed by left and right border sequences that is recognized by virulence genes to be copied and delivered into a plant cell by Agrobacterium.

“bp” or “base pair” refers to a molecule comprising two chemical bases bonded to one another forming a. For example, a DNA molecule consists of two winding strands, wherein each strand has a backbone made of an alternating deoxyribose and phosphate groups. Attached to each deoxyribose is one of four bases, i.e., adenine (A), cytosine (C), guanine (G), or thymine (T), wherein adenine forms a base pair with thymine, and cytosine forms a base pair with guanine.

“C-terminus” or “C-terminal” refers to the free carboxyl group (i.e., —COOH) that is positioned on the terminal end of a polypeptide.

“cDNA” or “copy DNA” or “complementary DNA” refers to a molecule that is complementary to a molecule of RNA. In some embodiments, cDNA may be either single-stranded or double-stranded. In some embodiments, cDNA can be a double-stranded DNA synthesized from a single stranded RNA template in a reaction catalyzed by a reverse transcriptase. In yet other embodiments, “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein. In some embodiments, “cDNA” refers to a DNA that is complementary to and derived from an mRNA template.

“CEW” refers to Corn earworm.

“Cleavable Linker” see Linker.

“Cloning” refers to the process and/or methods concerning the insertion of a DNA segment (e.g., usually a gene of interest, for example a KPV) from one source and recombining it with a DNA segment from another source (e.g., usually a vector, for example, a plasmid) and directing the recombined DNA, or “recombinant DNA” to replicate, usually by transforming the recombined DNA into a bacteria or yeast host.

“Coding sequence” or “CDS” refers to a polynucleotide or nucleic acid sequence that can be transcribed (e.g., in the case of DNA) or translated (e.g., in the case of mRNA) into a peptide, polypeptide, or protein, when placed under the control of appropriate regulatory sequences and in the presence of the necessary transcriptional and/or translational molecular factors. The boundaries of the coding sequence are determined by a translation start codon at the 5′ (amino) terminus and a translation stop codon at the 3′ (carboxy) terminus. A transcription termination sequence will usually be located 3′ to the coding sequence. In some embodiments, a coding sequence may be flanked on the 5′ and/or 3′ ends by untranslated regions. Generally, those having ordinary skill in the art distinguish the terms “coding sequence from the terms “open reading frame” and “ORF,” based upon the fact that the broadest definition of “open reading frame” simply contemplates a series of codons that does not contain a stop codon. Accordingly, while an ORF may contain introns, the coding sequence is distinguished by referring to those nucleotides (e.g., concatenated exons) that can be divided into codons that are actually translated into amino acids by the ribosomal translation machinery (i.e., a coding sequence does not contain introns); however, as used herein, the terms “coding sequence”; “CDS”; “open reading frame”; and “ORF,’ are used interchangeably, and all refer to a polynucleotide or nucleic acid sequence that can be transcribed (e.g., in the case of DNA) or translated (e.g., in the case of mRNA) into a peptide, polypeptide, or protein, when placed under the control of appropriate regulatory sequences and in the presence of the necessary transcriptional and/or translational molecular factors.

“Codon optimization” refers to the production of a gene in which one or more endogenous, native, and/or wild-type codons are replaced with codons that ultimately still code for the same amino acid, but that are of preference in the corresponding host.

“Combination” refers to the result of combining two or more separate components. Thus, as used herein, a “combination” refers to an association of two or more separate components, e.g., a KPV and an additional component. Accordingly, in some embodiments, a combination can refer to the association of a first KPV, and one or more additional KPVs; wherein the first KPV and one or more additional KPVs are the same or different. In some embodiments, the combination can be, e.g., a mixture, or as part of a composition further comprising one or more excipients. In some embodiments, a combination can refer to the simultaneous, separate, or sequential application of two or more separate components (e.g., a first KPV, and one or more additional KPVs; wherein the first KPV and one or more additional KPVs are the same or different). For example, in some embodiments, a “combination” refers to the result of a simultaneous application of both a first KPV, and one or more additional KPVs; wherein the first KPV and one or more additional KPVs are the same or different. In another embodiment, a “combination” refers to the result of a separate application of a first KPV, and one or more additional KPVs; wherein the first KPV and one or more additional KPVs are the same or different. In a further embodiments, a “combination” refers to the result of a sequential application of two or more separate components, e.g., a first application of a first KPV, followed by a second application of one or more additional KPVs (wherein the first KPV and one or more additional KPVs are the same or different), or vice versa. Where the application is sequential or separate, the delay in applying the second component should not be such as to lose the beneficial effect of the combination.

“Complementary” refers to the topological compatibility or matching together of interacting surfaces of two polynucleotides as understood by those of skill in the art. Thus, two sequences are “complementary” to one another if they are capable of hybridizing to one another to form a stable anti-parallel, double-stranded nucleic acid structure. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions. Thus, the polynucleotide whose sequence 5′-TATAC-3′ is complementary to a polynucleotide whose sequence is 5′-GTATA-3′.

“Conditioned medium” means the cell culture medium which has been used by cells and is enriched with cell derived materials but does not contain cells.

“Conservative amino acid substitutions” refers to amino acid substitutions in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); polar, negatively charged residues and their amides (e.g., aspartic acid, asparagine, glutamic, acid, glutamine; uncharged polar side chains (e.g., glycine, asparagine, glutamine, serine, threonine, tyrosine, cysteine); small aliphatic, nonpolar or slightly polar residues (e.g., Alanine, serine, threonine, proline, glycine); nonpolar side chains (e.g., alanine, valine, leucine, isoleucine, proline, phenylalanine, methionine, tryptophan); large aliphatic, nonpolar residues (e.g., methionine, leucine, isoleucine, valine, cystine); beta-branched side chains (e.g., threonine, valine, isoleucine); aromatic side chains (e.g., tyrosine, phenylalanine, tryptophan, histidine); large aromatic side chains (e.g., tyrosine, phenylalanine, tryptophan).

Amino acid substitutions may be made in non-conserved regions that retain function. In general, such substitutions would not be made for conserved amino acid residues or for amino acid residues residing within a conserved motif, where such residues are essential for protein activity. Examples of residues that are conserved and that may be essential for protein activity include, for example, residues that are identical between all proteins contained in an alignment of similar or related toxins to the sequences of the embodiments (e.g., residues that are identical in an alignment of homologs). Examples of residues that are conserved but that may allow conservative amino acid substitutions and still retain activity include, for example, residues that have only conservative substitutions between all proteins contained in an alignment of similar or related toxins to the sequences of the embodiments (e.g., residues that have only conservative substitutions between all proteins contained in the alignment of the homologs). However, one of skill in the art would understand that functional variants may have minor conserved or non-conserved alterations in the conserved residues. Guidance as to appropriate amino acid substitutions that do not affect biological activity of the protein of interest may be found in the model of Dayhoff, et al., (1978) Atlas of Protein Sequence and Structure (Natl. Biomed. Res. Found., Washington, D.C.), the disclosure of which is incorporated herein by reference. Additional amino acid substitution models are likewise known in the art, e.g., JTT/WAG).

In making such changes, the hydropathic index of amino acids may be considered. The importance of the hydropathic amino acid index in conferring interactive biologic function on a protein is generally understood in the art (Kyte and Doolittle, (1982) J Mol Biol. 157(1):105-32). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It is known in the art that certain amino acids may be substituted by other amino acids having a similar hydropathic index or score and still result in a protein with similar biological activity, i.e., still obtain a biological functionally equivalent protein. Each amino acid has been assigned a hydropathic index on the basis of its hydrophobicity and charge characteristics (Kyte and Doolittle, ibid). These are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9) and arginine (−4.5). In making such changes, the substitution of amino acids whose hydropathic indices are within +2 is preferred, those which are within +1 are particularly preferred, and those within +0.5 are even more particularly preferred.

It is also understood in the art that the substitution of like amino acids can be made effectively on the basis of hydrophilicity. U.S. Pat. No. 4,554,101, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0.+0.1); glutamate (+3.0.+0.1); serine (+0.3); asparagine (+0.2); glutamine (+0.2); glycine (0); threonine (−0.4); proline (−0.5.+0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

“Copy number” refers to the number of identical copies of a vector, an expression cassette, an amplification unit, a gene or indeed any defined nucleotide sequence, that are present in a host cell at any time. For example, in some embodiments, a gene or another defined chromosomal nucleotide sequence may be present in one, two, or more copies on the chromosome. An autonomously replicating vector may be present in one, or several hundred copies per host cell.

“Culture” or “cell culture” refers to the maintenance of cells in an artificial, in vitro environment.

“Culturing” refers to the propagation of organisms on or in various kinds of media. For example, the term “culturing” can mean growing a population of cells under suitable conditions in a liquid or solid medium. In some embodiments, culturing refers to fermentative recombinant production of a heterologous polypeptide of interest and/or other desired end products (typically in a vessel or reactor).

“Cystine” refers to an oxidized cysteine-dimer. Cystines are sulfur-containing amino acids obtained via the oxidation of two cysteine molecules, and are linked with a disulfide bond.

“Defined medium” means a medium that is composed of known chemical components but does not contain crude proteinaceous extracts or by-products such as yeast extract or peptone.

“Degeneracy” or “codon degeneracy” refers to the phenomenon that one amino acid can be encoded by different nucleotide codons. Thus, the nucleic acid sequence of a nucleic acid molecule that encodes a protein or polypeptide can vary due to degeneracies. As a result of the degeneracy of the genetic code, many nucleic acid sequences can encode a given polypeptide with a particular activity; such functionally equivalent variants are contemplated herein.

“Derived” or “derived from” refers to obtaining a peptide, polypeptide, protein or polynucleotide from a known and/or originating peptide, polypeptide, protein or polynucleotide. Thus, as used herein, the term “derived from” encompasses, without limitation: a protein or polynucleotide that is isolated or obtained directly from an originating source (e.g. an organism, such as a one or more species belonging to the Atracidae family); a synthetic or recombinantly generated protein or polynucleotide that is identical, substantially related to, or modified from, a protein or polynucleotide from an known/originating source; or protein or polynucleotide that is made from a protein or polynucleotide of an known/originating source or a fragment thereof. The term “substantially related”, as used herein, means that the protein may have been modified by chemical, physical or other means (e.g. sequence modification).

Accordingly, “derived” can refer to either directly or indirectly obtaining a protein or polynucleotide from a known and/or originating protein or polynucleotide. For example, in some embodiments, “derived” can refer to obtaining a protein or polynucleotide from a known and/or originating protein or polynucleotide by looking at the sequence of a known/originating protein or polynucleotide and preparing a protein or polynucleotide having a sequence similar, at least in part, to the sequence of the known and/or originating protein or polynucleotide. In yet other embodiments, “derived” can refer to obtaining a protein or polynucleotide from a known and/or originating protein or polynucleotide by isolating a protein or polynucleotide from an organism that is related to a known protein or polynucleotide. Other methods of “deriving” a protein or polynucleotide from a known protein or polynucleotide are known to one of skill in the art.

In some embodiments, “derived” in the context of a protein (e.g., “a protein derived from an organism”) describes a condition wherein said protein was originally identified in an organism, and has been reproduced therefrom via isolation from the organism, or through synthetic or recombinant means.

“Disulfide bond” or “disulfide bridges” refers to a covalent bond between two cysteine amino acids derived by the coupling of two thiol groups on their side chains. In some embodiments, a disulfide bond occurs via the oxidative folding of two different thiol groups (—SH) present in a polypeptide. In some embodiments, a polypeptide can comprise at least six different thiol groups (i.e., six cysteine residues each containing a thiol group); thus, in some embodiments, a polypeptide can form zero, one, two, three, or more intramolecular disulfide bonds.

“Double expression cassette” refers to two KPV expression cassettes contained on the same vector.

“Double transgene peptide expression vector” or “double transgene expression vector” means a yeast expression vector that contains two copies of the KPV expression cassette.

“DNA” refers to deoxyribonucleic acid, comprising a polymer of one or more deoxyribonucleotides or nucleotides (i.e., adenine [A], guanine [G], thymine [T], or cytosine [C]), which can be arranged in single-stranded or double-stranded form. For example, one or more nucleotides creates a polynucleotide.

“dNTPs” refers to the nucleoside triphosphates that compose DNA and RNA.

“Endogenous” refers to a polynucleotide, peptide, polypeptide, protein, or process that naturally occurs and/or exists in an organism, e.g., a molecule or activity that is already present in the host cell before a particular genetic manipulation.

“Enhancer element” refers to a DNA sequence operably linked to a promoter, which can exert increased transcription activity on the promoter relative to the transcription activity that results from the promoter in the absence of the enhancer element.

“ER” or “Endoplasmic reticulum” is a subcellular organelle common to all eukaryotes where some post translation modification processes occur.

“ERSP” or “Endoplasmic reticulum signal peptide” is an N-terminus sequence of amino acids that—during protein translation of the mRNA molecule encoding a KPV—is recognized and bound by a host cell signal-recognition particle, which moves the protein translation ribosome/mRNA complex to the ER in the cytoplasm. The result is the protein translation is paused until it docks with the ER where it continues and the resulting protein is injected into the ER.

“ersp” refers to a polynucleotide encoding the peptide, ERSP.

“ER trafficking” means transportation of a cell expressed protein into ER for post-translational modification, sorting and transportation.

“Excipient” refers to any agriculturally acceptable additive, carrier, surfactant, emulsifier, thickener, preservative, solvent, disintegrant, glidant, lubricant, diluent, filler, bulking agent, binder, emollient, stiffening agent, chelating agent, stabilizer, solubilizing agents, dispersing agent, suspending agent, antioxidant, antiseptic, wetting agent, humectant, fragrant, suspending agents, pigments, colorants, isotonic agents, viscosity enhancing agents, mucoadhesive agents, and/or any combination thereof, that can be added to an agricultural composition, preparation, and/or formulation, which may be useful in achieving a desired modification to the characteristics of the agricultural composition, preparation, and/or formulation. Such modifications include, but are not limited to, physical stability, chemical stability, pesticidal efficacy, and/or any combination thereof.

“Expression cassette” refers to (1) a DNA sequence of interest, e.g., a polynucleotide operable to encode a KPV; and one or more of the following: (2) promoters, terminators, and/or enhancer elements; (3) an appropriate mRNA stabilizing polyadenylation signal; (4) an internal ribosome entry site (IRES); (5) introns; and/or (6) post-transcriptional regulatory elements. The combination (1) with at least one of (2)-(6) is called an “expression cassette.” In some embodiments, there can be numerous expression cassettes cloned into a vector. For example, in some embodiments, there can be a first expression cassette comprising a polynucleotide operable to encode a KPV. In alternative embodiments, there are two expression cassettes, each comprising a polynucleotide operable to encode a KPV (i.e., a double expression cassette). In other embodiments, there are three expression cassettes operable to encode a KPV (i.e., a triple expression cassette). In some embodiments, a double expression cassette can be generated by subcloning a second expression cassette into a vector containing a first expression cassette. In some embodiments, a triple expression cassette can be generated by subcloning a third expression cassette into a vector containing a first and a second expression cassette. Methods concerning expression cassettes and cloning techniques are well-known in the art and described herein. See also KPV expression cassette.

“FECT” means a transient plant expression system using Foxtail mosaic virus with elimination of coating protein gene and triple gene block.

“Fermentation beer” refers to spent fermentation medium, i.e., fermentation medium supernatant after removal of organisms, that has been inoculated with and consumed by a transformed host cell (e.g., a yeast cell operable to express a KPV of the present disclosure). In some embodiments, fermentation beer refers to the solution that is recovered following the fermentation of the transformed host cell. The term “fermentation” refers broadly to the enzymatic and anaerobic or aerobic breakdown of organic substances (e.g., a carbon substrate) nutrient substances by microorganisms under controlled conditions (e.g., temperature, oxygen, pH, nutrients, and the like) to produce fermentation products (e.g., one or more peptides of the present disclosure). While fermentation typically describes processes that occur under anaerobic conditions, as used herein it is not intended that the term be solely limited to strict anaerobic conditions, as the term “fermentation” used herein may also occur processes that occur in the presence of oxygen.

“GFP” means green fluorescent protein from the jellyfish, Aequorea victoria.

“Growth medium” refers to a nutrient medium used for growing cells in vitro.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. Thus, in some embodiments, the term “homologous” refers to the sequence similarity between two polypeptide molecules, or between two nucleic acid molecules. When a position in both of the two compared sequences is occupied by the same base or amino acid monomeric subunit, e.g., if a position in each of two DNA molecules is occupied by adenine, then the molecules are homologous at that position. The homology between two sequences is a function of the number of matching or homologous positions shared by the two sequences. For example, if 6 of 10 of the positions in two sequences are matched or homologous then the two sequences are 60% homologous. By way of example, the DNA sequences ATTGCC and TATGGC share 50% homology.

There may be partial homology, or complete homology and thus identical. “Sequence identity” refers to a measure of relatedness between two or more nucleic acid sequences or two or more polypeptide sequences, and is given as a percentage with reference to the total comparison length. The identity calculation takes into account those nucleotide residues or amino acid residues that are identical and in the same relative positions in their respective larger sequences.

“Homologous recombination” refers to the event of substitution of a segment of DNA by another one that possesses identical regions (homologous) or nearly so. For example, in some embodiments, “homologous recombination” refers to a type of genetic recombination in which nucleotide sequences are exchanged between two similar or identical molecules of DNA. Briefly, homologous recombination is most widely used by cells to accurately repair harmful breaks that occur on both strands of DNA, known as double-strand breaks. Although homologous recombination varies widely among different organisms and cell types, most forms involve the same basic steps: after a double-strand break occurs, sections of DNA around the 5′ ends of the break are cut away in a process called resection. In the strand invasion step that follows, an overhanging 3′ end of the broken DNA molecule then “invades” a similar or identical DNA molecule that is not broken. After strand invasion, the further sequence of events may follow either of two main pathways, i.e., the double-strand break repair pathway, or the synthesis-dependent strand annealing pathway. Homologous recombination is conserved across all three domains of life as well as viruses, suggesting that it is a nearly universal biological mechanism. For example, in some embodiments, homologous recombination can occur using a site-specific integration (SSI) sequence, whereby there is a strand exchange crossover event between nucleic acid sequences substantially similar in nucleotide composition. These crossover events can take place between sequences contained in the targeting construct of the invention (i.e., the SSI sequence) and endogenous genomic nucleic acid sequences (e.g., the polynucleotide encoding the peptide subunit). In addition, in some embodiments, it is possible that more than one site-specific homologous recombination event can occur, which would result in a replacement event in which nucleic acid sequences contained within the targeting construct have replaced specific sequences present within the endogenous genomic sequences.

“Hybridize” refers to the annealing of one single-stranded polynucleotide to another polynucleotide based on the well-understood principle of sequence complementarity. In some embodiments, the other polynucleotide is a single-stranded polynucleotide. The propensity for hybridization between polynucleotides depends on the temperature and ionic strength of their milieu, the length of the polynucleotides, and the degree of complementarity. The effect of these parameters on hybridization are well known in the art.

“Hybridization” refers to any process by which a strand of polynucleotide binds with a complementary strand through base pairing. Two single-stranded polynucleotides “hybridize” when they form a double-stranded duplex. Thus, as used herein, the term “hybridize” refers to the annealing of one single-stranded polynucleotide to another polynucleotide based on the well-understood principle of sequence complementarity. In some embodiments, the other polynucleotide is a single-stranded polynucleotide. The propensity for hybridization between polynucleotides depends on the temperature and ionic strength of their milieu, the length of the polynucleotides, and the degree of complementarity. The effect of these parameters on hybridization are well known in the art. When two single-stranded polynucleotides hybridize and form a double-stranded duplex, the region of double-strandedness can include the full-length of one or both of the single-stranded polynucleotides, or all of one single stranded polynucleotide and a subsequence of the other single stranded polynucleotide, or the region of double-strandedness can include a subsequence of each polynucleotide. Hybridization also includes the formation of duplexes which contain certain mismatches, provided that the two strands are still forming a double stranded helix. See “Stringent hybridization conditions” below.

“IC₅₀” or “IC50” refers to half-maximal inhibitory concentration, which is a measurement of how much of an agent is needed to inhibit a biological process by half, thus providing a measure of potency of said agent.

“Identity” refers to a relationship between two or more polypeptide sequences or two or more polynucleotide sequences, as determined by comparing said sequences. The term “identity” also means the degree of sequence relatedness between polypeptide or polynucleotide sequences, as the case may be, as determined by the match between strings of such sequences. “Identity” and “similarity” can be readily calculated by any one of the myriad methods known to those having ordinary skill in the art, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994, Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988), the disclosures of which are incorporated herein by reference in their entireties. Furthermore, methods to determine identity and similarity are codified in publicly available computer programs. For example in some embodiments, methods to determine identity and similarity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990), the disclosures of which are incorporated herein by reference in their entireties.

“in vivo” refers to in the living body of a plant or animal (e.g., an animal, plant or a cell) and to processes or reactions that occur within the living body of a plant or animal.

“Inactive” refers to a condition wherein something is not in a state of use, e.g., lying dormant and/or not working. For example, when used in the context of a gene or when referring to a gene, the term inactive means said gene is no longer actively synthesizing a gene product, having said gene product translated into a protein, or otherwise having the gene perform its normal function. For example, in some embodiments, the term inactive can refer the failure of a gene to transcribe RNA, a failure of RNA processing (e.g., pre-mRNA processing; RNA splicing; or other post-transcriptional modifications); interference with non-coding RNA maturation; interference with RNA export (e.g., from the nucleus to the cytoplasm); interference with translation; protein folding; translocation; protein transport; and/or inhibition and/or interference with any of the molecules polynucleotides, peptides, polypeptides, proteins, transcription factors, regulators, inhibitors, or other factors that take part in any of the aforementioned processes.

“Inhibiting” or “inhibit” or “combating” or “combat” or “controlling” or “control,” or any variation of these terms, refers to making something (e.g., the number of pests, the functions and/or activities of the pest, and/or the deleterious effect of the pest on a plant or animal susceptible to attack thereof) less in size, amount, intensity, or degree. For example, in some embodiments, the application of a pesticidally effective amount of a KPV or agriculturally acceptable salt thereof, or an agricultural composition comprising a KPV or agriculturally acceptable salt thereof, to (i) the pest, a locus of the pest, a food supply of the pest, a habitat of the pest, or a breeding ground of the pest; (ii) a plant, a seed, a plant part, a locus of a plant, or an environment of a plant that is susceptible to an attack by the pest; (iii) an animal, a locus of an animal, or an environment of an animal susceptible to an attack by the pest; or (iv) a combination thereof, results in the following effect: a decrease in the number of pests, or inhibition of the pest's activities (e.g., the pest dies stops or slows its movement; stops or slows its feeding; stops or slows its growth; becomes confused, e.g., with regard to navigation, locating food, sleeping behaviors, and/or mating; fails to pupate if applicable; interferes with reproduction of the pest; and/or precludes the pest from producing offspring and/or precludes the insect from producing fertile offspring) relative to the number of pests or activities thereof that had not been exposed to a pesticidally effective amount of a KPV or agriculturally acceptable salt thereof, or an agricultural composition comprising a KPV or agriculturally acceptable salt thereof.

In some embodiments, combating, controlling, or inhibiting a pest, includes any measurable decrease or complete inhibition to achieve a desired result. For example, there may be a decrease of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, in the number of pests or the activities thereof treated with peptides and/or compositions of the present disclosure, compared to untreated pests. About as used herein means within +10%, preferably +5% of a given value.

Thus, in some embodiments, the terms “combating, controlling, or inhibiting a pest,” refers to a decrease in the number of pests, or an inhibition of the activities of the pests (e.g., movement; feeding; growth; level of awareness or alertness, e.g., with regard to navigation, locating food, sleeping behaviors, and/or mating; pupation if applicable; reproduction; ability to produce offspring and/or ability to produce fertile offspring) that have received a pesticidally effective amount of a KPV of the present disclosure, or an agricultural composition thereof, that is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 2.25%, at least about 2.5%, at least about 2.75%, at least about 3%, at least about 3.25%, at least about 3.5%, at least about 3.75%, at least about 4%, at least about 4.25%, at least about 4.5%, at least about 4.75%, at least about 5%, at least about 5.25%, at least about 5.5%, at least about 5.75%, at least about 6%, at least about 6.25%, at least about 6.5%, at least about 6.75%, at least about 7%, at least about 7.25%, at least about 7.5%, at least about 7.75%, at least about 8%, at least about 8.25%, at least about 8.5%, at least about 8.75%, at least about 9%, at least about 9.25%, at least about 9.5%, at least about 9.75%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 80%, at least about 85%, at least about 90%, at least about 95%, at least about 100%, or a greater than a 100%, relative to the number of pests, or the inhibition of activities of the pests (e.g., movement; feeding; growth; level of awareness or alertness, e.g., with regard to navigation, locating food, sleeping behaviors, and/or mating; pupation if applicable; reproduction; ability to produce offspring and/or ability to produce fertile offspring) that have not received a pesticidally effective amount of a KPV of the present disclosure, or an agricultural composition thereof.

“Inoperable” refers to the condition of a thing not functioning, malfunctioning, or no longer able to function. For example, when used in the context of a gene or when referring to a gene, the term inoperable means said gene is no longer able to operate as it normally would, either permanently or transiently. For example, “inoperable,” in some embodiments, means that a gene is no longer able to synthesize a gene product, having said gene product translated into a protein, or is otherwise unable to gene perform its normal function. For example, in some embodiments, the term inoperable can refer the failure of a gene to transcribe RNA, a failure of RNA processing (e.g., pre-mRNA processing; RNA splicing; or other post-transcriptional modifications); interference with non-coding RNA maturation; interference with RNA export (e.g., from the nucleus to the cytoplasm); interference with translation; protein folding; translocation; protein transport; and/or inhibition and/or interference with any of the molecules polynucleotides, peptides, polypeptides, proteins, transcription factors, regulators, inhibitors, or other factors that take part in any of the aforementioned processes.

“Insects” includes all organisms in the class “Insecta.” The term “pre-adult” insects refers to any form of an organism prior to the adult stage, including, for example, eggs, larvae, and nymphs. As used herein, the term “insect refers to any arthropod and nematode, including acarids, and insects known to infest all crops, vegetables, and trees and includes insects that are considered pests in the fields of forestry, horticulture and agriculture. Examples of specific crops that might be protected with the methods disclosed herein are soybean, corn, cotton, alfalfa and the vegetable crops. A list of specific crops and insects is enclosed herein.

“Insect gut environment” or “gut environment” means the specific pH and proteinase conditions found within the fore, mid or hind gut of an insect or insect larva.

“Insect hemolymph environment” means the specific pH and proteinase conditions of found within an insect or insect larva.

“Intervening linker” refers to a short peptide sequence in the protein separating different parts of the protein, or a short DNA sequence that is placed in the reading frame in the ORF to separate the upstream and downstream DNA sequences. For example, in some embodiments, an intervening linker may be used allowing proteins to achieve their independent secondary and tertiary structure formation during translation. In some embodiments, the intervening linker can be either resistant or susceptible to cleavage in plant cellular environments, in the insect and/or lepidopteran gut environment, and in the insect hemolymph and lepidopteran hemolymph environment.

“Isolated” refers to separating a thing and/or a component from its natural environment, e.g., a toxin isolated from a given genus or species means that toxin is separated from its natural environment.

“kb” refers to kilobase, i.e., 1000 bases. As used herein, the term “kb” means a length of nucleic acid molecules. For example, 1 kb refers to a nucleic acid molecule that is 1000 nucleotides long. A length of double-stranded DNA that is 1 kb long, contains two thousand nucleotides (i.e., one thousand on each strand). Alternatively, a length of single-stranded RNA that is 1 kb long, contains one thousand nucleotides.

“KD₅₀” or “Knockdown dose 50” or “paralytic dose 50” or “PD₅₀” refers to the median dose required to cause paralysis or cessation of movement in 50% of a population, for example, and without limitation, a population of Musca domestica (common housefly), or a population of Aedes aegypti (mosquito).

“kDa” refers to kilodalton, a unit equaling 1,000 daltons; a “dalton” or “Da” is a unit of molecular weight (MW).

“Knock in” or “knock-in” or “knocks-in” or “knocking-in” refers to the replacement of an endogenous gene with an exogenous or heterologous gene, or part thereof. For example, in some embodiments, the term “knock-in” refers to the introduction of a nucleic acid sequence encoding a desired protein to a target gene locus by homologous recombination, thereby causing the expression of the desired protein. In some embodiments, a “knock-in” mutation can modify a gene sequence to create a loss-of-function or gain-of-function mutation. The term “knock-in” can refer to the procedure by which a exogenous or heterologous polynucleotide sequence or fragment thereof is introduced into the genome, (e.g., “they performed a knock-in” or “they knocked-in the heterologous gene”), or the resulting cell and/or organism (e.g., “the cell is a “knock-in” or “the animal is a “knock-in”).

“Knock out” or “knockout” or “knock-out” or “knocks-out” or “knocking-out” refers to a partial or complete suppression of the expression gene product (e.g., mRNA) of a protein encoded by an endogenous DNA sequence in a cell. In some embodiments, the “knock-out”can be effectuated by targeted deletion of a whole gene, or part of a gene encoding a peptide, polypeptide, or protein. As a result, the deletion may render a gene inactive, partially inactive, inoperable, partly inoperable, or otherwise reduce the expression of the gene or its products in any cell in the whole organism and/or cell in which it is normally expressed. The term “knock-out” can refer to the procedure by which an endogenous gene is made completely or partially inactive or inoperable (e.g., “they performed a knock-out” or “they knocked-out the endogenous gene”), or the resulting cell and/or organism (e.g., “the cell is a “knock-out” or “the animal is a “knock-out”).

“KPV” or “Kappa Peptide Variant” or “Kappa Variant Peptide” or “Kappa mutant” or “Kappa mutant peptide” or “engineered, non-naturally occurring pesticidal KPV” or “engineered KPV” (all used interchangeably) refer to peptides having one or more mutations relative to the WT Kappa peptide having an amino acid sequence:

(SEQ ID NO: 1)
AICTGADRPCAACCPCCPGTSCKAESNGVSYCRKDEP (NCBI
Accession No. P82228).

In some embodiments, a KPV can have an amino acid sequence that is at least 90%, 95%, 96%, 97%, 97%, 98%, 99%, or 100% identical to the amino acid sequence according to Formula (I):

Formula (I)
X₁-X₂-X₃-X₄-X₅-X₆-X₇-X₈-X₉-X₁₀-X₁₁-X₁₂-X₁₃-X₁₄-X₁₅-X₁₆-
X₁₇-C-X₁₈-P-X₁₉-X₂₀-X₂₁-X₂₂-X₂₃-X₂₄-X₂₅-X₂₆-X₂₇-X₂₈-G-X₂₉-
X₃₀-X₃₁-X₃₂-X₃₃-X₃₄-X₃₅-X₃₆-X₃₇

wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

“KPV expression cassette” refers to one or more regulatory elements such as promoters; enhancer elements; mRNA stabilizing polyadenylation signal; an internal ribosome entry site (IRES); introns; post-transcriptional regulatory elements; and a polynucleotide operable to encode a KPV, e.g., a KPV ORF. For example, one example of a KPV expression cassette is one or more segments of DNA that contains a polynucleotide segment operable to express a KPV, a ADH1 promoter, a LAC4 terminator, and an alpha-MF secretory signal. An KPV expression cassette contains all of the nucleic acids necessary to encode a KPV or a KPV-pesticidal protein.

“KPV ORF” refers to a polynucleotide operable to encode a KPV, or a KPV-pesticidal protein.

“KPV ORF diagram” refers to the composition of one or more KPV ORFs, as written out in diagram or equation form. For example, a “KPV ORF diagram” can be written out as using acronyms or short-hand references to the DNA segments contained within the expression ORF. Accordingly, in one example, a “KPV ORF diagram” may describe the polynucleotide segments encoding the ERSP, LINKER, STA, and KPV, by diagramming in equation form the DNA segments as “ersp” (i.e., the polynucleotide sequence that encodes the ERSP polypeptide); “linker” or “L” (i.e., the polynucleotide sequence that encodes the LINKER polypeptide); “sta” (i.e., the polynucleotide sequence that encodes the STA polypeptide), and “kpv” (i.e., the polynucleotide sequence encoding a KPV), respectively. An example of a KPV ORF diagram is “ersp-sta-(linkerᵢ-kpvⱼ)_{N},” or “ersp-(kpvⱼ-linkerᵢ)_{N}-sta” and/or any combination of the DNA segments thereof.

“KPV-pesticidal protein” or “KPV-pesticidal polypeptide” or “pesticidal protein” or “pesticidal polypeptide” refers to any protein, peptide, polypeptide, amino acid sequence, configuration, or arrangement, comprising: (1) at least one KPV, or two or more KPVs; and (2) additional peptides, polypeptides, or proteins that are not a KPV. For example, in some embodiments, a KPV-pesticidal protein can be a fusion protein comprising at least one KPV, and at least one additional peptide or protein that is not a KPV, wherein at least one KPV is linked directly or indirectly to a non-KPV protein.

In some embodiments, a KPV-pesticidal protein can be a fusion protein comprising at least one KPV, and at least one additional peptide, polypeptide, or protein that is not a KPV, wherein at least one KPV is linked directly or indirectly to a non-KPV protein; wherein the at least one additional peptides, polypeptides, or proteins have the ability to increase the mortality and/or inhibit the growth of insects when the insects are exposed to a KPV-pesticidal protein, relative to a KPV alone; increase the expression of said KPV-pesticidal protein, e.g., in a host cell or an expression system; and/or affect the post-translational processing of the KPV-pesticidal protein.

In some embodiments, a KPV-pesticidal protein can be a polymer comprising two or more KPVs. In some embodiments, a KPV-pesticidal protein can be a polymer comprising two or more KPVs, wherein the KPVs are operably linked via a linker peptide, e.g., a cleavable and/or non-cleavable linker. In some embodiments, a KPV-pesticidal protein can refer to a one or more KPVs operably linked with one or more proteins such as a stabilizing domain (STA); an endoplasmic reticulum signaling protein (ERSP); an insect cleavable or insect non-cleavable linker (L); and/or any other combination thereof. In some embodiments, a KPV-pesticidal protein can be a non-naturally occurring protein comprising (1) a KPV; and (2) additional peptides, polypeptides, or proteins, e.g., an ERSP; a linker; a STA; a UBI; or a histidine tag or similar marker.

“KPV construct” refers to the three-dimensional arrangement/orientation of peptides, polypeptides, and/or motifs of operably linked polypeptide segments (e.g., a KPV-pesticidal protein). For example, a KPV ORF can include one or more of the following components or motifs: a KPV; an endoplasmic reticulum signal peptide (ERSP); a linker peptide (L); a translational stabilizing protein (STA); or any combination thereof. And, as used herein, the term “KPV construct” is used to describe the designation and/or orientation of the structural motif. In other words, the KPV construct describes the arrangement and orientation of the components or motifs contained within a given KPV ORF. For example, in some embodiments, a KPV construct describes, without limitation, the orientation of one of the following KPV-pesticidal proteins: ERSP-KPV; ERSP-(KPV)_{N}; ERSP-KPV-L; ERSP-(KPV)_{N}-L; ERSP-(KPV-L)_{N}; ERSP-L-KPV; ERSP-L-(KPV)_{N}; ERSP-(L-KPV)_{N}; ERSP-STA-KPV; ERSP-STA-(KPV)_{N}; ERSP-KPV-STA; ERSP-(KPV)_{N}-STA; ERSP-(STA-KPV)_{N}; ERSP-(KPV-STA)_{N}; ERSP-L-KPV-STA; ERSP-L-STA-KPV; ERSP-L-(KPV-STA)_{N}; ERSP-L-(STA-KPV)_{N}; ERSP-L-(KPV)_{N}-STA; ERSP-(L-KPV)_{N}-STA; ERSP-(L-STA-KPV)_{N}; ERSP-(L-KPV-STA)_{N}; ERSP-(L-STA)_{N}-KPV; ERSP-(L-KPV)_{N}-STA; ERSP-STA-L-KPV; ERSP-STA-KPV-L; ERSP-STA-L-(KPV)_{N}; ERSP-(STA-L)_{N}-KPV; ERSP-STA-(L-KPV)_{N}; ERSP-(STA-L-KPV)_{N}; ERSP-STA-(KPV)_{N}-L; ERSP-STA-(KPV-L)_{N}; ERSP-(STA-KPV)_{N}-L; ERSP-(STA-KPV-L)_{N}; ERSP-KPV-L-STA; ERSP-KPV-STA-L; ERSP-(KPV)_{N}-STA-L ERSP-(KPV-L)_{N}-STA; ERSP-(KPV-STA)_{N}-L; ERSP-(KPV-L-STA)_{N}; or ERSP-(KPV-STA-L)_{N}; wherein N is an integer ranging from 1 to 200. See also “Structural motif.”

“KPV polynucleotide” refers to the polynucleotide sequence that encodes any KPV. The term “KPV polynucleotide” when used to describe the KPV polynucleotide sequence, e.g., such as one contained in a KPV open reading frame (ORF), its inclusion in a vector, and/or when describing the polynucleotides encoding an pesticidal protein, is written in lowercase and italicized, e.g., “kpv” and/or “kpv”.

“l” or “linker” refers to a nucleotide encoding intervening linker peptide.

“L” or “LINKER” in the proper context refers to an intervening linker peptide, which links a translational stabilizing protein (STA) with an additional polypeptide, e.g., a KPV, and/or multiple KPV. When referring to amino acids, “L” can also mean leucine.

“LAC4 terminator” or “Lac4 terminator” refers to a DNA segment comprised of the transcriptional terminator sequence derived from the K. lactis β-galactosidase gene.

“LD₂₀” refers to a dose required to kill 20% of a population.

“LD₅₀” refers to lethal dose 50 which means the dose required to kill 50% of a population.

“Lepidopteran gut environment” means the specific pH and proteinase conditions of found within the fore, mid or hind gut of a lepidopteran insect or larva.

“Lepidopteran hemolymph environment” means the specific pH and proteinase conditions of found within lepidopteran insect or larva.

“Linker” or “LINKER” or “peptide linker” or “L” or “intervening linker” refers to a short peptide sequence operable to link two peptides together. Linker can also refer to a short DNA sequence that is placed in the reading frame of an ORF to separate an upstream and downstream DNA sequences. In some embodiments, a linker can be cleavable by an insect protease. In some embodiments, a linker may allow proteins to achieve their independent secondary and tertiary structure formation during translation. In some embodiments, the linker can be either resistant or susceptible to cleavage in plant cellular environments, in the insect and/or lepidopteran gut environment, and/or in the insect hemolymph and lepidopteran hemolymph environment. In some embodiments, a linker can be cleaved by a protease, e.g., in some embodiments, a linker can be cleaved by a plant protease (e.g., papain, bromelain, ficin, actinidin, zingibain, and/or cardosins), an insect protease, a fungal protease, a vertebrate protease, an invertebrate protease, a bacteria protease, a mammal protease, a reptile protease, or an avian protease. In some embodiments, a linker can be cleavable or non-cleavable. In some embodiments, a linker comprises a binary or tertiary region, wherein each region is cleavable by at least two types of proteases: one of which is an insect and/or nematode protease and the other one of which is a human protease. In some embodiments, a linker can have one of (at least) three roles: to cleave in the insect gut environment, to cleave in the plant cell, or to be designed not to intentionally cleave.

“Locus of a pest” refers to the habitat of a pest; food supply of a pest; breeding ground of a pest; area traveled by or inhabited by a pest; material infested, eaten, used by a pest; and/or any environment in which a pest inhabits, uses, is present in, or is expected to be. In some embodiments, the locus of a pest includes, without limitation, a pest habitat; a pest food supply; a pest breeding ground; a pest area; a pest environment; any surface or location that may be frequented and/or infested by a pest; any plant or animal, or a locus of a plant or animal, susceptible to attack by a pest; and/or any surface or location where a pest may be found, may be expected to be found, or is likely to be attacked by a pest.

“Locus of a plant” refers to any place in which a plant is growing; any place where plant propagation materials of a plant are sown; any place where plant propagation materials of a plant will be placed into the soil; or any area where plants are stored, including without limitation, live plants and/or harvested plants, leaves, seeds, fruits, or parts thereof.

“Locus of an animal” refers to any place where animals live, eat, breed, sleep, or otherwise are present in.

“Medium” (plural “media”) refers to a nutritive solution for culturing cells in cell culture.

“MOA” refers to mechanism of action.

“Molecular weight (MW)” refers to the mass or weight of a molecule, and is typically measured in “daltons (Da)” or kilodaltons (kDa). In some embodiments, MW can be calculated using sodium dodecyl sulfate polyacrylamide gel electrophoresis (SDS-PAGE), analytical ultracentrifugation, or light scattering. In some embodiments, the SDS-PAGE method is as follows: the sample of interest is separated on a gel with a set of molecular weight standards. The sample is run, and the gel is then processed with a desired stain, followed by destaining for about 2 to 14 hours. The next step is to determine the relative migration distance (Rf) of the standards and protein of interest. The migration distance can be determined using the following equation:

$$Rf = \frac{Migration distance of the protein}{Migration distance of the dye front} \qquad Formula ( a )$$

Next, the logarithm of the MW can be determined based on the values obtained for the bands in the standard; e.g., in some embodiments, the logarithm of the molecular weight of an SDS-denatured polypeptide and its relative migration distance (Rf) is plotted into a graph. After plotting the graph, interpolating the value derived will provide the molecular weight of the unknown protein band.

“Motif” refers to a polynucleotide or polypeptide sequence that is implicated in having some biological significance and/or exerts some effect or is involved in some biological process.

“Multiple cloning site” or “MCS” refers to a segment of DNA found on a vector that contains numerous restriction sites in which a DNA sequence of interest can be inserted.

“Mutant” refers to an organism, DNA sequence, amino acid sequence, peptide, polypeptide, or protein, that has an alteration or variation (for example, in the nucleotide sequence or the amino acid sequence), which causes said organism and/or sequence to be different from the naturally occurring or wild-type organism, wild-type sequence, and/or reference sequence with which the mutant is being compared. In some embodiments, this alteration or variation can be one or more nucleotide and/or amino acid substitutions or modifications (e.g., deletion or addition). In some embodiments, the one or more amino acid substitutions or modifications can be conservative; here, such a conservative amino acid substitution and/or modification in a “mutant” does not substantially diminish the activity of the mutant in relation to its non-mutant form. For example, in some embodiments, a “mutant” possesses one or more, for example, one to seven conservative amino acid substitutions when compared to a peptide with a disclosed and/or claimed sequence, as indicated by a SEQ ID NO.

“N-terminus” or “N-terminal” refers to the free amine group (i.e., —NH2) that is positioned on beginning or start of a polypeptide.

“NCBI” refers to the National Center for Biotechnology Information.

“nm” refers to nanometers.

“Non-Polar amino acid” is an amino acid that is weakly hydrophobic and includes glycine, alanine, proline, valine, leucine, isoleucine, phenylalanine and methionine. Glycine or gly is the most preferred non-polar amino acid for the dipeptides of this invention.

“Normalized peptide yield” means the peptide yield in the conditioned medium divided by the corresponding cell density at the point the peptide yield is measured. The peptide yield can be represented by the mass of the produced peptide in a unit of volume, for example, mg per liter or mg/L, or by the UV absorbance peak area of the produced peptide in the HPLC chromatograph, for example, mAu·sec. The cell density can be represented by visible light absorbance of the culture at wavelength of 600 nm (OD600).

“OD” refers to optical density. Typically, OD is measured using a spectrophotometer. When measuring growth over time of a cell population, OD600 is preferable to UV spectroscopy; this is because at a 600 nm wavelength, the cells will not be harmed as they would under too much UV light.

“OD660 nm” or “OD₆₆₀ₙₘ” refers to optical densities of a liquid sample measured (for example, yeast cell culture) when measured in a spectrophotometer at 660 nanometers (nm).

“One letter code” means the peptide sequence which is listed in its one letter code to distinguish the various amino acids in the primary structure of a protein: alanine=A, arginine=R, asparagine=N, aspartic acid=D, asparagine or aspartic acid=B, cysteine=C, glutamic acid=E, glutamine=Q, glutamine or glutamic acid=Z, glycine=G, histidine=H, isoleucine=I, leucine=L, lysine=K, methionine=M, phenylalanine=F, proline=P, serine=S, threonine=T, tryptophan=W, tyrosine=Y, and valine=V.

“Open reading frame” or “ORF” refers to a length of RNA or DNA sequence, between a translation start signal (e.g., AUG or ATG, respectively) and any one or more of the known termination codons, which encodes one or more polypeptide sequences. Put another way, the ORF describes the frame of reference as seen from the point of view of a ribosome translating the RNA code, insofar that the ribosome is able to keep reading (i.e., adding amino acids to the nascent protein) because it has not encountered a stop codon. Thus, “open reading frame” or “ORF” refers to the amino acid sequence encoded between translation initiation and termination codons of a coding sequence. Here, the terms “initiation codon” and “termination codon” refer to a unit of three adjacent nucleotides (i.e., a codon) in a coding sequence that specifies initiation and chain termination, respectively, of protein synthesis (mRNA translation).

In some embodiments, an ORF is a continuous stretch of codons that begins with a start codon (usually ATG for DNA, and AUG for RNA) and ends at a stop codon (usually UAA, UAG or UGA). In other embodiments, an ORF can be length of RNA or DNA sequence, between a translation start signal (e.g., AUG or ATG) and any one or more of the known termination codons, wherein said length of RNA or DNA sequence encodes one or more polypeptide sequences. In some other embodiments, an ORF can be a DNA sequence encoding a protein which begins with an ATG start codon and ends with a TGA, TAA or TAG stop codon. ORF can also mean the translated protein that the DNA encodes. Generally, those having ordinary skill in the art distinguish the terms “open reading frame” and “ORF,” from the term “coding sequence,” based upon the fact that the broadest definition of “open reading frame” simply contemplates a series of codons that does not contain a stop codon. Accordingly, while an ORF may contain introns, the coding sequence is distinguished by referring to those nucleotides (e.g., concatenated exons) that can be divided into codons that are actually translated into amino acids by the ribosomal translation machinery (i.e., a coding sequence does not contain introns); however, as used herein, the terms “coding sequence”; “CDS”; “open reading frame”; and “ORF,’ are used interchangeably.

“Operable” refers to the ability to be used, the ability to do something, and/or the ability to accomplish some function or result. For example, in some embodiments, “operable” refers to the ability of a polynucleotide, DNA sequence, RNA sequence, or other nucleotide sequence or gene to encode a peptide, polypeptide, and/or protein. For example, in some embodiments, a polynucleotide may be operable to encode a protein, which means that the polynucleotide contains information that imbues it with the ability to create a protein (e.g., by transcribing mRNA, which is in turn translated to protein).

“Operably linked” refers to a juxtaposition wherein the components so described are in a relationship permitting them to function in their intended manner. For example, in some embodiments, operably linked can refer to two or more DNA, peptide, or polypeptide sequences. In other embodiments, operably linked can mean that the two adjacent DNA sequences are placed together such that the transcriptional activation of one DNA sequence can act on the other DNA sequence. In yet other embodiments, the term “operably linked” can refer to two or more peptides and/or polypeptides, wherein said two or more peptides and/or polypeptides are connected in such a way as to yield a single polypeptide chain; alternatively, the term operably linked can refer to two or more peptides that are connected in such a way that one peptide exerts some effect on the other. In yet other embodiments, operably linked can refer to two adjacent DNA sequences are placed together such that the transcriptional activation of one can act on the other.

“Out-recombined” or “out-recombination” refers to the removal of a gene and/or polynucleotide sequence (e.g., an endogenous gene, a transgene, a heterologous polynucleotide, etc.) that is flanked by two site-specific recombination sites (e.g., the 5′- and 3′-nucleotide sequence of a target gene that is homologous to the homology arms of a target vector) during in vivo homologous recombination. In some embodiments, the term “out-recombined” refers to the process wherein an endogenous gene is removed, e.g., during homologous recombination. In other embodiments, the term “out-recombined” refers to the process wherein a heterologous polynucleotide is removed via molecular mechanisms intrinsic to the host cell.

“Pest” refers to any organism (e.g., including, but is not limited to: insects, arachnids, crustaceans, snails, nematodes, flatworms, roundworms, pinworms, hookworms, tapeworms, trypanosomes, schistosomes, botflies, fleas, ticks, mites, lice and the like) that is annoying, troublesome, detrimental, a nuisance, attacks, is directly or indirectly harmful and/or causes damage or destruction, for example, to people, plants, crops, pets, and/or livestock.

In some embodiments, a pest can be an organism (e.g., an insect) that directly or indirectly harms people, plants, crops, pets, and/or livestock.

In some embodiments, a pest can have a harmful direct effect on plant. For example, in some embodiments, a pest can be insect (e.g., a phytophagous insect pest) that causes a harmful direct effect on a plant by feeding on the plant leaves.

In yet other embodiments, a pest can be an insect (e.g., a phytopathogenic insect) that causes a harmful indirect effect on a plant, e.g., via transmission of a disease agent (e.g., a virus, bacteria, etc.) from the insect to the plant. Accordingly, in some embodiments, the pest serves as a vector for pathogen transmission to plants.

In some embodiments, a pest can be a phytophagous insect that consumes, eats, or otherwise injures a seed or plant grown therefrom, or any part of a plant, e.g., plant tissues, plant cells, plant parts, plant organs (e.g., leaves, stems, roots, etc.), seeds, propagules, embryos and progeny of the same.

In some embodiments, a pest can be an insect or nematode that causes a harmful direct effect on people, pets, and/or livestock, e.g., via transmission of a disease agent (e.g., a virus, bacteria, etc.) from the pest to the people, pets, and/or livestock. Accordingly, in some embodiments, the pest serves as a vector for pathogen transmission to people, pets, and/or livestock.

In some embodiments, a pest includes, but is not limited to, any plant pest of the Phyla Nematoda, Arthropoda, and/or Mollusca. In various embodiments, a plant pest or pest can include insects, nematodes, non-insect arthropod pests, including, but not limited to: thrips, aphids, whiteflies, mites, including spider mites and other plant arthropods pests that are known to defoliate and generally attack plants, for example, beet flea beetles, Chaetocnema concinna, beet leafhoppers, Circulifer tenellus; beet leafminers, Pegomya betae; sugarbeet root aphids, Pemphigus betae; garden symphylans, Scutigerella immaculata; lucerne fleas, Sminthurus viridis; sugarbeet root maggots, Tetanops myopaeformis, wireworms, corn rootworms (Diabrotica spp.), corn silk flies (Euxesta stigmatias), European corn borers (Ostrinia nubilalis), stalk borers (Diatraea spp.), aphids, billbugs (Sphenophorus spp.), fall armyworms (Spodoptera frugiperda), corn earworms (Helicoverpa zea), cutworms, wireworms, velvet bean caterpillars, Anticarsia gemmatalis Hübner; soybean loopers, Chrysodeixis includens Walker; leaf beetles such as Cerotoma spp. and Epilachna varivestis Mulsant; tortricid pod and stem borers, Crocidosema spp.; bollworm, Helicoverpa spp., armyworms, Spodoptera sp., pod-feeding stink bugs, soybean aphids, Aphis glycines, Matsumura; and the bean stem flies Melanagromyza soja Zehntner, thrips, ticks, and the like.

As used herein, “pesticidal” is generally used to refer to the ability of a combination or composition of the present disclosure, to increase mortality or inhibit growth rate of insects.

“Pesticidal activity” means that upon or after exposing the pest to a KPV peptide or KVP containing composition, or a combination of the present disclosure, the pest either dies stops or slows its movement; stops or slows its feeding; stops or slows its growth; becomes confused (e.g., with regard to navigation, locating food, sleeping behaviors, and/or mating); fails to pupate; interferes with reproduction; and/or precludes the insect from producing offspring and/or precludes the insect from producing fertile offspring, or any combination of the foregoing. Pests can include

“Pesticidal effect” refers to the removal or the reduction of harm of pests. The concept of “pesticidal effect” includes reducing of the target pest, killing of pests (extermination), pest proliferation inhibition, pest development inhibition, pest growth inhibition, repelling of pests (repellence), reducing of the survival rate of the target pest and the removal or the reduction of harm of pests (for example, inhibition of ingestion capacity of agricultural pests). “Pesticidal effect” includes killing of any individual or group of insects and non-insect arthropod pests.

“Pesticidally-effective amount” refers to an amount of (1) a KPV, a KPV-pesticidal protein, or an agriculturally acceptable salt thereof; and/or (2) an pesticidal composition comprising: a KPV, a KPV-pesticidal protein, or an agriculturally acceptable salt thereof, and an excipient; that is sufficient to: inhibit a pest (e.g., an arthropod plant pest, such as an insect pest); bring about the death of at least one pest; noticeably reduce or decrease pest growth, feeding, or normal physiological development; inhibit or decrease the normal pest cellular processes, including maintenance and growth; and/or attenuate or decrease the severity of a pest infestation. This amount will vary depending on such factors including but not limited to: the specific target pest to be controlled; the specific environment, location, plant, crop, or agricultural site to be treated; the environmental conditions, method, rate, concentration, stability, and quantity applied. Further, those having ordinary skill in the art will recognize that the pesticidally-effective amount may also vary with respect to climatic conditions, environmental considerations, and/or frequency of application and/or severity of pest infestation. In some embodiments, pesticidally-effective amounts can be measured by use of assays that measure the reduction in growth or decline in pest populations. One measure of reduction can be to express the decrease in population in logarithmic scale typical of a specific microbial species. That is, a 1 log reduction is equivalent to a 90% reduction versus a control, a 2 log reduction is a 99% reduction, etc.

As used herein, a “pest resistance” trait is a characteristic of a transgenic plant, transgenic animal, transgenic host or transgenic symbiont that causes the plant, animal, host, or symbiont to be resistant to attack from a pest that typically is capable of inflicting damage or loss to the plant, animal, host or symbiont. Such pest resistance can arise from a natural mutation or more typically from incorporation of recombinant DNA that confers pest resistance. To impart insect resistance to a transgenic plant a recombinant DNA, for example, encoding a KVP of the present invention, can, for example, be introgressed into a plant, or part thereof using plant transformations methods known in the art. For example, a KVP cDNA or dsDNA is introgressed into a desired plant, and is subsequently transcribed into a RNA molecule that forms an mRNA or a dsRNA molecule within the tissues or fluids of the recombinant plant, or the peptide is expressed in the plant, or part thereof, under suitable conditions, for example, the plant, or part thereof, having the appropriate transcription and translation signals and/or machinery to express the introduced KVP peptide nucleic acid in situ.

“Pharmaceutically acceptable salt” is synonymous with agriculturally acceptable salt, and as used herein refers to a compound that is modified by making acid or base salts thereof.

“Plant” shall mean whole plants, plant tissues, plant cells, plant parts, plant organs (e.g., leaves, stems, roots, etc.), seeds, propagules, embryos and progeny of the same. Plant cells can be differentiated or undifferentiated (e.g. callus, suspension culture cells, protoplasts, leaf cells, root cells, phloem cells, and pollen).

“Plant transgenic protein” means a protein from a heterologous species that is expressed in a plant after the DNA or RNA encoding it was delivered into one or more of the plant cells.

“Plant-incorporated protectant” or “PIP” means a pesticidal protein produced by transgenic plants, and the genetic material necessary for the plant to produce the protein.

“Plant cleavable linker” means a cleavable linker peptide, or a nucleotide encoding a cleavable linker peptide, which contains a plant protease recognition site and can be cleaved during the protein expression process in the plant cell.

“Plant regeneration media” means any media that contains the necessary elements and vitamins for plant growth and plant hormones necessary to promote regeneration of a cell into an embryo which can germinate and generate a plantlet derived from tissue culture. Often the media contains a selectable agent to which the transgenic cells express a selection gene that confers resistance to the agent.

“Plant transformation” can include numerous methods for plant transformation that have been developed, including biological and physical plant transformation protocols. See, for example, Miki et al., “Procedures for Introducing Foreign DNA into Plants,” in Methods in Plant Molecular Biology and Biotechnology, Glick and Thompson Eds., CRC Press, Inc., Boca Raton, pp. 67-88 (1993), and Armstrong, “The First Decade of Maize Transformation: A Review and Future Perspective,” Maydica, 44:101-109 (1999). In addition, expression vectors and in vitro culture methods for plant cell or tissue transformation and regeneration of plants are available. See, for example, Gruber, et al., “Vectors for Plant Transformation,” in Methods in Plant Molecular Biology and Biotechnology, Glick and Thompson Eds., CRC Press, Inc., Boca Raton, pp. 89-119 (1993). A genetic trait (e.g. expression of a KVP as disclosed herein) which has been engineered into the genome of a particular plant susceptible to pesticidal activity, may then be moved into the genome of another variety using traditional breeding techniques that are well known in the plant breeding arts. For example, a backcrossing approach is commonly used to move a transgene from a transformed soybean variety into an already developed soybean variety, and the resulting backcross conversion plant would then comprise the transgene(s). Various genetic elements can be introduced into the plant genome using transformation. These elements include, but are not limited to, genes, coding sequences, inducible, constitutive and tissue specific promoters, enhancing sequences, and signal and targeting sequences. For example, see the traits, genes, and transformation methods listed in U.S. Pat. No. 6,118,055.

“Plasmid” refers to a DNA segment that acts as a carrier for a gene of interest, and, when transformed or transfected into an organism, can replicate and express the DNA sequence contained within the plasmid independently of the host organism. Plasmids are a type of vector, and can be “cloning vectors” (i.e., simple plasmids used to clone a DNA fragment and/or select a host population carrying the plasmid via some selection indicator) or “expression plasmids” (i.e., plasmids used to produce large amounts of polynucleotides and/or polypeptides).

“Polar amino acid” is an amino acid that is polar and includes serine, threonine, cysteine, asparagine, glutamine, histidine, tryptophan and tyrosine; preferred polar amino acids are serine, threonine, cysteine, asparagine and glutamine; with serine being most highly preferred.

“Polynucleotide” refers to a polymeric-form of nucleotides (e.g., ribonucleotides, deoxyribonucleotides, or analogs thereof) of any length; e.g., a sequence of two or more ribonucleotides or deoxyribonucleotides. As used herein, the term “polynucleotide” includes double- and single-stranded DNA, as well as double- and single-stranded RNA; it also includes modified and unmodified forms of a polynucleotide (modifications to and of a polynucleotide, for example, can include methylation, phosphorylation, and/or capping). In some embodiments, a polynucleotide can be one of the following: a gene or gene fragment (for example, a probe, primer, EST, or SAGE tag); genomic DNA; genomic DNA fragment; exon; intron; messenger RNA (mRNA); transfer RNA; ribosomal RNA; ribozyme; cDNA; recombinant polynucleotide; branched polynucleotide; plasmid; vector; isolated DNA of any sequence; isolated RNA of any sequence; nucleic acid probe; primer or amplified copy of any of the foregoing.

In yet other embodiments, a polynucleotide can refer to a polymeric-form of nucleotides operable to encode the open reading frame of a gene.

In some embodiments, a polynucleotide can refer to cDNA.

In some embodiments, polynucleotides can have any three-dimensional structure and may perform any function, known or unknown. The structure of a polynucleotide can also be referenced to by its 5′- or 3′-end or terminus, which indicates the directionality of the polynucleotide. Adjacent nucleotides in a single-strand of polynucleotides are typically joined by a phosphodiester bond between their 3′ and 5′ carbons. However, different internucleotide linkages could also be used, such as linkages that include a methylene, phosphoramidate linkages, etc. This means that the respective 5′ and 3′ carbons can be exposed at either end of the polynucleotide, which may be called the 5′ and 3′ ends or termini. The 5′ and 3′ ends can also be called the phosphoryl (PO₄) and hydroxyl (OH) ends, respectively, because of the chemical groups attached to those ends. The term polynucleotide also refers to both double- and single-stranded molecules. Unless otherwise specified or required, any embodiment that makes or uses a polynucleotide encompasses both the double-stranded form and each of two complementary single-stranded forms known or predicted to make up the double-stranded form.

In some embodiments, a polynucleotide can include modified nucleotides, such as methylated nucleotides and nucleotide analogs (including nucleotides with non-natural bases, nucleotides with modified natural bases such as aza- or deaza-purines, etc.). If present, modifications to the nucleotide structure can be imparted before or after assembly of the polynucleotide.

In some embodiments, a polynucleotide can also be further modified after polymerization, such as by conjugation with a labeling component. Additionally, the sequence of nucleotides in a polynucleotide can be interrupted by non-nucleotide components. One or more ends of the polynucleotide can be protected or otherwise modified to prevent that end from interacting in a particular way (e.g. forming a covalent bond) with other polynucleotides.

In some embodiments, a polynucleotide can be composed of a specific sequence of four nucleotide bases: adenine (A); cytosine (C); guanine (G); and thymine (T). Uracil (U) can also be present, for example, as a natural replacement for thymine when the polynucleotide is RNA. Uracil can also be used in DNA. Thus, the term “sequence” refers to the alphabetical representation of a polynucleotide or any nucleic acid molecule, including natural and non-natural bases.

The term “RNA molecule” or ribonucleic acid molecule refers to a polynucleotide having a ribose sugar rather than deoxyribose sugar and typically uracil rather than thymine as one of the pyrimidine bases. An RNA molecule of the invention is generally single-stranded, but can also be double-stranded. In the context of an RNA molecule from an RNA sample, the RNA molecule can include the single-stranded molecules transcribed from DNA in the cell nucleus, mitochondrion or chloroplast, which have a linear sequence of nucleotide bases that is complementary to the DNA strand from which it is transcribed.

In some embodiments, a polynucleotide can further comprise one or more heterologous regulatory elements. For example, in some embodiments, the regulatory element is one or more promoters; enhancers; silencers; operators; splicing signals; polyadenylation signals; termination signals; RNA export elements, internal ribosomal entry sites (IRES); poly-U sequences; or combinations thereof.

“Post-transcriptional regulatory elements” are DNA segments and/or mechanisms that affect mRNA after it has been transcribed. Mechanisms of post-transcriptional mechanisms include splicing events; capping, splicing, and addition of a Poly (A) tail, and other mechanisms known to those having ordinary skill in the art.

“Promoter” refers to a region of DNA to which RNA polymerase binds and initiates the transcription of a gene.

“Protein” has the same meaning as “peptide” and/or “polypeptide” in this document.

“Ratio” refers to the quantitative relation between two amounts showing the number of times one value contains or is contained within the other.

“Reading frame” refers to one of the six possible reading frames, three in each direction, of the double stranded DNA molecule. The reading frame that is used determines which codons are used to encode amino acids within the coding sequence of a DNA molecule. In some embodiments, a reading frame is a way of dividing the sequence of nucleotides in a polynucleotide and/or nucleic acid (e.g., DNA or RNA) into a set of consecutive, non-overlapping triplets.

“Recombinant DNA” or “rDNA” refers to DNA that is comprised of two or more different DNA segments.

“Recombinant vector” means a DNA plasmid vector into which foreign DNA has been inserted.

“Regulatory elements” refers to a genetic element that controls some aspect of the expression and/or processing of nucleic acid sequences. For example, in some embodiments, a regulatory element can be found at the transcriptional and post-transcriptional level. Regulatory elements can be cis-regulatory elements (CREs), or trans-regulatory elements (TREs). In some embodiments, a regulatory element can be one or more promoters; enhancers; silencers; operators; splicing signals; polyadenylation signals; termination signals; RNA export elements, internal ribosomal entry sites (IRES); poly-U sequences; and/or other elements that influence gene expression, for example, in a tissue-specific manner; temporal-dependent manner; to increase or decrease expression; and/or to cause constitutive expression.

“Restriction enzyme” or “restriction endonuclease” refers to an enzyme that cleaves DNA at a specified restriction site. For example, a restriction enzyme can cleave a plasmid at an EcoRI, SacII or BstXI restriction site allowing the plasmid to be linearized, and the DNA of interest to be ligated.

“Restriction site” refers to a location on DNA comprising a sequence of 4 to 8 nucleotides, and whose sequence is recognized by a particular restriction enzyme.

“Selection gene” means a gene which confers an advantage for a genetically modified organism to grow under the selective pressure.

“sp.” refers to species.

“ssp.” or “subsp.” refers to subspecies.

“Subcloning” or “subcloned” refers to the process of transferring DNA from one vector to another, usually advantageous vector. For example, polynucleotide encoding a mutant KPV can be subcloned into a pLB102 plasmid subsequent to selection of yeast colonies transformed with pKLAC1 plasmids.

“SSI” is an acronym that is context dependent. In some contexts, it can refer to “site-specific integration,” which is used to refer to a sequence that will permit in vivo homologous recombination to occur at a specific site within a host organism's genome. Thus, in some embodiments, the term “site-specific integration” refers to the process directing a transgene to a target site in a host-organism's genome, allowing the integration of genes of interest into pre-selected genome locations of a host-organism. However, in other contexts, SSI can refer to “surface spraying indoors,” which is a technique of applying a variable volume sprayable volume of an insecticide onto surfaces where vectors rest, such as on walls, windows, floors and ceilings.

“STA” or “Translational stabilizing protein” or “stabilizing domain” or “stabilizing protein” (used interchangeably herein) means a peptide or protein with sufficient tertiary structure that it can accumulate in a cell without being targeted by the cellular process of protein degradation. The protein can be between 5 and 50 amino acids long. The translational stabilizing protein is coded by a DNA sequence for a protein that is operably linked with a sequence encoding an pesticidal protein or a KPV in the ORF. The operably-linked STA can either be upstream or downstream of the KPV and can have any intervening sequence between the two sequences (STA and KPV) as long as the intervening sequence does not result in a frame shift of either DNA sequence. The translational stabilizing protein can also have an activity which increases delivery of the KPV across the gut wall and into the hemolymph of the insect.

“sta” means a nucleotide encoding a translational stabilizing protein.

“Stringent hybridization” or “stringent hybridization conditions” refers to conditions under which a polynucleotide (e.g., a nucleic acid probe, primer or oligonucleotide) will hybridize to its target sequence, typically in a complex mixture of nucleic acids, but not to other sequences. In some embodiments, the term “stringent hybridization” or “stringent hybridization conditions” refers to the conditions under which a probe will hybridize to its target sequence to a detectably greater degree than to other sequences (e.g., at least 2-fold, 5-fold, or 10-fold over background).

Stringent hybridization conditions are sequence- and length-dependent, and will be different in different circumstances. Similarly, stringent hybridization conditions depend on % (percent)-identity (or %-mismatch) over a certain length of nucleotide residues. Generally, longer sequences hybridize specifically at higher temperatures than shorter sequences. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 or 500 nucleotides in length

For example, in some embodiments, stringent hybridization conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides), and at least about 60° C. for long probes (e.g., greater than 50 nucleotides). In some embodiments, stringent hybridization conditions may also be achieved with the addition of destabilizing agents such as formamide.

In some embodiments, low stringency hybridization conditions include hybridization with a buffer solution of 30 to 35% formamide, 1 M NaCl, 1% SDS (sodium dodecyl sulfate) at 37° C., and a wash in 1× to 2×SSC (20×SSC-3.0 M NaCl/0.3 M trisodium citrate) at 50-55° C. In some embodiments, moderate stringency hybridization conditions include hybridization in 40 to 45% formamide, 1.0 M NaCl, 1% SDS at 37° C., and a wash in 0.5× to 1×SSC at 55-60° C. In some embodiments, high stringency hybridization conditions include hybridization in 50% formamide, 1 M NaCl, 1% SDS at 37° C., and a final wash in 0.1×SSC at 60 to 65° C. for at least about 20 minutes. Optionally, wash buffers may comprise about 0.1% to about 1% SDS. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ (thermal melting point) can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, “% form” is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Washes are typically performed at least until equilibrium is reached and a low background level of hybridization is achieved, such as for 2 hours, 1 hour, or 30 minutes.

In some embodiments, the Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C.

In some embodiments, stringent hybridization conditions are selected to be about 5° C. lower than the thermal melting point (Tₘ) for the specific sequence at a defined ionic strength and pH. However, in some embodiments, stringent hybridization conditions can utilize a hybridization and/or wash temperature that is about 1° C., 2° C., 3° C., or 4° C. lower than the thermal melting point (Tₘ).

Using the equation, hybridization and wash compositions, and desired Tₘ, those of ordinary skill will understand that variations in the stringency of hybridization and/or wash solutions are inherently described. An exemplary description of the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York); the disclosures of which are incorporated herein by reference in their entireties.

In some embodiments, a polynucleotide of the present disclosure can stringently hybridize to a polynucleotide encoding a KPV, or a complementary nucleotide sequence thereof, with the proviso that the KPV does not comprise the amino acid sequence of SEQ ID NOs: 1 or 2253.

“Structural motif” refers to the three-dimensional arrangement of peptides and/or polypeptides, and/or the arrangement of operably linked polypeptide segments. For example, the polypeptide comprising ERSP-STA-L-KPV has an ERSP motif, an STA motif, a LINKER motif, and a KPV polypeptide motif.

“Susceptible to attack by a pest(s),” refer to plants, or human or animal patients or subjects, susceptible to a pest or a pest infections.

“Toxin” refers to a venom and/or a poison, especially a protein or conjugated protein produced by certain animals, higher plants, and pathogenic bacteria. Generally, the term “toxin” is reserved natural products, e.g., molecules and peptides found in scorpions, spiders, snakes, poisonous mushrooms, etc., whereas the term “toxicant” is reserved for man-made products and/or artificial products e.g., man-made chemical pesticides. However, as used herein, the terms “toxin” and “toxicant” are used synonymously

“Transfection” and “transformation” both refer to the process of introducing exogenous and/or heterologous DNA or RNA (e.g., a vector containing a polynucleotide that encodes a CRIP) into a host organism (e.g., a prokaryote or a eukaryote). Generally, those having ordinary skill in the art sometimes reserve the term “transformation” to describe processes where exogenous and/or heterologous DNA or RNA are introduced into a bacterial cell; and reserve the term “transfection” for processes that describe the introduction of exogenous and/or heterologous DNA or RNA into eukaryotic cells. However, as used herein, the term “transformation” and “transfection” are used synonymously, regardless of whether a process describes the introduction exogenous and/or heterologous DNA or RNA into a prokaryote (e.g., bacteria) or a eukaryote (e.g., yeast, plants, or animals).

“Transgene” means a heterologous and/or exogenous polynucleotide sequence that is transformed into an organism and/or a cell therefrom.

“Transgenic host cell” or “host cell” means a cell which is transformed with a gene and has been selected for its transgenic status via an additional selection gene.

“Transgenic plant” means a plant that has been derived from a single cell that was transformed with foreign DNA such that every cell in the plant contains that transgene.

“Transient expression system” means an Agrobacterium tumefaciens-based system which delivers DNA encoding a disarmed plant virus into a plant cell where it is expressed. The plant virus has been engineered to express a protein of interest at high concentrations, up to 40% of the total soluble protein (TSP).

“Triple expression cassette refers to three KPV expression cassettes contained on the same vector.

“TRBO” means a transient plant expression system using Tobacco mosaic virus with removal of the viral coating protein gene.

“Trypsin cleavage” means an in vitro assay that uses the protease enzyme trypsin (which recognizes exposed lysine and arginine amino acid residues) to separate a cleavable linker at that cleavage site. It also means the act of the trypsin enzyme cleaving that site.

“TSP” or “total soluble protein” means the total amount of protein that can be extracted from a plant tissue sample and solubilized into the extraction buffer.

“var.” refers to varietas or variety. The term “var.” is used to indicate a taxonomic category that ranks below the species level and/or subspecies (where present). In some embodiments, the term “var.” represents members differing from others of the same subspecies or species in minor but permanent or heritable characteristics.

“Vector” refers to the DNA segment that accepts a heterologous polynucleotide operable to encode a peptide of interest (e.g., KPV). The heterologous polynucleotide is known as an “insert” or “transgene.”

“Wild type” or “WT” or “wild-type” or “wildtype” refer to the phenotype and/or genotype (i.e., the appearance or sequence) of an organism, polynucleotide sequence, and/or polypeptide sequence, as it is found and/or observed in its naturally occurring state or condition.

“Yield” refers to the production of a peptide, and increased yields can mean increased amounts of production, increased rates of production, and an increased average or median yield and increased frequency at higher yields. The term “yield” when used in reference to plant crop growth and/or production, as in “yield of the plant” refers to the quality and/or quantity of biomass produced by the plant.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, and formulation. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp1-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigão, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000); each of these references are incorporated herein by reference in their entireties.

Although the disclosure of the invention has been described in detail for purposes of clarity and understanding, it will be obvious to those with skill in the art that certain modifications can be practiced within the scope of the appended claims. All publications and patent documents cited herein are hereby incorporated by reference in their entirety for all purposes to the same extent as if each were so individually denoted.

Throughout this specification, unless the context requires otherwise, the word “comprise,” or variations such as “comprises” or “comprising,” will be understood to imply the inclusion of a stated step or element or integer or group of steps or elements or integers but not the exclusion of any other step or element or integer or group of elements or integers.

All patent applications, patents, and printed publications referred to herein are incorporated by reference in their entirety to the same extent as if each individual publication, patent, or patent application was specifically and individually indicated to be incorporated by reference in its entirety. And, all patent applications, patents, and printed publications cited herein are incorporated herein by reference in the entireties, except for any definitions, subject matter disclaimers, or disavowals, and except to the extent that the incorporated material is inconsistent with the express disclosure herein, in which case the language in this disclosure controls.

### Pesticidal Kappa Peptide Variants (KPVs)

Biological pesticidal agents, e.g., pesticidal agents based on, or derived from, natural sources (such as an organism or product therefrom), may confer crop protection by suppressing disease incidence, and/or reducing the number of pathogenic insects or the severity of symptoms caused by the same. Thus, biological pesticidal agents represent a desirable replacement of traditional agrochemicals, and can be used to avoid or remediate the adverse effects thereof.

The present disclosure provides an engineered, non-naturally occurring pesticidal KPVs, or an agriculturally acceptable salt thereof; agricultural compositions thereof, further comprising an excipient; and methods of making and using the same.

In some embodiments, a KPV comprises a DNA sequence, polynucleotide, amino acid sequence, peptide, polypeptide, or protein, that has an alteration, variation, or modification (for example, in the nucleotide sequence or the amino acid sequence), relative to a wild-type or originating DNA sequence, polynucleotide, amino acid sequence, peptide, polypeptide, or protein from which the KPV was derived.

In some embodiments, a KPV comprises an alteration, variation, or modification that can be one or more nucleotide and/or amino acid substitutions or modifications (e.g., deletion or addition), in a DNA sequence, polynucleotide, amino acid sequence, peptide, polypeptide, or protein, relative to the wild-type or originating DNA sequence, polynucleotide, amino acid sequence, peptide, polypeptide, or protein from which the KPV was derived.

An exemplary originating protein from which KPVs can be derived (i.e., an originating protein that is mutated, wherein one or more amino acid substitutions, deletions, or additions to the originating protein results) is the protein “WT-Kappa,” which has an amino acid sequence:

(SEQ ID NO: 1)
“AICTGADRPCAACCPCCPGTSCKAESNGVSYCRKDEP”.

### Illustrative KPVs

In some embodiments, a Kappa Peptide Variant (KPV) can be a mutant or variant that differs from wild type Kappa protein (SEQ ID NO: 1), e.g., in some embodiments, this variance can be an amino acid substitution, amino acid deletion/insertion, or a change to the polynucleotide encoding the KPV. The result of this variation is a non-naturally occurring polypeptide and/or polynucleotide sequence encoding the same, relative to WT Kappa, that possesses pesticidal activity against one or more pest species.

In some embodiments, a Kappa Peptide Variant (KPV), or an agriculturally acceptable salt thereof, having pesticidal activity against one or more insect species, comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise or consist of the amino acid sequence of SEQ ID NOs: 1 or 253.

In some embodiments, a Kappa Peptide Variant (KPV), or an agriculturally acceptable salt thereof, having pesticidal activity against one or more insect species, comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV further comprises 1, 2, 3, 4, or 5 amino acid substitutions that are conservative amino acid substitutions.

A summary of illustrative KPVs are provided in the table below.

TABLE 1
Summary of illustrative KPVs. Table 1 provides a summary of KPVs
having at least one mutation that confers at least one novel property
to the KPV relative to WT Kappa. The properties include: (1) increased
expression; (2) increased activity; or (3) a combination thereof,
relative to SEQ ID NO: 1

| SEQ ID NO | Name | Amino Acid Sequence |
|---|---|---|
| 2 | KPV2 | GICTGADRPCAACCPCCPGTSCKTESNGVSYCRKD |
| 3 | KPV3 | SICTGADRPCAACCPCCPGTSCKAGVNGVAYCRKDEP |
| 4 | KPV4 | AICTGADRPCAACCPCCPGTSCKAEANGVSRCRKDEP |
| 5 | KPV5 | AICTGADRPCAACCPCCPGTSCKAFSNGVSYCRKDEP |
| 6 | KPV1 | AICTGADRPCAACCPCCPGTSCKAEWNGVSYCRKDEP |
| 7 | KPV7 | AICTGADRPCAAICPCCPGTSCKAESNGVSYCRKDEP |
| 10 | KPV10 | AICTGIDRPCAACCPCCPGTSCKAESNGVSYCRKDEP |
| 11 | KPV11 | AICTGADRPCAACCPCCPGTSCKAESNGVSHCRKDEP |
| 12 | KPV12 | AICTGADRPCAACCPCCPGTSSKAESNGVCYCRKDEP |
| 13 | KPV13 | AICTEADRPCHACCPCCPGTSCKAESNGVRYCRKDEP |
| 16 | KPV16 | AICTGADRPCAICCPCCPGTSCKAESNGVSYCRKDEP |
| 17 | KPV17 | AICEGADRPCAACCPCCPGTSCKAESNGVSYCRKDEP |
| 18 | KPV18 | AICTGADRPCAACCPCCPGTSCKAESRGVSYCRKDEP |
| 19 | KPV19 | AICTGADRPCAACCPCCPGTSCKAESNGVSYGRKDEP |
| 23 | KPV23 | AICTGADRPCAACCPCCPGTSCKYESNGVSYCRKDEP |
| 25 | KPV25 | AICTGADRPCAACNPCCPGTSCKAESNGVSYCRKMEP |
| 30 | KPV30 | AICTGADRPYAACCPCCPGTSCKAESNGVSYCRKDEP |
| 31 | KPV31 | AICTGADRPKAACCPCCPGTSCKAESNGVSYCRKDEP |
| 35 | KPV35 | AICTGADRPCAACCPCCPGTSCKAESNGVSYCRKNEP |
| 39 | KPV39 | AICTKADRPCAACCPCCPGTSCKAASNGVSYCRKDEP |
| 40 | KPV40 | AVCTGADRPCAACCPCCPGTSCKAESNGVSYCRKDEP |
| 45 | KPV45 | AICTGADRPCAACCPCCPGSSCKAEGNGVSYCRKDEP |
| 46 | KPV46 | AICTGADRPCAACCPCCPGTSCKAEPNGVSYCRRDEP |
| 48 | KPV48 | AICTGADRPCAACCPCCPGTSCKAESNGVSYCRKDVP |
| 50 | KPV50 | AICTGADRPCAACCPCCPGTSCKAEPNGVSYCRKDEP |
| 54 | KPV54 | AICTGADRPCAACCPCCPGTSCKAESNGVSFCRKDEP |
| 55 | KPV55 | AICTDADRPCAACCPCCPGTSCKAESNGVSYCRKDEP |
| 56 | KPV56 | AIYTGADRPCAACCPCCPGTSCKAESNGVSNCRKDEP |
| 57 | KPV57 | AICTGADRPCAAGCPCCPGTSCKAESNGVSYCRKDEP |
| 60 | KPV60 | AICTGADRPCAACCPCCPGTSCKAESVGVSYCRKDEP |
| 66 | KPV66 | AICRGADRPCAACCPCCPGTSCKAESNGVSYCRKDEP |
| 68 | KPV68 | AICTGADRPCAACCPCCPGTSCWAESNGVSYCRKDEP |
| 70 | KPV70 | AICTGADRPCAACCPCYPGTSCKAESNGVSYCRKDEP |
| 71 | KPV71 | AICTGADRPCAAECPCCPGTSCKAESNGVSYCRKDEP |
| 73 | KPV73 | AICTGADGPCAACCPCCPGTSCKAESNGVSYCRKDES |
| 74 | KPV74 | AICTGADSPCAACCPCCPGTTCKAESNGVSYCRKDEP |
| 76 | KPV76 | AICTGADRPCAACCPCCPGTSCKAETNGVSYCRKDEP |
| 77 | KPV77 | AICTGADRPCAACCPCCPGTSCKAESNGVSYCRGDEP |
| 78 | KPV78 | AICTGADRPCSACCPCCPGHSCKAESNGVSYCRKDEP |
| 79 | KPV79 | AICTGADRPRAACCPCCPGTSCKAESNGVSYCRKDEP |
| 85 | KPV85 | AICTGADRPCAACCPCCPGTSCKAESNGVSYCRADEP |
| 102 | KPV102 | AICTGADRPCAACCPCCPGTSCKAESNGVSYCRNDEP |
| 110 | KPV110 | AICTGEDRPCAACCPCCPGTSCKAESNGVSYCRKDEP |
| 114 | KPV114 | AICTGADRPCAACCPCCPGTSCFAESNGVSYCRKDEP |
| 124 | KPV124 | AICTGADRPCAACCPCCPGTSCKAEANGVSYCRKDEP |
| 126 | KPV126 | AICTGADRPCAACCPCCPGTSCKAESNGVSYCRLDEP |
| 140 | KPV140 | AICTGADRPCAACCPCCPRTSCKAESNGVSYCRKDEP |
| 146 | KPV146 | AICTGADRPCAACCPCCPGTSCKAESNGVSYCRMDEP |
| 147 | KPV147 | AICTGADRPCAACCPCCPGISCKAESNGVSYCRKDEP |
| 149 | KPV149 | AICTGADRPCAACCPCCPGTSCKAEKNGVSYCRKDEP |
| 151 | KPV151 | WICTGADRPCAACCPCCPGTSCKAESNGVSYCRKDEP |
| 152 | KPV152 | AICTGADRACAACCPCCPGTSCKAESNGVSYCRKDEP |
| 157 | KPV157 | AICTGADRPCAACCPCCPGTSCKAESNGVSYCREDEP |
| 158 | KPV158 | AICTGADRPCAACCPCCPGQSCKAESNGVSYCRKDEP |
| 160 | KPV160 | AICTGADRPCAACCPCCPGTSCKAESNGVSYCRVDEP |
| 161 | KPV161 | AICTGADRPCAACCPCCPGTSCKAHSNGVSYCRKDEP |
| 162 | KPV162 | AICTGADRPCAACCPCCPGTSCKAESNGVSYCRKDES |
| 164 | KPV164 | AVCTGADRPCAACCPCCPGTSCKAESNGVWYCRKDEP |
| 167 | KPV167 | AICTGADRPCAACCPCCPGTSCKAESNGVYYCRKDEP |
| 168 | KPV168 | AICTGADRPCAACCPCCPGTSCKAESLGVSYCRKDEP |
| 170 | KPV170 | AICTGADRPCAACCPCCPPTSCKAESNGVSYCRKDEP |
| 173 | KPV173 | AICTGADRPCAACCMCCPGTSCKAESNGVSYCRKDEP |
| 176 | KPV176 | AICTGADRPCAACCPCCPGTSCKWESNGVSYCRKDEP |
| 178 | KPV178 | AICTGADRPCAACCPCCPGTSCKAEGNGVSYCRKDEP |
| 180 | KPV180 | AICTGADRPCAACCPCCPGTSCKAWSNGVSYCRKDEP |
| 183 | KPV183 | AICTGANRPCAAGCPCCPGTSCKAESNGVSYCRKDEP |
| 184 | KPV184 | AICTGADRPCAACCPCCPGTSCKAESNGVSYCRKDEQ |
| 185 | KPV185 | AICTGADRPCAAFCPCCPGTSCKAESNGVSYCRKDEP |
| 186 | KPV186 | AICTGADRPCAACCPCCPGTSCKAESWGVSYCRKDEP |
| 189 | KPV189 | AICTGADRPCAACCPCCPGTSCKAESNGVSYCQKDEP |
| 191 | KPV191 | AICTGADRPCAACCPCCPGTTCQAESNGVSYCRKDEP |
| 192 | KPV192 | AICTGIDRPCAACCPCCPGTSCKAESNGVSYCRKDKP |
| 195 | KPV195 | AICTGAGRPCAACCPCCPGTSCKAESNGVSYCRKDEP |
| 199 | KPV199 | AACTGADRPCAACCPCCPGTSCKAESNGVSYCRKDEP |
| 204 | KPV204 | AIHTGADRPCAACCPCCPGTSCDAESNGVSYCRKDEP |
| 205 | KPV205 | AICTGADRPCAACCPCCPGTSCAAESNGVSYCRKDEP |
| 206 | KPV206 | AICTGADRPCAACCPCCPGTSCDAESNGVSYCRKDEP |
| 209 | KPV209 | AICTGADRPCAACCPCCPGTSCIAESNGVSYCRKDEP |
| 210 | KPV210 | AICTGADRPCAACCPCCPGTSCLAESNGVSYCRKDEP |
| 211 | KPV211 | AICTGADRPCAACCPCCPGTSCMAESNGVSYCRKDEP |
| 212 | KPV212 | AICTGADRPCAACCPCCPGTSCNAESNGVSYCRKDEP |
| 215 | KPV215 | AICTGADRPCAACCPCCPGTSCRAESNGVSYCRKDEP |
| 216 | KPV216 | AICTGADRPCAACCPCCPGTSCVAESNGVSYCRKDEP |
| 217 | KPV217 | AICTGADRPCAACCPCCPGTSCYAESNGVSYCRKDEP |
| 218 | KPV218 | AICTGADRPCAACCPCCPGTSCKAESNGVSYCRDDEP |
| 220 | KPV220 | AICTGADRPCAACCPCCPGTSCKAESNGVSYCRHDEP |
| 221 | KPV221 | AICTGADRPCAACCPCCPGTSCKAESNGVSYCRIDEP |
| 222 | KPV222 | AICTGADRPCAACCPCCPGTSCKAESNGVSYCRPDEP |
| 223 | KPV223 | AICTGADRPCAACCPCCPGTSCKAESNGVSYCRQDEP |
| 224 | KPV224 | AICTGADRPCAACCPCCPGTSCKAESNGVSYCRSDEP |
| 225 | KPV225 | AICTGADRPCAACCPCCPGTSCKAESNGVSYCRTDEP |
| 226 | KPV226 | VICTGADRPCAACCPCCPGTSCKAESNGVSYCRKDEP |
| 227 | KPV227 | LICTGADRPCAACCPCCPGTSCKAESNGVSYCRKDEP |
| 228 | KPV228 | SICTGADRPCAACCPCCPGTSCKAGVNGVAYCRKD |
| 229 | KPV229 | AICTGADRPCAACCPCCPGTSCKAEANGVSRCRKD |
| 230 | KPV230 | AICTGADRPCAACCPCCPGTSCKAEVNGVSYCRKDEP |
| 231 | KPV231 | SICTGADRPCAACCPCCPGTSCKAESNGVSYCRKDEP |
| 232 | KPV232 | DICTGADRPCAACCPCCPGTSCKAESNGVSYCRKDEP |
| 233 | KPV233 | AICTGADRPCAACCPCCPGTSCKAESNGVSLCRKDEP |
| 235 | KPV235 | AICTGADKPCAACCPCCPGTSCKAESNGVSYCRKDEP |
| 236 | KPV236 | AICTGADHPCAACCPCCPGTSCKAESNGVSYCRKDEP |
| 237 | KPV237 | AICTGADYPCAACCPCCPGTSCKAESNGVSYCRKDEP |
| 238 | KPV238 | AICTGADLPCAACCPCCPGTSCKAESNGVSYCRKDEP |
| 239 | KPV239 | AICTGADMPCAACCPCCPGTSCKAESNGVSYCRKDEP |
| 240 | KPV240 | AICTGADSPCAACCPCCPGTSCKAESNGVSYCRKDEP |
| 243 | KPV243 | AICTGADRPCGGCCPCCPGTSCKAESNGVSYCRKDEP |
| 244 | KPV244 | AICTGADRPCGACCPCCPGTSCKAESNGVSYCRKDEP |
| 245 | KPV245 | AICTGADRPCAGCCPCCPGTSCKAESNGVSYCRKDEP |
| 246 | KPV246 | AICTGADRPCAACCPCCPGTKCKAESNGVSYCRKDEP |
| 247 | KPV247 | AICTGADRPCAACCPCCPGTRCKAESNGVSYCRKDEP |
| 248 | KPV248 | AICTGADGPCAACCPCCPGTSCKAESNGVSYCRKDEP |
| 249 | KPV249 | AICTGADRPCAACCPCCPGTSCKTESNGVSYCRKD |
| 250 | KPV250 | PICTGADRPCAACCPCCPGTSCKAESNGVSYCRKDEP |
| 251 | KPV251 | EAAICTGADRPCAACCPCCPGTSCKAESNGVSYCRKD |
| 252 | KPV252 | NSAICTGADRPCAACCPCCPGTSCKAESNGVSYCRKDEP |
| 253 | KPV253 | GSAICTGADRPCAACCPCCPGTSCKAESNGVSYCRKDEP |
| 254 | KPV254 | GSAICTGADRPCAACCPCCPGTSCKAESNGVSYCRKD |
| 255 | KPV255 | EAAVCTGADRPCAACCPCCPGTSCKAESNGVSYCRKDEP |
| 256 | KPV256 | GSQYCTGADRPCAACCPCCPGTSCKAESNGVSYCRKA |
| 257 | KPV257 | GICTGADRPCAACCPCCPGTSCKAESNGVWYCRKDEP |
| 258 | KPV258 | AICTGADRPCGACCPCCPGTSCKAESNGVSYCRA |
| 259 | KPV259 | GICTGADRPCGACCPCCPGTSCKAESNGVSYCRKDEP |
| 260 | KPV260 | AICTPADRPCAACCPCCPGTSCKAESNGVSYCRKDEP |
| 263 | KPV263 | AICTGADRPCAACCPCCPGTSCSAESNGVSYCRKDEP |
| 264 | KPV264 | AICTGADRPCAACCPCCPGTSCQAASNGVSYCRKDEP |
| 265 | KPV265 | NAICTGADRPCAACCPCCPGTSCYAESNGVSYCRKDEP |
| 269 | KPV269 | AICTGADRPCAACCPCCPGTSCKAESNGVWYCRKDEP |
| 270 | KPV270 | AICTGADRPCAACCPCCPGTSCKATSNGVSYCRKDEP |
| 274 | KPV274 | GAICTGADRPCAACCPCCPGTSCKAESNGVSYCRKDEP |
| 275 | KPV275 | GAICTGADRPCAACCPCCPGTSCKAESNGVSYCRKD |
| 276 | KPV276 | GSICTGADRPCAACCPCCPGTSCKAESNGVSYCRKDEP |
| 277 | KPV277 | SICTGADRPCAACCPCCPGTSCKAFSNGVSYCRKD |
| 278 | KPV278 | SICTGADRPCAACCPCCPGTSCYAESNGVSYCRKDEP |
| 279 | KPV279 | AICTGADRPCAACCPCCPGTSCYAESNGVSYCRKD |
| 280 | KPV280 | SICTGADRPCAACCPCCPGTSCKAESNGVSWCRKDEP |
| 282 | KPV282 | AICTGADRPCAACCPCCPGTSCKAMSNGVSYCRKDEP |
| 283 | KPV283 | GICTGADRPCAACCPCCPGTSCKAESNGVSYCRKDEP |
| 284 | KPV284 | AICTGADRPCAACCPCCPGTSCKASSNGVSYCRKDEP |
| 285 | KPV285 | LICTGADRPCAACCPCCPGTSCYAESNGVSYCRKD |
| 286 | KPV286 | AICTGADRPCAACCPCCPGTSCKAESNGVSYCRKAEP |
| 287 | KPV287 | AICTGADRPCAACCPCCPGTSCKAESNGVSYCRKEEP |
| 288 | KPV288 | AICTGADRPCAACCPCCPGTSCKAESNGVSYCRKGEP |
| 289 | KPV289 | AICTGADRPCAACCPCCPGTSCKAESNGVSYCRKHEP |
| 290 | KPV290 | AICTGADRPCAACCPCCPGTSCKAESNGVSYCRKIEP |
| 291 | KPV291 | AICTGADRPCAACCPCCPGTSCKAESNGVSYCRKKEP |
| 292 | KPV292 | AICTGADRPCAACCPCCPGTSCKAESNGVSYCRKMEP |
| 294 | KPV294 | AICTGADRPCAACCPCCPGTSCKAESNGVSYCRKQEP |
| 295 | KPV295 | AICTGADRPCAACCPCCPGTSCKAESNGVSYCRKREP |
| 296 | KPV296 | AICTGADRPCAACCPCCPGTSCKAESNGVSYCRKSEP |
| 297 | KPV297 | AICTGADRPCAACCPCCPGTSCKAESNGVSYCRKVEP |
| 299 | KPV299 | AICTGADRPCAACCPCCPGTSCKAESNGVSYCRKYEP |
| 300 | KPV300 | AICTGADRPCAACCPCCPGTSCKQEANGVSLCRKDEP |
| 302 | KPV302 | SICTGADRPCAACCPCCPGTSCQIWSNGVSYCRKDEP |
| 303 | KPV303 | AICTGADRPCAACCPCCPGTSCKYESLGVSYCRKDEP |
| 304 | KPV304 | AICTGADRPCAACCPCCPGTSCTARQNGVPYCRKDEP |
| 305 | KPV305 | AICTGADRPCAACCPCCPGTSCKSTGNGAGYCRKDEP |
| 306 | KPV306 | AICTGADRPCAACCPCCPGTSCKAGSAGAFYCRKDEP |
| 308 | KPV308 | AICTGADRPCAACCPCCPGTSCPDESEGVYYCRKDEP |
| 312 | KPV312 | AICTGADRPCAACCPCCPGTSCKADHWGVPYCRKDEP |
| 313 | KPV313 | SICTGADRPCAACCPCCPGTSCKEEIEGPSYCRKDEP |
| 315 | KPV315 | AICTGADRPCAACCPCCPGTSCKAEDNGGTYCRKDEP |
| 316 | KPV316 | SICTGADRPCAACCPCCPGTSCSFESGGVSYCRKDEP |
| 317 | KPV317 | AICTGADRPCAACCPCCPGTSCKSESNGSYYCRKDEP |
| 318 | KPV318 | AICTGANRPCAACCPCCPGTSCKAESNGVSYCRKDEP |
| 319 | KPV319 | AICTGANRPCAACCPCCPGTSCVAESNGVSYCRKDEP |
| 320 | KPV320 | AICTGADRPCAACCPCCPGTSCKTESNGVSYCRKDEP |
| 321 | KPV321 | AICTGADRPCAACCPCCPGTSCKAESNGVSYCRA |
| 322 | KPV322 | AICTGADRPCAACCPCCPGTSCKAESNGVSYCRKD |
| 323 | KPV323 | ALCTGADRPCAACCPCCPGTSCKAESNGVSYCRKDEP |
| 325 | KPV325 | AICTGADRPCAACCPCCPGTSCQGPESNGVSYCRKDEP |
| 327 | KPV327 | QACTPADQPCAACCPCCSGASCTEQLNENGHTVYYCRA |
| 328 | KPV328 | AICTGADRPCAACCPCCPGTSCQGPESNGVSYCRA |
| 329 | KPV329 | AICTGADRPCAACCPCCPGTSCAGPESNGVSYCRKDEP |
| 331 | KPV331 | AICTGADRPCAACCPCCPGTSCKGPESNGVSYCRKDEP |
| 332 | KPV332 | AICTGADRPCAACCPCCPGTSCNGPESNGVSYCRKDEP |
| 334 | KPV334 | NAICTGADRPCAACCPCCPGTSCQGPESNGVSYCRKDEP |
| 343 | KPV343 | TICTGADRPCAACCPCCPGTSCTEPESNGVSYCRND |
| 344 | KPV344 | AICTGADRPCAACCPCCPGTSCTEPESNGVSYCRND |
| 346 | KPV346 | SICTGADRPCAACCPCCPGTSCHKADSNGLSYCRKDEP |
| 347 | KPV347 | SICTGADRPCAACCPCCPGTSCIKRESNGVSYCRKDEP |
| 348 | KPV348 | AICTGADRPCAACCPCCPGTSCTKRESNGVKYCRKDEP |
| 350 | KPV350 | TICSGGDRPRAACCPCCPGTSSGKARVNGVFYCRKDEP |
| 354 | KPV354 | AICTGADRPCAACCPCCPGTSCTKAESNGISYCRKDEP |
| 355 | KPV355 | SICTGADRPCAACCPCCPGTSCQKDDHNGVGYCRKDEP |
| 358 | KPV358 | AICTGADRPCAACCPCCPGTSCKAESNGVSYCRVDEP |

In some embodiments, the present disclosure comprises, consists essentially of, or consists of, a Kappa Variant Peptide (KPV), or an agriculturally acceptable salt thereof, having pesticidal activity (e.g. insecticidal activity) against one or more insect species, said KPV, or agriculturally acceptable salt thereof, comprises, consists essentially of, or consists of an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences provided in the foregoing Table 1; wherein the KPV comprises at least one amino acid substitution relative to the amino acid sequence set forth in SEQ ID NO: 1, with the proviso that the KPV does not comprise the amino acid sequence of SEQ ID NOs: 1 or 253.

In some embodiments, the present disclosure comprises, consists essentially of, or consists of, a Kappa Variant Peptide (KPV) having pesticidal activity against one or more insect species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences provided in the foregoing Table 1; wherein the KPV comprises at least one amino acid substitution relative to the amino acid sequence set forth in SEQ ID NO: 1, with the proviso that the KPV does not comprise the amino acid sequence of SEQ ID NOs: 1 or 253; and wherein the KPV further comprises 1, 2, 3, 4, or 5 amino acid substitutions that are conservative amino acid substitutions.

In some embodiments, the KPV comprises, consists essentially of, or consists of, an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; wherein the KPV comprises at least one amino acid substitution relative to the amino acid sequence set forth in SEQ ID NO: 1, with the proviso that the KPV does not comprise the amino acid sequence of SEQ ID NOs: 1 or 253.

In some embodiments, the KPV comprises, consists essentially of, or consists of, an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; wherein the KPV comprises at least one amino acid substitution relative to the amino acid sequence set forth in SEQ ID NO: 1, with the proviso that the KPV does not comprise the amino acid sequence of SEQ ID NOs: 1 or 253; and wherein the KPV further comprises 1, 2, 3, 4, or 5 amino acid substitutions that are conservative amino acid substitutions.

In some embodiments, the KPV consists essentially of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; wherein the KPV comprises at least one amino acid substitution relative to the amino acid sequence set forth in SEQ ID NO: 1, with the proviso that the KPV does not comprise the amino acid sequence of SEQ ID NOs: 1 or 253.

In some embodiments, the KPV consists essentially of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the KPV further comprises 1, 2, 3, 4, or 5 amino acid substitutions that are conservative amino acid substitutions.

In some embodiments, the KPV consists of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; wherein the KPV comprises at least one amino acid substitution relative to the amino acid sequence set forth in SEQ ID NO: 1, with the proviso that the KPV does not comprise the amino acid sequence of SEQ ID NOs: 1 or 253.

In some embodiments, the KPV consists of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; wherein the KPV comprises at least one amino acid substitution relative to the amino acid sequence set forth in SEQ ID NO: 1, with the proviso that the KPV does not comprise the amino acid sequence of SEQ ID NOs: 1 or 253; and wherein the KPV further comprises 1, 2, 3, 4, or 5 amino acid substitutions that are conservative amino acid substitutions.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCAACCPCCPGTSCKTESNGVSYCRKD” (SEQ ID NO: 2); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAGVNGVAYCRKDEP” (SEQ ID NO: 3); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEANGVSRCRKDEP” (SEQ ID NO: 4); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAFSNGVSYCRKDEP” (SEQ ID NO: 5); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEWNGVSYCRKDEP” (SEQ ID NO: 6); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAICPCCPGTSCKAESNGVSYCRKDEP” (SEQ ID NO: 7); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGIDRPCAACCPCCPGTSCKAESNGVSYCRKDEP” (SEQ ID NO: 10); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSHCRKDEP” (SEQ ID NO: 11); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSSKAESNGVCYCRKDEP” (SEQ ID NO: 12); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTEADRPCHACCPCCPGTSCKAESNGVRYCRKDEP” (SEQ ID NO: 13); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAICCPCCPGTSCKAESNGVSYCRKDEP” (SEQ ID NO: 16); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICEGADRPCAACCPCCPGTSCKAESNGVSYCRKDEP” (SEQ ID NO: 17); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESRGVSYCRKDEP” (SEQ ID NO: 18); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYGRKDEP” (SEQ ID NO: 19); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKYESNGVSYCRKDEP” (SEQ ID NO: 23); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACNPCCPGTSCKAESNGVSYCRKMEP” (SEQ ID NO: 25); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPYAACCPCCPGTSCKAESNGVSYCRKDEP” (SEQ ID NO: 30); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPKAACCPCCPGTSCKAESNGVSYCRKDEP” (SEQ ID NO: 31); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKNEP” (SEQ ID NO: 35); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTKADRPCAACCPCCPGTSCKAASNGVSYCRKDEP” (SEQ ID NO: 39); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AVCTGADRPCAACCPCCPGTSCKAESNGVSYCRKDEP” (SEQ ID NO: 40); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGSSCKAEGNGVSYCRKDEP” (SEQ ID NO: 45); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEPNGVSYCRRDEP” (SEQ ID NO: 46); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKDVP” (SEQ ID NO: 48); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEPNGVSYCRKDEP” (SEQ ID NO: 50); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSFCRKDEP” (SEQ ID NO: 54); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTDADRPCAACCPCCPGTSCKAESNGVSYCRKDEP” (SEQ ID NO: 55); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AIYTGADRPCAACCPCCPGTSCKAESNGVSNCRKDEP” (SEQ ID NO: 56); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAGCPCCPGTSCKAESNGVSYCRKDEP” (SEQ ID NO: 57); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESVGVSYCRKDEP” (SEQ ID NO: 60); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICRGADRPCAACCPCCPGTSCKAESNGVSYCRKDEP” (SEQ ID NO: 66); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCWAESNGVSYCRKDEP” (SEQ ID NO: 68); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCYPGTSCKAESNGVSYCRKDEP” (SEQ ID NO: 70); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAECPCCPGTSCKAESNGVSYCRKDEP” (SEQ ID NO: 71); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADGPCAACCPCCPGTSCKAESNGVSYCRKDES” (SEQ ID NO: 73); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADSPCAACCPCCPGTTCKAESNGVSYCRKDEP” (SEQ ID NO: 74); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAETNGVSYCRKDEP” (SEQ ID NO: 76); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRGDEP” (SEQ ID NO: 77); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCSACCPCCPGHSCKAESNGVSYCRKDEP” (SEQ ID NO: 78); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPRAACCPCCPGTSCKAESNGVSYCRKDEP” (SEQ ID NO: 79); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRADEP” (SEQ ID NO: 85); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRNDEP” (SEQ ID NO: 102); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGEDRPCAACCPCCPGTSCKAESNGVSYCRKDEP” (SEQ ID NO: 110); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCFAESNGVSYCRKDEP” (SEQ ID NO: 114); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEANGVSYCRKDEP” (SEQ ID NO: 124); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRLDEP” (SEQ ID NO: 126); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPRTSCKAESNGVSYCRKDEP” (SEQ ID NO: 140); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRMDEP” (SEQ ID NO: 146); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGISCKAESNGVSYCRKDEP” (SEQ ID NO: 147); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEKNGVSYCRKDEP” (SEQ ID NO: 149); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “WICTGADRPCAACCPCCPGTSCKAESNGVSYCRKDEP” (SEQ ID NO: 151); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRACAACCPCCPGTSCKAESNGVSYCRKDEP” (SEQ ID NO: 152); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCREDEP” (SEQ ID NO: 157); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGQSCKAESNGVSYCRKDEP” (SEQ ID NO: 158); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRVDEP” (SEQ ID NO: 160); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAHSNGVSYCRKDEP” (SEQ ID NO: 161); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKDES” (SEQ ID NO: 162); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AVCTGADRPCAACCPCCPGTSCKAESNGVWYCRKDEP” (SEQ ID NO: 164); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVYYCRKDEP” (SEQ ID NO: 167); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESLGVSYCRKDEP” (SEQ ID NO: 168); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPPTSCKAESNGVSYCRKDEP” (SEQ ID NO: 170); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCMCCPGTSCKAESNGVSYCRKDEP” (SEQ ID NO: 173); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKWESNGVSYCRKDEP” (SEQ ID NO: 176); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEGNGVSYCRKDEP” (SEQ ID NO: 178); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAWSNGVSYCRKDEP” (SEQ ID NO: 180); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGANRPCAAGCPCCPGTSCKAESNGVSYCRKDEP” (SEQ ID NO: 183); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKDEQ” (SEQ ID NO: 184); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAFCPCCPGTSCKAESNGVSYCRKDEP” (SEQ ID NO: 185); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESWGVSYCRKDEP” (SEQ ID NO: 186); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCQKDEP” (SEQ ID NO: 189); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTTCQAESNGVSYCRKDEP” (SEQ ID NO: 191); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGIDRPCAACCPCCPGTSCKAESNGVSYCRKDKP” (SEQ ID NO: 192); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGAGRPCAACCPCCPGTSCKAESNGVSYCRKDFP” (SEQ ID NO: 195); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AACTGADRPCAACCPCCPGTSCKAESNGVSYCRKDEP” (SEQ ID NO: 199); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AIHTGADRPCAACCPCCPGTSCDAESNGVSYCRKDEP” (SEQ ID NO: 204); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCAAESNGVSYCRKDEP” (SEQ ID NO: 205); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCDAESNGVSYCRKDEP” (SEQ ID NO: 206); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCIAESNGVSYCRKDEP” (SEQ ID NO: 209); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCLAESNGVSYCRKDEP” (SEQ ID NO: 210); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCMAESNGVSYCRKDEP” (SEQ ID NO: 211); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCNAESNGVSYCRKDEP” (SEQ ID NO: 212); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCRAESNGVSYCRKDEP” (SEQ ID NO: 215); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCVAESNGVSYCRKDEP” (SEQ ID NO: 216); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCYAESNGVSYCRKDEP” (SEQ ID NO: 217); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRDDEP” (SEQ ID NO: 218); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRHDEP” (SEQ ID NO: 220); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRIDEP” (SEQ ID NO: 221); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRPDEP” (SEQ ID NO: 222); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRQDEP” (SEQ ID NO: 223); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRSDEP” (SEQ ID NO: 224); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRTDEP” (SEQ ID NO: 225); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “VICTGADRPCAACCPCCPGTSCKAESNGVSYCRKDEP” (SEQ ID NO: 226); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “LICTGADRPCAACCPCCPGTSCKAESNGVSYCRKDEP” (SEQ ID NO: 227); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAGVNGVAYCRKD” (SEQ ID NO: 228); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEANGVSRCRKD” (SEQ ID NO: 229); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEVNGVSYCRKDEP” (SEQ ID NO: 230); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAESNGVSYCRKDEP” (SEQ ID NO: 231); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “DICTGADRPCAACCPCCPGTSCKAESNGVSYCRKDEP” (SEQ ID NO: 232); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSLCRKDEP” (SEQ ID NO: 233); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADKPCAACCPCCPGTSCKAESNGVSYCRKDEP” (SEQ ID NO: 235); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADHPCAACCPCCPGTSCKAESNGVSYCRKDEP” (SEQ ID NO: 236); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADYPCAACCPCCPGTSCKAESNGVSYCRKDEP” (SEQ ID NO: 237); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADLPCAACCPCCPGTSCKAESNGVSYCRKDEP” (SEQ ID NO: 238); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADMPCAACCPCCPGTSCKAESNGVSYCRKDEP” (SEQ ID NO: 239); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADSPCAACCPCCPGTSCKAESNGVSYCRKDEP” (SEQ ID NO: 240); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCGGCCPCCPGTSCKAESNGVSYCRKDEP” (SEQ ID NO: 243); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCGACCPCCPGTSCKAESNGVSYCRKDEP” (SEQ ID NO: 244); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAGCCPCCPGTSCKAESNGVSYCRKDEP” (SEQ ID NO: 245); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTKCKAESNGVSYCRKDEP” (SEQ ID NO: 246); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTRCKAESNGVSYCRKDEP” (SEQ ID NO: 247); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADGPCAACCPCCPGTSCKAESNGVSYCRKDEP” (SEQ ID NO: 248); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKTESNGVSYCRKD” (SEQ ID NO: 249); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “PICTGADRPCAACCPCCPGTSCKAESNGVSYCRKDEP” (SEQ ID NO: 250); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “EAAICTGADRPCAACCPCCPGTSCKAESNGVSYCRKD” (SEQ ID NO: 251); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “NSAICTGADRPCAACCPCCPGTSCKAESNGVSYCRKDEP” (SEQ ID NO: 252); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSAICTGADRPCAACCPCCPGTSCKAESNGVSYCRKDEP” (SEQ ID NO: 253); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSAICTGADRPCAACCPCCPGTSCKAESNGVSYCRKD” (SEQ ID NO: 254); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “EAAVCTGADRPCAACCPCCPGTSCKAESNGVSYCRKDEP” (SEQ ID NO: 255); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSQYCTGADRPCAACCPCCPGTSCKAESNGVSYCRKA” (SEQ ID NO: 256); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCAACCPCCPGTSCKAESNGVWYCRKDEP” (SEQ ID NO: 257); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCGACCPCCPGTSCKAESNGVSYCRA” (SEQ ID NO: 258); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCGACCPCCPGTSCKAESNGVSYCRKDEP” (SEQ ID NO: 259); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTPADRPCAACCPCCPGTSCKAESNGVSYCRKDEP” (SEQ ID NO: 260); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCSAESNGVSYCRKDEP” (SEQ ID NO: 263); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCQAASNGVSYCRKDEP” (SEQ ID NO: 264); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “NAICTGADRPCAACCPCCPGTSCYAESNGVSYCRKDEP” (SEQ ID NO: 265); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVWYCRKDEP” (SEQ ID NO: 269); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKATSNGVSYCRKDEP” (SEQ ID NO: 270); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GAICTGADRPCAACCPCCPGTSCKAESNGVSYCRKDEP” (SEQ ID NO: 274); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GAICTGADRPCAACCPCCPGTSCKAESNGVSYCRKD” (SEQ ID NO: 275); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSICTGADRPCAACCPCCPGTSCKAESNGVSYCRKDEP” (SEQ ID NO: 276); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAFSNGVSYCRKD” (SEQ ID NO: 277); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCYAESNGVSYCRKDEP” (SEQ ID NO: 278); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCYAESNGVSYCRKD” (SEQ ID NO: 279); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAESNGVSWCRKDEP” (SEQ ID NO: 280); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAMSNGVSYCRKDEP” (SEQ ID NO: 282); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCAACCPCCPGTSCKAESNGVSYCRKDEP” (SEQ ID NO: 283); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKASSNGVSYCRKDEP” (SEQ ID NO: 284); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “LICTGADRPCAACCPCCPGTSCYAESNGVSYCRKD” (SEQ ID NO: 285); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKAEP” (SEQ ID NO: 286); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKEEP” (SEQ ID NO: 287); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKGEP” (SEQ ID NO: 288); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKHEP” (SEQ ID NO: 289); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKIEP” (SEQ ID NO: 290); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKKEP” (SEQ ID NO: 291); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKMEP” (SEQ ID NO: 292); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKQEP” (SEQ ID NO: 294); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKREP” (SEQ ID NO: 295); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKSEP” (SEQ ID NO: 296); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKVEP” (SEQ ID NO: 297); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKYEP” (SEQ ID NO: 299); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKQEANGVSLCRKDEP” (SEQ ID NO: 300); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCQIWSNGVSYCRKDEP” (SEQ ID NO: 302); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKYESLGVSYCRKDEP” (SEQ ID NO: 303); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTARQNGVPYCRKDEP” (SEQ ID NO: 304); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKSTGNGAGYCRKDEP” (SEQ ID NO: 305); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAGSAGAFYCRKDEP” (SEQ ID NO: 306); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCPDESEGVYYCRKDEP” (SEQ ID NO: 308); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKADHWGVPYCRKDEP” (SEQ ID NO: 312); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKEEIEGPSYCRKDEP” (SEQ ID NO: 313); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEDNGGTYCRKDEP” (SEQ ID NO: 315); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCSFESGGVSYCRKDEP” (SEQ ID NO: 316); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKSESNGSYYCRKDEP” (SEQ ID NO: 317); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGANRPCAACCPCCPGTSCKAESNGVSYCRKDEP” (SEQ ID NO: 318); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGANRPCAACCPCCPGTSCVAESNGVSYCRKDEP” (SEQ ID NO: 319); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKTESNGVSYCRKDEP” (SEQ ID NO: 320); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRA” (SEQ ID NO: 321); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKD” (SEQ ID NO: 322); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “ALCTGADRPCAACCPCCPGTSCKAESNGVSYCRKDEP” (SEQ ID NO: 323); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCQGPESNGVSYCRKDEP” (SEQ ID NO: 325); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “QACTPADQPCAACCPCCSGASCTEQLNENGHTVYYCRA” (SEQ ID NO: 327); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCQGPESNGVSYCRA” (SEQ ID NO: 328); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCAGPESNGVSYCRKDEP” (SEQ ID NO: 329); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKGPESNGVSYCRKDEP” (SEQ ID NO: 331); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCNGPESNGVSYCRKDEP” (SEQ ID NO: 332); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “NAICTGADRPCAACCPCCPGTSCQGPESNGVSYCRKDEP” (SEQ ID NO: 334); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “TICTGADRPCAACCPCCPGTSCTEPESNGVSYCRND” (SEQ ID NO: 343); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTEPESNGVSYCRND” (SEQ ID NO: 344); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCHKADSNGLSYCRKDEP” (SEQ ID NO: 346); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCIKRESNGVSYCRKDEP” (SEQ ID NO: 347); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTKRESNGVKYCRKDEP” (SEQ ID NO: 348); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “TICSGGDRPRAACCPCCPGTSSGKARVNGVFYCRKDEP” (SEQ ID NO: 350); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTKAESNGISYCRKDEP” (SEQ ID NO: 354); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCQKDDHNGVGYCRKDEP” (SEQ ID NO: 355); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a KPV of the present disclosure can comprise, consist essentially of, or consist of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRVDEP” (SEQ ID NO: 358); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, the engineered, non-naturally occurring pesticidal KPV, or an agriculturally acceptable salt thereof of the present disclosure do not comprise any: mutations, amino acid substitutions, amino acid additions, or amino acid deletions. In some embodiments, the engineered, non-naturally occurring pesticidal KPV, or an agriculturally acceptable salt thereof comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence of any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, and does not comprise any: mutations, amino acid substitutions, amino acid additions, or amino acid deletions relative to the amino acid sequence of any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In addition, the present disclosure describes a KPV having an amino acid sequence that is at least 80%, 85%, 90%, 90%, 95%, 96%, 97%, 98%, 99% or 100% identical to an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358. In some embodiments, an illustrative KPV includes a peptide that has 1, 2, 3, 4, 5, 6, or 7 conservative amino acid substitutions at any position along the entire length of the peptide of any one peptide sequence as set forth in SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a Kappa Peptide Variant (KPV), or an agriculturally acceptable salt thereof, having pesticidal activity against one or more insect species, comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (II): X₁—I—C-T-G-A-X₂—R—P—C-A-A-C—C—P—C—C—P-G-T-S—C—X₃—X₄—X₅—X₆—N-G-V-X₇—X₈—C—R—X₉—X₁₀—X₁₁—X₁₂; wherein the KPV comprises at least one amino acid substitution relative to the amino acid sequence set forth in SEQ ID NO: 1; X₁ is A, G, V, L, or S; X₂ is D or G; X₃ is K, I, L, Q, or Y; X₄ is A or T; X₅ is E, F, M, or A; X₆ is S or A; X₇ is S or W; X₈ is Y or R; X₉ is K, E, T, or A; X₁₀ is D, A, G, H, K, M, Q, S, V, or absent; X₁₁ is E, F or absent; X₁₂ is P, S or absent; or an agriculturally acceptable salt thereof, with the proviso that the KPV does not comprise the amino acid sequence of SEQ ID NOs: 1 or 253.

In some embodiments, a Kappa Peptide Variant (KPV), or an agriculturally acceptable salt thereof, having pesticidal activity against one or more insect species, comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (II): X₁—I—C-T-G-A-X₂—R—P—C-A-A-C—C—P—C—C—P-G-T-S—C—X₃—X₄—X₅—X₆—N-G-V-X₇—X₈—C—R—X₉—X₁₀—X₁₁—X₁₂; wherein the KPV comprises at least one amino acid substitution relative to the amino acid sequence set forth in SEQ ID NO: 1; X₁ is A, G, V, L, or S; X₂ is D or G; X₃ is K, I, L, Q, or Y; X₄ is A or T; X₅ is E, F, M, or A; X₆ is S or A; X₇ is S or W; X₈ is Y or R; X₉ is K, E, T, or A; X₁₀ is D, A, G, H, K, M, Q, S, V, or absent; X₁₁ is E, F or absent; X₁₂ is P, S or absent; or an agriculturally acceptable salt thereof, with the proviso that the KPV does not comprise the amino acid sequence of SEQ ID NOs: 1 or 253; wherein the KPV further comprises 1, 2, 3, 4, or 5 amino acid substitutions that are conservative amino acid substitutions.

In some embodiments, a KPV of the present disclosure can comprise, consist essentially of, or consist of, a homopolymer or heteropolymer of two or more KPVs, wherein the amino acid sequence of each KPV is the same or different.

In some embodiments, a KPV of the present disclosure can comprise, consist essentially of, or consist of, a KPV that is a fused protein comprising two or more KPVs separated by a cleavable or non-cleavable linker, and wherein the amino acid sequence of each KPV may be the same or different.

In some embodiments, the linker is a cleavable linker.

In some embodiments, the linker has an amino acid sequence as set forth in any one of SEQ ID NOs: 360-369.

In some embodiments, the linker is cleavable inside at least one of (i) the gut or hemolymph of an insect, and (ii) cleavable inside the gut of a mammal.

Detailed methods concerning linkers are described below.

### Polynucleotides Encoding KPVs

In some embodiments, the present disclosure comprises, consists essentially of, or consists of, a polynucleotide operable to encode a Kappa Peptide Variant (KPV).

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253.

In some embodiments, a polynucleotide is operable to encode a KPV that comprises, consists essentially of, or consists of, an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or a complementary nucleotide sequence thereof.

Nucleotide sequence homologs, e.g., KPVs encoded by polynucleotides that hybridize to each or any of the sequences disclosed in this application under stringent hybridization conditions, are also an embodiment of the present disclosure. The present disclosure also provides a method for detecting a first polynucleotide that hybridizes to a second polynucleotide, wherein the first polynucleotide (or its reverse complement sequence) encodes a KPV or fragment thereof, and hybridizes to the second polynucleotide. In such case, the second polynucleotide can be any of the polynucleotides operable to encode a KPV of the present disclosure, under stringent hybridization conditions.

In some embodiments, a polynucleotide of the present disclosure is operable to encode a Kappa Peptide Variant (KPV) having pesticidal activity against one or more insect species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences provided in the foregoing Table 1; wherein the KPV comprises at least one amino acid substitution relative to the amino acid sequence set forth in SEQ ID NO: 1, with the proviso that the KPV does not comprise the amino acid sequence of SEQ ID NOs: 1 or 253.

In some embodiments, a polynucleotide of the present disclosure is operable to encode a KPV that comprises an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or a complementary nucleotide sequence thereof.

In some embodiments, a polynucleotide of the present disclosure is operable to encode a KPV that consists essentially of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or a complementary nucleotide sequence thereof.

In some embodiments, a polynucleotide of the present disclosure is operable to encode a KPV that consists of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or a complementary nucleotide sequence thereof.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCAACCPCCPGTSCKTESNGVSYCRKD” (SEQ ID NO: 2); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAGVNGVAYCRKDEP” (SEQ ID NO: 3); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEANGVSRCRKDEP” (SEQ ID NO: 4); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAFSNGVSYCRKDEP” (SEQ ID NO: 5); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEWNGVSYCRKDEP” (SEQ ID NO: 6); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAICPCCPGTSCKAESNGVSYCRKDEP” (SEQ ID NO: 7); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGIDRPCAACCPCCPGTSCKAESNGVSYCRKDEP” (SEQ ID NO: 10); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSHCRKDEP” (SEQ ID NO: 11); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSSKAESNGVCYCRKDEP” (SEQ ID NO: 12); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTEADRPCHACCPCCPGTSCKAESNGVRYCRKDEP” (SEQ ID NO: 13); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAICCPCCPGTSCKAESNGVSYCRKDEP” (SEQ ID NO: 16); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICEGADRPCAACCPCCPGTSCKAESNGVSYCRKDEP” (SEQ ID NO: 17); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESRGVSYCRKDEP” (SEQ ID NO: 18); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYGRKDEP” (SEQ ID NO: 19); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKYESNGVSYCRKDEP” (SEQ ID NO: 23); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACNPCCPGTSCKAESNGVSYCRKMEP” (SEQ ID NO: 25); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPYAACCPCCPGTSCKAESNGVSYCRKDEP” (SEQ ID NO: 30); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPKAACCPCCPGTSCKAESNGVSYCRKDEP” (SEQ ID NO: 31); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKNEP” (SEQ ID NO: 35); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTKADRPCAACCPCCPGTSCKAASNGVSYCRKDEP” (SEQ ID NO: 39); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AVCTGADRPCAACCPCCPGTSCKAESNGVSYCRKDEP” (SEQ ID NO: 40); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGSSCKAEGNGVSYCRKDEP” (SEQ ID NO: 45); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEPNGVSYCRRDEP” (SEQ ID NO: 46); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKDVP” (SEQ ID NO: 48); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEPNGVSYCRKDEP” (SEQ ID NO: 50); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSFCRKDEP” (SEQ ID NO: 54); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTDADRPCAACCPCCPGTSCKAESNGVSYCRKDEP” (SEQ ID NO: 55); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AIYTGADRPCAACCPCCPGTSCKAESNGVSNCRKDEP” (SEQ ID NO: 56); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAGCPCCPGTSCKAESNGVSYCRKDEP” (SEQ ID NO: 57); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESVGVSYCRKDEP” (SEQ ID NO: 60); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICRGADRPCAACCPCCPGTSCKAESNGVSYCRKDEP” (SEQ ID NO: 66); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCWAESNGVSYCRKDEP” (SEQ ID NO: 68); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCYPGTSCKAESNGVSYCRKDEP” (SEQ ID NO: 70); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAECPCCPGTSCKAESNGVSYCRKDEP” (SEQ ID NO: 71); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADGPCAACCPCCPGTSCKAESNGVSYCRKDES” (SEQ ID NO: 73); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADSPCAACCPCCPGTTCKAESNGVSYCRKDEP” (SEQ ID NO: 74); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAETNGVSYCRKDEP” (SEQ ID NO: 76); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRGDEP” (SEQ ID NO: 77); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCSACCPCCPGHSCKAESNGVSYCRKDEP” (SEQ ID NO: 78); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPRAACCPCCPGTSCKAESNGVSYCRKDEP” (SEQ ID NO: 79); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRADEP” (SEQ ID NO: 85); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRNDEP” (SEQ ID NO: 102); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGEDRPCAACCPCCPGTSCKAESNGVSYCRKDEP” (SEQ ID NO: 110); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCFAESNGVSYCRKDEP” (SEQ ID NO: 114); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEANGVSYCRKDEP” (SEQ ID NO: 124); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRLDEP” (SEQ ID NO: 126); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPRTSCKAESNGVSYCRKDEP” (SEQ ID NO: 140); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRMDEP” (SEQ ID NO: 146); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGISCKAESNGVSYCRKDEP” (SEQ ID NO: 147); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEKNGVSYCRKDEP” (SEQ ID NO: 149); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “WICTGADRPCAACCPCCPGTSCKAESNGVSYCRKDEP” (SEQ ID NO: 151); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRACAACCPCCPGTSCKAESNGVSYCRKDEP” (SEQ ID NO: 152); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCREDEP” (SEQ ID NO: 157); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGQSCKAESNGVSYCRKDEP” (SEQ ID NO: 158); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRVDEP” (SEQ ID NO: 160); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAHSNGVSYCRKDEP” (SEQ ID NO: 161); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKDES” (SEQ ID NO: 162); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AVCTGADRPCAACCPCCPGTSCKAESNGVWYCRKDEP” (SEQ ID NO: 164); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVYYCRKDEP” (SEQ ID NO: 167); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESLGVSYCRKDEP” (SEQ ID NO: 168); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPPTSCKAESNGVSYCRKDEP” (SEQ ID NO: 170); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCMCCPGTSCKAESNGVSYCRKDEP” (SEQ ID NO: 173); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKWESNGVSYCRKDEP” (SEQ ID NO: 176); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEGNGVSYCRKDEP” (SEQ ID NO: 178); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAWSNGVSYCRKDEP” (SEQ ID NO: 180); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGANRPCAAGCPCCPGTSCKAESNGVSYCRKDEP” (SEQ ID NO: 183); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKDEQ” (SEQ ID NO: 184); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAFCPCCPGTSCKAESNGVSYCRKDEP” (SEQ ID NO: 185); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESWGVSYCRKDEP” (SEQ ID NO: 186); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCQKDEP” (SEQ ID NO: 189); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTTCQAESNGVSYCRKDEP” (SEQ ID NO: 191); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGIDRPCAACCPCCPGTSCKAESNGVSYCRKDKP” (SEQ ID NO: 192); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGAGRPCAACCPCCPGTSCKAESNGVSYCRKDFP” (SEQ ID NO: 195); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AACTGADRPCAACCPCCPGTSCKAESNGVSYCRKDEP” (SEQ ID NO: 199); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AIHTGADRPCAACCPCCPGTSCDAESNGVSYCRKDEP” (SEQ ID NO: 204); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCAAESNGVSYCRKDEP” (SEQ ID NO: 205); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCDAESNGVSYCRKDEP” (SEQ ID NO: 206); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCIAESNGVSYCRKDEP” (SEQ ID NO: 209); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCLAESNGVSYCRKDEP” (SEQ ID NO: 210); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCMAESNGVSYCRKDEP” (SEQ ID NO: 211); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCNAESNGVSYCRKDEP” (SEQ ID NO: 212); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCRAESNGVSYCRKDEP” (SEQ ID NO: 215); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCVAESNGVSYCRKDEP” (SEQ ID NO: 216); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCYAESNGVSYCRKDEP” (SEQ ID NO: 217); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRDDEP” (SEQ ID NO: 218); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRHDEP” (SEQ ID NO: 220); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRIDEP” (SEQ ID NO: 221); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRPDEP” (SEQ ID NO: 222); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRQDEP” (SEQ ID NO: 223); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRSDEP” (SEQ ID NO: 224); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRTDEP” (SEQ ID NO: 225); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “VICTGADRPCAACCPCCPGTSCKAESNGVSYCRKDEP” (SEQ ID NO: 226); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “LICTGADRPCAACCPCCPGTSCKAESNGVSYCRKDEP” (SEQ ID NO: 227); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAGVNGVAYCRKD” (SEQ ID NO: 228); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEANGVSRCRKD” (SEQ ID NO: 229); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253. In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEVNGVSYCRKDEP” (SEQ ID NO: 230); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAESNGVSYCRKDEP” (SEQ ID NO: 231); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “DICTGADRPCAACCPCCPGTSCKAESNGVSYCRKDEP” (SEQ ID NO: 232); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSLCRKDEP” (SEQ ID NO: 233); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADKPCAACCPCCPGTSCKAESNGVSYCRKDEP” (SEQ ID NO: 235); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADHPCAACCPCCPGTSCKAESNGVSYCRKDEP” (SEQ ID NO: 236); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADYPCAACCPCCPGTSCKAESNGVSYCRKDEP” (SEQ ID NO: 237); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADLPCAACCPCCPGTSCKAESNGVSYCRKDEP” (SEQ ID NO: 238); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADMPCAACCPCCPGTSCKAESNGVSYCRKDEP” (SEQ ID NO: 239); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADSPCAACCPCCPGTSCKAESNGVSYCRKDEP” (SEQ ID NO: 240); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCGGCCPCCPGTSCKAESNGVSYCRKDEP” (SEQ ID NO: 243); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCGACCPCCPGTSCKAESNGVSYCRKDEP” (SEQ ID NO: 244); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAGCCPCCPGTSCKAESNGVSYCRKDEP” (SEQ ID NO: 245); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTKCKAESNGVSYCRKDEP” (SEQ ID NO: 246); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTRCKAESNGVSYCRKDEP” (SEQ ID NO: 247); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADGPCAACCPCCPGTSCKAESNGVSYCRKDEP” (SEQ ID NO: 248); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKTESNGVSYCRKD” (SEQ ID NO: 249); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “PICTGADRPCAACCPCCPGTSCKAESNGVSYCRKDEP” (SEQ ID NO: 250); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “EAAICTGADRPCAACCPCCPGTSCKAESNGVSYCRKD” (SEQ ID NO: 251); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “NSAICTGADRPCAACCPCCPGTSCKAESNGVSYCRKDEP” (SEQ ID NO: 252); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSAICTGADRPCAACCPCCPGTSCKAESNGVSYCRKDEP” (SEQ ID NO: 253); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSAICTGADRPCAACCPCCPGTSCKAESNGVSYCRKD” (SEQ ID NO: 254); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “EAAVCTGADRPCAACCPCCPGTSCKAESNGVSYCRKDEP” (SEQ ID NO: 255); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSQYCTGADRPCAACCPCCPGTSCKAESNGVSYCRKA” (SEQ ID NO: 256); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCAACCPCCPGTSCKAESNGVWYCRKDEP” (SEQ ID NO: 257); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCGACCPCCPGTSCKAESNGVSYCRA” (SEQ ID NO: 258); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCGACCPCCPGTSCKAESNGVSYCRKDEP” (SEQ ID NO: 259); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTPADRPCAACCPCCPGTSCKAESNGVSYCRKDEP” (SEQ ID NO: 260); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCSAESNGVSYCRKDEP” (SEQ ID NO: 263); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCQAASNGVSYCRKDEP” (SEQ ID NO: 264); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “NAICTGADRPCAACCPCCPGTSCYAESNGVSYCRKDEP” (SEQ ID NO: 265); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVWYCRKDEP” (SEQ ID NO: 269); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKATSNGVSYCRKDEP” (SEQ ID NO: 270); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GAICTGADRPCAACCPCCPGTSCKAESNGVSYCRKDEP” (SEQ ID NO: 274); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GAICTGADRPCAACCPCCPGTSCKAESNGVSYCRKD” (SEQ ID NO: 275); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSICTGADRPCAACCPCCPGTSCKAESNGVSYCRKDEP” (SEQ ID NO: 276); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAFSNGVSYCRKD” (SEQ ID NO: 277); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCYAESNGVSYCRKDEP” (SEQ ID NO: 278); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCYAESNGVSYCRKD” (SEQ ID NO: 279); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAESNGVSWCRKDEP” (SEQ ID NO: 280); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAMSNGVSYCRKDEP” (SEQ ID NO: 282); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCAACCPCCPGTSCKAESNGVSYCRKDEP” (SEQ ID NO: 283); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKASSNGVSYCRKDEP” (SEQ ID NO: 284); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “LICTGADRPCAACCPCCPGTSCYAESNGVSYCRKD” (SEQ ID NO: 285); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253. In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKAEP” (SEQ ID NO: 286); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKEEP” (SEQ ID NO: 287); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKGEP” (SEQ ID NO: 288); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKHEP” (SEQ ID NO: 289); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKIEP” (SEQ ID NO: 290); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKKEP” (SEQ ID NO: 291); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKMEP” (SEQ ID NO: 292); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKQEP” (SEQ ID NO: 294); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKREP” (SEQ ID NO: 295); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKSEP” (SEQ ID NO: 296); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKVEP” (SEQ ID NO: 297); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKYEP” (SEQ ID NO: 299); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKQEANGVSLCRKDEP” (SEQ ID NO: 300); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCQIWSNGVSYCRKDEP” (SEQ ID NO: 302); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKYESLGVSYCRKDEP” (SEQ ID NO: 303); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTARQNGVPYCRKDEP” (SEQ ID NO: 304); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKSTGNGAGYCRKDEP” (SEQ ID NO: 305); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAGSAGAFYCRKDEP” (SEQ ID NO: 306); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCPDESEGVYYCRKDEP” (SEQ ID NO: 308); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKADHWGVPYCRKDEP” (SEQ ID NO: 312); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKEEIEGPSYCRKDEP” (SEQ ID NO: 313); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEDNGGTYCRKDEP” (SEQ ID NO: 315); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCSFESGGVSYCRKDEP” (SEQ ID NO: 316); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKSESNGSYYCRKDEP” (SEQ ID NO: 317); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGANRPCAACCPCCPGTSCKAESNGVSYCRKDEP” (SEQ ID NO: 318); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGANRPCAACCPCCPGTSCVAESNGVSYCRKDEP” (SEQ ID NO: 319); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKTESNGVSYCRKDEP” (SEQ ID NO: 320); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRA” (SEQ ID NO: 321); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKD” (SEQ ID NO: 322); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “ALCTGADRPCAACCPCCPGTSCKAESNGVSYCRKDEP” (SEQ ID NO: 323); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCQGPESNGVSYCRKDEP” (SEQ ID NO: 325); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “QACTPADQPCAACCPCCSGASCTEQLNENGHTVYYCRA” (SEQ ID NO: 327); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCQGPESNGVSYCRA” (SEQ ID NO: 328); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCAGPESNGVSYCRKDEP” (SEQ ID NO: 329); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKGPESNGVSYCRKDEP” (SEQ ID NO: 331); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCNGPESNGVSYCRKDEP” (SEQ ID NO: 332); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “NAICTGADRPCAACCPCCPGTSCQGPESNGVSYCRKDEP” (SEQ ID NO: 334); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “TICTGADRPCAACCPCCPGTSCTEPESNGVSYCRND” (SEQ ID NO: 343); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTEPESNGVSYCRND” (SEQ ID NO: 344); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCHKADSNGLSYCRKDEP” (SEQ ID NO: 346); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCIKRESNGVSYCRKDEP” (SEQ ID NO: 347); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTKRESNGVKYCRKDEP” (SEQ ID NO: 348); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “TICSGGDRPRAACCPCCPGTSSGKARVNGVFYCRKDEP” (SEQ ID NO: 350); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTKAESNGISYCRKDEP” (SEQ ID NO: 354); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCQKDDHNGVGYCRKDEP” (SEQ ID NO: 355); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRVDEP” (SEQ ID NO: 358); wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (II): X₁—I-C-T-G-A-X₂—R—P—C-A-A-C—C—P—C—C—P-G-T-S—C—X₃—X₄—X₅—X₆—N-G-V-X₇—X₈—C—R—X₉—X₁₀—X₁₁—X₁₂; wherein the KPV comprises at least one amino acid substitution relative to the amino acid sequence set forth in SEQ ID NO: 1; X₁ is A, G, V, L, or S; X₂ is D or G; X₃ is K, I, L, Q, or Y; X₄ is A or T; X₅ is E, F, M, or A; X₆ is S or A; X₇ is S or W; X₈ is Y or R; X₉ is K, E, T, or A; X₁₀ is D, A, G, H, K, M, Q, S, V, or absent; X₁₁ is E, F or absent; X₁₂ is P, S or absent; or a complementary nucleotide sequence thereof, with the proviso that the KPV does not comprise the amino acid sequence of SEQ ID NOs: 1 or 253.

In some embodiments, a polynucleotide of the present disclosure is operable to encode a KPV that comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (II): X₁—I—C-T-G-A-X₂—R—P—C-A-A-C—C—P—C—C—P-G-T-S—C—X₃—X₄—X₅—X₆—N-G-V-X₇—X₈—C—R—X₉—X₁₀—X₁₁—X₁₂; wherein the KPV comprises at least one amino acid substitution relative to the amino acid sequence set forth in SEQ ID NO: 1; X₁ is A, G, V, L, or S; X₂ is D or G; X₃ is K, I, L, Q, or Y; X₄ is A or T; X₅ is E, F, M, or A; X₆ is S or A; X₇ is S or W; X₈ is Y or R; X₉ is K, E, T, or A; X₁₀ is D, A, G, H, K, M, Q, S, V, or absent; X₁₁ is E, F or absent; X₁₂ is P, S or absent; or a complementary nucleotide sequence thereof, with the proviso that the KPV does not comprise the amino acid sequence of SEQ ID NOs: 1 or 253; wherein the KPV further comprises 1, 2, 3, 4, or 5 amino acid substitutions that are conservative amino acid substitutions.

### Stringent Hybridization

In some embodiments, a polynucleotide of the present disclosure can stringently hybridize to a polynucleotide encoding a KPV, or a complementary sequence thereof, said KPV comprising, consisting essentially of, or consisting of, an amino acid sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253.

In some embodiments, a polynucleotide of the present disclosure can stringently hybridize to a polynucleotide encoding a KPV, or a complementary sequence thereof, said KPV comprising, consisting essentially of, or consisting of, an amino acid sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (II): X₁—I—C-T-G-A-X₂—R—P—C-A-A-C—C—P—C—C—P-G-T-S—C—X₃—X₄—X₅—X₆—N-G-V-X₇—X₈—C—R—X₉—X₁₀—X₁₁—X₁₂; wherein the KPV comprises at least one amino acid substitution relative to the amino acid sequence set forth in SEQ ID NO: 1; X₁ is A, G, V, L, or S; X₂ is D or G; X₃ is K, I, L, Q, or Y; X₄ is A or T; X₅ is E, F, M, or A; X₆ is S or A; X₇ is S or W; X₈ is Y or R; X₉ is K, E, T, or A; X₁₀ is D, A, G, H, K, M, Q, S, V, or absent; X₁₁ is E, F or absent; X₁₂ is P, S or absent; with the proviso that the KPV does not comprise the amino acid sequence of SEQ ID NOs: 1 or 253.

In some embodiments, a polynucleotide of the present disclosure can stringently hybridize to a polynucleotide encoding a KPV, or a complementary sequence thereof, said KPV comprising, consisting essentially of, or consisting of, an amino acid sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (II): X₁—I—C-T-G-A-X₂—R—P—C-A-A-C—C—P—C—C—P-G-T-S—C—X₃—X₄—X₅—X₆—N-G-V-X₇—X₈—C—R—X₉—X₁₀—X₁₁—X₁₂; wherein the KPV comprises at least one amino acid substitution relative to the amino acid sequence set forth in SEQ ID NO: 1; X₁ is A, G, V, L, or S; X₂ is D or G; X₃ is K, I, L, Q, or Y; X₄ is A or T; X₅ is E, F, M, or A; X₆ is S or A; X₇ is S or W; X₈ is Y or R; X₉ is K, E, T, or A; X₁₀ is D, A, G, H, K, M, Q, S, V, or absent; X₁₁ is E, F or absent; X₁₂ is P, S or absent; with the proviso that the KPV does not comprise the amino acid sequence of SEQ ID NOs: 1 or 253; wherein the KPV further comprises 1, 2, 3, 4, or 5 amino acid substitutions that are conservative amino acid substitutions.

In some embodiments, a polynucleotide of the present disclosure comprises, consists essentially of, or consists of, a polynucleotide segment encoding a KPV or fragment thereof, wherein: (a) said KPV comprises an amino acid sequence set forth in SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; or (b) said KPV comprises an amino acid sequence having at least 80%, or 85%, or 90%, or 95%, or 98%, or 99%, or about 100% amino acid sequence identity to SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In one embodiment, the present disclosure provides a method comprising contacting a sample of nucleic acids with a nucleic acid probe that hybridizes under stringent hybridization conditions with a polynucleotide comprising a polynucleotide segment encoding a KPV or fragment thereof as provided herein, and does not hybridize under such hybridization conditions with a polynucleotide that does not comprise the segment, wherein the probe is homologous or complementary to a polynucleotide encoding any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or a polynucleotide encoding a KPV comprising an amino acid sequence having at least 80%, or 85%, or 90%, or 95%, or 98%, or 99%, or about 100% amino acid sequence identity to SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358. The method may further comprise (a) subjecting the sample and probe to stringent hybridization conditions; and (b) detecting hybridization of the probe with polynucleotide of the sample.

### KPV-Pesticidal Proteins

In some embodiments, a KPV-pesticidal protein can be any protein, peptide, polypeptide, amino acid sequence, configuration, construct, or arrangement, comprising: (1) at least one KPV, or two or more KPVs; and (2) one or more additional non-KPV peptides, polypeptides, or proteins. For example, in some embodiments, these additional non-KPV peptides, polypeptides, or proteins may have the ability to increase the mortality and/or inhibit the growth of insects exposed to the KPV-pesticidal protein, relative to the KPV alone; increase the expression of the KPV-pesticidal protein, e.g., in a host cell; and/or affect the post-translational processing of the KPV-pesticidal protein.

In some embodiments, a KPV-pesticidal protein can be a polymer comprising two or more KPVs. In yet other embodiments, a KPV-pesticidal protein can be a polymer comprising two or more KPVs, wherein the KPVs are operably linked via a linker peptide, e.g., a cleavable and/or a non-cleavable linker. Here, the linker peptide falls under the category of the additional non-KPV peptide described above.

In some embodiments, a KPV-pesticidal protein can refer to a one or more KPVs operably linked with one or more proteins such as a stabilizing domain (STA); an endoplasmic reticulum signaling protein (ERSP); an insect cleavable or insect non-cleavable linker (L); and/or any other combination thereof.

In some embodiments, a KPV-pesticidal protein can be a polymer of amino acids that, when properly folded or in its most natural thermodynamic state, exerts an pesticidal activity against one or more insects.

In some embodiments, a KPV-pesticidal protein can be a polymer comprising two or more KPVs that are different. In other embodiments, an pesticidal protein can be a polymer of two or more KPVs that are the same.

In yet other embodiments, a KPV-pesticidal protein can comprise one or more KPVs, and one or more peptides, polypeptides, or proteins, that may assist in the KPV-pesticidal protein's folding.

In some embodiments, a KPV-pesticidal protein can comprise one or more KPVs, and one or more peptides, polypeptides, or proteins, wherein the one or more peptides, polypeptides, or proteins are protein tags that help stability or solubility. In other embodiments, the peptides, polypeptides, or proteins can be protein tags that aid in affinity purification.

In some embodiments, a KPV-pesticidal protein can refer to a one or more KPVs operably linked with one or more proteins such as a stabilizing domain (STA); an endoplasmic reticulum signaling protein (ERSP); an insect cleavable or insect non-cleavable linker; one or more heterologous peptides; one or more additional polypeptides; and/or any other combination thereof. In some embodiments, an pesticidal protein can comprise a one or more KPVs as disclosed herein.

In some embodiments, a KPV-pesticidal protein can comprise a KPV homopolymer, e.g., two or more KPV monomers that are the same KPV. In some embodiments, the pesticidal protein can comprise a KPV heteropolymer, e.g., two or more KPV monomers, wherein the KPV monomers are different.

In some embodiments, a KPV-pesticidal protein can comprise, consist essentially of, or consist of one or more KPVs having an amino acid sequence set forth in SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein the KPV comprises at least one amino acid substitution relative to the amino acid sequence set forth in SEQ ID NO: 1, with the proviso that the KPV does not comprise the amino acid sequence of SEQ ID NO: 1; or an agriculturally acceptable salt thereof.

In some embodiments, the KPV-pesticidal protein may comprise a KPV having an amino acid sequence having at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% amino acid sequence identity to of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; wherein the KPV comprises at least one amino acid substitution relative to the amino acid sequence set forth in SEQ ID NO: 1, or an agriculturally acceptable salt thereof; with the proviso that the KPV does not comprise the amino acid sequence of SEQ ID NOs: 1 or 253.

Examples of linkers include, but not limited to, the following sequences: IGER (SEQ ID NO:377), EEKKN, (SEQ ID NO:378), and ETMFKHGL (SEQ ID NO:379), or combinations thereof.

In some embodiments, the linker can be one or more of the following: ALKFLV (SEQ ID NO: 360), ALKLFV (SEQ ID NO: 361), IFVRLR (SEQ ID NO: 362), LFAAPF (SEQ ID NO: 363), ALKFLVGS (SEQ ID NO: 364), ALKLFVGS (SEQ ID NO: 365), IFVRLRGS (SEQ ID NO: 366), LFAAPFGS (SEQ ID NO: 367), LFVRLRGS (SEQ ID NO: 368), and/or LGERGS (SEQ ID NO: 369).

Exemplary methods for the generation of cleavable and non-cleavable linkers can be found in U.S. patent application Ser. No. 15/727,277; and PCT Application No. PCT/US2013/030042, the disclosures of which are incorporated herein by reference in their entireties.

Exemplary ERSPs and STAs and their methods of use are provided in U.S. Pat. No. 9,567,381, the disclosure of which is incorporated herein by reference in its entirety.

Detailed methods concerning ERSPs, STAs, and linkers, are described below.

### Methods for Producing Pesticidal KPVS

In some illustrative embodiments, the engineered, non-naturally occurring pesticidal KPV, or an agriculturally acceptable salt thereof of the present disclosure can be produced and/or obtained via any method well known in the art of peptide synthesis.

### Recombinant Pesticidal KPVs

In some embodiments, the engineered, non-naturally occurring pesticidal KPV of the present disclosure can be produced using recombinant methods.

Obtaining a KPV from a chemically synthesized DNA polynucleotide sequence and/or a wild-type DNA polynucleotide sequence that has been altered via mutagenesis can be achieved by cloning the DNA sequence into an appropriate vector. There are a variety of expression vectors available, host organisms, and cloning strategies known to those having ordinary skill in the art. For example, the vector can be a plasmid, which can introduce a heterologous gene and/or expression cassette into yeast cells to be transcribed and translated. The term “vector” is used to refer to a carrier nucleic acid molecule into which a nucleic acid sequence can be inserted for introduction into a cell where it can be replicated. A vector may contain “vector elements” such as an origin of replication (ORI); a gene that confers antibiotic resistance to allow for selection; multiple cloning sites; a promoter region; a selection marker for non-bacterial transfection; and a primer binding site. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference in their entireties. In addition to encoding a KPV polynucleotide, a vector may encode a targeting molecule. A targeting molecule is one that directs the desired nucleic acid to a particular tissue, cell, or other location.

In some embodiments, the present disclosure comprises, consists essentially of, or consists of, a vector comprising a polynucleotide operable to encode a KPV of the present disclosure.

In some embodiments, an engineered, non-naturally occurring pesticidal KPV of the present disclosure can be created using any known method for producing a peptide or protein. For example, in some embodiments, and without limitation, an engineered, non-naturally occurring pesticidal KPV of the present disclosure can be created using a recombinant expression system, such as yeast expression system or a bacterial expression system. However, those having ordinary skill in the art will recognize that other methods of protein production are available.

In some embodiments, an engineered, non-naturally occurring pesticidal KPV of the present disclosure can be produced using a recombinant expression system.

The recombinant expression of an engineered, non-naturally occurring pesticidal KPV of the present disclosure is practicable in a wide variety of host cells. In some embodiments, the host cell can be any host cell that satisfies the requirements of the end-user. For example, in some embodiments, a user may desire to use one specific type of host cell (e.g., a yeast cell or a bacteria cell) as opposed to another; the preference of a given host cell can range from desired codon usage, availability and cost, among other considerations.

In some embodiments, a vector of the present disclosure comprises a polynucleotide operable to encode a KPV comprising, consisting essentially of, or consisting of, an amino acid sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253.

In some embodiments, a vector of the present disclosure comprises a polynucleotide or complementary sequence thereof, that can stringently hybridize to a polynucleotide or segment thereof operable to encode a KPV, said KPV comprising, consisting essentially of, or consisting of, an amino acid sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253.

In some embodiments, a vector of the present disclosure comprises a polynucleotide operable to encode a KPV, said KPV comprising, consisting essentially of, or consisting of, an amino acid sequence that is at least is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a polynucleotide operable to encode a KPV or a KPV-pesticidal protein, or a complementary nucleotide sequence thereof, can be transformed into a host cell.

In some embodiments, a polynucleotide operable to encode a KPV or a KPV-pesticidal protein, or a complementary nucleotide sequence thereof, can be cloned into a vector, and transformed into a host cell.

In some embodiments, a KPV ORF can be transformed into a host cell. In some embodiments, a KPV ORF can be cloned into a vector (e.g., a plasmid) and subsequently transformed into a host cell.

In addition to a polynucleotide sequence operable to encode a KPV (e.g., a KPV ORF) or a KPV-pesticidal protein, additional DNA segments known as regulatory elements can be cloned into a vector that allow for enhanced expression of the foreign DNA or transgene; examples of such additional DNA segments include (1) promoters, terminators, and/or enhancer elements; (2) an appropriate mRNA stabilizing polyadenylation signal; (3) an internal ribosome entry site (IRES); (4) introns; and (5) post-transcriptional regulatory elements. The combination of a DNA segment of interest (e.g., kpv) with any one of the foregoing cis-acting elements is called an “expression cassette.”

In some embodiments, an expression cassette or KPV expression cassette can contain one or more polynucleotides operable to encode one or more KPVs, and/or one or more KPV-pesticidal proteins.

In some embodiments, an expression cassette or KPV expression cassette can contain one or more polynucleotides operable to encode one or more KPVs, and/or one or more KPV-pesticidal proteins; and, optionally, one or more additional regulatory elements such as: (1) promoters, terminators, and/or enhancer elements; (2) an appropriate mRNA stabilizing polyadenylation signal; (3) an internal ribosome entry site (IRES); (4) introns; and (5) post-transcriptional regulatory elements.

In some embodiments, a single expression cassette can contain one or more of the aforementioned regulatory elements, and a polynucleotide operable to express a KPV. For example, in some embodiments, a KPV expression cassette can comprise polynucleotide operable to encode a KPV, and an α-MF signal; Kex2 site; LAC4 terminator; ADN1 promoter; and an acetamidase (amdS) selection marker—flanked by LAC4 promoters on the 5′-end and 3′-end.

In some embodiments, there can be numerous expression cassettes cloned into a vector. For example, in some embodiments, there can be a first expression cassette comprising a polynucleotide operable to express a KPV. In alternative embodiments, there are two expression cassettes operable to encode a KPV (i.e., a double expression cassette). In other embodiments, there are three expression cassettes operable to encode a KPV (i.e., a triple expression cassette).

In some embodiments, a double expression cassette can be generated by subcloning a second KPV expression cassette into a vector containing a first KPV expression cassette.

In some embodiments, a triple expression cassette can be generated by subcloning a third KPV expression cassette into a vector containing a first and a second KPV expression cassette.

In some embodiments, one, two, three, or more expression cassettes can be cloned into a vector, wherein each expression cassette comprises: (1) a DNA sequence of interest, e.g., a polynucleotide operable to encode a KPV; and one or more of the following: (2) promoters, terminators, and/or enhancer elements; (3) an appropriate mRNA stabilizing polyadenylation signal; (4) an internal ribosome entry site (IRES); (5) introns; and/or (6) post-transcriptional regulatory elements.

In some embodiments, one, two, three, or more expression cassettes can be cloned into a vector, wherein each expression cassette comprises a polynucleotide encoding a KPV, wherein each of the KPVs are the same or different.

In some embodiments, one, two, three, or more expression cassettes can be cloned into a vector, wherein each expression cassette comprises a polynucleotide encoding a KPV ORF, wherein each of the KPV ORFs are the same or different.

Methods of cloning, recombinant expression, the preparation of vectors, and transformation techniques are known in the art. For example, in some embodiments, a KPV polynucleotide can be cloned into a vector (for example, a cloning vector or an expression vector known in the art) using a variety of cloning strategies, and commercial cloning kits and materials readily available to those having ordinary skill in the art, such as the SnapFast; Gateway; TOPO; Gibson; LIC; InFusionHD; or Electra strategies. See Janke et al., A versatile toolbox for PCR-based tagging of yeast genes: new fluorescent proteins, more markers and promoter substitution cassettes. Yeast. 2004 August; 21(11): 947-62; and Adams et al. Methods in Yeast Genetics. Cold Spring Harbor, NY, 1997, the disclosures of which are incorporated herein by reference in their entireties.

In some embodiments, a polynucleotide encoding a KPV or multiple copies of KPVs (either the same or different) can be cloned into a vector such as a plasmid, cosmid, virus (bacteriophage, animal viruses, and plant viruses), and/or artificial chromosome (e.g., YACs).

In some embodiments, a polynucleotide encoding a KPV can be inserted into a vector, for example, a plasmid vector using E. coli as a host, using methods known in the art.

In some embodiments a polynucleotide encoding a KPV (e.g., a KPV ORF), along with other DNA segments together composing a KPV expression cassette can be designed for secretion from host yeast cells. An illustrative method of designing a KPV expression cassette is as follows: the cassette can begin with a signal peptide sequence, followed by a DNA sequence encoding a Kex2 cleavage site (Lysine-Arginine), and subsequently followed by the KPV polynucleotide transgene (KPV ORF), with the addition of glycine-serine codons at the 5′-end, and finally a stop codon at the 3′-end. All these elements will then be expressed to a fusion peptide in yeast cells as a single open reading frame (ORF). An α-mating factor (αMF) signal sequence is most frequently used to facilitate metabolic processing of the recombinant pesticidal peptides through the endogenous secretion pathway of the recombinant yeast, i.e. the expressed fusion peptide will typically enter the Endoplasmic Reticulum, wherein the α-mating factor signal sequence is removed by signal peptidase activity, and then the resulting pro-pesticidal peptide will be trafficked to the Golgi Apparatus, in which the Lysine-Arginine dipeptide mentioned above is completely removed by Kex2 endoprotease, after which the mature, polypeptide (i.e., KPV), is secreted out of the cells.

In some embodiments, polypeptide expression levels in recombinant yeast cells can be enhanced by optimizing the codons based on the specific host yeast species. Naturally occurring frequencies of codons observed in endogenous open reading frames of a given host organism need not necessarily be optimized for high efficiency expression. Furthermore, different yeast species (for example, Kluyveromyces lactis, Pichia pastoris, Saccharomyces cerevisiae, etc.) have different optimal codons for high efficiency expression. Hence, codon optimization should be considered for the KPV expression cassette, including the sequence elements encoding the signal sequence, the Kex2 cleavage site and the KPV, because they are initially translated as one fusion peptide in the recombinant yeast cells.

In some embodiments, a codon-optimized KPV expression cassette can be ligated into a yeast-specific expression vectors for yeast expression. There are many expression vectors available for yeast expression, including episomal vectors and integrative vectors, and they are usually designed for specific yeast strains. One should carefully choose the appropriate expression vector in view of the specific yeast expression system which will be used for the peptide production. In some embodiments, integrative vectors can be used, which integrate into chromosomes of the transformed yeast cells and remain stable through cycles of cell division and proliferation.

In some embodiments, the expression vectors or cloning vectors can contain E. coli elements for DNA preparation in E. coli, for example, E. coli replication origin, antibiotic selection marker, etc. In some embodiments, vectors can contain an array of the sequence elements needed for expression of the transgene of interest, for example, transcriptional promoters, terminators, yeast selection markers, integrative DNA sequences homologous to host yeast DNA, etc. There are many suitable yeast promoters available, including natural and engineered promoters, for example, yeast promoters such as pLAC4, pAOX1, pUPP, pADH1, pTEF, pGal1, etc., and others, can be used in some embodiments.

In some embodiments, selection methods such as acetamide prototrophy selection; zeocin-resistance selection; geneticin-resistance selection; nourseothricin-resistance selection; uracil deficiency selection; and/or other selection methods may be used. For example, in some embodiments, the Aspergillus nidulans amdS gene can be used as selectable marker. Exemplary methods for the use of selectable markers can be found in U.S. Pat. No. 6,548,285 (filed Apr. 3, 1997); U.S. Pat. No. 6,165,715 (filed Jun. 22, 1998); and U.S. Pat. No. 6,110,707 (filed Jan. 17, 1997), the disclosures of which are incorporated herein by reference in their entireties.

In some embodiments, a polynucleotide encoding a KPV can be inserted into other commercially available plasmids and/or vectors that are readily available to those having skill in the art, e.g., plasmids are available from Addgene (a non-profit plasmid repository); GenScript®; Takara®; Qiagen®; and Promega™.

In some embodiments, a polynucleotide operable to encode an engineered, non-naturally occurring pesticidal KPV of the present disclosure as provided in Table 1 (SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358) can be cloned into a vector using a variety of cloning strategies, and commercial cloning kits and materials readily available to those having ordinary skill in the art. For example, a polynucleotide which encodes the engineered, non-naturally occurring pesticidal KPV can be cloned into a vector using such strategies as the SnapFast; Gateway; TOPO; Gibson; LIC; InFusionHD; or Electra strategies. There are numerous commercially available vectors that can be used to produce an engineered, non-naturally occurring pesticidal KPV, or an agriculturally acceptable salt thereof of the present disclosure. See Janke et al., A versatile toolbox for PCR-based tagging of yeast genes: new fluorescent proteins, more markers and promoter substitution cassettes. Yeast. 2004 August; 21(11): 947-62; see also, Adams et al. Methods in Yeast Genetics. Cold Spring Harbor, NY, 1997, the disclosures of which are incorporated herein by reference in its entireties.

In some embodiments, a polynucleotide encoding an engineered, non-naturally occurring pesticidal KPV of the present disclosure can be cloned into a vector such as a plasmid, cosmid, virus (bacteriophage, animal viruses, and plant viruses), and/or artificial chromosome (e.g., YACs).

In some embodiments, a polynucleotide encoding an engineered, non-naturally occurring pesticidal KPV of the present disclosure can be inserted into other commercially available plasmids and/or vectors that are readily available to those having skill in the art, e.g., plasmids are available from Addgene (a non-profit plasmid repository); GenScript®; Takara®; Qiagen®; and Promega™.

Additional exemplary methods of producing recombinant peptides are provided in PCT Application Nos. PCT/US2013/030042, PCT/US2017/055596, PCT/US2019/051093, PCT/US2021/028254, PCT/US2021/030277, the disclosures of which is incorporated herein by reference in their entireties.

In some embodiments, a yeast cell transformed with one or more KPV expression cassettes can produce a KPV in a yeast culture with a yield of: at least 30 mg/L, at least 40 mg/L, at least 50 mg/L, at least 60 mg/L, at least 70 mg/L, at least 80 mg/L, at least 90 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, at least 190 mg/L 200 mg/L, at least 500 mg/L, at least 750 mg/L, at least 1,000 mg/L, at least 1,250 mg/L, at least 1,500 mg/L, at least 1,750 mg/L, at least 2,000 mg/L, at least 2,500 mg/L, at least 3,000 mg/L, at least 3,500 mg/L, at least 4,000 mg/L, at least 4,500 mg/L, at least 5,000 mg/L, at least 5,500 mg/L, at least at least 6,000 mg/L, at least 6,500 mg/L, at least 7,000 mg/L, at least 7,500 mg/L, at least 8,000 mg/L, at least 8,500 mg/L, at least 9,000 mg/L, at least 9,500 mg/L, at least 10,000 mg/L, at least 11,000 mg/L, at least 12,000 mg/L, at least 12,500 mg/L, at least 13,000 mg/L, at least 14,000 mg/L, at least 15,000 mg/L, at least 16,000 mg/L, at least 17,000 mg/L, at least 17,500 mg/L, at least 18,000 mg/L, at least 19,000 mg/L, at least 20,000 mg/L, at least 25,000 mg/L, at least 30,000 mg/L, at least 40,000 mg/L, at least 50,000 mg/L, at least 60,000 mg/L, at least 70,000 mg/L, at least 80,000 mg/L, at least 90,000 mg/L, or at least 100,000 mg/L of KPV per liter of medium.

In some embodiments, one or more expression cassettes comprising a polynucleotide operable to express a KPV can be inserted into a vector, resulting in a yield ranging from about 100 mg/L of KPV to about 100,000 mg/L; from about 110 mg/L to about 100,000 mg/L; from about 120 mg/L to about 100,000 mg/L; from about 130 mg/L to about 100,000 mg/L; from about 140 mg/L to about 100,000 mg/L; from about 150 mg/L to about 100,000 mg/L; from about 160 mg/L to about 100,000 mg/L; from about 170 mg/L to about 100,000 mg/L; from about 180 mg/L to about 100,000 mg/L; from about 190 mg/L to about 100,000 mg/L; from about 200 mg/L to about 100,000 mg/L; from about 250 mg/L to about 100,000 mg/L; from about 500 mg/L to about 100,000 mg/L; from about 750 mg/L to about 100,000 mg/L; from about 1000 mg/L to about 100,000 mg/L; from about 1000 mg/L to about 100,000 mg/L; from about 1500 mg/L to about 100,000 mg/L; from about 2000 mg/L to about 100,000 mg/L; from about 2500 mg/L to about 100,000 mg/L; from about 3000 mg/L to about 100,000 mg/L; from about 3500 mg/L to about 100,000 mg/L; from about 4000 mg/L to about 100,000 mg/L; from about 4500 mg/L to about 100,000 mg/L; from about 5000 mg/L to about 100,000 mg/L; from about 5500 mg/L to about 100,000 mg/L; from about 6000 mg/L to about 100,000 mg/L; from about 6500 mg/L to about 100,000 mg/L; from about 7000 mg/L to about 100,000 mg/L; from about 7500 mg/L to about 100,000 mg/L; from about 8000 mg/L to about 100,000 mg/L; from about 8500 mg/L to about 100,000 mg/L; from about 9000 mg/L to about 100,000 mg/L; from about 9500 mg/L to about 100,000 mg/L; from about 10000 mg/L to about 100,000 mg/L; from about 10500 mg/L to about 100,000 mg/L; from about 11000 mg/L to about 100,000 mg/L; from about 11500 mg/L to about 100,000 mg/L; from about 12000 mg/L to about 100,000 mg/L; from about 12500 mg/L to about 100,000 mg/L; from about 13000 mg/L to about 100,000 mg/L; from about 13500 mg/L to about 100,000 mg/L; from about 14000 mg/L to about 100,000 mg/L; from about 14500 mg/L to about 100,000 mg/L; from about 15000 mg/L to about 100,000 mg/L; from about 15500 mg/L to about 100,000 mg/L; from about 16000 mg/L to about 100,000 mg/L; from about 16500 mg/L to about 100,000 mg/L; from about 17000 mg/L to about 100,000 mg/L; from about 17500 mg/L to about 100,000 mg/L; from about 18000 mg/L to about 100,000 mg/L; from about 18500 mg/L to about 100,000 mg/L; from about 19000 mg/L to about 100,000 mg/L; from about 19500 mg/L to about 100,000 mg/L; from about 20000 mg/L to about 100,000 mg/L; from about 20500 mg/L to about 100,000 mg/L; from about 21000 mg/L to about 100,000 mg/L; from about 21500 mg/L to about 100,000 mg/L; from about 22000 mg/L to about 100,000 mg/L; from about 22500 mg/L to about 100,000 mg/L; from about 23000 mg/L to about 100,000 mg/L; from about 23500 mg/L to about 100,000 mg/L; from about 24000 mg/L to about 100,000 mg/L; from about 24500 mg/L to about 100,000 mg/L; from about 25000 mg/L to about 100,000 mg/L; from about 25500 mg/L to about 100,000 mg/L; from about 26000 mg/L to about 100,000 mg/L; from about 26500 mg/L to about 100,000 mg/L; from about 27000 mg/L to about 100,000 mg/L; from about 27500 mg/L to about 100,000 mg/L; from about 28000 mg/L to about 100,000 mg/L; from about 28500 mg/L to about 100,000 mg/L; from about 29000 mg/L to about 100,000 mg/L; from about 29500 mg/L to about 100,000 mg/L; from about 30000 mg/L to about 100,000 mg/L; from about 30500 mg/L to about 100,000 mg/L; from about 31000 mg/L to about 100,000 mg/L; from about 31500 mg/L to about 100,000 mg/L; from about 32000 mg/L to about 100,000 mg/L; from about 32500 mg/L to about 100,000 mg/L; from about 33000 mg/L to about 100,000 mg/L; from about 33500 mg/L to about 100,000 mg/L; from about 34000 mg/L to about 100,000 mg/L; from about 34500 mg/L to about 100,000 mg/L; from about 35000 mg/L to about 100,000 mg/L; from about 35500 mg/L to about 100,000 mg/L; from about 36000 mg/L to about 100,000 mg/L; from about 36500 mg/L to about 100,000 mg/L; from about 37000 mg/L to about 100,000 mg/L; from about 37500 mg/L to about 100,000 mg/L; from about 38000 mg/L to about 100,000 mg/L; from about 38500 mg/L to about 100,000 mg/L; from about 39000 mg/L to about 100,000 mg/L; from about 39500 mg/L to about 100,000 mg/L; from about 40000 mg/L to about 100,000 mg/L; from about 40500 mg/L to about 100,000 mg/L; from about 41000 mg/L to about 100,000 mg/L; from about 41500 mg/L to about 100,000 mg/L; from about 42000 mg/L to about 100,000 mg/L; from about 42500 mg/L to about 100,000 mg/L; from about 43000 mg/L to about 100,000 mg/L; from about 43500 mg/L to about 100,000 mg/L; from about 44000 mg/L to about 100,000 mg/L; from about 44500 mg/L to about 100,000 mg/L; from about 45000 mg/L to about 100,000 mg/L; from about 45500 mg/L to about 100,000 mg/L; from about 46000 mg/L to about 100,000 mg/L; from about 46500 mg/L to about 100,000 mg/L; from about 47000 mg/L to about 100,000 mg/L; from about 47500 mg/L to about 100,000 mg/L; from about 48000 mg/L to about 100,000 mg/L; from about 48500 mg/L to about 100,000 mg/L; from about 49000 mg/L to about 100,000 mg/L; from about 49500 mg/L to about 100,000 mg/L; from about 50000 mg/L to about 100,000 mg/L; from about 50500 mg/L to about 100,000 mg/L; from about 51000 mg/L to about 100,000 mg/L; from about 51500 mg/L to about 100,000 mg/L; from about 52000 mg/L to about 100,000 mg/L; from about 52500 mg/L to about 100,000 mg/L; from about 53000 mg/L to about 100,000 mg/L; from about 53500 mg/L to about 100,000 mg/L; from about 54000 mg/L to about 100,000 mg/L; from about 54500 mg/L to about 100,000 mg/L; from about 55000 mg/L to about 100,000 mg/L; from about 55500 mg/L to about 100,000 mg/L; from about 56000 mg/L to about 100,000 mg/L; from about 56500 mg/L to about 100,000 mg/L; from about 57000 mg/L to about 100,000 mg/L; from about 57500 mg/L to about 100,000 mg/L; from about 58000 mg/L to about 100,000 mg/L; from about 58500 mg/L to about 100,000 mg/L; from about 59000 mg/L to about 100,000 mg/L; from about 59500 mg/L to about 100,000 mg/L; from about 60000 mg/L to about 100,000 mg/L; from about 60500 mg/L to about 100,000 mg/L; from about 61000 mg/L to about 100,000 mg/L; from about 61500 mg/L to about 100,000 mg/L; from about 62000 mg/L to about 100,000 mg/L; from about 62500 mg/L to about 100,000 mg/L; from about 63000 mg/L to about 100,000 mg/L; from about 63500 mg/L to about 100,000 mg/L; from about 64000 mg/L to about 100,000 mg/L; from about 64500 mg/L to about 100,000 mg/L; from about 65000 mg/L to about 100,000 mg/L; from about 65500 mg/L to about 100,000 mg/L; from about 66000 mg/L to about 100,000 mg/L; from about 66500 mg/L to about 100,000 mg/L; from about 67000 mg/L to about 100,000 mg/L; from about 67500 mg/L to about 100,000 mg/L; from about 68000 mg/L to about 100,000 mg/L; from about 68500 mg/L to about 100,000 mg/L; from about 69000 mg/L to about 100,000 mg/L; from about 69500 mg/L to about 100,000 mg/L; from about 70000 mg/L to about 100,000 mg/L; from about 70500 mg/L to about 100,000 mg/L; from about 71000 mg/L to about 100,000 mg/L; from about 71500 mg/L to about 100,000 mg/L; from about 72000 mg/L to about 100,000 mg/L; from about 72500 mg/L to about 100,000 mg/L; from about 73000 mg/L to about 100,000 mg/L; from about 73500 mg/L to about 100,000 mg/L; from about 74000 mg/L to about 100,000 mg/L; from about 74500 mg/L to about 100,000 mg/L; from about 75000 mg/L to about 100,000 mg/L; from about 75500 mg/L to about 100,000 mg/L; from about 76000 mg/L to about 100,000 mg/L; from about 76500 mg/L to about 100,000 mg/L; from about 77000 mg/L to about 100,000 mg/L; from about 77500 mg/L to about 100,000 mg/L; from about 78000 mg/L to about 100,000 mg/L; from about 78500 mg/L to about 100,000 mg/L; from about 79000 mg/L to about 100,000 mg/L; from about 79500 mg/L to about 100,000 mg/L; from about 80000 mg/L to about 100,000 mg/L; from about 80500 mg/L to about 100,000 mg/L; from about 81000 mg/L to about 100,000 mg/L; from about 81500 mg/L to about 100,000 mg/L; from about 82000 mg/L to about 100,000 mg/L; from about 82500 mg/L to about 100,000 mg/L; from about 83000 mg/L to about 100,000 mg/L; from about 83500 mg/L to about 100,000 mg/L; from about 84000 mg/L to about 100,000 mg/L; from about 84500 mg/L to about 100,000 mg/L; from about 85000 mg/L to about 100,000 mg/L; from about 85500 mg/L to about 100,000 mg/L; from about 86000 mg/L to about 100,000 mg/L; from about 86500 mg/L to about 100,000 mg/L; from about 87000 mg/L to about 100,000 mg/L; from about 87500 mg/L to about 100,000 mg/L; from about 88000 mg/L to about 100,000 mg/L; from about 88500 mg/L to about 100,000 mg/L; from about 89000 mg/L to about 100,000 mg/L; from about 89500 mg/L to about 100,000 mg/L; from about 90000 mg/L to about 100,000 mg/L; from about 90500 mg/L to about 100,000 mg/L; from about 91000 mg/L to about 100,000 mg/L; from about 91500 mg/L to about 100,000 mg/L; from about 92000 mg/L to about 100,000 mg/L; from about 92500 mg/L to about 100,000 mg/L; from about 93000 mg/L to about 100,000 mg/L; from about 93500 mg/L to about 100,000 mg/L; from about 94000 mg/L to about 100,000 mg/L; from about 94500 mg/L to about 100,000 mg/L; from about 95000 mg/L to about 100,000 mg/L; from about 95500 mg/L to about 100,000 mg/L; from about 96000 mg/L to about 100,000 mg/L; from about 96500 mg/L to about 100,000 mg/L; from about 97000 mg/L to about 100,000 mg/L; from about 97500 mg/L to about 100,000 mg/L; from about 98000 mg/L to about 100,000 mg/L; from about 98500 mg/L to about 100,000 mg/L; from about 99000 mg/L to about 100,000 mg/L; or from about 99500 mg/L to about 100,000 mg/L of KPV per liter of medium (supernatant of yeast fermentation broth).

In some In some embodiments, one or more expression cassettes comprising a polynucleotide operable to express a KPV can be inserted into a vector, resulting in a yield ranging from about 20 mg/L to about 100,000 mg/L; from about 30 mg/L to about 100,000 mg/L; from about 40 mg/L to about 100,000 mg/L; from about 50 mg/L to about 100,000 mg/L; from about 60 mg/L to about 100,000 mg/L; from about 70 mg/L to about 100,000 mg/L; from about 80 mg/L to about 100,000 mg/L; from about 90 mg/L to about 100,000 mg/L; from about 100 mg/L of KPV to about 100,000 mg/L; from about 100 mg/L to about 99500 mg/L; from about 100 mg/L to about 99000 mg/L; from about 100 mg/L to about 98500 mg/L; from about 100 mg/L to about 98000 mg/L; from about 100 mg/L to about 97500 mg/L; from about 100 mg/L to about 97000 mg/L; from about 100 mg/L to about 96500 mg/L; from about 100 mg/L to about 96000 mg/L; from about 100 mg/L to about 95500 mg/L; from about 100 mg/L to about 95000 mg/L; from about 100 mg/L to about 94500 mg/L; from about 100 mg/L to about 94000 mg/L; from about 100 mg/L to about 93500 mg/L; from about 100 mg/L to about 93000 mg/L; from about 100 mg/L to about 92500 mg/L; from about 100 mg/L to about 92000 mg/L; from about 100 mg/L to about 91500 mg/L; from about 100 mg/L to about 91000 mg/L; from about 100 mg/L to about 90500 mg/L; from about 100 mg/L to about 90000 mg/L; from about 100 mg/L to about 89500 mg/L; from about 100 mg/L to about 89000 mg/L; from about 100 mg/L to about 88500 mg/L; from about 100 mg/L to about 88000 mg/L; from about 100 mg/L to about 87500 mg/L; from about 100 mg/L to about 87000 mg/L; from about 100 mg/L to about 86500 mg/L; from about 100 mg/L to about 86000 mg/L; from about 100 mg/L to about 85500 mg/L; from about 100 mg/L to about 85000 mg/L; from about 100 mg/L to about 84500 mg/L; from about 100 mg/L to about 84000 mg/L; from about 100 mg/L to about 83500 mg/L; from about 100 mg/L to about 83000 mg/L; from about 100 mg/L to about 82500 mg/L; from about 100 mg/L to about 82000 mg/L; from about 100 mg/L to about 81500 mg/L; from about 100 mg/L to about 81000 mg/L; from about 100 mg/L to about 80500 mg/L; from about 100 mg/L to about 80000 mg/L; from about 100 mg/L to about 79500 mg/L; from about 100 mg/L to about 79000 mg/L; from about 100 mg/L to about 78500 mg/L; from about 100 mg/L to about 78000 mg/L; from about 100 mg/L to about 77500 mg/L; from about 100 mg/L to about 77000 mg/L; from about 100 mg/L to about 76500 mg/L; from about 100 mg/L to about 76000 mg/L; from about 100 mg/L to about 75500 mg/L; from about 100 mg/L to about 75000 mg/L; from about 100 mg/L to about 74500 mg/L; from about 100 mg/L to about 74000 mg/L; from about 100 mg/L to about 73500 mg/L; from about 100 mg/L to about 73000 mg/L; from about 100 mg/L to about 72500 mg/L; from about 100 mg/L to about 72000 mg/L; from about 100 mg/L to about 71500 mg/L; from about 100 mg/L to about 71000 mg/L; from about 100 mg/L to about 70500 mg/L; from about 100 mg/L to about 70000 mg/L; from about 100 mg/L to about 69500 mg/L; from about 100 mg/L to about 69000 mg/L; from about 100 mg/L to about 68500 mg/L; from about 100 mg/L to about 68000 mg/L; from about 100 mg/L to about 67500 mg/L; from about 100 mg/L to about 67000 mg/L; from about 100 mg/L to about 66500 mg/L; from about 100 mg/L to about 66000 mg/L; from about 100 mg/L to about 65500 mg/L; from about 100 mg/L to about 65000 mg/L; from about 100 mg/L to about 64500 mg/L; from about 100 mg/L to about 64000 mg/L; from about 100 mg/L to about 63500 mg/L; from about 100 mg/L to about 63000 mg/L; from about 100 mg/L to about 62500 mg/L; from about 100 mg/L to about 62000 mg/L; from about 100 mg/L to about 61500 mg/L; from about 100 mg/L to about 61000 mg/L; from about 100 mg/L to about 60500 mg/L; from about 100 mg/L to about 60000 mg/L; from about 100 mg/L to about 59500 mg/L; from about 100 mg/L to about 59000 mg/L; from about 100 mg/L to about 58500 mg/L; from about 100 mg/L to about 58000 mg/L; from about 100 mg/L to about 57500 mg/L; from about 100 mg/L to about 57000 mg/L; from about 100 mg/L to about 56500 mg/L; from about 100 mg/L to about 56000 mg/L; from about 100 mg/L to about 55500 mg/L; from about 100 mg/L to about 55000 mg/L; from about 100 mg/L to about 54500 mg/L; from about 100 mg/L to about 54000 mg/L; from about 100 mg/L to about 53500 mg/L; from about 100 mg/L to about 53000 mg/L; from about 100 mg/L to about 52500 mg/L; from about 100 mg/L to about 52000 mg/L; from about 100 mg/L to about 51500 mg/L; from about 100 mg/L to about 51000 mg/L; from about 100 mg/L to about 50500 mg/L; from about 100 mg/L to about 50000 mg/L; from about 100 mg/L to about 49500 mg/L; from about 100 mg/L to about 49000 mg/L; from about 100 mg/L to about 48500 mg/L; from about 100 mg/L to about 48000 mg/L; from about 100 mg/L to about 47500 mg/L; from about 100 mg/L to about 47000 mg/L; from about 100 mg/L to about 46500 mg/L; from about 100 mg/L to about 46000 mg/L; from about 100 mg/L to about 45500 mg/L; from about 100 mg/L to about 45000 mg/L; from about 100 mg/L to about 44500 mg/L; from about 100 mg/L to about 44000 mg/L; from about 100 mg/L to about 43500 mg/L; from about 100 mg/L to about 43000 mg/L; from about 100 mg/L to about 42500 mg/L; from about 100 mg/L to about 42000 mg/L; from about 100 mg/L to about 41500 mg/L; from about 100 mg/L to about 41000 mg/L; from about 100 mg/L to about 40500 mg/L; from about 100 mg/L to about 40000 mg/L; from about 100 mg/L to about 39500 mg/L; from about 100 mg/L to about 39000 mg/L; from about 100 mg/L to about 38500 mg/L; from about 100 mg/L to about 38000 mg/L; from about 100 mg/L to about 37500 mg/L; from about 100 mg/L to about 37000 mg/L; from about 100 mg/L to about 36500 mg/L; from about 100 mg/L to about 36000 mg/L; from about 100 mg/L to about 35500 mg/L; from about 100 mg/L to about 35000 mg/L; from about 100 mg/L to about 34500 mg/L; from about 100 mg/L to about 34000 mg/L; from about 100 mg/L to about 33500 mg/L; from about 100 mg/L to about 33000 mg/L; from about 100 mg/L to about 32500 mg/L; from about 100 mg/L to about 32000 mg/L; from about 100 mg/L to about 31500 mg/L; from about 100 mg/L to about 31000 mg/L; from about 100 mg/L to about 30500 mg/L; from about 100 mg/L to about 30000 mg/L; from about 100 mg/L to about 29500 mg/L; from about 100 mg/L to about 29000 mg/L; from about 100 mg/L to about 28500 mg/L; from about 100 mg/L to about 28000 mg/L; from about 100 mg/L to about 27500 mg/L; from about 100 mg/L to about 27000 mg/L; from about 100 mg/L to about 26500 mg/L; from about 100 mg/L to about 26000 mg/L; from about 100 mg/L to about 25500 mg/L; from about 100 mg/L to about 25000 mg/L; from about 100 mg/L to about 24500 mg/L; from about 100 mg/L to about 24000 mg/L; from about 100 mg/L to about 23500 mg/L; from about 100 mg/L to about 23000 mg/L; from about 100 mg/L to about 22500 mg/L; from about 100 mg/L to about 22000 mg/L; from about 100 mg/L to about 21500 mg/L; from about 100 mg/L to about 21000 mg/L; from about 100 mg/L to about 20500 mg/L; from about 100 mg/L to about 20000 mg/L; from about 100 mg/L to about 19500 mg/L; from about 100 mg/L to about 19000 mg/L; from about 100 mg/L to about 18500 mg/L; from about 100 mg/L to about 18000 mg/L; from about 100 mg/L to about 17500 mg/L; from about 100 mg/L to about 17000 mg/L; from about 100 mg/L to about 16500 mg/L; from about 100 mg/L to about 16000 mg/L; from about 100 mg/L to about 15500 mg/L; from about 100 mg/L to about 15000 mg/L; from about 100 mg/L to about 14500 mg/L; from about 100 mg/L to about 14000 mg/L; from about 100 mg/L to about 13500 mg/L; from about 100 mg/L to about 13000 mg/L; from about 100 mg/L to about 12500 mg/L; from about 100 mg/L to about 12000 mg/L; from about 100 mg/L to about 11500 mg/L; from about 100 mg/L to about 11000 mg/L; from about 100 mg/L to about 10500 mg/L; from about 100 mg/L to about 10000 mg/L; from about 100 mg/L to about 9500 mg/L; from about 100 mg/L to about 9000 mg/L; from about 100 mg/L to about 8500 mg/L; from about 100 mg/L to about 8000 mg/L; from about 100 mg/L to about 7500 mg/L; from about 100 mg/L to about 7000 mg/L; from about 100 mg/L to about 6500 mg/L; from about 100 mg/L to about 6000 mg/L; from about 100 mg/L to about 5500 mg/L; from about 100 mg/L to about 5000 mg/L; from about 100 mg/L to about 4500 mg/L; from about 100 mg/L to about 4000 mg/L; from about 100 mg/L to about 3500 mg/L; from about 100 mg/L to about 3000 mg/L; from about 100 mg/L to about 2500 mg/L; from about 100 mg/L to about 2000 mg/L; from about 100 mg/L to about 1500 mg/L; from about 100 mg/L to about 1000 mg/L; from about 100 mg/L to about 1000 mg/L; from about 100 mg/L to about 750 mg/L; from about 100 mg/L to about 500 mg/L; from about 100 mg/L to about 250 mg/L; from about 100 mg/L to about 100 mg/L; or from about 100 mg/L to about 110 mg/L of KPV per liter of medium (supernatant of yeast fermentation broth).

### Chemically Synthesizing KPVs

In some embodiments, the polynucleotide sequence encoding an engineered, non-naturally occurring pesticidal KPV of the present disclosure can be chemically synthesized using commercially available polynucleotide synthesis services, such as those offered by GENEWIZ® (e.g., TurboGENE™; PriorityGENE; and FragmentGENE), or SIGMA-ALDRICH® (e.g., Custom DNA and RNA Oligos Design and Order Custom DNA Oligos). Exemplary method for generating DNA and or custom chemically synthesized polynucleotides are well known in the art, and are illustratively provided in U.S. Pat. No. 5,736,135, Ser. No. 08/389,615, filed on Feb. 13, 1995, the disclosure of which is incorporated herein by reference in its entirety. See also Agarwal, et al., Chemical synthesis of polynucleotides. Angew Chem Int Ed Engl. 1972 June; 11(6): 451-9; Ohtsuka et al., Recent developments in the chemical synthesis of polynucleotides. Nucleic Acids Res. 1982 Nov. 11; 10(21): 6553-6570; Sondek & Shortle. A general strategy for random insertion and substitution mutagenesis: substoichiometric coupling of trinucleotide phosphoramidites. Proc Natl Acad Sci USA. 1992 Apr. 15; 89(8): 3581-3585; Beaucage S. L., et al., Advances in the Synthesis of Oligonucleotides by the Phosphoramidite Approach. Tetrahedron, Elsevier Science Publishers, Amsterdam, NL, vol. 48, No. 12, 1992, pp. 2223-2311; Agrawal (1993) Protocols for Oligonucleotides and Analogs: Synthesis and Properties; Methods in Molecular Biology Vol. 20, the disclosures of which are incorporated herein by reference in their entirety.

Chemically synthesizing polynucleotides allows for a DNA sequence to be generated that is tailored to produce a desired peptide based on the arrangement of nucleotides within said sequence (i.e., the arrangement of cytosine [C], guanine [G], adenine [A] or thymine [T] molecules); the mRNA sequence that is transcribed from the chemically synthesized DNA polynucleotide can be translated to a sequence of amino acids, each amino acid corresponding to a codon in the mRNA sequence.

Obtaining an engineered, non-naturally occurring pesticidal KPV of the present disclosure from a chemically synthesized DNA polynucleotide sequence and/or a wild-type DNA polynucleotide sequence can be achieved by cloning the DNA sequence into an appropriate vector. There are a variety of expression vectors available, host organisms, and cloning strategies known to those having ordinary skill in the art. For example, the vector can be a plasmid, which can introduce a heterologous gene and/or expression cassette into yeast cells to be transcribed and translated. The term “vector” is used to refer to a carrier nucleic acid molecule into which a nucleic acid sequence can be inserted for introduction into a cell where it can be replicated. A vector may contain “vector elements” such as an origin of replication (ORI); a gene that confers antibiotic resistance to allow for selection; multiple cloning sites; a promoter region; a selection marker for non-bacterial transfection; and a primer binding site. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference. In addition to synthesizing a polynucleotide operable to encode an engineered, non-naturally occurring pesticidal KPV of the present disclosure, a vector may also encode a targeting molecule. A targeting molecule is one that directs the desired nucleic acid to a particular tissue, cell, or other location.

### Chemically Synthesizing Peptides

Peptide synthesis or the chemical synthesis of peptides and/or polypeptides can be used to produce or synthesize an engineered, non-naturally occurring pesticidal KPV of the present disclosure. These methods can be performed by those having ordinary skill in the art, and/or through the use of commercial vendors (e.g., GenScript®; Piscataway, New Jersey). For example, in some embodiments, chemical peptide synthesis can be achieved using Liquid phase peptide synthesis (LPPS), or solid phase peptide synthesis (SPPS).

In some embodiments, peptide synthesis can generally be achieved by using a strategy wherein the coupling the carboxyl group of a subsequent amino acid to the N-terminus of a preceding amino acid generates the nascent polypeptide chain—a process that is opposite to the type of polypeptide synthesis that occurs in nature.

Peptide deprotection is an important first step in the chemical synthesis of polypeptides. Peptide deprotection is the process in which the reactive groups of amino acids are blocked through the use of chemicals in order to prevent said amino acid's functional group from taking part in an unwanted or non-specific reaction or side reaction; in other words, the amino acids are “protected” from taking part in these undesirable reactions.

Prior to synthesizing the peptide chain, the amino acids must be “deprotected” to allow the chain to form (i.e., amino acids to bind). Chemicals used to protect the N-termini include 9-fluorenylmethoxycarbonyl (Fmoc), and tert-butoxycarbonyl (Boc), each of which can be removed via the use of a mild base (e.g., piperidine) and a moderately strong acid (e.g., trifluoracetic acid (TFA)), respectively.

The C-terminus protectant required is dependent on the type of chemical peptide synthesis strategy used: e.g., LPPS requires protection of the C-terminal amino acid, whereas SPPS does not owing to the solid support which acts as the protecting group. Side chain amino acids require the use of several different protecting groups that vary based on the individual peptide sequence and N-terminal protection strategy; typically, however, the protecting group used for side chain amino acids are based on the tert-butyl (tBu) or benzyl (Bzl) protecting groups.

Amino acid coupling is the next step in a peptide synthesis procedure. To effectuate amino acid coupling, the incoming amino acid's C-terminal carboxylic acid must be activated: this can be accomplished using carbodiimides such as diisopropylcarbodiimide (DIC), or dicyclohexylcarbodiimide (DCC), which react with the incoming amino acid's carboxyl group to form an O-acylisourea intermediate. The O-acylisourea intermediate is subsequently displaced via nucleophilic attack via the primary amino group on the N-terminus of the growing peptide chain. The reactive intermediate generated by carbodiimides can result in the racemization of amino acids. To avoid racemization of the amino acids, reagents such as 1-hydroxybenzotriazole (HOBt) are added in order to react with the O-acylisourea intermediate. Other couple agents that may be used include 2-(1H-benzotriazol-1-yl)-1,1,3,3-tetramethyluronium hexafluorophosphate (HBTU), and benzotriazol-1-yl-oxy-tris(dimethylamino)phosphonium hexafluorophosphate (BOP), with the additional activating bases. Finally, amino acid deprotection and coupling.

At the end of the synthesis process, removal of the protecting groups from the polypeptide must occur—a process that usually occurs through acidolysis. Determining which reagent is required for peptide cleavage is a function of the protection scheme used and overall synthesis method. For example, in some embodiments, hydrogen bromide (HBr); hydrogen fluoride (HF); or trifluoromethane sulfonic acid (TFMSA) can be used to cleave Bzl and Boc groups. Alternatively, in other embodiments, a less strong acid such as TFA can effectuate acidolysis of tBut and Fmoc groups. Finally, peptides can be purified based on the peptide's physiochemical characteristics (e.g., charge, size, hydrophobicity, etc.). Techniques that can be used to purify peptides include Purification techniques include Reverse-phase chromatography (RPC); Size-exclusion chromatography; Partition chromatography; High-performance liquid chromatography (HPLC); and Ion exchange chromatography (IEC).

Exemplary methods of peptide synthesis can be found in Anderson G. W. and McGregor A. C. (1957) T-butyloxycarbonylamino acids and their use in peptide synthesis. Journal of the American Chemical Society. 79, 6180-3; Carpino L. A. (1957) Oxidative reactions of hydrazines. Iv. Elimination of nitrogen from 1,1-disubstituted-2-arenesulfonhydrazides1-4. Journal of the American Chemical Society. 79, 4427-31; Mckay F. C. and Albertson N. F. (1957) New amine-masking groups for peptide synthesis. Journal of the American Chemical Society. 79, 4686-90; Merrifield R. B. (1963) Solid phase peptide synthesis. I. The synthesis of a tetrapeptide. Journal of the American Chemical Society. 85, 2149-54; Carpino L. A. and Han G. Y. (1972) 9-fluorenylmethoxycarbonyl amino-protecting group. The Journal of Organic Chemistry. 37, 3404-9; and A Lloyd-Williams P. et al. (1997) Chemical approaches to the synthesis of peptides and proteins. Boca Raton: CRC Press. 278; U.S. Pat. No. 3,714,140 (filed Mar. 16, 1971); U.S. Pat. No. 4,411,994 (filed Jun. 8, 1978); U.S. Pat. No. 7,785,832 (filed Jan. 20, 2006); U.S. Pat. No. 8,314,208 (filed Feb. 10, 2006); and U.S. Pat. No. 10,442,834 (filed Oct. 2, 2015); and United States Patent Application 2005/0165215 (filed Dec. 23, 2004), the disclosures of which are incorporated herein by reference in their entireties.

Any of the methods described herein can be used to generate an engineered, non-naturally occurring pesticidal KPV described herein, e.g., an engineered, non-naturally occurring pesticidal KPV consisting of an amino acid sequence set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or by a polynucleotide sequence encoding the same.

### Cell Culture and Transformation Techniques

The terms “transformation” and “transfection” both describe the process of introducing exogenous and/or heterologous polynucleotide (e.g., DNA or RNA) to a host organism. Generally, those having ordinary skill in the art sometimes reserve the term “transformation” to describe processes where exogenous and/or heterologous polynucleotide (e.g., DNA or RNA) are introduced into a bacterial cell; and reserve the term “transfection” for processes that describe the introduction of exogenous and/or heterologous polynucleotide (e.g., DNA or RNA) into eukaryotic cells. However, as used herein, the term “transformation” and “transfection” are used synonymously, regardless of whether a process describes the introduction exogenous and/or heterologous DNA or RNA into a prokaryote (e.g., bacteria) or a eukaryote (e.g., yeast, plants, or animals).

In some embodiments, a host organism can be transformed with a polynucleotide operable to encode a KPV. In some embodiments, the host organism can be an microorganism, e.g., a cell.

In some embodiments, a vector comprising a KPV expression cassette can be cloned into an expression plasmid and transformed into a host cell. In some embodiments, the host cell can be selected from any host cell described herein.

In some embodiments, a host cell can be transformed using the following methods: electroporation; cell squeezing; microinjection; impalefection; the use of hydrostatic pressure; sonoporation; optical transfection; continuous infusion; lipofection; through the use of viruses such as adenovirus, adeno-associated virus, lentivirus, herpes simplex virus, and retrovirus; the chemical phosphate method; endocytosis via DEAE-dextran or polyethylenimine (PEI); protoplast fusion; hydrodynamic deliver; magnetofection; nucleoinfection; and/or others. Exemplary methods regarding transfection and/or transformation techniques can be found in Makrides (2003), Gene Transfer and Expression in Mammalian Cells, Elvesier; Wong, T K & Neumann, E. Electric field mediated gene transfer. Biochem. Biophys. Res. Commun. 107, 584-587 (1982); Potter & Heller, Transfection by Electroporation. Curr Protoc Mol Biol. 2003 May; CHAPTER: Unit-9.3; Kim & Eberwine, Mammalian cell transfection: the present and the future. Anal Bioanal Chem. 2010 August; 397 (8): 3173-3178, each of these references are incorporated herein by reference in their entireties.

Electroporation is an exemplary method for transforming host cells. Electroporation is a technique in which electricity is applied to cells causing the cell membrane to become permeable; this in turn allows exogenous DNA to be introduced into the cells. Electroporation is readily known to those having ordinary skill in the art, and the tools and devices required to achieve electroporation are commercially available (e.g., Gene Pulser Xcell™ Electroporation Systems, Bio-Rad®; Neon® Transfection System for Electroporation, Thermo-Fisher Scientific; and other tools and/or devices). Exemplary methods of electroporation are illustrated in Potter & Heller, Transfection by Electroporation. Curr Protoc Mol Biol. 2003 May; CHAPTER: Unit-9.3; Saito (2015) Electroporation Methods in Neuroscience. Springer press; Pakhomov et al., (2017) Advanced Electroporation Techniques in Biology and Medicine. Taylor & Francis; the disclosure of which is incorporated herein by reference in its entirety.

In some embodiments, electroporation can be used transform a cell with one or more vectors containing a polynucleotide operable to encode one or more KPVs or KPV-pesticidal proteins. For example, in some embodiments, electroporation can be used transform a cell with one or more vectors containing one or more KPV expression cassettes.

### Exemplary Description of Yeast Transformation and Culture Methods

In some embodiments, electroporation can be used transform a yeast cell with one or more vectors containing one or more KPV expression cassettes, which can produce KPV in a yeast culture with a yield of: at least 10 mg/L, at least 20 mg/L, at least 30 mg/L, at least 40 mg/L, at least 50 mg/L, at least 60 mg/L, at least 70 mg/L, at least 80 mg/L, at least 90 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, at least 190 mg/L 200 mg/L, at least 500 mg/L, at least 750 mg/L, at least 1,000 mg/L, at least 1,250 mg/L, at least 1,500 mg/L, at least 1,750 mg/L, at least 2,000 mg/L, at least 2,500 mg/L, at least 3,000 mg/L, at least 3,500 mg/L, at least 4,000 mg/L, at least 4,500 mg/L, at least 5,000 mg/L, at least 5,500 mg/L, at least at least 6,000 mg/L, at least 6,500 mg/L, at least 7,000 mg/L, at least 7,500 mg/L, at least 8,000 mg/L, at least 8,500 mg/L, at least 9,000 mg/L, at least 9,500 mg/L, at least 10,000 mg/L, at least 11,000 mg/L, at least 12,000 mg/L, at least 12,500 mg/L, at least 13,000 mg/L, at least 14,000 mg/L, at least 15,000 mg/L, at least 16,000 mg/L, at least 17,000 mg/L, at least 17,500 mg/L, at least 18,000 mg/L, at least 19,000 mg/L, at least 20,000 mg/L, at least 25,000 mg/L, at least 30,000 mg/L, at least 40,000 mg/L, at least 50,000 mg/L, at least 60,000 mg/L, at least 70,000 mg/L, at least 80,000 mg/L, at least 90,000 mg/L, or at least 100,000 mg/L of KPV per liter of medium.

In some embodiments, electroporation can be used to introduce a vector containing a polynucleotide encoding a KPV into yeast, for example, in some embodiments, a KPV expression cassette cloned into a plasmid, and transformed into yeast cells via electroporation.

In some embodiments, a KPV expression cassette cloned into a plasmid, and transformed a host cell (e.g., a yeast cell) via electroporation can be accomplished by inoculating about 10-200 mL of yeast extract peptone dextrose (YEPD) with a suitable yeast species, for example, Kluyveromyces lactis, Kluyveromyces marxianus, Saccharomyces cerevisiae, Pichia pastoris, and others yeast species as described herein.

In some embodiments, electroporation can be used to introduce a vector containing a polynucleotide encoding a KPV into yeast, for example, a KPV cloned into a plasmid, and transformed into K. lactis cells via electroporation. Methods of K. lactis transformation with plasmids is known in the art, and described herein.

In some embodiments, using the illustrated methods described herein, i.e., vectors of the present disclosure utilizing yeast, and methods transformation and fermentation, may result in production of KPV in amounts of: at least 10 mg/L, at least 20 mg/L, at least 30 mg/L, at least 40 mg/L, at least 50 mg/L, at least 60 mg/L, at least 70 mg/L, at least 80 mg/L, at least 90 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, at least 190 mg/L 200 mg/L, at least 500 mg/L, at least 750 mg/L, at least 1,000 mg/L, at least 1,250 mg/L, at least 1,500 mg/L, at least 1,750 mg/L, at least 2,000 mg/L, at least 2,500 mg/L, at least 3,000 mg/L, at least 3,500 mg/L, at least 4,000 mg/L, at least 4,500 mg/L, at least 5,000 mg/L, at least 5,500 mg/L, at least at least 6,000 mg/L, at least 6,500 mg/L, at least 7,000 mg/L, at least 7,500 mg/L, at least 8,000 mg/L, at least 8,500 mg/L, at least 9,000 mg/L, at least 9,500 mg/L, at least 10,000 mg/L, at least 11,000 mg/L, at least 12,000 mg/L, at least 12,500 mg/L, at least 13,000 mg/L, at least 14,000 mg/L, at least 15,000 mg/L, at least 16,000 mg/L, at least 17,000 mg/L, at least 17,500 mg/L, at least 18,000 mg/L, at least 19,000 mg/L, at least 20,000 mg/L, at least 25,000 mg/L, at least 30,000 mg/L, at least 40,000 mg/L, at least 50,000 mg/L, at least 60,000 mg/L, at least 70,000 mg/L, at least 80,000 mg/L, at least 90,000 mg/L, or at least 100,000 mg/L of KPV per liter of medium.

In some embodiments, electroporation can be used to introduce a vector containing a polynucleotide encoding a KPV into plant protoplasts by incubating sterile plant material in a protoplast solution (e.g., around 8 mL of 10 mM 2-[N-morpholino]ethanesulfonic acid (MES), pH 5.5; 0.01% (w/v) pectylase; 1% (w/v) macerozyme; 40 mM CaCl₂; and 0.4 M mannitol). Methods of introducing a vector containing a polynucleotide into plant protoplasts are known to those having ordinary skill in the art.

### Host Cells and Host Organisms

The methods, compositions, KPVs, and KPV-pesticidal proteins of the present disclosure may be implemented in any host organism. For example, in some embodiments, the host organism can be a cell. In some embodiments, the cell can be, e.g., a eukaryotic or prokaryotic cell.

In some embodiments, the host cell used to produce a KPV or KPV-pesticidal protein is a prokaryote. For example, in some embodiments, the host cell may be an Archaebacteria or Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus.

In some embodiments, the host cell used to produce a KPV or KPV-pesticidal protein may be a unicellular cell. For example, in some embodiments, the host cell may be bacterial cells such as gram positive bacteria.

In some embodiments, the host cell may be a bacteria selected from the following genera consisting of: Candidatus Chloracidobacterium, Arthrobacter, Corynebacterium, Frankia, Micrococcus, Mycobacterium, Propionibacterium, Streptomyces, Aquifex Bacteroides, Porphyromonas, Bacteroides, Porphyromonas, Flavobacterium, Chlamydia, Prosthecobacter, Verrucomicrobium, Chloroflexus, Chroococcus, Merismopedia, Synechococcus, Anabaena, Nostoc, Spirulina, Trichodesmium, Pleurocapsa, Prochlorococcus, Prochloron, Bacillus, Listeria, Staphylococcus, Clostridium, Dehalobacter, Epulopiscium, Ruminococcus, Enterococcus, Lactobacillus, Streptococcus, Erysipelothrix, Mycoplasma, Leptospirillum, Nitrospira, Thermodesulfobacterium, Gemmata, Pirellula, Planctomyces, Caulobacter, Agrobacterium, Bradyrhizobium, Brucella, Methylobacterium, Prosthecomicrobium, Rhizobium, Rhodopseudomonas, Sinorhizobium, Rhodobacter, Roseobacter, Acetobacter, Rhodospirillum, Rickettsia, Rickettsia conorii, Mitochondria, Wolbachia, Erythrobacter, Erythromicrobium, Sphingomonas, Alcaligenes, Burkholderia, Leptothrix, Sphaerotilus, Thiobacillus, Neisseria, Nitrosomonas, Gallionella, Spirillum, Azoarcus, Aeromonas, Succinomonas, Succinivibrio, Ruminobacter, Nitrosococcus, Thiocapsa, Enterobacter, Escherichia, Klebsiella, Salmonella, Shigella, Wigglesworthia, Yersinia, Coxiella, Legionella, Halomonas, Pasteurella, Acinetobacter, Azotobacter, Pseudomonas, Psychrobacter, Beggiatoa, Thiomargarita, Vibrio, Xanthomonas, Bdellovibrio, Campylobacter, Helicobacter, Myxococcus, Desulfosarcina, Geobacter, Desulfuromonas, Borrelia, Leptospira, Treponema, Petrotoga, Thermotoga, Deinococcus, or Thermus.

In some embodiments, the host cell used to produce a KPV or KPV-pesticidal protein may be selected from one of the following bacteria species: Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, Bacillus thuringiensis, Streptomyces lividans, Streptomyces murinus, Streptomyces coelicolor, Streptomyces albicans, Streptomyces griseus, Streptomyces plicatosporus, Escherichia albertii, Escherichia blattae, Escherichia coli, Escherichia fergusonii, Escherichia hermannii, Escherichia senegalensis, Escherichia vulneris, Pseudomonas abietaniphila, Pseudomonas agarici, Pseudomonas agarolyticus, Pseudomonas alcaliphila, Pseudomonas alginovora, Pseudomonas andersonii, Pseudomonas antarctica, Pseudomonas asplenii, Pseudomonas azelaica, Pseudomonas batumici, Pseudomonas borealis, Pseudomonas brassicacearum, Pseudomonas chloritidismutans, Pseudomonas cremoricolorata, Pseudomonas diterpeniphila, Pseudomonas filiscindens, Pseudomonas frederiksbergensis, Pseudomonas gingeri, Pseudomonas graminis, Pseudomonas grimontii, Pseudomonas halodenitrificans, Pseudomonas halophila, Pseudomonas hibiscicola, Pseudomonas hydrogenovora, Pseudomonas indica, Pseudomonas japonica, Pseudomonas jessenii, Pseudomonas kilonensis, Pseudomonas koreensis, Pseudomonas lini, Pseudomonas lurida, Pseudomonas lutea, Pseudomonas marginata, Pseudomonas meridiana, Pseudomonas mesoacidophila, Pseudomonas pachastrellae, Pseudomonas palleroniana, Pseudomonas parafulva, Pseudomonas pavonanceae, Pseudomonas proteolyica, Pseudomonas psychrophila, Pseudomonas psychrotolerans, Pseudomonas pudica, Pseudomonas rathonis, Pseudomonas reactans, Pseudomonas rhizosphaerae, Pseudomonas salmononii, Pseudomonas thermaerum, Pseudomonas thermocarboxydovorans, Pseudomonas thermotolerans, Pseudomonas thivervalensis, Pseudomonas umsongensis, Pseudomonas vancouverensis, Pseudomonas wisconsinensis, Pseudomonas xanthomarina Pseudomonas xiamenensis, Pseudomonas aeruginosa, Pseudomonas alcaligenes, Pseudomonas anguilliseptica, Pseudomonas citronellolis, Pseudomonas flavescens, Pseudomonas jinjuensis, Pseudomonas mendocina, Pseudomonas nitroreducens, Pseudomonas oleovorans, Pseudomonas pseudoalcaligenes, Pseudomonas resinovorans, Pseudomonas straminae, Pseudomonas aurantiaca, Pseudomonas chlororaphis, Pseudomonas fragi, Pseudomonas lundensis, Pseudomonas taetrolens Pseudomonas azotoformans, Pseudomonas brenneri, Pseudomonas cedrina, Pseudomonas congelans, Pseudomonas corrugata, Pseudomonas costantinii, Pseudomonas extremorientalis, Pseudomonas fluorescens, Pseudomonas fulgida, Pseudomonas gessardii, Pseudomonas libanensis, Pseudomonas mandelii, Pseudomonas marginalis, Pseudomonas mediterranea, Pseudomonas migulae, Pseudomonas mucidolens, Pseudomonas orientalis, Pseudomonas poae, Pseudomonas rhodesiae, Pseudomonas synxantha, Pseudomonas tolaasii, Pseudomonas trivialis, Pseudomonas veronii Pseudomonas denitrificans, Pseudomonas pertucinogena, Pseudomonas fulva, Pseudomonas monteilii, Pseudomonas mosselii, Pseudomonas oryzihabitans, Pseudomonas plecoglossicida, Pseudomonas putida, Pseudomonas balearica, Pseudomonas luteola, or Pseudomonas stutzeri. Pseudomonas avellanae, Pseudomonas cannabina, Pseudomonas caricapapyae, Pseudomonas cichorii, Pseudomonas coronafaciens, Pseudomonas fuscovaginae, Pseudomonas tremae, or Pseudomonas viridiflava.

In some embodiments, the host cell used to produce a KPV or KPV-pesticidal protein can be eukaryote.

In some embodiments, the host cell used to produce a KPV or KPV-pesticidal protein may be a cell belonging to the clades: Opisthokonta; Viridiplantae (e.g., algae and plant); Amebozoa; Cercozoa; Alveolata; Marine flagellates; Heterokonta; Discicristata; or Excavata.

In some embodiments, the procedures and methods described herein can be accomplished using a host cell that is, e.g., a Metazoan, a Choanoflagellata, or a fungi.

In some embodiments, the procedures and methods described here can be accomplished using a host cell that is a fungi. For example, in some embodiments, the host cell may be a cell belonging to the eukaryote phyla: Ascomycota, Basidiomycota, Chytridiomycota, Microsporidia, or Zygomycota.

In some embodiments, the procedures and methods described here can be accomplished using a host cell that is a fungi belonging to one of the following genera: Aspergillus, Cladosporium, Magnaporthe, Morchella, Neurospora, Penicillium, Saccharomyces, Cryptococcus, or Ustilago.

In some embodiments, the procedures and methods described here can be accomplished using a host cell that is a fungi belonging to one of the following species: Saccharomyces cerevisiae, Saccharomyces boulardi, Saccharomyces uvarum; Aspergillus flavus, A. terreus, A. awamori; Cladosporium elatum, Cladosporium Herbarum, Cladosporium Sphaerospermum, and Cladosporium cladosporioides; Magnaporthe grise, Magnaporthe oryzae, Magnaporthe rhizophila; Morchella deliciosa, Morchella esculenta, Morchella conica; Neurospora crassa, Neurospora intermedia, Neurospora tetrasperma; Penicillium notatum, Penicillium chrysogenum, Penicillium roquefortii, or Penicillium simplicissimum.

In some embodiments, the host cell used to produce a KPV or KPV-pesticidal protein may be a fungi belonging to one of the following genera: Aspergillus, Cladosporium, Magnaporthe, Morchella, Neurospora, Penicillium, Saccharomyces, Cryptococcus, or Ustilago.

In some embodiments, the host cell used to produce a KPV or KPV-pesticidal protein may be a member of the Saccharomycesaceae family. For example, in some embodiments, the host cell may be one of the following genera within the Saccharomycesaceae family: Brettanomyces, Candida, Citeromyces, Cyniclomyces, Debaryomyces, Issatchenkia, Kazachstania, Kluyveromyces, Komagataella, Kuraishia, Lachancea, Lodderomyces, Nakaseomyces, Pachysolen, Pichia, Saccharomyces, Spathaspora, Tetrapisispora, Vanderwaltozyma, Torulaspora, Williopsis, Zygosaccharomyces, or Zygotorulaspora.

In some embodiments, the host cell used to produce a KPV or KPV-pesticidal protein may be one of the following: Aspergillus flavus, Aspergillus terreus, Aspergillus awamori, Cladosporium elatum, Cladosporium herbarum, Cladosporium sphaerospermum, Cladosporium cladosporioides, Magnaporthe grisea, Magnaporthe oryzae, Magnaporthe rhizophila, Morchella deliciosa, Morchella esculenta, Morchella conica, Neurospora crassa, Neurospora intermedia, Neurospora tetrasperma, Penicillium notatum, Penicillium chrysogenum, Penicillium roquefortii, or Penicillium simplicissimum.

In some embodiments, the host cell used to produce a KPV or KPV-pesticidal protein may be a species within the Candida genus. For example, the host cell may be one of the following: Candida albicans, Candida ascalaphidarum, Candida amphixiae, Candida antarctica, Candida argentea, Candida atlantica, Candida atmosphaerica, Candida auris, Candida blankii, Candida blattae, Candida bracarensis, Candida bromeliacearum, Candida carpophila, Candida carvajalis, Candida cerambycidarum, Candida chauliodes, Candida corydalis, Candida dosseyi, Candida dubliniensis, Candida ergatensis, Candida fructus, Candida glabrata, Candida fermentati, Candida guilliermondii, Candida haemulonii, Candida humilis, Candida insectamens, Candida insectorum, Candida intermedia, Candida jeffresii, or Candida kefyr.

In some embodiments, the host cell used to produce a KPV or KPV-pesticidal protein may be any species within the genera Kluyveromyces.

In some embodiments, the host cell used to produce a KPV or KPV-pesticidal protein may be a species in the genera Kluyveromyces, e.g., the host cell may be one of the following: Kluyveromyces aestuarii, Kluyveromyces dobzhanskii, Kluyveromyces lactis, Kluyveromyces marxianus, Kluyveromyces nonfermentans, or Kluyveromyces wickerhamii.

In some embodiments, the host cell used to produce a KPV or KPV-pesticidal protein may be a species within the Pichia genus. For example, the host cell may be one of the following: Pichia farinose, Pichia anomala, Pichia heedii, Pichia guilliermondii, Pichia kluyveri, Pichia membranifaciens, Pichia norvegensis, Pichia ohmeri, Pichia pastoris, Pichia methanolica, or Pichia subpelliculosa.

In some embodiments, the host cell used to produce a KPV or KPV-pesticidal protein may be a species within the Saccharomyces genus. For example, the host cell may be one of the following: Saccharomyces arboricolus, Saccharomyces bayanus, Saccharomyces bulderi, Saccharomyces cariocanus, Saccharomyces cariocus, Saccharomyces cerevisiae, Saccharomyces cerevisiae var boulardii, Saccharomyces chevalieri, Saccharomyces dairenensis, Saccharomyces ellipsoideus, Saccharomyces eubayanus, Saccharomyces exiguous, Saccharomyces florentinus, Saccharomyces fragilis, Saccharomyces kudriavzevii, Saccharomyces martiniae, Saccharomyces mikatae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, or Saccharomyces zonatus.

In some embodiments, the procedures and methods described here can be accomplished using a host cell that is a Kluyveromyces lactis, Kluyveromyces marxianus, Saccharomyces cerevisiae, Pichia pastoris, Pichia methanolica, Schizosaccharomyces pombe, or Hansenula anomala.

The use of yeast cells as a host organism to generate recombinant KPV is an exceptional method, well known to those having ordinary skill in the art. In some embodiments, the methods and compositions described herein can be performed with any species of yeast, including but not limited to any species of the genus Saccharomyces, Pichia, Kluyveromyces, Hansenula, Yarrowia or Schizosaccharomyces and the species Saccharomyces includes any species of Saccharomyces, for example Saccharomyces cerevisiae species selected from following strains: INVSc1, YNN27, S150-2B, W303-1B, CG25, W3124, JRY188, BJ5464, AH22, GRF18, W303-1A and BJ3505. In some embodiments, members of the Pichia species including any species of Pichia, for example the Pichia species, Pichia pastoris, for example, the Pichia pastoris is selected from following strains: Bg08, Y-11430, X-33, GS115, GS190, JC220, JC254, GS200, JC227, JC300, JC301, JC302, JC303, JC304, JC305, JC306, JC307, JC308, YJN165, KM71, MC100-3, SMD1163, SMD1165, SMD1168, GS241, MS105, any pep4 knock-out strain and any prb1 knock-out strain, as well as Pichia pastoris selected from following strains: Bg08, X-33, SMD1168 and KM71. In some embodiments, any Kluyveromyces species can be used to accomplish the methods described here, including any species of Kluyveromyces, for example, Kluyveromyces lactis, and we teach that the stain of Kluyveromyces lactis can be but is not required to be selected from following strains: GG799, YCT306, YCT284, YCT389, YCT390, YCT569, YCT598, NRRL Y-1140, MW98-8C, MS1, CBS293.91, Y721, MD2/1, PM6-7A, WM37, K6, K7, 22AR1, 22A295-1, SD11, MG1/2, MSK110, JA6, CMK5, HP101, HP108 and PM6-3C, in addition to Kluyveromyces lactis species is selected from GG799, YCT306 and NRRL Y-1140.

In some embodiments, the host cell used to produce a KPV or a KPV-pesticidal protein can be an Aspergillus oryzae.

In some embodiments, the host cell used to produce a KPV or a KPV-pesticidal protein can be an Aspergillus japonicas.

In some embodiments, the host cell used to produce a KPV or a KPV-pesticidal protein can be an Aspergillus niger.

In some embodiments, the host cell used to produce a KPV or a KPV-pesticidal protein can be a Bacillus licheniformis.

In some embodiments, the host cell used to produce a KPV or a KPV-pesticidal protein can be a Bacillus subtilis.

In some embodiments, the host cell used to produce a KPV or a KPV-pesticidal protein can be a Trichoderma reesei.

In some embodiments, the procedures and methods described here can be accomplished with any species of yeast, including but not limited to any species of Hansenula species including any species of Hansenula and preferably Hansenula polymorpha. In some embodiments, the procedures and methods described here can be accomplished with any species of yeast, including but not limited to any species of Yarrowia species for example, Yarrowia lipolytica. In some embodiments, the procedures and methods described here can be accomplished with any species of yeast, including but not limited to any species of Schizosaccharomyces species including any species of Schizosaccharomyces and preferably Schizosaccharomyces pombe.

In some embodiments, the host cell used to produce a KPV or a KPV-pesticidal protein can be a species selected from: Saccharomyces cerevisiae, Pichia pastoris, Hansenula polymorpha, Yarrowia lipolytica, Arxula adeninivorans, Kluyveromyces lactis, or Schizosaccharomyces pombe.

In some illustrative embodiments, yeast species such as Kluyveromyces lactis, Saccharomyces cerevisiae, Pichia pastoris, and others, can be used as a host organism. Yeast cell culture techniques are well known to those having ordinary skill in the art. Exemplary methods of yeast cell culture can be found in Evans, Yeast Protocols. Springer (1996); Bill, Recombinant Protein Production in Yeast. Springer (2012); Hagan et al., Fission Yeast: A Laboratory Manual, CSH Press (2016); Konishi et al., Improvement of the transformation efficiency of Saccharomyces cerevisiae by altering carbon sources in pre-culture. Biosci Biotechnol Biochem. 2014; 78(6): 1090-3; Dymond, Saccharomyces cerevisiae growth media. Methods Enzymol. 2013; 533:191-204; Looke et al., Extraction of genomic DNA from yeasts for PCR-based applications. Biotechniques. 2011 May; 50(5):325-8; and Romanos et al., Culture of yeast for the production of heterologous proteins. Curr Protoc Cell Biol. 2014 Sep. 2; 64:20.9.1-16, the disclosures of which are incorporated herein by reference in their entireties.

Recipes for yeast cell fermentation media and stocks are described herein.

### Yeast Strains

The present disclosure contemplates the creation of yeast strains operable to express a KPV or a KPV-pesticidal protein. For example, in some embodiments, a host cell can be transformed with a polynucleotide operable to encode a KPV (e.g., by using any of the vectors described herein). In some embodiments, that host cell can be yeast strain.

In some embodiments, a yeast strain can be produced by preparing a vector comprising a first expression cassette comprising a polynucleotide operable to express a KPV or complementary nucleotide sequence thereof.

In some embodiments, a yeast strain of the present disclosure comprises, consists essentially of, or consists of: a first expression cassette comprising a polynucleotide operable to encode a KPV comprising, consisting essentially of, or consisting of, an amino acid sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or complementary nucleotide sequence thereof, with the proviso that the KPV does not comprise the amino acid sequence of SEQ ID NOs: 1 or 253.

In some embodiments, the yeast strain comprises a polynucleotide which enables the synthesis of a KPV, wherein the KPV comprises, consists essentially of, or consists of, an amino sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; wherein the KPV comprises at least one amino acid substitution relative to the amino acid sequence set forth in SEQ ID NO: 1, or a complementary nucleotide sequence thereof; with the proviso that the KPV does not comprise the amino acid sequence of SEQ ID NOs: 1 or 253.

In some embodiments, the yeast strain is selected from any species belonging to the genera Saccharomyces, Pichia, Kluyveromyces, Hansenula, Yarrowia or Schizosaccharomyces.

In some embodiments, the yeast cell is selected from the group consisting of Kluyveromyces lactis, Kluyveromyces marxianus, Saccharomyces cerevisiae, and Pichia pastoris.

In some embodiments, the yeast cell is Kluyveromyces lactis or Kluyveromyces marxianus.

In some embodiments, a yeast strain can be operable to express a KPV or KPV-pesticidal protein, wherein the KPV is a homopolymer or heteropolymer of two or more KPVs, wherein the amino acid sequence of each KPV is the same or different.

In some embodiments, a yeast strain can be operable to express a KPV or KPV-pesticidal protein, wherein the KPV is a fused protein comprising two or more KPVs separated directly or indirectly by a cleavable or non-cleavable linker, and wherein the amino acid sequence of each KPV may be the same or different.

In some embodiments, the linker is a cleavable linker.

In some embodiments, the linker has an amino acid sequence as set forth in any one of SEQ ID NOs: 360-369.

In some embodiments, the linker is cleavable inside at least one of (i) the gut or hemolymph of an insect, and (ii) cleavable inside the gut of a mammal.

In some embodiments, a yeast strain can be operable to express a KPV or KPV-pesticidal protein, wherein the vector is a plasmid comprising an alpha-MF signal.

In some embodiments, a yeast strain can be operable to express a KPV or KPV-pesticidal protein, wherein the vector is transformed into a yeast strain.

In some embodiments, a yeast strain can be operable to express a KPV or KPV-pesticidal protein, wherein the yeast strain is selected from any species of the genera Saccharomyces, Pichia, Kluyveromyces, Hansenula, Yarrowia or Schizosaccharomyces.

In some embodiments, a yeast strain can be operable to express a KPV or KPV-pesticidal protein, wherein the yeast strain is selected from the group consisting of Kluyveromyces lactis, Kluyveromyces marxianus, Saccharomyces cerevisiae, and Pichia pastoris.

In some embodiments, a yeast strain can be operable to express a KPV or KPV-pesticidal protein, wherein the yeast strain is Kluyveromyces lactis.

In some embodiments, a yeast strain can be operable to express a KPV or KPV-pesticidal protein, wherein expression of the KPV in the medium results in the expression of a single KPV in the medium.

In some embodiments, a yeast strain can be operable to express a KPV or KPV-pesticidal protein, wherein expression of the KPV in the medium results in the expression of a KPV polymer comprising two or more KPV polypeptides in the medium.

In some embodiments, a yeast strain can be operable to express a KPV or KPV-pesticidal protein, wherein the vector comprises two or three expression cassettes, each expression cassette operable to encode the KPV of the first expression cassette.

In some embodiments, a yeast strain can be operable to express a KPV or KPV-pesticidal protein, wherein the vector comprises two or three expression cassettes, each expression cassette operable to encode the KPV of the first expression cassette, or a KPV of a different expression cassette.

In some embodiments, a yeast strain can be operable to express a KPV or KPV-pesticidal protein, wherein the expression cassette is operable to encode a KPV as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

Any of the aforementioned methods, and/or any of the methods described herein, can be used to produce one or more of the KPVs or KPV-pesticidal proteins as described herein. For example, any of the methods described herein can be used to produce one or more of the KPVs described in the present disclosure, e.g., KPVs having the amino acid sequence of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, which are likewise described herein.

### Yeast Transformation, KPV Purification, and Analysis

An exemplary method of yeast transformation is as follows: first, expression vectors carrying a KPV ORF are transformed into yeast cells; the expression vectors are usually linearized by specific restriction enzyme cleavage to facilitate chromosomal integration via homologous recombination. The linear expression vector is then transformed into yeast cells by a chemical or electroporation method of transformation and integrated into the targeted locus of the yeast genome by homologous recombination. The integration can happen at the same chromosomal locus multiple times; therefore, the genome of a transformed yeast cell can contain multiple copies of KPV expression cassettes. The successfully transformed yeast cells can be identified using growth conditions that favor a selection marker engineered into the expression vector and co-integrated into yeast chromosomes with the KPV ORF; examples of such markers include, but are not limited to, acetamide prototrophy, zeocin resistance, geneticin resistance, nourseothricin resistance, and uracil prototrophy.

Selection makers are well known in the art, and any of these well-known selection markers can be implemented in the present disclosure. For example, in some embodiments, a selection marker can be a positive selection marker, or negative selection marker. Positive selection markers permit the selection for cells in which the gene product of the marker is expressed. This generally comprises contacting cells with an appropriate agent that, but for the expression of the positive selection marker, kills or otherwise selects against the cells. An exemplary method of using selection markers is disclosed in U.S. Pat. No. 5,464,764, the disclosure of which is incorporated herein by reference in its entirety. Additional exemplary descriptions and methods concerning selection markers are provided in Wigler et al., Cell 11:223 (1977); Szybalska & Szybalski, Proc. Natl. Acad. Sci. USA 48:202 (1992); Lowy et al., Cell 22:817 (1980); Wigler et al., Natl. Acad. Sci. USA 77:357 (1980); O'Hare et al., Proc. Natl. Acad. Sci. USA 78:1527 (1981); Mulligan & Berg, Proc. Natl. Acad. Sci. USA 78:2072 (1981); Wu and Wu, Biotherapy 3:87-95 (1991); Tolstoshev, Ann. Rev. Pharmacol. Toxicol. 32:573-596 (1993); Mulligan, Science 260:926-932 (1993); Morgan and Anderson, Ann. Rev. Biochem. 62:191-217 (1993); Santerre et al., Gene 30:147 (1984); Ausubel et al. (eds.), Current Protocols in Molecular Biology, John Wiley & Sons, N Y (1993); Kriegler, Gene Transfer and Expression, A Laboratory Manual, Stockton Press, N Y (1990); in Chapters 12 and 13, Dracopoli et al. (eds), Current Protocols in Human Genetics, John Wiley & Sons, N Y (1994); Colberre-Garapin et al., J. Mol. Biol. 150:1 (1981); U.S. Pat. No. 6,548,285 (filed Apr. 3, 1997); U.S. Pat. No. 6,165,715 (filed Jun. 22, 1998); and U.S. Pat. No. 6,110,707 (filed Jan. 17, 1997), the disclosures of which are incorporated by reference herein in their entireties.

Due to the influence of unpredictable and variable factors—such as epigenetic modification of genes and networks of genes, and variation in the number of integration events that occur in individual cells in a population undergoing a transformation procedure—individual yeast colonies of a given transformation process will differ in their capacities to produce a KPV ORF. Therefore, transgenic yeast colonies carrying the KPV transgenes should be screened for high yield strains. Two effective methods for such screening—each dependent on growth of small-scale cultures of the transgenic yeast to provide conditioned media samples for subsequent analysis—use reverse-phase HPLC or housefly injection procedures to analyze conditioned media samples from the positive transgenic yeast colonies.

The transgenic yeast cultures can be obtained, e.g., using 14 mL round bottom polypropylene culture tubes with 5 to 10 mL defined medium added to each tube, or in 48-well deep well culture plates with 2.2 mL defined medium added to each well. The defined medium, not containing crude proteinaceous extracts or by-products such as yeast extract or peptone, is used for the cultures to reduce the protein background in the conditioned media harvested for the later screening steps. The cultures are performed at the optimal temperature, for example, 23.5° C. for K. lactis, for about 5-6 days, until the maximum cell density is reached. KPVs will now be produced by the transformed yeast cells and secreted out of cells to the growth medium. To prepare samples for the screening, cells are removed from the cultures by centrifugation and the supernatants are collected as the conditioned media, which are then cleaned by filtration through 0.22 μm filter membrane and then made ready for strain screening.

In some embodiments, positive yeast colonies transformed with KPV can be screened via reverse-phase HPLC (rpHPLC) screening of putative yeast colonies. In this screening method, an HPLC analytic column with bonded phase of C18 can be used. Acetonitrile and water are used as mobile phase solvents, and a UV absorbance detector set at 220 nm is used for the peptide detection. Appropriate amounts of the conditioned medium samples are loaded into the rpHPLC system and eluted with a linear gradient of mobile phase solvents. The corresponding peak area of the pesticidal peptide in the HPLC chromatograph is used to quantify the KPV concentrations in the conditioned media. Known amounts of pure KPV are run through the same rpHPLC column with the same HPLC protocol to confirm the retention time of the peptide and to produce a standard peptide HPLC curve for the quantification.

An exemplary reverse-phase HPLC screening process of positive K. lactis cells is as follows: a KPV ORF can be inserted into the expression vector, pKLAC1, and transformed into the K. lactis strain, YCT306, from New England Biolabs, Ipswich, MA, USA. pKLAC1 vector is an integrative expression vector. Once the KPV transgenes were cloned into pKLAC1 and transformed into YCT306, their expression was controlled by the LAC4 promoter. The resulting transformed colonies produced pre-propeptides comprising an α-mating factor signal peptide, a Kex2 cleavage site and mature KPVs. The α-Mating factor signal peptide guides the pre-propeptides to enter the endogenous secretion pathway, and mature KPVs are released into the growth media.

In some embodiments, codon optimization for KPV expression can be performed in two rounds, for example, in the first round, based on some common features of high expression DNA sequences, multiple variants of the KPV ORF, expressing an α-Mating factor signal peptide, a Kex2 cleavage site and the KPV, are designed and their expression levels are evaluated in the YCT306 strain of K. lactis, resulting in an initial K. lactis expression algorithm; in a second round of optimization, additional variant KPV ORFs can be designed based on the initial K. lactis expression algorithm to further fine-tuned the K. lactis expression algorithm, and identify the best ORF for KPV expression in K. lactis. In some embodiments, the resulting DNA sequence from the foregoing optimization can have an open reading frame encoding an α-MF signal peptide, a Kex2 cleavage site and a KPV, which can be cloned into the pKLAC1 vector using Hind III and Not I restriction sites, resulting in KPV expression vectors.

In some embodiments, the yeast, Pichia pastoris, can be transformed with a KPV expression cassette. An exemplary method for transforming P. pastoris is as follows: yeast vectors can be used to transform a KPV expression cassette into P. pastoris. The vectors can be obtained from commercial vendors known to those having ordinary skill in the art. In some embodiments, the vectors can be integrative vectors, and may use the uracil phosphoribosyltransferase promoter (pUPP) to enhance the heterologous transgene expression. In some embodiments, the vectors may offer different selection strategies; e.g., in some embodiments, the only difference between the vectors can be that one vector may provide G418 resistance to the host yeast, while the other vector may provide Zeocin resistance. In some embodiments, pairs of complementary oligonucleotides, encoding the KPV may be designed and synthesized for subcloning into the two yeast expression vectors. Hybridization reactions can be performed by mixing the corresponding complementary oligonucleotides to a final concentration of 20 μM in 30 mM NaCl, 10 mM Tris-Cl (all final concentrations), pH 8, and then incubating at 95° C. for 20 min, followed by a 9-hour incubation starting at 92° C. and ending at 17° C., with 3° C. drops in temperature every 20 min. The hybridization reactions will result in DNA fragments encoding KPV. The two P. pastoris vectors can be digested with BsaI-HF restriction enzymes, and the double stranded DNA products of the reactions are then subcloned into the linearized P. pastoris vectors using standard procedures. Following verification of the sequences of the subclones, plasmid aliquots can be transfected by electroporation into a P. pastoris strain (e.g., Bg08). The resulting transformed yeast, can be selected based on resistance (e.g., in this example, to Zeocin or G418) conferred by elements engineered into the vectors.

Methods of protein purification are well-known in the art, and any known method can be employed to purify and/or recover KPVs of the present disclosure. For example, in some embodiments, the following procedures are exemplary of suitable purification procedures: fractionation on immunoaffinity or ion-exchange columns; ethanol precipitation; reverse phase HPLC; chromatography on silica, or on a cation-exchange resin such as DEAE; chromatofocusing; SDS-PAGE; ammonium sulfate precipitation; gel filtration using, for example, Sephadex G-75; and the like. In some embodiments, proteins of the present disclosure can be purified using one of the following; affinity chromatography; ion exchange chromatography; filtration; electrophoresis; hydrophobic interaction chromatography; gel filtration chromatography; reverse phase chromatography; concanavalin A chromatography; and differential solubilization.

Exemplary methods of protein purification are provided in: U.S. Pat. Nos. 6,339,142; 7,585,955; 8,946,395; 9,067,990; 10,246,484; and Marshak et al., “Strategies for Protein Purification and Characterization—A Laboratory Course Manual” CSHL Press (1996); the disclosures of which are incorporated herein by reference in their entireties.

### Peptide Yield Screening and Evaluation

Peptide yield can be determined by any of the methods known to those of skill in the art (e.g., capillary gel electrophoresis (CGE), Western blot analysis, and the like). Activity assays, as described herein and known in the art, can also provide information regarding peptide yield. In some embodiments, these or any other methods known in the art can be used to evaluate peptide yield.

### Quantification Assays

In some embodiments, and without limitation, KPV peptide yield can be measured using: HPLC; Mass spectrometry (MS) and related techniques; LC/MS/MS; reverse phase protein arrays (RPPA); immunohistochemistry; ELISA; suspension bead array, mass spectrometry; dot blot; SDS-PAGE; capillary gel electrophoresis (CGE); Western blot analysis; Bradford assay; measuring UV absorption at 260 nm; Lowry assay; Smith copper/bicinchoninic assay; a secretion assay; Pierce protein assay; Biuret reaction; and the like. Exemplary methods of protein quantification are provided in Stoscheck, C. 1990 “Quantification of Protein” Methods in Enzymology, 182:50-68; Lowry, O. Rosebrough, A., Farr, A. and Randall, R. 1951 J. Biol. Chem. 193:265; Smith, P. et al., (1985) Anal. Biochem. 150:76-85; Bradford, M. 1976 “A Rapid and Sensitive Method for the Quantitation of Microgram Quantities of Protein Utilizing the Principle of Protein-Dye Binding” Anal. Biochem. 72:248-254; Cabib, E. and Polacheck, I. 1984 “Protein assay for dilute solutions.” Methods in Enzymology, 104:318-328; Turcanu, Victor; Williams, Neil A. (2001). “Cell identification and isolation on the basis of cytokine secretion: A novel tool for investigating immune responses.” Nature Medicine. 7 (3): 373-376; and U.S. Pat. No. 6,391,649; the disclosures of which are incorporated herein by reference in their entireties.

In other embodiments, KPV peptide yield can be quantified and/or assessed using methods that include, without limitation: recombinant protein mass per volume of culture (e.g., gram or milligrams protein per liter culture); percent or fraction of recombinant protein insoluble precipitate obtained after cell lysis determined in (e.g., recombinant protein extracted supernatant in an amount/amount of protein in the insoluble components); percentage or fraction of active protein (e.g., an amount/analysis of the active protein for use in protein amount); total cell protein (tcp) percentage or fraction; and/or the amount of protein/cell and the dry biomass of a percentage or ratio.

In some embodiments, wherein yield is expressed in terms of culture volume, the culture cell density may be taken into account, particularly when yields between different cultures are being compared.

In some embodiments, the present disclosure provides a method of producing a heterologous polypeptide that is at least about 5%, at least about 10%, about 15%, about 20%, about 25%, about 30%, about 40%, about 45%, about 50%, about 55%, about 60%, about 65%, about 70%, about 75%, or greater of total cell protein (tcp). “Percent total cell protein” is the amount of heterologous polypeptide in the host cell as a percentage of aggregate cellular protein. The determination of the percent total cell protein is well known in the art.

“Total cell protein (tcp)” or “Percent total cell protein (% tcp)” is the amount of protein or polypeptide in the host cell as a percentage of aggregate cellular protein. Methods for the determination of the percent total cell protein are well known in the art.

In some embodiments, HPLC can be used to quantify peptide yield. For example, in some embodiments, peptide yield can be quantified using an Agilent 1100 HPLC system equipped with an Onyx monolithic 4.5×100 mm, C18 reverse-phase analytical HPLC column and an auto-injector. An illustrative use of the Agilent 1100 HPLC system equipped with an Onyx monolithic 4.5×100 mm, C18 reverse-phase analytical HPLC column and an auto-injector is as follows: filtered conditioned media samples from transformed K. lactis cells are analyzed using Agilent 1100 HPLC system equipped with an Onyx monolithic 4.5×100 mm, C18 reverse-phase analytical HPLC column and an auto-injector by analyzing HPLC grade water and acetonitrile containing 0.1% trifluoroacetic acid, constituting the two mobile phase solvents used for the HPLC analyses; the peak areas of both the KPV or KPV-pesticidal protein are analyzed using HPLC chromatographs, and then used to calculate the peptide concentration in the conditioned media, which can be further normalized to the corresponding final cell densities (as determined by OD600 measurements) as normalized peptide yield.

### Activity Assays

In some embodiments, positive yeast colonies transformed with KPV or KPV-pesticidal protein can be screened using a housefly injection assay. KPV or KPV-pesticidal protein can paralyze/kill houseflies when injected in measured doses through the body wall of the dorsal thorax. The efficacy of the KPV or KPV-pesticidal protein can be defined by the median paralysis/lethal dose of the peptide (PD₅₀/LD₅₀), which causes 50% knock-down ratio or mortality of the injected houseflies respectively. The pure KPV or KPV-pesticidal protein is normally used in the housefly injection assay to generate a standard dose-response curve, from which a PD₅₀/LD₅₀ value can be determined. Using a PD₅₀/LD₅₀ value from the analysis of a standard dose-response curve of the pure KPV or KPV-pesticidal protein, quantification of the KPV or KPV-pesticidal protein produced by the transformed yeast can be achieved using a housefly injection assay performed with serial dilutions of the corresponding conditioned media.

An exemplary housefly injection bioassay is as follows: conditioned media is serially diluted to generate full dose-response curves from the housefly injection bioassay. Before injection, adult houseflies (Musca domestica) are immobilized with CO₂, and 12-18 mg houseflies are selected for injection. A microapplicator, loaded with a 1 cc syringe and 30-gauge needle, is used to inject 0.5 μL per fly, doses of serially diluted conditioned media samples into houseflies through the body wall of the dorsal thorax. The injected houseflies are placed into closed containers with moist filter paper and breathing holes on the lids, and they are examined by knock-down ratio or by mortality scoring at 24 hours post-injection. Normalized yields are calculated. Peptide yield means the peptide concentration in the conditioned media in units of mg/L. However, peptide yields are not always sufficient to accurately compare the strain production rate. Individual strains may have different growth rates, hence when a culture is harvested, different cultures may vary in cell density. A culture with a high cell density may produce a higher concentration of the peptide in the media, even though the peptide production rate of the strain is lower than another strain which has a higher production rate. Accordingly, the term “normalized yield” is created by dividing the peptide yield with the cell density in the corresponding culture and this allows a better comparison of the peptide production rate between strains. The cell density is represented by the light absorbance at 600 nm with a unit of “A” (Absorbance unit).

Screening yeast colonies that have undergone a transformation with a polynucleotide operable to encode a KPV or KPV-pesticidal protein can identify the high yield yeast strains from hundreds of potential colonies. These strains can be fermented in bioreactor to achieve at least up to 4 g/L or at least up to 3 g/L or at least up to 2 g/L yield of the KPV or KPV-pesticidal protein when using optimized fermentation media and fermentation conditions described herein. The higher rates of production (expressed in mg/L) can be anywhere from about 100 mg/L to about 100,000 mg/L; or from about 100 mg/L to about 90,000 mg/L; or from about 100 mg/L to about 80,000 mg/L; or from about 100 mg/L to about 70,000 mg/L; or from about 100 mg/L to about 60,000 mg/L; or from about 100 mg/L to about 50,000 mg/L; or from about 100 mg/L to about 40,000 mg/L; or from about 100 mg/L to about 30,000 mg/L; or from about 100 mg/L to about 20,000 mg/L; or from about 100 mg/L to about 17,500 mg/L; or from about 100 mg/L to about 15,000 mg/L; or from about 100 mg/L to about 12,500 mg/L; or from about 100 mg/L to about 10,000 mg/L; or from about 100 mg/L to about 9,000 mg/L; or from about 100 mg/L to about 8,000 mg/L; or from about 100 mg/L to about 7,000 mg/L; or from about 100 mg/L to about 6,000 mg/L; or from about 100 mg/L to about 5,000 mg/L; or from about 100 mg/L to about 3,000 mg/L; or from about 100 mg/L to 2,000 mg/L; or from about 100 mg/L to 1,500 mg/L; or from about 100 mg/L to 1,000 mg/L; or from about 100 mg/L to 750 mg/L; or from about 100 mg/L to 500 mg/L; or from about 150 mg/L to 100,000 mg/L; or from about 200 mg/L to 100,000 mg/L; or from about 300 mg/L to 100,000 mg/L; or from about 400 mg/L to 100,000 mg/L; or from about 500 mg/L to 100,000 mg/L; or from about 750 mg/L to 100,000 mg/L; or from about 1,000 mg/L to 100,000 mg/L; or from about 1,250 mg/L to 100,000 mg/L; or from about 1,500 mg/L to 100,000 mg/L; or from about 2,000 mg/L to 100,000 mg/L; or from about 2,500 mg/L to 100,000 mg/L; or from about 3,000 mg/L to 100,000 mg/L; or from about 3,500 mg/L to 100,000 mg/L; or from about 4,000 mg/L to 100,000 mg/L; or from about 4,500 mg/L to 100,000 mg/L; or from about 5,000 mg/L to 100,000 mg/L; or from about 6,000 mg/L to 100,000 mg/L; or from about 7,000 mg/L to 100,000 mg/L; or from about 8,000 mg/L to 100,000 mg/L; or from about 9,000 mg/L to 100,000 mg/L; or from about 10,000 mg/L to 100,000 mg/L; or from about 12,500 mg/L to 100,000 mg/L; or from about 15,000 mg/L to 100,000 mg/L; or from about 17,500 mg/L to 100,000 mg/L; or from about 20,000 mg/L to 100,000 mg/L; or from about 30,000 mg/L to 100,000 mg/L; or from about 40,000 mg/L to 100,000 mg/L; or from about 50,000 mg/L to 100,000 mg/L; or from about 60,000 mg/L to 100,000 mg/L; or from about 70,000 mg/L to 100,000 mg/L; or from about 80,000 mg/L to 100,000 mg/L; or from about 90,000 mg/L to 100,000 mg/L; or any range of any value provided or even greater yields than can be achieved with a peptide before conversion, using the same or similar production methods that were used to produce the peptide before conversion.

### Detecting Peptide Degradation and Stability

Proteins, polypeptides, and peptides degrade in both biological samples and in solution (e.g., cell culture and/or during fermentation).

Methods of detecting peptide degradation are well known in the art. Any of the well-known methods of detecting peptide degradation (e.g., during fermentation) may be employed here.

In some embodiments, stability can be evaluated qualitatively and/or quantitatively in a variety of different ways, including evaluation of aggregate formation (for example using size exclusion chromatography, by measuring turbidity, and/or by visual inspection). In other embodiments, stability can be evaluated by assessing charge heterogeneity using ion exchange chromatography (IEC), size exclusion chromatography (HP-SEC), SDS-PAGE analysis to compare reduced and intact proteins. In other embodiments, evaluating biological activity or binding function can be used.

In some embodiments, peptide degradation can be detected using isotope labeling techniques; liquid chromatography/mass spectrometry (LC/MS); HPLC; radioactive amino acid incorporation and subsequent detection, e.g., via scintillation counting; the use of a reporter protein, e.g., a protein that can be detected (e.g., by fluorescence, spectroscopy, luminometry, etc.); fluorescent intensity of one or more bioluminescent proteins and/or fluorescent proteins and/or fusions thereof; pulse-chase analysis (e.g., pulse-labeling a cell with radioactive amino acids and following the decay of the labeled protein while chasing with unlabeled precursor, and arresting protein synthesis and measuring the decay of total protein levels with time); cycloheximide-chase assays; and the use of non-isotope labeled techniques; all of which are known to those having ordinary skill in the art.

In some embodiments, an assay can be used to detect peptide degradation, wherein a sample is contacted with a non-fluorescent compound that is operable to react with free primary amine in said sample produced via the degradation of a peptide, and which then produces a fluorescent signal that can be quantified and compared to a standard. Examples of non-fluorescent compounds that can be utilized as fluorescent tags for free amines according to the present disclosure are 3-(4-carboxybenzoyl) quinoline-2-carboxaldehyde (CBQCA), fluorescamine, and o-phthaldialdehyde.

In some embodiments, the method to determine the readout signal from the reporter protein depends from the nature of the reporter protein. For example, for fluorescent reporter proteins, the readout signal corresponds to the intensity of the fluorescent signal. The readout signal may be measured using spectroscopy-, fluorometry-, photometry-, and/or luminometry-based methods and detection systems, for example. Such methods and detection systems are well known in the art.

In some embodiments, standard immunological procedures known to those having ordinary skill in the art can be used to detect peptide degradation. For example, in some embodiments, peptide degradation can be detected in a sample using immunoassays that employ a detectable antibody. Such immunoassays include, for example, agglutination assays, ELISA, Pandex microfluorimetric assay, flow cytometry, serum diagnostic assays, and immunohistochemical staining procedures, all of which are well-known in the art. In some embodiments, the levels (e.g., of fluorescence) in one sample can be compared to a standard. An antibody can be made detectable by various means well known in the art. For example, a detectable marker can be directly or indirectly attached to the antibody. Useful markers include, for example, radionucleotides, enzymes, fluorogens, chromogens and chemiluminescent labels.

Exemplary methods of detecting peptide degradation is provided in U.S. Pat. Nos. 5,766,927; 7,504,253; 9,201,073; 9,429,566; U.S. patent application Ser. No. 20/120,028286; Eldeeb et al., A molecular toolbox for studying protein degradation in mammalian cells. J Neurochem. 2019 November; 151 (4): 520-533; and Buchanan et al., Cycloheximide Chase Analysis of Protein Degradation in Saccharomyces cerevisiae. J Vis Exp. 2016; (110): 53975, the disclosures of which are incorporated herein by reference in their entireties.

Exemplary methods of assessing stability of peptides are provided in Peptide and Protein Drug Delivery, 247-301, Vincent Lee Ed., Marcel Dekker, Inc., New York, N.Y., 1991; and Jones, A., Adv. Drug Delivery Rev. 10:29-90 (1993); the disclosures of which are incorporated herein by reference in their entireties.

Exemplary methods of measuring deamidation include imaged capillary isoelectric focusing (icIEF), as described in U.S. Pat. No. 11,248,031, the disclosure of which is incorporated herein by reference in its entirety. Other exemplary methods are described in U.S. Pat. Nos. 11,280,791; 10,875,914; and 10,570,185, the disclosures of which are incorporated herein by reference in their entireties.

### Culture and Fermentation Conditions

Cell culture techniques are well-known in the art. In some embodiments, the culture method and/or materials will necessarily require adaption based on the host cell selected; and, such adaptions (e.g., modifying pH, temperature, medium contents, and the like) are well known to those having ordinary skill in the art. In some embodiments, any known culture technique may be employed to produce a KPV or KPV-pesticidal protein of the present disclosure.

Exemplary culture methods are provided in U.S. Pat. Nos. 3,933,590; 3,946,780; 4,988,623; 5,153,131; 5,153,133; 5,155,034; 5,316,905; 5,330,908; 6,159,724; 7,419,801; 9,320,816; 9,714,408; and 10,563,169; the disclosures of which are incorporated herein by reference in their entireties.

### Yeast Culture

Yeast cell culture techniques are well known to those having ordinary skill in the art. Exemplary methods of yeast cell culture can be found in Evans, Yeast Protocols. Springer (1996); Bill, Recombinant Protein Production in Yeast. Springer (2012); Hagan et al., Fission Yeast: A Laboratory Manual, CSH Press (2016); Konishi et al., Improvement of the transformation efficiency of Saccharomyces cerevisiae by altering carbon sources in pre-culture. Biosci Biotechnol Biochem. 2014; 78(6): 1090-3; Dymond, Saccharomyces cerevisiae growth media. Methods Enzymol. 2013; 533:191-204; Looke et al., Extraction of genomic DNA from yeasts for PCR-based applications. Biotechniques. 2011 May; 50(5): 325-8; and Romanos et al., Culture of yeast for the production of heterologous proteins. Curr Protoc Cell Biol. 2014 Sep. 2; 64:20.9.1-16, the disclosures of which are incorporated herein by reference in their entireties.

Yeast can be cultured in a variety of media, e.g., in some embodiments, yeast can be cultured in minimal medium; YPD medium; yeast synthetic drop-out medium; Yeast Nitrogen Base (YNB with or without amino acids); YEPD medium; ADE D medium; ADE DS″ medium; LEU D medium; HIS D medium; or Mineral salts medium.

In some embodiments, yeast can be cultured in minimal medium. In some embodiments, minimal medium ingredients can comprise: 2% Sugar; Phosphate Buffer, pH 6.0; Magnesium Sulfate; Calcium Chloride; Ammonium Sulfate; Sodium Chloride; Potassium Chloride; Copper Sulfate; Manganese Sulfate; Zinc Chloride; Potassium Iodide; Cobalt Chloride; Sodium Molybdate; Boric Acid; Iron Chloride; Biotin; Calcium pantothenate; Thiamine; Myo-inositol; Nicotinic Acid; and Pyridoxine.

In some embodiments, yeast can be cultured in YPD medium. YPD medium comprises a bacteriological peptone, yeast extract, and glucose.

In some embodiments, yeast can be cultured in yeast synthetic drop-out medium, which can be used to differentiate auxotrophic mutant strains that cannot grow without a specific medium component transformed with a plasmid that allows said transformant to grow on a medium lacking the required component.

In some embodiments, yeast can be cultured using Yeast Nitrogen Base (YNB with or without amino acids), which comprises nitrogen, vitamins, trace elements, and salts.

In some embodiments, the medium can be YEPD medium, e.g., a medium comprising 2% D-glucose, 2% BACTO Peptone (Difco Laboratories, Detroit, MI), 1% BACTO yeast extract (Difco), 0.004% adenine, and 0.006% L-leucine; or, a variation thereof, wherein the carbon source is a sugar alcohol, e.g., glycerol or sorbitol

In some embodiments, the medium can be ADE D medium, e.g., a medium comprising 0.056%-Ade-Trp-Thr powder, 0.67% yeast nitrogen base without amino acids, 2% D-glucose, and 0.5% 200× tryptophan, threonine solution; or, a variation thereof, wherein the carbon source is a sugar alcohol, e.g., glycerol or sorbitol

In some embodiments, the medium can be ADE DS″ medium, e.g., a medium comprising 0.056%-Ade-Trp-Thr powder, 0.67% yeast nitrogen base without amino acids, 2% D-glucose, 0.5% 200× tryptophan, threonine solution, and 18.22% D-sorbitol; or, a variation thereof, wherein the carbon source is entirely a sugar alcohol, e.g., glycerol or sorbitol

In some embodiments, the medium can be LEU D medium e.g., a medium comprising 0.052%-Leu-Trp-Thr powder, 0.67% yeast nitrogen base without amino acids, 2% D-glucose, and 0.5% 200× tryptophan, threonine solution; or, a variation thereof, wherein the carbon source is a sugar alcohol, e.g., glycerol or sorbitol.

In some embodiments, the medium can be HIS D medium, e.g., a medium comprising 0.052%-His-Trp-Thr powder, 0.67% yeast nitrogen base without amino acids, 2% D-glucose, and 0.5% 200× tryptophan, threonine solution; or, a variation thereof, wherein the carbon source is a sugar alcohol, e.g., glycerol or sorbitol.

In some embodiments, a mineral salts medium can be used. Mineral salts media consists of mineral salts and a carbon source such as, e.g., glucose, sucrose, or glycerol. Examples of mineral salts media include, e.g., M9 medium, Pseudomonas medium (ATCC 179), and Davis and Mingioli medium. See, Davis & Mingioli (1950) J. Bact. 60:17-28. The mineral salts used to make mineral salts media include those selected from among, e.g., potassium phosphates, ammonium sulfate or chloride, magnesium sulfate or chloride, and trace minerals such as calcium chloride, borate, and sulfates of iron, copper, manganese, and zinc. Typically, no organic nitrogen source, such as peptone, tryptone, amino acids, or a yeast extract, is included in a mineral salts medium. Instead, an inorganic nitrogen source is used and this may be selected from among, e.g., ammonium salts, aqueous ammonia, and gaseous ammonia. A mineral salts medium will typically contain glucose or glycerol as the carbon source.

In comparison to mineral salts media, minimal media can also contain mineral salts and a carbon source, but can be supplemented with, e.g., low levels of amino acids, vitamins, peptones, or other ingredients, though these are added at very minimal levels. Media can be prepared using the methods described in the art, e.g., in U.S. Pat. App. Pub. No. 2006/0040352, the disclosure of which is incorporated herein by reference in its entirety. Details of cultivation procedures and mineral salts media useful in the methods of the present disclosure are described by Riesenberg, D et al., 1991, “High cell density cultivation of Escherichia coli at controlled specific growth rate,” J. Biotechnol. 20 (1): 17-27, the disclosure of which is incorporated herein by reference in its entirety.

In some embodiments, Kluyveromyces lactis are grown in minimal media supplemented with 2% glucose, galactose, sorbitol, or glycerol as the sole carbon source. Cultures are incubated at 30° C. until mid-log phase (24-48 hours) for β-galactosidase measurements, or for 6 days at 23.5° C. for heterologous protein expression.

In some embodiments, yeast cells can be cultured in 48-well Deep-well plates, sealed after inoculation with sterile, air-permeable cover. Colonies of yeast, for example, K. lactis cultured on plates can be picked and inoculated the deep-well plates with 2.2 mL media per well, composed of DMSor. Inoculated deep-well plates can be grown for 6 days at 23.5° C. with 280 rpm shaking in a refrigerated incubator-shaker. On day 6 post-inoculation, conditioned media should be harvested by centrifugation at 4000 rpm for 10 minutes, followed by filtration using filter plate with 0.22 μM membrane, with filtered media are subject to HPLC analyses.

In some embodiments, yeast species such as Kluyveromyces lactis, Saccharomyces cerevisiae, Pichia pastoris, and others, can be used as a host organism, and/or the yeast to be modified using the methods described herein.

Temperature and pH conditions will vary depending on the stage of culture and the host cell species selected. Variables such as temperature and pH in cell culture are readily known to those having ordinary skill in the art.

The pH level is important in the culturing of yeast. One of skill in the art will appreciate that the culturing process includes not only the start of the yeast culture but the maintenance of the culture as well. The yeast culture may be started at any pH level, however, since the media of a yeast culture tends to become more acidic (i.e., lowering the pH) over time, care must be taken to monitor the pH level during the culturing process.

In some embodiments of the invention, the yeast is grown in a medium at a pH level that is dictated based on the species of yeast used, the stage of culture, and/or the temperature. Thus, in some embodiments, the pH level can fall within a range from about 2 to about 10. Those having ordinary skill in the art will recognize that the optimum pH for most microorganisms is near the neutral point (pH 7.0). However, in some embodiments, some fungal species prefer an acidic environment: accordingly, in some embodiments, the pH can range from 2 to 6.5. In some embodiments, the pH can range from about 4 to about 4.5. Some fungal species (e.g., molds) can grow can grow in a pH of from about 2 to about 8.5, but favor an acid pH. See Mountney & Gould, Practical food microbiology and technology. 1988. Ed. 3; and Pena et al., Effects of high medium pH on growth, metabolism and transport in Saccharomyces cerevisiae. FEMS Yeast Res. 2015 March; 15(2):fou005.

In other embodiments, the pH is about 5.7 to 5.9, 5.8 to 6.0, 5.9 to 6.1, 6.0 to 6.2, 6.1 to 6.3, 6.2 to 6.5, 6.4 to 6.7, 6.5 to 6.8, 6.6 to 6.9, 6.7 to 7.0, 6.8 to 7.1, 6.9 to 7.2, 7.0 to 7.3, 7.1 to 7.4, 7.2 to 7.5, 7.3 to 7.6, 7.4 to 7.7, 7.5 to 7.8, 7.6 to 7.9, 7.7 to 8.0, 7.8 to 8.1, 7.9 to 8.2, 8.0 to 8.3, 8.1 to 8.4, 8.2 to 8.5, 8.3 to 8.6, 8.4 to 8.7, or 8.5 to 8.8.

In some embodiments, the pH of the medium can be at least 5.5. In other aspects, the medium can have a pH level of about 5.5. In other aspects, the medium can have a pH level of between 4 and 8. In some cases, the culture is maintained at a pH level of between 5.5 and 8. In other aspects, the medium has a pH level of between 6 and 8. In some cases, medium has a pH level that is maintained at a pH level of between 6 and 8. In some embodiments, the yeast is grown and/or maintained at a pH level of between 6.1 and 8.1. In some embodiments, the yeast is grown and/or maintained at a pH level of between 6.2 and 8.2. In some embodiments, the yeast is grown and/or maintained at a pH level of between 6.3 and 8.3. In some embodiments, the yeast is grown and/or maintained at a pH level of between 6.4 and 8.4. In some embodiments, the yeast is grown and/or maintained at a pH level of between 5.5 and 8.5. In some embodiments, the yeast is grown and/or maintained at a pH level of between 6.5 and 8.5. In some embodiments, the yeast is grown at a pH level of about 5.6, 5.7, 5.8 or 5.9. In some embodiments, the yeast is grown at a pH level of about 6. In some embodiments, the yeast is grown at a pH level of about 6.5. In some embodiments, the yeast is grown at a pH level of about 6, 6.1, 6.2, 6.3, 6.4, 6.5, 6.6, 6.7, 6.8, 6.9 or 7.0. In some embodiments, the yeast is grown at a pH level of about 7, 7.1, 7.2, 7.3, 7.4, 7.5, 7.6, 7.7, 7.8, 7.9, or 8.0. In some embodiments, the yeast is grown at a level of above 8.

In some embodiments, the pH of the medium can range from a pH of 2 to 8.5. In certain embodiments, the pH is about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, or 8.8.

Exemplary methods of yeast culture can be found in U.S. Pat. No. 5,436,136, entitled “Repressible yeast promoters” (filed Dec. 20, 1991; assignee Ciba-Geigy Corporation); U.S. Pat. No. 6,645,739, entitled “Yeast expression systems, methods of producing polypeptides in yeast, and compositions relating to same” (filed Jul. 26, 2001; assignee Phoenix Pharmacologies, Inc., Lexington, KY); and U.S. Pat. No. 10,023,836, entitled “Medium for yeasts” (filed Aug. 23, 2013; assignee Yamaguchi University); the disclosures of which are incorporated herein by reference in their entireties.

### Fermentation

The present disclosure contemplates the culture of host organisms in any fermentation format. For example, batch, fed-batch, semi-continuous, and continuous fermentation modes may be employed herein.

Fermentation may be performed at any scale. The methods and techniques contemplated according to the present disclosure are useful for recombinant protein expression at any scale. Thus, in some embodiments, e.g., microliter-scale, milliliter scale, centiliter scale, and deciliter scale fermentation volumes may be used, and 1 Liter scale and larger fermentation volumes can be used.

In some embodiments, the fermentation volume is at or above about 1 Liter. For example, in some embodiments, the fermentation volume is about 1 liter to about 100 liters. In some embodiments, the fermentation volume is about 1 liter, about 2 liters, about 3 liters, about 4 liters, about 5 liters, about 6 liters, about 7 liters, about 8 liters, about 9 liters, or about 10 liters. In some embodiments, the fermentation volume is about 1 liter to about 5 liters, about 1 liter to about 10 liters, about 1 liter to about 25 liters, about 1 liter to about 50 liters, about 1 liter to about 75 liters, about 10 liters to about 25 liters, about 25 liters to about 50 liters, or about 50 liters to about 100 liters In other embodiments, the fermentation volume is at or above 5 Liters, 10 Liters, 15 Liters, 20 Liters, 25 Liters, 50 Liters, 75 Liters, 100 Liters, 200 Liters, 500 Liters, 1,000 Liters, 2,000 Liters, 5,000 Liters, 10,000 Liters, or 50,000 Liters.

In some embodiments, the fermentation medium can be a nutrient solution used for growing and or maintaining cells. Without limitation, this solution ordinarily provides at least one component from one or more of the following categories: (1) an energy source, usually in the form of a carbon source, e.g., glucose; (2) all essential amino acids, and usually the basic set of twenty amino acids; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range.

In some embodiments, the fermentation medium can be the same as the cell culture medium or any other media described herein. In some embodiments, the fermentation medium can be different from the cell culture medium. In some embodiments, the fermentation medium can be modified in order to accommodate the large-scale production of proteins.

In some embodiments, the fermentation medium can be supplemented electively with one or more components from any of the following categories: (1) hormones and other growth factors such as, serum, insulin, transferrin, and the like; (2) salts, for example, magnesium, calcium, and phosphate; (3) buffers, such as HEPES; (4) nucleosides and bases such as, adenosine, thymidine, etc.; (5) protein and tissue hydrolysates, for example peptone or peptone mixtures which can be obtained from purified gelatin, plant material, or animal byproducts; (6) antibiotics, such as gentamycin; and (7) cell protective agents, for example pluronic polyol.

In some embodiments, the pH of the fermentation medium can be maintained using pH buffers and methods known to those of skill in the art. Control of pH during fermentation can also can be achieved using aqueous ammonia. In some embodiments, the pH of the fermentation medium will be selected based on the preferred pH of the organism used. Thus, in some embodiments, and depending on the host cell and temperature, the pH can range from about to 1 to about 10.

In some embodiments, the pH of the fermentation medium can range from a pH of 2 to 8.5. In certain embodiments, the pH of the fermentation medium is about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, or 8.8.

In other embodiments, the pH of the fermentation medium is about 5.7 to 5.9, 5.8 to 6.0, 5.9 to 6.1, 6.0 to 6.2, 6.1 to 6.3, 6.2 to 6.5, 6.4 to 6.7, 6.5 to 6.8, 6.6 to 6.9, 6.7 to 7.0, 6.8 to 7.1, 6.9 to 7.2, 7.0 to 7.3, 7.1 to 7.4, 7.2 to 7.5, 7.3 to 7.6, 7.4 to 7.7, 7.5 to 7.8, 7.6 to 7.9, 7.7 to 8.0, 7.8 to 8.1, 7.9 to 8.2, 8.0 to 8.3, 8.1 to 8.4, 8.2 to 8.5, 8.3 to 8.6, 8.4 to 8.7, or 8.5 to 8.8.

In some embodiments, e.g., where Escherichia coli (E. coli) is used, the optimal pH range of the fermentation medium is between 6.5 and 7.5.

In other embodiments, e.g., where a yeast strain is used, the pH can range from about 4.0 to 8.0.

In some embodiments, neutral pH, i.e., a pH of about 7.0 can be used.

Those having ordinary skill in the art will recognize that during fermentation, the pH levels may drift as result of conversion and production of substrates and metabolic compounds.

In some embodiments, the fermentation medium can be supplemented with a buffer or other chemical in order to avoid changes to the pH. For example, in some embodiments, the addition of Ca(OH)₂, CaCO₃, NaOH, or NH₄OH can be added to the fermentation medium to neutralize the production of acidic compounds that occur, e.g., in some yeast species during industrial processes.

Temperature is another important consideration in the fermentation process; and, like pH considerations, temperature will depend on the type of host cell selected.

In some embodiments, the fermentation temperature is maintained at about 4° C. to about 42° C. In certain embodiments, the fermentation temperature is about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., or about 42° C.

In other embodiments, the fermentation temperature of the medium is maintained at about 25° C. to about 27° C., about 25° C. to about 28° C., about 25° C. to about 29° C., about 25° C. to about 30° C., about 25° C. to about 31° C., about 25° C. to about 32° C., about 25° C. to about 33° C., about 26° C. to about 28° C., about 26° C. to about 29° C., about 26° C. to about 30° C., about 26° C. to about 31° C., about 26° C. to about 32° C., about 27° C. to about 29° C., about 27° C. to about 30° C., about 27° C. to about 31° C., about 27° C. to about 32° C., about 26° C. to about 33° C., about 28° C. to about 30° C., about 28° C. to about 31° C., about 28° C. to about 32° C., about 29° C. to about 31° C., about 29° C. to about 32° C., about 29° C. to about 33° C., about 30° C. to about 32° C., about 30° C. to about 33° C., about 31° C. to about 33° C., about 31° C. to about 32° C., about 30° C. to about 33° C., or about 32° C. to about 33° C.

In other embodiments, the temperature is changed during fermentation, e.g., depending on the stage of fermentation.

Fermentation can be achieved with a variety of microorganisms known to those having ordinary skill in the art. Suitable microorganisms for up-scaled production of a KPV or KPV-pesticidal protein include any microorganism listed herein. In some embodiments, non-limiting examples of microorganisms include strains of the genus Saccharomyces spp. (including, but not limited to, S. cerevisiae (baker's yeast), S. distaticus, S. uvarum), the genus Kluyveromyces, (including, but not limited to, K. marxianus, K. fragilis), the genus Candida (including, but not limited to, C. pseudotropicalis, and C. brassicae), Pichia stipitis (a relative of Candida shehatae), the genus Clavispora (including, but not limited to, C. lusitaniae and C. opuntiae), the genus Pachysolen (including, but not limited to, P. tannophilus), the genus Bretannomyces (including, but not limited to, e.g., B. clausenii. Other suitable microorganisms include, for example, Zymomonas mobilis, Clostridium spp. (including, but not limited to, C. thermocellum; C. saccharobutylacetonicum, C. saccharobutylicum, C. Puniceum, C. beijernckii, and C. acetobutylicum), Moniliella pollinis, Moniliella megachiliensis, Lactobacillus spp. Yarrowia lipolytica, Aureobasidium sp., Trichosporonoides sp., Trigonopsis variabilis, Trichosporon sp., Moniliellaacetoabutans sp., Typhula variabilis, Candida magnolias, Ustilaginomycetes sp., Pseudozyma tsukubaensis, yeast species of genera Zygosaccharomyces, Debaryomyces, Hansenula and Pichia, and fungi of the dematioid genus Torula. See, e.g., Philippidis, G. P., 1996, Cellulose bioconversion technology, in Handbook on Bioethanol: Production and Utilization, Wyman, C. E., ed., Taylor & Francis, Washington, D.C., 179-212, the disclosure of which is incorporated herein by reference in its entirety.

Fermentation medium may be selected depending on the host cell and/or needs of the end-user. Any necessary supplements besides, e.g., carbon, may also be included at appropriate concentrations introduced alone or as a mixture with another supplement or medium such as a complex nitrogen source.

### Yeast Fermentation

Fermentation methods using yeast are well known to those having ordinary skill in the art. In some embodiments, batch fermentation can be used according to the methods provided herein; in other embodiments, continuous fermentation procedures can be used.

In some embodiments, the batch method of fermentation can be used to produce KPVs of the present disclosure. Briefly, the batch method of fermentation refers to a type of fermentation that is performed with a closed system, wherein the composition of the medium is determined at the beginning of the fermentation and is not subject to artificial alterations during the fermentation (i.e., the medium is inoculated with one or more yeast cells at the start of fermentation, and fermentation is allowed to proceed, uninterrupted by the user). Typically, in batch fermentation systems, the metabolite and biomass compositions of the system change constantly up to the time the fermentation is stopped. Within batch cultures, yeast cells pass through a static lag phase to a high growth log phase, and, finally, to a stationary phase, in which the growth rate is diminished or stopped. If untreated, yeast cells in the stationary phase will eventually die. In a batch method, yeast cells in log phase generally are responsible for the bulk of synthesis of end product.

In some embodiments, fed-batch fermentation can be used to produce KPVs of the present disclosure. Briefly, fed-batch fermentation is similar to typical batch method (described above), however, the substrate in the fed-batch method is added in increments as the fermentation progresses. Fed-batch fermentation is useful when catabolite repression may inhibit yeast cell metabolism, and when it is desirable to have limited amounts of substrate in the medium. Generally, the measurement of the substrate concentration in a fed-batch system is estimated on the basis of the changes of measurable factors reflecting metabolism, such as pH, dissolved oxygen, the partial pressure of waste gases (e.g., CO₂), and the like.

In some embodiments, the fed-batch fermentation procedure can be used to produce KPVs as follows: culturing a production organism (e.g., a modified yeast cell) in a 10 L bioreactor sparged with an N₂/CO₂ mixture, using 5 L broth containing 5 g/L potassium phosphate, 2.5 g/L ammonium chloride, 0.5 g/L magnesium sulfate, and 30 g/L corn steep liquor, and an initial first and second carbon source concentration of 20 g/L. As the modified yeast cells grow and utilize the carbon sources, additional 70% carbon source mixture is then fed into the bioreactor at a rate approximately balancing carbon source consumption. The temperature of the bioreactor is generally maintained at 30° C. Growth continues for approximately 24 hours or more, and the heterologous peptides reach a desired concentration, e.g., with the cell density being between about 5 and 10 g/L. Upon completion of the cultivation period, the fermenter contents can be passed through a cell separation unit such as a centrifuge to remove cells and cell debris, and the fermentation broth can be transferred to a product separations unit. Isolation of the heterologous peptides can take place by standard separations procedures well known in the art.

In some embodiments, continuous fermentation can be used to produce KPVs of the present disclosure. Briefly, continuous fermentation refers to fermentation with an open system, wherein a fermentation medium is added continuously to a bioreactor, and an approximately equal amount of conditioned medium is removed simultaneously for processing. Continuous fermentation generally maintains the cultures at a high density, in which yeast cells are primarily in log phase growth. Typically, continuous fermentation methods are performed to maintain steady state growth conditions, and yeast cell loss, due to medium withdrawal, should be balanced against the cell growth rate in the fermentation.

In some embodiments, the continuous fermentation method can be used to produce KPVs as follows: a modified yeast strain can be cultured using a bioreactor apparatus and a medium composition, albeit where the initial first and second carbon source is about, e.g., 30-50 g/L. When the carbon source is exhausted, feed medium of the same composition is supplied continuously at a rate of between about 0.5 L/hr and 1 L/hr, and liquid is withdrawn at the same rate. The heterologous peptide concentration in the bioreactor generally remains constant along with the cell density. Temperature is generally maintained at 30° C., and the pH is generally maintained at about 4.5 using concentrated NaOH and HCl, as required.

In some embodiments, when producing KPVs, the bioreactor can be operated continuously, for example, for about one month, with samples taken every day or as needed to assure consistency of the target chemical compound concentration. In continuous mode, fermenter contents are constantly removed as new feed medium is supplied. The exit stream, containing cells, medium, and heterologous peptides, can then be subjected to a continuous product separations procedure, with or without removing cells and cell debris, and can be performed by continuous separations methods well known in the art to separate organic products from peptides of interest.

In some embodiments, a yeast cell operable to express a KPV or KPV-pesticidal protein can be grown, e.g., using a fed batch process in aerobic bioreactor. Briefly, reactors are filled to about 20% to about 70% capacity with medium comprising a carbon source and other reagents. Temperature and pH is maintained using one or more chemicals as described herein. Oxygen level is maintained by sparging air intermittently in concert with agitation.

For example, in some embodiments, the present disclosure provides a method of using a fed batch process in aerobic bioreactor, wherein the reactor is filled to about 20%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; or 70% capacity.

In some embodiments, the present disclosure provides a fed batch fermentation method using an aerobic bioreactor to produce KPVs, wherein the medium is a rich culture medium. For example, in some embodiments, the carbon source can be glucose, sorbitol, glycerol, galactose, or lactose.

In some embodiments, the amount of glucose can be about 2 g/L; 3 g/L; 4 g/L; 5 g/L; 6 g/L; 7 g/L; 8 g/L; 9 g/L; 10 g/L; 11 g/L; 12 g/L; 13 g/L; 14 g/L; 15 g/L; 16 g/L; 17 g/L; 18 g/L; 19 g/L; 20 g/L; 21 g/L; 22 g/L; 23 g/L; 24 g/L; 25 g/L; 26 g/L; 27 g/L; 28 g/L; 29 g/L; or 30 g/L of the medium.

In some embodiments, the amount of sorbitol can be about 2 g/L; 3 g/L; 4 g/L; 5 g/L; 6 g/L; 7 g/L; 8 g/L; 9 g/L; 10 g/L; 11 g/L; 12 g/L; 13 g/L; 14 g/L; 15 g/L; 16 g/L; 17 g/L; 18 g/L; 19 g/L; 20 g/L; 21 g/L; 22 g/L; 23 g/L; 24 g/L; 25 g/L; 26 g/L; 27 g/L; 28 g/L; 29 g/L; or 30 g/L of the medium.

In some embodiments, the amount of lactose can be about 2 g/L; 3 g/L; 4 g/L; 5 g/L; 6 g/L; 7 g/L; 8 g/L; 9 g/L; 10 g/L; 11 g/L; 12 g/L; 13 g/L; 14 g/L; 15 g/L; 16 g/L; 17 g/L; 18 g/L; 19 g/L; 20 g/L; 21 g/L; 22 g/L; 23 g/L; 24 g/L; 25 g/L; 26 g/L; 27 g/L; 28 g/L; 29 g/L; or 30 g/L of the medium.

In some embodiments, the present disclosure provides a fed batch fermentation method using an aerobic bioreactor, wherein the medium is supplemented with one or more of phosphoric acid, calcium sulfate, potassium sulfate, magnesium sulfate heptahydrate, potassium hydroxide, and/or corn steep liquor.

In some embodiments, the medium can be supplemented with phosphoric acid in an amount of about 2 g/L; 3 g/L; 4 g/L; 5 g/L; 6 g/L; 7 g/L; 8 g/L; 9 g/L; 10 g/L; 11 g/L; 12 g/L; 13 g/L; 14 g/L; 15 g/L; 16 g/L; 17 g/L; 18 g/L; 19 g/L; 20 g/L; 21 g/L; 22 g/L; 23 g/L; 24 g/L; 25 g/L; 26 g/L; 27 g/L; 28 g/L; 29 g/L; or 30 g/L to the medium.

In some embodiments, the medium can be supplemented with calcium sulfate in an amount of about 0.05 g/L; 0.15 g/L; 0.25 g/L; 0.35 g/L; 0.45 g/L; 0.55 g/L; 0.65 g/L; 0.75 g/L; 0.85 g/L; 0.95 g/L; 1.05 g/L; 1.15 g/L; 1.25 g/L; 1.35 g/L; 1.45 g/L; 1.55 g/L; 1.65 g/L; 1.75 g/L; 1.85 g/L; 1.95 g/L; 2.05 g/L; 2.15 g/L; 2.25 g/L; 2.35 g/L; 2.45 g/L; 2.55 g/L; 2.65 g/L; 2.75 g/L; 2.85 g/L; or 2.95 g/L to the medium.

In some embodiments, the medium can be supplemented with potassium sulfate in an amount of about 2 g/L; 2.5 g/L; 3 g/L; 3.5 g/L; 4 g/L; 4.5 g/L; 5 g/L; 5.5 g/L; 6 g/L; 6.5 g/L; 7 g/L; 7.5 g/L; 8 g/L; 8.5 g/L; 9 g/L; 9.5 g/L; 10 g/L; 10.5 g/L; 11 g/L; 11.5 g/L; 12 g/L; 12.5 g/L; 13 g/L; 13.5 g/L; 14 g/L; 14.5 g/L; 15 g/L; 15.5 g/L; 16 g/L; 16.5 g/L; 17 g/L; 17.5 g/L; 18 g/L; 18.5 g/L; 19 g/L; 19.5 g/L; or 20 g/L to the medium.

In some embodiments, the medium can be supplemented with magnesium sulfate heptahydrate in an amount of about 0.25 g/L; 0.5 g/L; 0.75 g/L; 1 g/L; 1.25 g/L; 1.5 g/L; 1.75 g/L; 2 g/L; 2.25 g/L; 2.5 g/L; 2.75 g/L; 3 g/L; 3.25 g/L; 3.5 g/L; 3.75 g/L; 4 g/L; 4.25 g/L; 4.5 g/L; 4.75 g/L; 5 g/L; 5.25 g/L; 5.5 g/L; 5.75 g/L; 6 g/L; 6.25 g/L; 6.5 g/L; 6.75 g/L; 7 g/L; 7.25 g/L; 7.5 g/L; 7.75 g/L; 8 g/L; 8.25 g/L; 8.5 g/L; 8.75 g/L; 9 g/L; 9.25 g/L; 9.5 g/L; 9.75 g/L; 10 g/L; 10.25 g/L; 10.5 g/L; 10.75 g/L; 11 g/L; 11.25 g/L; 11.5 g/L; 11.75 g/L; 12 g/L; 12.25 g/L; 12.5 g/L; 12.75 g/L; 13 g/L; 13.25 g/L; 13.5 g/L; 13.75 g/L; 14 g/L; 14.25 g/L; 14.5 g/L; 14.75 g/L; or 15 g/L to the medium.

In some embodiments, the medium can be supplemented with potassium hydroxide in an amount of about 0.25 g/L; 0.5 g/L; 0.75 g/L; 1 g/L; 1.25 g/L; 1.5 g/L; 1.75 g/L; 2 g/L; 2.25 g/L; 2.5 g/L; 2.75 g/L; 3 g/L; 3.25 g/L; 3.5 g/L; 3.75 g/L; 4 g/L; 4.25 g/L; 4.5 g/L; 4.75 g/L; 5 g/L; 5.25 g/L; 5.5 g/L; 5.75 g/L; 6 g/L; 6.25 g/L; 6.5 g/L; 6.75 g/L; or 7 g/L to the medium.

In some embodiments, the medium can be supplemented with corn steep liquor in an amount of about 5 g/L; 6 g/L; 7 g/L; 8 g/L; 9 g/L; 10 g/L; 11 g/L; 12 g/L; 13 g/L; 14 g/L; 15 g/L; 16 g/L; 17 g/L; 18 g/L; 19 g/L; 20 g/L; 21 g/L; 22 g/L; 23 g/L; 24 g/L; 25 g/L; 26 g/L; 27 g/L; 28 g/L; 29 g/L; 30 g/L; 31 g/L; 32 g/L; 33 g/L; 34 g/L; 35 g/L; 36 g/L; 37 g/L; 38 g/L; 39 g/L; 40 g/L; 41 g/L; 42 g/L; 43 g/L; 44 g/L; 45 g/L; 46 g/L; 47 g/L; 48 g/L; 49 g/L; 50 g/L; 51 g/L; 52 g/L; 53 g/L; 54 g/L; 55 g/L; 56 g/L; 57 g/L; 58 g/L; 59 g/L; 60 g/L; 61 g/L; 62 g/L; 63 g/L; 64 g/L; 65 g/L; 66 g/L; 67 g/L; 68 g/L; 69 g/L; or 70 g/L to the medium.

In some embodiments, the temperature of the reactor can be maintained between about 15° C. and about 45° C. In some embodiments, the reactor can have a temperature of about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

In some embodiments, the pH can have a level of about 3 to about 6. In some embodiments, the pH can be 3.0, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, or 6.0.

In some embodiments, the pH can be maintained at a constant level via the addition of one or more chemicals. For example, in some embodiments, ammonium hydroxide can be added to maintain pH. In some embodiments, ammonium hydroxide can be added to a level of ammonium hydroxide in the medium that is about 5%, 6%, 7%, 8%, 9%, 10%, 11%, 12%, 13%, 14%, 15%, 16%, 17%, 18%, 19%, or 20%, of ammonium hydroxide

In some embodiments, oxygen levels can be maintained by sparging. For example, in some embodiments, dissolved oxygen can be maintained at a constant level by sparging air between 0.5-1.5 volume/volume/min and by increasing agitation to maintain a set point of 10-30%.

In some embodiments, inoculation of the reactor can be accomplished based on an overnight seed culture comprising from about 2.5 g/L to about 50 g/L of a carbon source, e.g., glucose, sorbitol, or lactose. In some embodiments, the overnight seed culture can comprise corn steep liquor, e.g., from about 2.5 g/L to about 50 g/L of corn steep liquor.

In some embodiments, the inoculation percentage can range from about 5-20% of initial fill volume. Following inoculation, the reactor can be fed with from about a 50% to about an 80% solution of the selected carbon source up until the reactor is filled and/or the desired supernatant peptide concentration is achieved. In some embodiments, the time required to fill the reactor can range from about 86 hours to about 160 hours. In some embodiments, the quantity required to reach the desired peptide concentration can range from about 0.8 g/L to about 1.2 g/L. Upon completion of the fermentation, the contents can be passed through a cell separation unit and optionally concentrated, depending on intended use of the material.

Recipes for yeast fermentation media are provided herein, and known in the art.

KPV Incorporation into Plants or Parts Thereof

The KPVs described herein, and/or an pesticidal protein comprising at least one KPV as described herein, can be incorporated into plants, plant tissues, plant cells, plant seeds, and/or plant parts thereof, for either the stable, or transient expression of a KPV or a KPV-pesticidal protein, and/or a polynucleotide sequence encoding the same.

In some embodiments, the KPV or KPV-pesticidal protein can be incorporated into a plant using recombinant techniques known in the art. In some embodiments, the KPV or KPV-pesticidal protein may be in the form of an pesticidal protein which may comprise one or more KPV monomers.

As used herein, with respect to transgenic plants, plant tissues, plant cells, and plant seeds, the term “KPV” also encompasses a KPV-pesticidal protein, and an “KPV polynucleotide” is similarly also used to encompass a polynucleotide or group of polynucleotides operable to express and/or encode an pesticidal protein comprising one or more KPVs.

The goal of incorporating a KPV into plants is to deliver KPVs and/or KPV-pesticidal proteins to the pest via the pest's consumption of the transgenic KPV expressed in a plant tissue consumed by the insect. Upon the consumption of the KPV by the insect from its food (e.g., via an insect feeding upon a transgenic plant transformed with a KPV), the consumed KPV may have the ability to inhibit the growth, impair the movement, or even kill an insect. Accordingly, transgenic plants expressing a KPV polynucleotide and/or a KPV polypeptide may express said KPV polynucleotide/polypeptide in a variety of plant tissues, including but not limited to: the epidermis (e.g., mesophyll); periderm; phloem; xylem; parenchyma; collenchyma; sclerenchyma; and primary and secondary meristematic tissues. For example, in some embodiments, a polynucleotide sequence encoding a KPV can be operably linked to a regulatory region containing a phosphoenolpyruvate carboxylase promoter, resulting in the expression of a KPV in a plant's mesophyll tissue.

Transgenic plants expressing a KPV and/or a polynucleotide operable to express KPV can be generated by any one of the various methods and protocols well known to those having ordinary skill in the art; such methods of the invention do not require that a particular method for introducing a nucleotide construct to a plant be used, only that the nucleotide construct gains access to the interior of at least one cell of the plant. Methods for introducing nucleotide constructs into plants are known in the art including, but not limited to, stable transformation methods, transient transformation methods, and virus-mediated methods. “Transgenic plants” or “transformed plants” or “stably transformed” plants or cells or tissues refers to plants that have incorporated or integrated exogenous nucleic acid sequences or DNA fragments into the plant cell. These nucleic acid sequences include those that are exogenous, or not present in the untransformed plant cell, as well as those that may be endogenous, or present in the untransformed plant cell. “Heterologous” generally refers to the nucleic acid sequences that are not endogenous to the cell or part of the native genome in which they are present, and have been added to the cell by infection, transfection, microinjection, electroporation, microprojection, or the like.

Transformation of plant cells can be accomplished by one of several techniques known in the art. Typically, a construct that expresses an exogenous or heterologous peptide or polypeptide of interest (e.g., a KPV), would contain a promoter to drive transcription of the gene, as well as a 3′ untranslated region to allow transcription termination and polyadenylation. The design and organization of such constructs is well known in the art. In some embodiments, a gene can be engineered such that the resulting peptide is secreted, or otherwise targeted within the plant cell to a specific region and/or organelle. For example, the gene can be engineered to contain a signal peptide to facilitate transfer of the peptide to the endoplasmic reticulum. It may also be preferable to engineer the plant expression cassette to contain an intron, such that mRNA processing of the intron is required for expression.

Typically, a plant expression cassette can be inserted into a plant transformation vector. This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as “binary vectors.” Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the KPV are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

In general, plant transformation methods involve transferring heterologous DNA into target plant cells (e.g. immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grows into a mature plant and produces fertile seeds. See Hiei et al. (1994) The Plant Journal 6:271-282; and Ishida et al. (1996) Nature Biotechnology 14:745-750), the disclosures of which are incorporated herein by reference in their entireties. Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997) Maydica 42:107-120, the disclosures of which is incorporated herein by reference in its entirety. Because the transformed material contains many cells, both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

Transformation protocols as well as protocols for introducing nucleotide sequences into plants may vary depending on the type of plant or plant cell, i.e., monocot or dicot, targeted for transformation. Generation of transgenic plants may be performed by one of several methods, including, but not limited to, microinjection, electroporation, direct gene transfer, introduction of heterologous DNA by Agrobacterium into plant cells (Agrobacterium-mediated transformation), bombardment of plant cells with heterologous foreign DNA adhered to particles, ballistic particle acceleration, aerosol beam transformation, Lec1 transformation, and various other non-particle direct-mediated methods to transfer DNA. Exemplary transformation protocols are disclosed in U.S. Published Application No. 20010026941; U.S. Pat. No. 4,945,050; International Publication No. WO 91/00915; and U.S. Published Application No. 2002015066, the disclosures of which are incorporated herein by reference in their entireties.

Chloroplasts can also be readily transformed, and methods concerning the transformation of chloroplasts are known in the art. See, e.g., Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606, the disclosure of which is incorporated herein by reference in its entirety. The method of chloroplast transformation relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305, the disclosures of which is incorporated herein by reference in its entirety.

Following integration of heterologous foreign DNA into plant cells, one having ordinary skill may then apply a maximum threshold level of appropriate selection chemical/reagent (e.g., an antibiotic) in the medium to kill the untransformed cells, and separate and grow the putatively transformed cells that survive from this selection treatment by transferring said surviving cells regularly to a fresh medium. By continuous passage and challenge with appropriate selection, an artisan identifies and proliferates the cells that are transformed with the plasmid vector. Molecular and biochemical methods can then be used to confirm the presence of the integrated heterologous gene of interest into the genome of the transgenic plant.

The cells that have been transformed may be grown into plants in accordance with conventional methods known to those having ordinary skill in the art. See, for example, McCormick et al. (1986) Plant Cell Reports 5:81-84, the disclosure of which is incorporated herein by reference in its entirety. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a nucleotide construct of the invention, for example, an expression cassette of the invention, stably incorporated into their genome.

In various embodiments, the present disclosure provides a KPV-pesticidal protein, that act as substrates for insect proteinases, proteases and peptidases (collectively referred to herein as “proteases”) as described above.

In some embodiments, transgenic plants or parts thereof, that may be receptive to the expression of KPVs can include: alfalfa, banana, barley, bean, broccoli, cabbage, canola, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and a wheat plant.

In some embodiments the transgenic plant may be grown from cells that were initially transformed with the DNA constructs described herein. In other embodiments, the transgenic plant may express the encoded KPV in a specific tissue, or plant part, for example, a leaf, a stem a flower, a sepal, a fruit, a root, a seed, or combinations thereof.

In some embodiments, a plant, plant tissue, plant cell, plant seed, or part thereof, can be transformed with a KPV or a polynucleotide encoding the same, or a complementary nucleotide sequence thereof, said KPV comprising, consisting essentially of, or consisting of, an amino acid sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253.

In some embodiments, a plant, plant tissue, plant cell, or plant seed can be transformed with a KPV that comprises, consists essentially of, or consists of, an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a plant, plant tissue, plant cell, or plant seed can be transformed with a polynucleotide encoding a KPV, or a complementary nucleotide sequence thereof, wherein the KPV comprises, consists essentially of, or consists of, an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, the plant, plant tissue, plant cell, plant seed, or part thereof, can be transformed with a KPV or a polynucleotide encoding the same, wherein the polynucleotide is operable to encode a homopolymer or heteropolymer of two or more KPVs, wherein the amino acid sequence of each KPV is the same or different.

In some embodiments, the plant, plant tissue, plant cell, plant seed, or part thereof, can be transformed with a KPV or a polynucleotide encoding the same, wherein the polynucleotide is operable to encode a KPV that is a fused protein comprising two or more KPVs separated by a cleavable or non-cleavable linker, and wherein the amino acid sequence of each KPV may be the same or different.

In some embodiments, the linker is a cleavable linker.

In some embodiments, the linker has an amino acid sequence as set forth in any one of SEQ ID NOs: 360-369.

In some embodiments, the linker is cleavable inside at least one of (i) the gut or hemolymph of an insect, and (ii) cleavable inside the gut of a mammal.

In some embodiments, the plant, plant tissue, plant cell, or plant seed can be transformed with a KPV wherein the KPV has an amino acid sequence of any of the aforementioned KPVs (e.g., one or more the KPVs enumerated in Table 1), or a polynucleotide encoding the same.

Any of the aforementioned methods, and/or any of the methods described herein, can be used to incorporate one or more of the KPVs or KPV-pesticidal proteins as described herein, into plants or plant parts thereof. For example, any of the methods described herein can be used to incorporate into plants one or more of the KPVs described in the present disclosure, e.g., KPVs having the amino acid sequence of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, which are likewise described herein.

Polynucleotide Incorporation into Plants, the Proteins Expressed Therefrom

A challenge regarding the expression of heterogeneous polypeptides in transgenic plants is maintaining the desired effect (e.g., pesticidal activity) of the introduced polypeptide upon expression in the host organism; one way to maintain such an effect is to increase the chance of proper protein folding through the use of an operably linked Endoplasmic Reticulum Signal Peptide (ERSP). Another method to maintain the effect of a transgenic protein is to incorporate a Translational Stabilizing Protein (STA).

Plants can be transiently or stably transfected with the DNA sequence that encodes a KPV or a KPV-pesticidal protein comprising one or more KPVs, using any of the transfection methods described above. Alternatively, plants can be transfected with a polynucleotide that encodes a KPV, wherein said KPV is operably linked to a polynucleotide operable to encode an Endoplasmic Reticulum Signal Peptide (ERSP); linker, Translational Stabilizing Protein (STA); or combination thereof. For example, in some embodiments, a transgenic plant or plant genome can be transformed with a polynucleotide sequence that encodes the Endoplasmic Reticulum Signal Peptide (ERSP); KPV; and/or intervening linker peptide (LINKER or L), thus causing mRNA transcribed from the heterogeneous DNA to be expressed in the transformed plant, and subsequently, said mRNA to be translated into a peptide.

### Endoplasmic Reticulum Signal Peptide (ERSP)

The subcellular targeting of a recombinant protein to the ER can be achieved through the use of an ERSP operably linked to said recombinant protein; this allows for the correct assembly and/or folding of such proteins, and the high level accumulation of these recombinant proteins in plants. Exemplary methods concerning the compartmentalization of host proteins into intracellular storage are disclosed in McCormick et al., Proc. Natl. Acad. Sci. USA 96 (2): 703-708, 1999; Staub et al., Nature Biotechnology 18:333-338, 2000; Conrad et al., Plant Mol. Biol. 38:101-109, 1998; and Stoger et al., Plant Mol. Biol. 42:583-590, 2000, the disclosures of which are incorporated herein by reference in their entireties. Accordingly, one way to achieve the correct assembly and/or folding of recombinant proteins, is to operably link an endoplasmic reticulum signal peptide (ERSP) to the recombinant protein of interest.

In some embodiments, a peptide comprising an Endoplasmic Reticulum Signal Peptide (ERSP) can be operably linked to a KPV (designated as ERSP-KPV), wherein said ERSP is the N-terminal of said peptide. In some embodiments, the ERSP peptide is between 3 to 60 amino acids in length, between 5 to 50 amino acids in length, between 20 to 30 amino acids in length.

In some embodiments, KPV ORF starts with an ersp at its 5′-end. For the KPV to be properly folded and functional when it is expressed from a transgenic plant, it must have an ersp nucleotide fused in frame with the polynucleotide encoding a KPV. During the cellular translation process, translated ERSP can direct the KPV being translated to insert into the Endoplasmic Reticulum (ER) of the plant cell by binding with a cellular component called a signal-recognition particle. Within the ER the ERSP peptide is cleaved by signal peptidase and the KPV is released into the ER, where the KPV is properly folded during the post-translation modification process, for example, the formation of disulfide bonds. Without any additional retention protein signals, the protein is transported through the ER to the Golgi apparatus, where it is finally secreted outside the plasma membrane and into the apoplastic space. KPV can accumulate at apoplastic space efficiently to reach the pesticidal dose in plants.

The ERSP peptide is at the N-terminal region of the plant-translated KPV complex and the ERSP portion is composed of about 3 to 60 amino acids. In some embodiments it is 5 to 50 amino acids. In some embodiments it is 10 to 40 amino acids but most often is composed of 15 to 20; 20 to 25; or 25 to 30 amino acids. The ERSP is a signal peptide so called because it directs the transportation of a protein. Signal peptides may also be called targeting signals, signal sequences, transit peptides, or localization signals. The signal peptides for ER trafficking are often 15 to 30 amino acid residues in length and have a tripartite organization, comprised of a core of hydrophobic residues flanked by a positively charged amino terminal and a polar, but uncharged carboxyterminal region. See Zimmermann, et al, “Protein translocation across the ER membrane,” Biochimica et Biohysica Acta, 2011, 1808:912-924, the disclosure of which is incorporated herein by reference in its entirety.

Many ERSPs are known. It is NOT required that the ERSP be derived from a plant ERSP, non-plant ERSPs will work with the procedures described herein. Many plant ERSPs are however well known and we describe some plant derived ERSPs here. For example, ins some embodiments, the ERSP can be a barley alpha-amylase signal peptide (BAAS), which is derived from the plant, Hordeum vulgare, and has an amino acid sequence as follows: “MANKHLSLSLFLVLLGLSASLASG” (SEQ ID NO:359).

In some embodiments, the ERSP can be a yeast ERSP. Exemplary yeast ERSPs are described in U.S. Pat. Nos. 6,897,043; 8,753,866; and 10,041,064; the disclosures of which are incorporated herein by reference in their entireties.

Plant ERSPs, which are selected from the genomic sequence for proteins that are known to be expressed and released into the apoplastic space of plants, include examples such as BAAS, carrot extensin, and tobacco PR1. The following references provide further descriptions, and are incorporated by reference herein in their entireties: De Loose, M. et al. “The extensin signal peptide allows secretion of a heterologous protein from protoplasts” Gene, 99 (1991) 95-100; De Loose, M. et al. described the structural analysis of an extension—encoding gene from Nicotiana plumbaginifolia, the sequence of which contains a typical signal peptide for translocation of the protein to the endoplasmic reticulum; Chen, M. H. et al. “Signal peptide-dependent targeting of a rice alpha-amylase and cargo proteins to plastids and extracellular compartments of plant cells” Plant Physiology, 2004 July; 135 (3): 1367-77. Epub 2004 Jul. 2. Chen, M. H. et al. studied the subcellular localization of α-amylases in plant cells by analyzing the expression of α-amylase, with and without its signal peptide, in transgenic tobacco. These references and others teach and disclose the signal peptide that can be used in the methods, procedures and peptide, protein and nucleotide complexes and constructs described herein.

In some embodiments, the ERSP can include, but is not limited to, one of the following: a BAAS; a tobacco extensin signal peptide; a modified tobacco extensin signal peptide; or a Jun a 3 signal peptide from Juniperus ashei. For example, in some embodiments, a plant can be transformed with a nucleotide that encodes any of the peptides that are described herein as Endoplasmic Reticulum Signal Peptides (ERSP), and a KPV.

The tobacco extensin signal peptide motif is another exemplary type of ERSP. See Memelink et al, the Plant Journal, 1993, V4: 1011-1022; and Pogue G P et al, Plant Biotechnology Journal, 2010, V8: 638-654, the disclosures of which are incorporated herein by reference in their entireties.

In some embodiments, a KPV ORF can have a nucleotide sequence operable to encode a tobacco extensin signal peptide motif. In one embodiment, the KPV ORF can encode an extensin motif according to SEQ ID NO: 370. In another embodiment, the KPV ORF can encode an extensin motif according to SEQ ID NO: 371.

An illustrative example of how to generate an embodiment with an extensin signal motif is as follows: A DNA sequence encoding an extensin motif is designed (for example, the DNA sequence shown in SEQ ID NO: 372 or SEQ ID NO: 373) using oligo extension PCR with four synthetic DNA primers; ends sites such as a restriction site, for example, a Pac I restriction site at the 5′-end, and a 5′-end of a GFP sequence at the 3′-end, can be added using PCR with the extensin DNA sequence serving as a template, and resulting in a fragment; the fragment is used as the forward PCR primer to amplify the DNA sequence encoding a KPV ORF, for example “gfp-l-kpv” contained in a pFECT vector, thus producing a KPV ORF encoding (from N′ to C′ terminal) “ERSP-GFP-L-KPV” wherein the ERSP is extensin. The resulting DNA sequence can then be cloned into Pac I and Avr II restriction sites of a FECT vector to generate the pFECT-KPV vector for transient plant expression of GFP fused KPV.

In some embodiments, an illustrative expression system can include the FECT expression vectors containing KPV ORF is transformed into Agrobacterium, GV3101, and the transformed GV3101 is injected into tobacco leaves for transient expression of KPV ORF.

### Translational Stabilizing Protein (STA)

A translational stabilizing protein (STA) can increase the amount of KPV in plant tissues. One of the KPV ORFs, i.e., ERSP-KPV, may be sufficient to express a properly folded KPV in the transfected plant; however, in some embodiments, effective protection of a plant from pest damage may require that the plant expressed KPV accumulate. With transfection of a properly constructed KPV ORF, a transgenic plant can express and accumulate greater amounts of the correctly folded KPV. When a plant accumulates greater amounts of properly folded KPV, it can more easily resist, inhibit, and/or kill the pests that attack and eat the plants. One method of increasing the accumulation of a polypeptide in transgenic tissues is through the use of a translational stabilizing protein (STA). The translational stabilizing protein can be used to significantly increase the accumulation of KPV in plant tissue, and thus increase the efficacy of a plant transfected with KPV with regard to pest resistance. The translational stabilizing protein is a protein with sufficient tertiary structure that it can accumulate in a cell without being targeted by the cellular process of protein degradation.

In some embodiments, the translational stabilizing protein can be a domain of another protein, or it can comprise an entire protein sequence. In some embodiments, the translational stabilizing protein can be between 5 and 50 amino acids, 50 to 250 amino acids (e.g., GNA), 250 to 750 amino acids (e.g., chitinase) and 750 to 1500 amino acids (e.g., enhancin).

One embodiment of the translational stabilizing protein can be a polymer of fusion proteins comprising at least one KPV. A specific example of a translational stabilizing protein is provided here to illustrate the use of a translational stabilizing protein. The example is not intended to limit the disclosure or claims in any way. Useful translational stabilizing proteins are well known in the art, and any proteins of this type could be used as disclosed herein. Procedures for evaluating and testing production of peptides are both known in the art and described herein. One example of one translational stabilizing protein is Green-Fluorescent Protein (GFP) (SEQ ID NO: 374; NCBI Accession No. P42212.1).

In some embodiments, a protein comprising an Endoplasmic Reticulum Signal Peptide (ERSP) can be operably linked to a KPV, which is in turn operably linked to a Translational Stabilizing Protein (STA). Here, this configuration is designated as ERSP-STA-KPV or ERSP-KPV-STA, wherein said ERSP is the N-terminal of said protein and said STA may be either on the N-terminal side (upstream) of the KPV, or of the C-terminal side (downstream) of the KPV. In some embodiments, a protein designated as ERSP-STA-KPV or ERSP-KPV-STA, comprising any of the ERSPs or KPVs described herein, can be operably linked to a STA, for example, any of the translational stabilizing proteins described, or taught by this document including GFP (Green Fluorescent Protein; SEQ ID NO: 116; NCBI Accession No. P42212), or Jun a 3, (Juniperus ashei; SEQ ID NO: 117; NCBI Accession No. P81295.1).

Additional examples of translational stabilizing proteins can be found in the following references, the disclosures of which are incorporated herein by reference in their entirety: Kramer, K. J. et al. “Sequence of a cDNA and expression of the gene encoding epidermal and gut chitinases of Manduca sexta” Insect Biochemistry and Molecular Biology, Vol. 23, Issue 6, September 1993, pp. 691-701. Kramer, K. J. et al. isolated and sequenced a chitinase-encoding cDNA from the tobacco hornworm, Manduca sexta. Hashimoto, Y. et al. “Location and nucleotide sequence of the gene encoding the viral enhancing factor of the Trichoplusia ni granulosis virus” Journal of General Virology, (1991), 72, 2645-2651. These references and others teach and disclose translational stabilizing proteins that can be used in the methods, procedures and peptide, protein and nucleotide complexes and constructs described herein.

In some embodiments, a KPV ORF can be transformed into a plant, for example, in the tobacco plant, Nicotiana benthamiana, using a KPV ORF that contains a STA. For example, in some embodiments, the STA can be Jun a 3. The mature Jun a 3 is a ˜30 kDa plant defending protein that is also an allergen for some people. Jun a 3 is produced by Juniperus ashei trees and can be used in some embodiments as a translational stabilizing protein (STA). In some embodiments, the Jun a 3 amino acid sequence can be the sequence shown in SEQ ID NO: 376. In other embodiments, the Jun a 3 amino acid sequence can be the sequence shown in SEQ ID NO: 375.

### Linkers

Linker proteins assist in the proper folding of the different motifs composing a KPV ORF. The KPV ORF described in this invention also incorporates polynucleotide sequences encoding intervening linker peptides between the polynucleotide sequences encoding the KPV (kpv) and the translational stabilizing protein (sta), or between polynucleotide sequence encoding multiple polynucleotide sequences encoding KPV, i.e., (l-kpv)_{N} or (kpv-l)_{N}, if the expression ORF involves multiple KPV domain expression. The intervening linker peptides (LINKERS or L or L_{INK}) separate the different parts of the expressed KPV construct, and help proper folding of the different parts of the complex during the expression process. In the expressed KPV construct, different intervening linker peptides can be involved to separate different functional domains. In some embodiments, the LINKER is attached to a KPV and this bivalent group can be repeated up to 10 (N=1-10) and possibly even more than 10 times (e.g., N=200) in order to facilitate the accumulation of properly folded KPV in the plant that is to be protected.

In some embodiments the intervening linker peptide can be between 1 and 30 amino acids in length. However, it is not necessarily an essential component in the expressed KPV in plants.

In some embodiments, the KPV-pesticidal protein comprises at least one KPV operably linked to a cleavable peptide. In other embodiments, the KPV-pesticidal protein comprises at least one KPV operably linked to a non-cleavable peptide.

A cleavable linker peptide can be designed to the KPV ORF to release the properly KPV from the expressed KPV complex in the transformed plant to improve the protection the KPV affords the plant with regard to pest damage. One type of the intervening linker peptide is the plant cleavable linker peptide. This type of linker peptides can be completely removed from the expressed KPV ORF complex during plant post-translational modification. Therefore, in some embodiments, the properly folded KPV linked by this type of intervening linker peptides can be released in the plant cells from the expressed KPV ORF complex during post-translational modification in the plant.

Another type of the cleavable intervening linker peptide is not cleavable during the expression process in plants. However, it has a protease cleavage site specific to serine, threonine, cysteine, aspartate proteases or metalloproteases. The type of cleavable linker peptide can be digested by proteases found in the insect and lepidopteran gut environment and/or the insect hemolymph and lepidopteran hemolymph environment to release the KPV in the insect gut or hemolymph. Using the information taught by this disclosure it should be a matter of routine for one skilled in the art to make or find other examples of LINKERS that will be useful in this invention.

In some embodiments, the KPV ORF can contain a cleavable type of intervening linker, for example, having the amino acid code of “IGER” (SEQ ID NO: 119). The molecular weight of this intervening linker or LINKER is 473.53 Daltons. In other embodiments, the intervening linker peptide (LINKER) can also be one without any type of protease cleavage site, i.e., an uncleavable intervening linker peptide, for example, the linker “EEKKN” (SEQ ID NO: 378) or “ETMEKHGL” (SEQ ID NO:379).

In some embodiments, the KPV ORK can comprise a linker having an amino acid sequence as set forth in any one of SEQ ID NOs: 360-369.

In some embodiments, the KPV-pesticidal protein can have two or more cleavable peptides, wherein the pesticidal protein comprises an insect cleavable linker (L), the insect cleavable linker being fused in frame with a construct comprising (KPV-L)ₙ, wherein “n” is an integer ranging from 1 to 200, or from 1 to 100, or from 1 to 10. In another embodiment, the KPV-pesticidal protein, and described herein, comprises an endoplasmic reticulum signal peptide (ERSP) operably linked with a KPV, which is operably linked with an insect cleavable linker (L) and/or a repeat construct (L-KPV), or (KPV-L)ₙ, wherein n is an integer ranging from 1 to 200, or from 1 to 100, or from 1 to 10.

In some embodiments, a protein comprising an Endoplasmic Reticulum Signal Peptide (ERSP) can be operably linked to a KPV and an intervening linker peptide (L or Linker); such a construct is designated as ERSP-L-KPV, or ERSP-KPV-L, wherein said ERSP is the N-terminal of said protein, and said L or Linker may be either on the N-terminal side (upstream) of the KPV, or the C-terminal side (downstream) of the KPV. A protein designated as ERSP-L-KPV, or ERSP-KPV-L, comprising any of the ERSPs or KPVs described herein, can have a Linker “L” that can be an uncleavable linker peptide, or a cleavable linker peptide, and which may be cleavable in a plant cells during protein expression process, or may be cleavable in an insect gut environment and/or hemolymph environment.

In some embodiments, a KPV-pesticidal protein can comprise any of the intervening linker peptides (LINKER or L) described herein, or taught by this document, including but not limited to following sequences: IGER (SEQ ID NO:119), EEKKN, (SEQ ID NO: 120), and ETMFKHGL (SEQ ID NO:121), or combinations thereof.

In some embodiments, the linker can be one or more of the following: ALKFLV (SEQ ID NO: 360), ALKLFV (SEQ ID NO: 361), IFVRLR (SEQ ID NO: 362), LFAAPF (SEQ ID NO: 363), ALKFLVGS (SEQ ID NO: 364), ALKLFVGS (SEQ ID NO: 365), IFVRLRGS (SEQ ID NO: 366), LFAAPFGS (SEQ ID NO: 367), LFVRLRGS (SEQ ID NO: 368), and/or LGERGS (SEQ ID NO: 369).

In various embodiments, an exemplary pesticidal protein can include a protein construct comprising: (ERSP)-(KPV-L)ₙ; (ERSP)-(L)-(KPV-L)ₙ; (ERSP)-(L-KPV)ₙ; (ERSP)-(L-KPV)ₙ-(L); wherein n is an integer ranging from 1 to 200 or from 1 to 100, or from 1 to 10. In various related embodiments described above, a KPV is the Kappa Peptide Variant, L is a non-cleavable or cleavable peptide, and n is an integer ranging from 1 to 200, preferably an integer ranging from 1 to 100, and more preferably an integer ranging from 1 to 10. In some embodiments, the KPV-pesticidal protein may contain KPV peptides that are the same or different, and insect cleavable peptides that are the same or different. In some embodiments, the C-terminal KPV is operably linked at its C-terminus with a cleavable peptide that is operable to be cleaved in an insect gut environment. In some embodiments, the N-terminal KPV is operably linked at its N-terminus with a cleavable peptide that is operable to be cleaved in an insect gut environment.

Some of the available proteases and peptidases found in the insect gut environment are dependent on the life-stage of the insect, as these enzymes are often spatially and temporally expressed. The digestive system of the insect is composed of the alimentary canal and associated glands. Food enters the mouth and is mixed with secretions that may or may not contain digestive proteases and peptidases. The foregut and the hind gut are ectodermal in origin. The foregut serves generally as a storage depot for raw food. From the foregut, discrete boluses of food pass into the midgut (mesenteron or ventriculus). The midgut is the site of digestion and absorption of food nutrients. Generally, the presence of certain proteases and peptidases in the midgut follow the pH of the gut. Certain proteases and peptidases in the human gastrointestinal system may include: pepsin, trypsin, chymotrypsin, elastase, carboxypeptidase, aminopeptidase, and dipeptidase.

The insect gut environment includes the regions of the digestive system in the herbivore species where peptides and proteins are degraded during digestion. Some of the available proteases and peptidases found in insect gut environments may include: (1) serine proteases; (2) cysteine proteases; (3) aspartic proteases, and (4) metalloproteases.

The two predominant protease classes in the digestive systems of phytophagous insects are the serine and cysteine proteases. Murdock et al. (1987) carried out an elaborate study of the midgut enzymes of various pests belonging to Coleoptera, while Srinivasan et al. (2008) have reported on the midgut enzymes of various pests belonging to Lepidoptera. Serine proteases are known to dominate the larval gut environment and contribute to about 95% of the total digestive activity in Lepidoptera, whereas the Coleopteran species have a wider range of dominant gut proteases, including cysteine proteases.

The papain family contains peptidases with a wide variety of activities, including endopeptidases with broad specificity (such as papain), endopeptidases with very narrow specificity (such as glycyl endopeptidases), aminopeptidases, dipeptidyl-peptidase, and peptidases with both endopeptidase and exopeptidase activities (such as cathepsins B and H). Other exemplary proteinases found in the midgut of various insects include trypsin-like enzymes, e.g. trypsin and chymotrypsin, pepsin, carboxypeptidase-B and aminotripeptidases.

Serine proteases are widely distributed in nearly all animals and microorganisms (Joanitti et al., 2006). In higher organisms, nearly 2% of genes code for these enzymes (Barrette-Ng et al., 2003). Being essentially indispensable to the maintenance and survival of their host organism, serine proteases play key roles in many biological processes. Serine proteases are classically categorized by their substrate specificity, notably by whether the residue at P1: trypsin-like (Lys/Arg preferred at P1), chymotrypsin-like (large hydrophobic residues such as Phe/Tyr/Leu at P1), or elastase-like (small hydrophobic residues such as Ala/Val at P1) (revised by Tyndall et. al., 2005). Serine proteases are a class of proteolytic enzymes whose central catalytic machinery is composed of three invariant residues, an aspartic acid, a histidine and a uniquely reactive serine, the latter giving rise to their name, the “catalytic triad”. The Asp-His-Ser triad can be found in at least four different structural contexts (Hedstrom, 2002). These four clans of serine proteases are typified by chymotrypsin, subtilisin, carboxypeptidase Y, and Clp protease. The three serine proteases of the chymotrypsin-like clan that have been studied in greatest detail are chymotrypsin, trypsin, and elastase. More recently, serine proteases with novel catalytic triads and dyads have been discovered for their roles in digestion, including Ser-His-Glu, Ser-Lys/His, His-Ser-His, and N-terminal Ser.

One class of well-studied digestive enzymes found in the gut environment of insects is the class of cysteine proteases. The term “cysteine protease” is intended to describe a protease that possesses a highly reactive thiol group of a cysteine residue at the catalytic site of the enzyme. There is evidence that many phytophagous insects and plant parasitic nematodes rely, at least in part, on midgut cysteine proteases for protein digestion. These include but are not limited to Hemiptera, especially squash bugs (Anasa tristis); green stink bug (Acrosternum hilare); Riptortus clavatus; and almost all Coleoptera examined to date, especially, Colorado potato beetle (Leptinotarsa deaemlineata); three-lined potato beetle (Lema trilineata); asparagus beetle (Crioceris asparagi); Mexican bean beetle (Epilachna varivestis); red flour beetle (Triolium castaneum); confused flour beetle (Tribolium confusum); the flea beetles (Chaetocnema spp., Haltica spp., and Epitrix spp.); corn rootworm (Diabrotica Spp.); cowpea weevil (Callosobruchus aculatue); boll weevil (Antonomus grandis); rice weevil (Sitophilus oryza); maize weevil (Sitophilus zeamais); granary weevil (Sitophilus granarius); Egyptian alfalfa weevil (Hypera postica); bean weevil (Acanthoseelides obtectus); lesser grain borer (Rhyzopertha dominica); yellow meal worm (Tenebrio molitor); Thysanoptera, especially, western flower thrips (Franklini ella occidentalis); Diptera, especially, leafminer spp. (Liriomyza trifolii); plant parasitic nematodes especially the potato cyst nematodes (Globodera spp.), the beet cyst nematode (Heterodera schachtii) and root knot nematodes (Meloidogyne spp.).

Another class of digestive enzymes is the aspartic proteases. The term “aspartic protease” is intended to describe a protease that possesses two highly reactive aspartic acid residues at the catalytic site of the enzyme and which is most often characterized by its specific inhibition with pepstatin, a low molecular weight inhibitor of nearly all known aspartic proteases. There is evidence that many phytophagous insects rely, in part, on midgut aspartic proteases for protein digestion most often in conjunction with cysteine proteases. These include but are not limited to Hemiptera especially (Rhodnius prolixus) and bedbug (Cimex spp.) and members of the families Phymatidae, Pentatomidae, Lygaeidae and Belostomatidae; Coleoptera, in the families of the Meloidae, Chrysomelidae, Coccinelidae and Bruchidae all belonging to the series Cucujiformia, especially, Colorado potato beetle (Leptinotarsa decemlineata) three-lined potato beetle (Lematri lineata); southern and western corn rootworm (Diabrotica undecimpunctata and D. virgifera), boll weevil (Anthonomus grandis), squash bug (Anasatristis); flea beetle (Phyllotreta crucifera), bruchid beetle (Callosobruchus maculatus), Mexican bean beetle (Epilachna varivestis), soybean leafminer (Odontota horni), margined blister beetle (Epicauta pestifera) and the red flour beetle (Triolium castaneum); Diptera, especially housefly (Musca domestica). See Terra and Ferreira (1994) Comn. Biochem. Physiol. 109B: 1-62; Wolfson and Murdock (1990) J. Chem. Ecol. 16:1089-1102.

Other examples of intervening linker peptides can be found in the following references, which are incorporated by reference herein in their entirety: a plant expressed serine proteinase inhibitor precursor was found to contain five homogeneous protein inhibitors separated by six same linker peptides, as disclosed in Heath et al. “Characterization of the protease processing sites in a multidomain proteinase inhibitor precursor from Nicotiana alata” European Journal of Biochemistry, 1995; 230:250-257. A comparison of the folding behavior of green fluorescent proteins through six different linkers is explored in Chang, H. C. et al. “De novo folding of GFP fusion proteins: high efficiency in eukaryotes but not in bacteria” Journal of Molecular Biology, 2005 Oct. 21; 353 (2): 397-409. An isoform of the human GalNAc-Ts family, GalNAc-T2, was shown to retain its localization and functionality upon expression in N. benthamiana plants by Daskalova, S. M. et al. “Engineering of N. benthamiana L. plants for production of N-acetylgalactosamine-glycosylated proteins” BMC Biotechnology, 2010 Aug. 24; 10:62. The ability of endogenous plastid proteins to travel through stromules was shown in Kwok, E. Y. et al. “GFP-labelled Rubisco and aspartate aminotransferase are present in plastid stromules and traffic between plastids” Journal of Experimental Botany, 2004 March; 55 (397): 595-604. Epub 2004 Jan. 30. A report on the engineering of the surface of the tobacco mosaic virus (TMV), virion, with a mosquito decapeptide hormone, trypsin-modulating oostatic factor (TMOF) was made by Borovsky, D. et al. “Expression of Aedes trypsin-modulating oostatic factor on the virion of TMV: A potential larvicide” Proc Natl Acad Sci, 2006 Dec. 12; 103 (50): 18963-18968. These references and others teach and disclose the intervening linkers that can be used in the methods, procedures and peptide, protein and nucleotide complexes and constructs described herein.

### The KPV ORF and KPV Constructs

“KPV ORF” refers to a nucleotide encoding a KPV, and/or one or more stabilizing proteins, secretory signals, or target directing signals, for example, ERSP or STA, and is defined as the nucleotides in the ORF that has the ability to be translated. Thus, a “KPV ORF diagram” refers to the composition of one or more KPV ORFs, as written out in diagram or equation form. For example, a “KPV ORF diagram” can be written out as using acronyms or short-hand references to the DNA segments contained within the expression ORF. Accordingly, in one example, a “KPV ORF diagram” may describe the polynucleotide segments encoding the ERSP, LINKER, STA, and KPV, by diagramming in equation form the DNA segments as “ersp” (i.e., the polynucleotide sequence that encodes the ERSP polypeptide); “linker” or “L” (i.e., the polynucleotide sequence that encodes the LINKER polypeptide); “sta” (i.e., the polynucleotide sequence that encodes the STA polypeptide), and “kpv” (i.e., the polynucleotide sequence encoding a KPV), respectively. An example of a KPV ORF diagram is “ersp-sta-(linkerᵢ-kpvⱼ)_{N},” or “ersp-(kpvⱼ-linkerᵢ)_{N}-sta” and/or any combination of the DNA segments thereof.

The following equations describe two examples of a KPV ORF that encodes an ERSP, a STA, a linker, and a KPV:

$$ersp - sta - l - kpv or ersp - kpv - l - sta$$

In some embodiments, the KPV open reading frame (ORF) described herein is a polynucleotide sequence that will enable the plant to express mRNA, which in turn will be translated into peptides that will folded properly, and/or accumulated to such an extent that said proteins provide a dose sufficient to inhibit and/or kill one or more pests. In one embodiment, an example of a protein KPV ORF can be a Kappa variant polynucleotide (kpv), an “ersp” (i.e., the polynucleotide sequence that encodes the ERSP polypeptide) a “linker” (i.e., the polynucleotide sequence that encodes the LINKER polypeptide), a “sta” (i.e., the polynucleotide sequence that encodes the STA polypeptide), or any combination thereof, and can be described in the following equation format:

$$e rsp - sta - {( {linker}_{i} - k p {v}_{j} )}_{n} , or ersp - {( kp {v}_{j} - {linker}_{i} )}_{n} - sta$$

The foregoing illustrative embodiment of a polynucleotide equation would result in the following protein complex being expressed: ERSP-STA-(LINKER_{I}-KPV_{J})_{N}, containing four possible peptide components with dash signs to separate each component. The nucleotide component of ersp is a polynucleotide segment encoding a plant endoplasmic reticulum trafficking signal peptide (ERSP). The component of sta is a polynucleotide segment encoding a translation stabilizing protein (STA), which helps the accumulation of the KPV expressed in plants, however, in some embodiments, the inclusion of sta may not be necessary in the KPV ORF. The component of linkerᵢ is a polynucleotide segment encoding an intervening linker peptide (L OR LINKER) to separate the KPV from other components contained in ORF, and from the translation stabilizing protein. The subscript letter “i” indicates that in some embodiments, different types of linker peptides can be used in the KPV ORF. The component “kpv” indicates the polynucleotide segment encoding the KPV. The subscript “j” indicates different polynucleotides may be included in the KPV ORF. For example, in some embodiments, the polynucleotide sequence can encode a KPV with a different amino acid substitution. The subscript “n” as shown in “(linkerᵢ-kpvⱼ)ₙ” indicates that the structure of the nucleotide encoding an intervening linker peptide and a KPV can be repeated “n” times in the same open reading frame in the same KPV ORF, where “n” can be any integrate number from 1 to 10; “n” can be from 1 to 10, specifically “n” can be 1, 2, 3, 4, or 5, and in some embodiments “n” is 6, 7, 8, 9 or 10. The repeats may contain polynucleotide segments encoding different intervening linkers (LINKER) and different KPVs. The different polynucleotide segments including the repeats within the same KPV ORF are all within the same translation frame. In some embodiments, the inclusion of a sta polynucleotide in the KPV ORF may not be required. For example, an ersp polynucleotide sequence can be directly be linked to the polynucleotide encoding a KPV variant polynucleotide without a linker.

In the foregoing exemplary equation, the polynucleotide “kpv” encoding the polypeptide “KPV” can be a polynucleotide sequence that encodes any KPV as described herein, e.g., a KPV comprising, consisting essentially of, or consisting of, an amino acid sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253.

In some embodiments, the kpv polynucleotide, or polynucleotide operable to encode a KPV, can encode a KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; wherein the KPV comprises at least one amino acid substitution relative to the amino acid sequence set forth in SEQ ID NO: 1, with the proviso that the KPV does not comprise the amino acid sequence of SEQ ID NOs: 1 or 253.

In some embodiments, a polynucleotide is operable to encode a KPV-pesticidal protein having the following KPV construct orientation and/or arrangement: ERSP-KPV; ERSP-(KPV)_{N}; ERSP-KPV-L; ERSP-(KPV)_{N}-L; ERSP-(KPV-L)_{N}; ERSP-L-KPV; ERSP-L-(KPV)_{N}; ERSP-(L-KPV)_{N}; ERSP-STA-KPV; ERSP-STA-(KPV)_{N}; ERSP-KPV-STA; ERSP-(KPV)_{N}-STA; ERSP-(STA-KPV)_{N}; ERSP-(KPV-STA)_{N}; ERSP-L-KPV-STA; ERSP-L-STA-KPV; ERSP-L-(KPV-STA)_{N}; ERSP-L-(STA-KPV)_{N}; ERSP-L-(KPV)_{N}-STA; ERSP-(L-KPV)_{N}-STA; ERSP-(L-STA-KPV)_{N}; ERSP-(L-KPV-STA)_{N}; ERSP-(L-STA)_{N}-KPV; ERSP-(L-KPV)_{N}-STA; ERSP-STA-L-KPV; ERSP-STA-KPV-L; ERSP-STA-L-(KPV)_{N}; ERSP-(STA-L)_{N}-KPV; ERSP-STA-(L-KPV)_{N}; ERSP-(STA-L-KPV)_{N}; ERSP-STA-(KPV)_{N}-L; ERSP-STA-(KPV-L)_{N}; ERSP-(STA-KPV)_{N}-L; ERSP-(STA-KPV-L)_{N}; ERSP-KPV-L-STA; ERSP-KPV-STA-L; ERSP-(KPV)_{N}-STA-L ERSP-(KPV-L)_{N}-STA; ERSP-(KPV-STA)_{N}-L; ERSP-(KPV-L-STA)_{N}; or ERSP-(KPV-STA-L)_{N}; wherein N is an integer ranging from 1 to 200.

Any of the aforementioned methods, and/or any of the methods described herein, can be used to incorporate into a plant or a plant part thereof, one or more polynucleotides operable to express any one or more of the KPVs or KPV-pesticidal proteins as described herein; e.g., one or more KPVs or KPV-pesticidal protein having the amino acid sequence of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, which are likewise described herein.

The present disclosure may be used for transformation of any plant species, including, but not limited to, monocots and dicots. Crops for which a transgenic approach or PEP would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers. Additional useful crops and plant species are provided below.

Transforming Plants with Polynucleotides

In some embodiments, the KPV ORFs and KPV constructs described above and herein can be cloned into any plant expression vector for KPV to be expressed in plants, either transiently or stably.

Transient plant expression systems can be used to promptly optimize the structure of the KPV ORF for some specific KPV expression in plants, including the necessity of some components, codon optimization of some components, optimization of the order of each component, etc. A transient plant expression vector is often derived from a plant virus genome. Plant virus vectors provide advantages in quick and high level of foreign gene expression in plant due to the infection nature of plant viruses. The full length of the plant viral genome can be used as a vector, but often a viral component is deleted, for example the coat protein, and transgenic ORFs are subcloned in that place. The KPV ORF can be subcloned into such a site to create a viral vector. These viral vectors can be introduced into plant mechanically since they are infectious themselves, for example through plant wound, spray-on etc. They can also be transfected into plants via agroinfection, by cloning the virus vector into the T-DNA of the crown gall bacterium, Agrobacterium tumefaciens, or the hairy root bacterium, Agrobacterium rhizogenes. The expression of the KPV in this vector is controlled by the replication of the RNA virus, and the virus translation to mRNA for replication is controlled by a strong viral promoter, for example, 35S promoter from Cauliflower mosaic virus. Viral vectors with KPV ORF are usually cloned into T-DNA region in a binary vector that can replicate itself in both E. coli strains and Agrobacterium strains. The transient transfection of a plant can be done by infiltration of the plant leaves with the Agrobacterium cells which contain the viral vector for KPV expression. In the transient transformed plant, it is common for the foreign protein expression to be ceased in a short period of time due to the post-transcriptional gene silencing (PTGS). Sometimes a PTGS suppressing protein gene is necessary to be co-transformed into the plant transiently with the same type of viral vector that drives the expression of with the KPV ORF. This improves and extends the expression of the KPV in the plant. The most commonly used PTGS suppressing protein is P19 protein discovered from tomato bushy stunt virus (TBSV).

In some embodiments, transient transfection of plants can be achieved by recombining a polynucleotide encoding a KPV with any one of the readily available vectors (see above and described herein), and confirmed, using a marker or signal (e.g., GFP emission). In some embodiments, a transiently transfected plant can be created by recombining a polynucleotide encoding a KPV with a DNA encoding a GFP-Hybrid fusion protein in a vector, and transfection said vector into a plant (e.g., tobacco) using different FECT vectors designed for targeted expression. In some embodiments, a polynucleotide encoding a KPV can be recombined with a pFECT vector for APO (apoplast localization) accumulation; a pFECT vector for CYTO (cytoplasm localization) accumulation; or pFECT with ersp vector for ER (endoplasm reticulum localization) accumulation.

An exemplary transient plant transformation strategy is agroinfection using a plant viral vector due to its high efficiency, ease, and low cost. In some embodiments, a tobacco mosaic virus overexpression system can be used to transiently transform plants with KPV. See TRBO, Lindbo J A, Plant Physiology, 2007, V145: 1232-1240, the disclosure of which is incorporated herein by reference in its entirety.

The TRBO DNA vector has a T-DNA region for agroinfection, which contains a CaMV 35S promoter that drives expression of the tobacco mosaic virus RNA without the gene encoding the viral coating protein. Moreover, this system uses the “disarmed” virus genome, therefore viral plant to plant transmission can be effectively prevented.

In another embodiment, the FECT viral transient plant expression system can be used to transiently transform plants with KPV. See Liu Z & Kearney C M, BMC Biotechnology, 2010, 10:88, the disclosure of which is incorporated herein by reference in its entirety. The FECT vector contains a T-DNA region for agroinfection, which contains a CaMV 35S promoter that drives the expression of the foxtail mosaic virus RNA without the genes encoding the viral coating protein and the triple gene block. Moreover, this system uses the “disarmed” virus genome, therefore viral plant to plant transmission can be effectively prevented. To efficiently express the introduced heterologous gene, the FECT expression system additionally needs to co-express P19, a RNA silencing suppressor protein from tomato bushy stunt virus, to prevent the post-transcriptional gene silencing (PTGS) of the introduced T-DNA (the TRBO expression system does not need co-expression of P19).

In some embodiments, the KPV ORF can be designed to encode a series of translationally fused structural motifs that can be described as follows: N′-ERSP-STA-L-KPV-C′ wherein the “N′” and “C′” indicating the N-terminal and C-terminal amino acids, respectively, and the ERSP motif can be the Barley Alpha-Amylase Signal peptide (BAAS) (SEQ ID NO:359); the stabilizing protein (STA) can be GFP (SEQ ID NO:374); the linker peptide “L” can be IGER (SEQ ID NO:377) In some embodiments, the ersp-sta-l-kpv ORF can chemically synthesized to include restrictions sites, for example a Pac I restriction site at its 5′-end, and an Avr II restriction site at its 3′-end. In some embodiments, the KPV ORF can be cloned into the Pac I and Avr II restriction sites of a FECT expression vector (pFECT) to create a KPV expression vector for the FECT transient plant expression system (pFECT-KPV). To maximize expression in the FECT expression system, some embodiments may have a FECT vector expressing the RNA silencing suppressor protein P19 (pFECT-P19) generated for co-transformation.

In some embodiments, a vector can be recombined for use in a TRBO transient plant expression system, for example, by performing a routine PCR procedure and adding a Not I restriction site to the 3′-end of the KPV ORF described above, and then cloning the KPV ORF into Pac I and Not I restriction sites of the TRBO expression vector (pTRBO-KPV).

In some embodiments, an Agrobacterium tumefaciens strain, for example, commercially available GV3101 cells, can be used for the transient expression of a KPV ORF in a plant tissue (e.g., tobacco leaves) using one or more transient expression systems, for example, the FECT and TRBO expression systems. An exemplary illustration of such a transient transfection protocol includes the following: an overnight culture of GV3101 can be used to inoculate 200 mL Luria-Bertani (LB) medium; the cells can be allowed to grow to log phase with OD600 between 0.5 and 0.8; the cells can then be pelleted by centrifugation at 5000 rpm for 10 minutes at 4° C.; cells can then be washed once with 10 mL prechilled TE buffer (Tris-HCl 10 mM, EDTA 1 mM, pH8.0), and then resuspended into 20 mL LB medium; GV3101 cell resuspension can then be aliquoted in 250 μL fractions into 1.5 mL microtubes; aliquots can then be snap-frozen in liquid nitrogen and stored at −80° C. freezer for future transformation. The pFECT-KPV and pTRBO-KPV vectors can then transformed into the competent GV3101 cells using a freeze-thaw method as follows: the stored competent GV3101 cells are thawed on ice and mixed with 1 to 5 μg pure DNA (pFECT-KPV or pTRBO-KPV vector). The cell-DNA mixture is kept on ice for 5 minutes, transferred to −80° C. for 5 minutes, and incubated in a 37° C. water bath for 5 minutes. The freeze-thaw treated cells are then diluted into 1 mL LB medium and shaken on a rocking table for 2 to 4 hours at room temperature. A 200 μL aliquot of the cell-DNA mixture is then spread onto LB agar plates with the appropriate antibiotics (10 μg/mL rifampicin, 25 μg/mL gentamycin, and 50 μg/mL kanamycin can be used for both pFECT-KPV transformation and pTRBO-KPV transformation) and incubated at 28° C. for two days. Resulting transformed colonies are then picked and cultured in 6 mL aliquots of LB medium with the appropriate antibiotics for transformed DNA analysis and making glycerol stocks of the transformed GV3101 cells.

In some embodiments, the transient transformation of plant tissues, for example, tobacco leaves, can be performed using leaf injection with a 3-mL syringe without needle. In one illustrative example, the transformed GV3101 cells are streaked onto an LB plate with the appropriate antibiotics (as described above) and incubated at 28° C. for two days. A colony of transformed GV3101 cells are inoculated to 5 ml of LB-MESA medium (LB media supplemented with 10 mM MES, and 20 μM acetosyringone) and the same antibiotics described above, and grown overnight at 28° C. The cells of the overnight culture are collected by centrifugation at 5000 rpm for 10 minutes and resuspended in the induction medium (10 mM MES, 10 mM MgCl₂, 100 μM acetosyringone) at a final OD600 of 1.0. The cells are then incubated in the induction medium for 2 hours to overnight at room temperature and are then ready for transient transformation of tobacco leaves. The treated cells can be infiltrated into the underside of attached leaves of Nicotiana benthamiana plants by injection, using a 3-mL syringe without a needle attached.

In some embodiments, the transient transformation can be accomplished by transfecting one population of GV3101 cells with pFECT-KPV or pTRBO-KPV and another population with pFECT-P19, mixing the two cell populations together in equal amounts for infiltration of tobacco leaves by injection with a 3-mL syringe.

Stable integration of polynucleotide operable to encode KPV is also possible with the present disclosure, for example, the KPV ORF can also be integrated into plant genome using stable plant transformation technology, and therefore KPVs can be stably expressed in plants and protect the transformed plants from generation to generation. For the stable transformation of plants, the KPV expression vector can be circular or linear. The KPV ORF, the KPV expression cassette, and/or the vector with polynucleotide encoding a KPV for stable plant transformation should be carefully designed for optimal expression in plants based on what is known to those having ordinary skill in the art, and/or by using predictive vector design tools such as Gene Designer 2.0 (Atum Bio); VectorBuilder (Cyagen); SnapGene® viewer; GeneArt™ Plasmid Construction Service (Thermo-Fisher Scientific); and/or other commercially available plasmid design services. See Tolmachov, Designing plasmid vectors. Methods Mol Biol. 2009; 542:117-29. The expression of KPV is usually controlled by a promoter that promotes transcription in some, or all the cells of the transgenic plant. The promoter can be a strong plant viral promoter, for example, the constitutive 35S promoter from Cauliflower Mosaic Virus (CaMV); it also can be a strong plant promoter, for example, the hydroperoxide lyase promoter (pHPL) from Arabidopsis thaliana; the Glycine max polyubiquitin (Gmubi) promoter from soybean; the ubiquitin promoters from different plant species (rice, corn, potato, etc.), etc. A plant transcriptional terminator often occurs after the stop codon of the ORF to halt the RNA polymerase and transcription of the mRNA. To evaluate the KPVs expression, a reporter gene can be included in the KPV expression vector, for example, beta-glucuronidase gene (GUS) for GUS straining assay, green fluorescent protein (GFP) gene for green fluorescence detection under UV light, etc. For selection of transformed plants, a selection marker gene is usually included in the KPV expression vector. In some embodiments, the marker gene expression product can provide the transformed plant with resistance to specific antibiotics, for example, kanamycin, hygromycin, etc., or specific herbicide, for example, glyphosate etc. If agroinfection technology is adopted for plant transformation, T-DNA left border and right border sequences are also included in the KPV expression vector to transport the T-DNA portion into the plant.

The constructed KPV expression vector can be transfected into plant cells or tissues using many transfection technologies. Agroinfection is a very popular way to transform a plant using an Agrobacterium tumefaciens strain or an Agrobacterium rhizogenes strain. Particle bombardment (also called Gene Gun, or Biolistics) technology is also very common method of plant transfection. Other less common transfection methods include tissue electroporation, silicon carbide whiskers, direct injection of DNA, etc. After transfection, the transfected plant cells or tissues placed on plant regeneration media to regenerate successfully transfected plant cells or tissues into transgenic plants.

Evaluation of a transformed plant can be accomplished at the DNA level, RNA level and protein level. A stably transformed plant can be evaluated at all of these levels and a transiently transformed plant is usually only evaluated at protein level. To ensure that the KPV ORF integrates into the genome of a stably transformed plant, the genomic DNA can be extracted from the stably transformed plant tissues for and analyzed using PCR or Southern blot. The expression of the KPV in the stably transformed plant can be evaluated at the RNA level, for example, by analyzing total mRNA extracted from the transformed plant tissues using northern blot or RT-PCR. The expression of the KPV in the transformed plant can also be evaluated in protein level directly. There are many ways to evaluate expression of KPV in a transformed plant. If a reporter gene included in the KPV ORF, a reporter gene assay can be performed, for example, in some embodiments a GUS straining assay for GUS reporter gene expression, a green fluorescence detection assay for GFP reporter gene expression, a luciferase assay for luciferase reporter gene expression, and/or other reporter techniques may be employed.

In some embodiments total protein can be extracted from the transformed plant tissues for the direct evaluation of the expression of the KPV using a Bradford assay to evaluate the total protein level in the sample.

In some embodiments, analytical HPLC chromatography technology, Western blot technique, or iELISA assay can be adopted to qualitatively or quantitatively evaluate the KPV in the extracted total protein sample from the transformed plant tissues. KPV expression can also be evaluated by using the extracted total protein sample from the transformed plant tissues in an insect bioassay, for example, in some embodiments, the transformed plant tissue or the whole transformed plant itself can be used in insect bioassays to evaluate KPV expression and its ability to provide protection for the plant.

In some embodiments, a plant, plant tissue, plant cell, plant seed, or part thereof of the present disclosure, can comprise one or more KPVs, or a polynucleotide encoding the same, said KPV comprising an amino acid sequence that is at least 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

### Confirming Successful Transformation

Following introduction of heterologous foreign DNA into plant cells, the transformation or integration of heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell (2001) Molecular Cloning: A Laboratory Manual. Cold Spring Harbor Laboratory Press, Cold Spring Harbor, N.Y.). PCR is carried out using oligonucleotide primers specific to the gene of interest or Agrobacterium vector background, etc.

Plant transformation may be confirmed by Southern blot analysis of genomic DNA (Sambrook and Russell, 2001, supra). In general, total DNA is extracted from the transformed plant, digested with appropriate restriction enzymes, fractionated in an agarose gel and transferred to a nitrocellulose or nylon membrane. The membrane or “blot” is then probed with, for example, radiolabeled ³²P target DNA fragment to confirm the integration of introduced gene into the plant genome according to standard techniques (Sambrook and Russell, 2001, supra).

In Northern blot analysis, RNA is isolated from specific tissues of transformed plant, fractionated in a formaldehyde agarose gel, and blotted onto a nylon filter according to standard procedures that are routinely used in the art (Sambrook and Russell, 2001, supra). Expression of RNA encoded by the polynucleotide encoding a KPV is then tested by hybridizing the filter to a radioactive probe derived from a KPV, by methods known in the art (Sambrook and Russell, 2001, supra).

Western blot, biochemical assays and the like may be carried out on the transgenic plants to confirm the presence of protein encoded by the KPV gene by standard procedures (Sambrook and Russell, 2001, supra) using antibodies that bind to one or more epitopes present on the KPV.

A number of markers have been developed to determine the success of plant transformation, for example, resistance to chloramphenicol, the aminoglycoside G418, hygromycin, or the like. Other genes that encode a product involved in chloroplast metabolism may also be used as selectable markers. For example, genes that provide resistance to plant herbicides such as glyphosate, bromoxynil, or imidazolinone may find particular use. Such genes have been reported (Stalker et al. (1985) J. Biol. Chem. 263:6310-6314 (bromoxynil resistance nitrilase gene); and Sathasivan et al. (1990) Nucl. Acids Res. 18:2188 (AHAS imidazolinone resistance gene). Additionally, the genes disclosed herein are useful as markers to assess transformation of bacterial, yeast, or plant cells. Methods for detecting the presence of a transgene in a plant, plant organ (e.g., leaves, stems, roots, etc.), seed, plant cell, propagule, embryo or progeny of the same are well known in the art. In one embodiment, the presence of the transgene is detected by testing for pesticidal activity.

Fertile plants expressing a KPV and/or a polynucleotide encoding the same may be tested for pesticidal activity, and the plants showing optimal activity selected for further breeding. Methods are available in the art to assay for pest activity. Generally, the protein is mixed and used in feeding assays. See, for example Marrone et al. (1985) J. of Economic Entomology 78:290-293.

In some embodiments, evaluating the success of a transient transfection procedure can be determined based on the expression of a reporter gene, for example, GFP. In some embodiments, GFP can be detected under UV light in tobacco leaves transformed with the FECT and/or TRBO vectors.

In some embodiments, KPV expression can be quantitatively evaluated in a plant (e.g., tobacco). An exemplary procedure that illustrates KPV quantification in a tobacco plant is as follows: 100 mg disks of transformed leaf tissue is collected by punching leaves with the large opening of a 1000 μL pipette tip. The collected leaf tissue is place into a 2 mL microtube with 5/32″ diameter stainless steel grinding balls, and frozen in −80° C. for 1 hour, and then homogenized using a Troemner-Talboys High Throughput Homogenizer. Next, 750 μL ice-cold TSP-SE1 extraction solutions (sodium phosphate solution 50 mM, 1:100 diluted protease inhibitor cocktail, EDTA 1 mM, DIECA 10 mM, PVPP 8%, pH 7.0) is added into the tube and vortexed. The microtube is then left still at room temperature for 15 minutes and then centrifuged at 16,000 g for 15 minutes at 4° C.; 100 μL of the resulting supernatant is taken and loaded into pre-Sephadex G-50-packed column in 0.45 μm Millipore MultiScreen filter microtiter plate with empty receiving Costar microtiter plate on bottom. The microtiter plates are then centrifuged at 800 g for 2 minutes at 4° C. The resulting filtrate solution, herein called total soluble protein extract (TSP extract) of the tobacco leaves, is then ready for the quantitative analysis.

In some embodiments, the total soluble protein concentration of the TSP extract can be estimated using Pierce Coomassie Plus protein assay. BSA protein standards with known concentrations can be used to generate a protein quantification standard curve. For example, 2 μL of each TSP extract can be mixed into 200 μL of the chromogenic reagent (CPPA reagent) of the Coomassie Plus protein assay kits and incubated for 10 minutes. The chromogenic reaction can then be evaluated by reading OD595 using a SpectroMax-M2 plate reader using SoftMax Pro as control software. The concentrations of total soluble proteins can be about 0.788±0.20 μg/μL or about 0.533±0.03 μg/μL in the TSP extract from plants transformed via FECT and TRBO, respectively, and the results can be used to calculate the percentage of the expressed KPV in the TSP (% TSP) for the iELISA assay

In some embodiments, an indirect ELISA (iELISA) assay can be used to quantitatively evaluate the KPV content in the tobacco leaves transiently transformed with the FECT and/or TRBO expression systems. An illustrative example of using iELISA to quantify KPV is as follows: 5 μL of the leaf TSP extract is diluted with 95 μL of CB2 solution (Immunochemistry Technologies) in the well of an Immulon 2HD 96-well plate, with serial dilutions performed as necessary; leaf proteins obtained from extract samples are then allowed to coat the well walls for 3 hours in the dark, at room temperature, and the CB2 solution is then subsequently removed; each well is washed twice with 200 μL PBS (Gibco); 150 μL blocking solution (Block BSA in PBS with 5% non-fat dry milk) is added into each well and incubated for 1 hour, in the dark, at room temperature; after the removal of the blocking solution, a PBS wash of the wells, 100 μL of primary antibodies directed against KPV (custom antibodies are commercially available from ProMab Biotechnologies, Inc.; GenScript®; or raised using the knowledge readily available to those having ordinary skill in the art); the antibodies diluted at 1:250 dilution in blocking solution are added to each well and incubated for 1 hour in the dark at room temperature; the primary antibody is removed and each well is washed with PBS 4 times; 100 μL of HRP-conjugated secondary antibody (i.e., antibody directed against host species used to generate primary antibody, used at 1:1000 dilution in the blocking solution) is added into each well and incubated for 1 hour in the dark at room temperature; the secondary antibody is removed and the wells are washed with PBS, 100 μL; substrate solution (a 1:1 mixture of ABTS peroxidase substrate solution A and solution B, KPL) is added to each well, and the chromogenic reaction proceeds until sufficient color development is apparent; 100 μL of peroxidase stop solution is added to each well to stop the reaction; light absorbance of each reaction mixture in the plate is read at 405 nm using a SpectroMax-M2 plate reader, with SoftMax Pro used as control software; serially diluted known concentrations of pure KPVs samples can be treated in the same manner as described above in the iELISA assay to generate a mass-absorbance standard curve for quantities analysis. The expressed KPV can be detected by iELISA at about 3.09±1.83 ng/μL in the leaf TSP extracts from the FECT transformed tobacco; and about 3.56±0.74 ng/μL in the leaf TSP extract from the TRBO transformed tobacco. Alternatively, the expressed KPV can be about 0.40% total soluble protein (% TSP) for FECT transformed plants and about 0.67% TSP in TRBO transformed plants.

In some embodiments, a plant, plant tissue, plant cell, plant seed, or part thereof, comprises a KPV or a polynucleotide encoding the same, or a complementary nucleotide sequence thereof, said KPV comprising, consisting essentially of, or consisting of, an amino acid sequence that is at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253.

In some embodiments, a plant, plant tissue, plant cell, or plant seed comprises a KPV that comprises, consists essentially of, or consists of, an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a plant, plant tissue, plant cell, or plant seed comprises a polynucleotide encoding a KPV, or a complementary nucleotide sequence thereof, wherein the KPV comprises, consists essentially of, or consists of, an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, the plant, plant tissue, plant cell, plant seed, or part thereof, comprises a KPV or a polynucleotide encoding the same, wherein the polynucleotide is operable to encode a homopolymer or heteropolymer of two or more KPVs, wherein the amino acid sequence of each KPV is the same or different.

In some embodiments, the plant, plant tissue, plant cell, plant seed, or part thereof, comprises a KPV or a polynucleotide encoding the same, wherein the polynucleotide is operable to encode a KPV that is a fused protein comprising two or more KPVs separated by a cleavable or non-cleavable linker, and wherein the amino acid sequence of each KPV may be the same or different.

In some embodiments, the linker is a cleavable linker.

In some embodiments, the linker has an amino acid sequence as set forth in any one of SEQ ID NOs: 360-369.

In some embodiments, the linker is cleavable inside at least one of (i) the gut or hemolymph of an insect, and (ii) cleavable inside the gut of a mammal.

In some embodiments, the plant, plant tissue, plant cell, or plant seed comprises a KPV wherein the KPV has an amino acid sequence of any of the aforementioned KPVs (e.g., one or more the KPVs enumerated in Table 1), or a polynucleotide encoding the same.

Any of the aforementioned methods, and/or any of the methods described herein, can be used to incorporate one or more of the KPVs or KPV-pesticidal proteins as described herein, into plants or plant parts thereof. For example, any of the methods described herein can be used to incorporate into plants one or more of the KPVs described in the present disclosure, e.g., KPVs having the amino acid sequence of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, which are likewise described herein.

### Pesticidal Formulations, Compositions, and Combinations

The present disclosure contemplates mixtures, combinations, compositions, products, and transgenic organisms that contain—or, in the case of transgenic organisms, express or otherwise produce—one or more KPVs, and/or one or more KPV-pesticidal proteins.

In some embodiments, the illustrative mixtures consists of: (1) a KPV, a KPV-pesticidal proteins, or an agriculturally acceptable salt thereof; and (2) an excipient (e.g., any of the excipients described herein).

In some embodiments, the mixtures of the present disclosure consist of: (1) one or more KPVs, one or more KPV-pesticidal proteins, or an agriculturally acceptable salt thereof; and (2) one or more excipients (e.g., any of the excipients described herein).

In some embodiments, the mixtures of the present disclosure consist of: (1) one or more KPVs, one or more KPV-pesticidal proteins, or an agriculturally acceptable salt thereof; and (2) one or more excipients (e.g., any of the excipients described herein); wherein either of the foregoing (1) or (2) can be used concomitantly, or sequentially.

Any of the combinations, mixtures, products, polypeptides and/or plants utilizing a KPV, or a KPV-pesticidal protein (as described herein), can be used to control pests, their growth, and/or the damage caused by their actions, especially their damage to plants.

Compositions comprising a KPV or a KPV-pesticidal protein, or an agriculturally acceptable salt thereof, and an excipient, can include agrochemical compositions. For example, in some embodiments, agrochemical compositions can include, but is not limited to, aerosols and/or aerosolized products (e.g., sprays, fumigants, powders, dusts, and/or gases); seed dressings; oral preparations (e.g., insect food, etc.); or a transgenic organisms (e.g., a cell, a plant, or an animal) expressing and/or producing a KPV or a KPV-pesticidal protein, either transiently and/or stably.

In some embodiments, the active ingredients of the present disclosure can be applied in the form of compositions and can be applied to the crop area or plant to be treated, simultaneously or in succession, with other non-active compounds. These compounds can be fertilizers, weed killers, cryoprotectants, surfactants, detergents, soaps, dormant oils, polymers, and/or time-release or biodegradable carrier formulations that permit long-term dosing of a target area following a single application of the formulation. One or more of these non-active compounds can be prepared, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, tackifiers, binders or fertilizers. Likewise, the formulations may be prepared into edible “baits” or fashioned into pest “traps” to permit feeding or ingestion by a target pest of the pesticidal formulation.

Methods of applying an active ingredient of the present disclosure or an agrochemical composition of the present disclosure that consists of a KPV or KPV-pesticidal protein or an agriculturally acceptable salt thereof, and an excipient, as produced by the methods described herein of the present disclosure, include leaf application, seed coating and soil application. In some embodiments, the number of applications and the rate of application depend on the intensity of infestation by the corresponding pest.

The composition comprising a KPV or a KPV-pesticidal protein or an agriculturally acceptable salt thereof and an excipient may be formulated as a powder, dust, pellet, granule, spray, emulsion, colloid, solution, or such like, and may be prepared by such conventional means as desiccation, lyophilization, homogenization, extraction, filtration, centrifugation, sedimentation, or concentration of a culture of cells comprising the polypeptide. In all such compositions that contain at least one such pesticidal polypeptide, the polypeptide may be present in a concentration of from about 1% to about 99% by weight.

In some embodiments, compositions containing KPVs or KPV-pesticidal proteins (or an agriculturally acceptable salt thereof) may be prophylactically applied to an environmental area to prevent infestation by a susceptible pest, for example, a lepidopteran and/or coleopteran pest, which may be killed or reduced in numbers in a given area by the methods of the invention. In some embodiments, the pest ingests, or comes into contact with, a pesticidally-effective amount of the polypeptide.

In some embodiments, the pesticide compositions described herein may be made by formulating either the KPV or KPV-pesticidal-protein or an agriculturally acceptable salt thereof transformed bacterial, yeast, or other cell, crystal and/or spore suspension, or isolated protein component with the desired agriculturally-acceptable carrier. The compositions may be formulated prior to administration in an appropriate means such as lyophilized, freeze-dried, desiccated, or in an aqueous carrier, medium or suitable diluent, such as saline and/or other buffer. In some embodiments, the formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. In some embodiments, the formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in U.S. Pat. No. 6,468,523, the disclosure of which is incorporated herein by reference in its entirety.

Any of the KPVs or KPV-pesticidal proteins described herein (e.g., a KPV having an amino acid sequence as set forth in SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof) can be used to create a mixture, a combination, and/or composition, wherein said mixture, combination, and/or composition consists of at least one KPV.

As used herein, a “combination” refers to the result of combining two or more separate components (e.g., a first component and one or more additional components). Thus, as used herein, a “combination” refers to an association of two or more separate components, e.g., a first KPV and one or more additional component (e.g., one or more additional KPVs, wherein said one or more additional KPVs are the same or different from the first KPV; or, in the case of a composition, one or more excipients). Accordingly, in some embodiments, a combination can refer to the association of a first KPV, and one or more additional KPVs; wherein the first KPV and one or more additional KPVs are the same or different.

In some embodiments, a combination can be a “mixture.” For example, in some embodiments, a mixture refers to a combination of a first component, and one or more additional components, wherein the first component and the one or more additional components are present together in a single entity (e.g., a single unit). Thus, in some embodiments, a mixture can comprise a first component, and one or more additional components, wherein the first component and the one or more additional components are present in admixture for simultaneous administration.

Accordingly, in some embodiments, a combination can refer to the association of a first KPV, and one or more additional KPVs; wherein the first KPV and one or more additional KPVs are the same or different; and wherein the first KPV and one or more additional KPVs are present in a single entity (e.g., an admixture for simultaneous administration).

In some embodiments, a combination can comprise a first component, and one or more additional components, wherein the first component and the one or more additional components, are present separately (e.g., more than one unit). For example, in some embodiments, a combination can comprise a first component, and one or more additional components, wherein the first component and the one or more additional components may be administered separately, sequentially, simultaneously, concurrently or chronologically staggered.

Thus, in some embodiments, a combination can refer to the association of a first KPV, and one or more additional KPVs; wherein the first KPV and one or more additional KPVs are the same or different; and wherein the first KPV and one or more additional KPVs are present separately (e.g., different units for separate, sequential, simultaneous, concurrent or chronologically-staggered administration).

In yet other embodiments, a combination can comprise a first component, and one or more additional components, wherein the first component and the one or more additional components, are present separately; and wherein the first component expressed in a plant, plant tissue, seed, or part thereof, and the one or more additional components are applied to the plant, plant tissue, seed, or part thereof (e.g., as a composition) or vice versa.

For example, in some embodiments, a combination comprises: (1) a first KPV, or polynucleotide encoding the same; and (2) one or more additional KPVs, or polynucleotides encoding the same; wherein the first KPV and one or more additional KPVs are the same or different; and wherein (1) is expressed in a transgenic plant, plant tissue, plant cell, plant seed, or part thereof; and (2) is applied to the transgenic plant, plant tissue, plant cell, plant seed, or part thereof, e.g., as a composition.

In another embodiments, a combination comprises: (1) a first KPV, or polynucleotide encoding the same; and (2) one or more additional KPVs, or polynucleotides encoding the same; wherein the first KPV and one or more additional KPVs are the same or different; and wherein (2) is expressed in a transgenic plant, plant tissue, plant cell, plant seed, or part thereof; and (1) is applied to the transgenic plant, plant tissue, plant cell, plant seed, or part thereof, e.g., as a composition.

In some embodiments, a combination can refer to the separate, sequential, simultaneous, concurrent or chronologically-staggered application of two or more separate components (e.g., a first KPV, and one or more additional KPVs; wherein the first KPV and one or more additional KPVs are the same or different). For example, in some embodiments, a “combination” refers to the result of a simultaneous application of both a first KPV, and one or more additional KPVs; wherein the first KPV and one or more additional KPVs are the same or different.

In another embodiment, a combination of the present disclosure refers to the result of a separate application of a first KPV, and one or more additional KPVs; wherein the first KPV and one or more additional KPVs are the same or different.

In a further embodiments, a combination of the present disclosure refers to the result of a sequential application of two or more separate components, e.g., a first application of a first KPV, followed by a second application of one or more additional KPVs (wherein the first KPV and one or more additional KPVs are the same or different), or vice versa. Where the application is sequential or separate, the delay in applying the second component should not be such as to lose the beneficial effect of the combination.

In some embodiments, a combination of the present disclosure can comprise a first KPV, and one or more additional KPVs (wherein the first KPV and one or more additional KPVs are the same or different), wherein the first KPV is expressed in a transgenic plant; and wherein the one or more additional KPVs are applied separately.

In some embodiments, a combination of the present disclosure can comprise a first KPV, and one or more additional KPVs (wherein the first KPV and one or more additional KPVs are the same or different), wherein the one or more additional KPVs are expressed in a transgenic plant; and wherein the first KPV is applied separately.

In some embodiments, a combination, mixture, or composition of the present disclosure, comprising a first component and one or more additional components, can be applied or administered separately, sequentially, simultaneously, concurrently or chronologically staggered. For example, in some embodiments, a combination, mixture, or composition of the present disclosure, comprising a first component and one or more additional components, can be applied or administered separately, wherein the first component and the one or more additional components are contained in separate units.

In some embodiments, a combination, mixture, or composition of the present disclosure, comprising a first component and one or more additional components, can be applied or administered sequentially, wherein the first component and the one or more additional components are administered in sequence, for example at an interval or intervals of minutes, hours, days or weeks (wherein the delay in administering the one or more additional components does not lose the beneficial effect of the combination).

In some embodiments, a combination, mixture, or composition of the present disclosure, comprising a first component and one or more additional components, can be applied or administered simultaneously, wherein the first component and the one or more additional components are administered at substantially the same time.

In some embodiments, a combination, mixture, or composition of the present disclosure, comprising a first component and one or more additional components, can be applied or administered contemporaneously, wherein the first component and the one or more additional components are administered closely in time, e.g., the first component is administered within from about 30 seconds, to within about one day before or after the one or more additional components; however, any contemporaneous time is useful given the delay in administering the one or more additional components does not lose the beneficial effect of the combination. In some embodiments, when administered contemporaneously, the first component and one and more additional are suitably administered at the same locus.

In some embodiments, a combination, mixture, or composition of the present disclosure, comprising a first component and one or more additional components, can be applied or administered sequentially, wherein the first component is applied just prior to, or just after the application the other one or more additional components.

In some embodiments, the combination can be, e.g., a mixture, or as part of a composition further comprising one or more excipients.

In some embodiments, a combination can refer to the simultaneous, separate, or sequential application of two or more separate components (e.g., a first KPV, and one or more additional KPVs; wherein the first KPV and one or more additional KPVs are the same or different); wherein the two or more separate components are not in admixture.

In some embodiments, the present disclosure comprises, consists essentially of, or consists of, a combination, a mixture, or a composition comprising, consisting essentially of, or consisting of, one or more KPVs, one or more KPV-pesticidal proteins, and/or combinations thereof.

In some embodiments, the invention contemplates a mixture of one or more KPVs, one or more KPV-pesticidal proteins, and/or combinations thereof. For example, in some embodiments, one or more KPVs, one or more KPV-pesticidal proteins, and/or combinations thereof, can be blended together in in varying proportions.

In some embodiments, the invention contemplates a combination of one or more KPVs, one or more KPV-pesticidal proteins, and/or combinations thereof. For example, in some embodiments, one or more KPVs, one or more KPV-pesticidal proteins, and/or combinations thereof, can be provided as a combination, e.g., in the same container, or in different containers.

In some embodiments, the invention contemplates a composition of one or more KPVs, one or more KPV-pesticidal proteins, and/or combinations thereof; and further comprising an excipient.

In some embodiments, the combination can be, e.g., a mixture, or as part of a composition wherein said composition further comprises one or more excipients. In some embodiments, a combination can refer to the simultaneous, separate, or sequential application of two or more separate components (e.g., a first KPV, and one or more additional KPVs; wherein the first KPV and one or more additional KPVs are the same or different).

In some embodiments, an illustrative mixture comprises at least two of: (1) one or more KPVs, or agriculturally acceptable salts thereof; (2) one or more KPV-pesticidal proteins, or agriculturally acceptable salts thereof; and/or (3) any combination thereof; wherein the KPV is the same or different; and the KPV and/or KPV-pesticidal protein are present in admixture for simultaneous administration (e.g., present in a single unit).

In some embodiments, an illustrative composition comprises: (1) a KPV, a KPV-pesticidal proteins, or an agriculturally acceptable salt thereof; and (2) at least one excipient (e.g., any of the excipients described herein).

In some embodiments, a composition of the present disclosure comprises: (1) one or more KPVs, one or more KPV-pesticidal proteins, or an agriculturally acceptable salt thereof; and (2) one or more excipients (e.g., any of the excipients described herein); wherein either of the foregoing (1) or (2) can be used concomitantly, or sequentially.

In some embodiments, a combination can comprise two or more KPVs and/or KPV-pesticidal proteins, wherein the two or more KPVs and/or KPV-pesticidal proteins are the same or different.

In some embodiments, the combination, mixture, or composition comprises, consists essentially of, or consists of, a Kappa Peptide Variant (KPV) having pesticidal activity against one or more insect species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences provided in Table 1; wherein the KPV comprises at least one amino acid substitution relative to the amino acid sequence set forth in SEQ ID NO: 1, with the proviso that the KPV does not comprise the amino acid sequence of SEQ ID NOs: 1 or 253.

In some embodiments, a combination, mixture, or composition of the present disclosure comprises, consists essentially of, or consists of, a KPV, or an agriculturally acceptable salt thereof, having pesticidal activity against one or more insect species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a combination of the present disclosure comprises, consists essentially of, or consists of, two or more KPVs having pesticidal activity against one or more insect species, each KPV independently comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a composition of the present disclosure comprises, consists essentially of, or consists of, at least one KPV having pesticidal activity against one or more insect species, and at least one excipient; wherein the KPV comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a composition of the present disclosure comprises, consists essentially of, or consists of, two or more KPVs having pesticidal activity against one or more insect species; and at least one excipient; wherein each KPV independently comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a Kappa Variant Peptide (KPV) having pesticidal activity against one or more pest species, said KPV or agriculturally acceptable salt thereof comprising or consisting of an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences provided in the foregoing Table 1; with the proviso that the KPV does not comprise the amino acid sequence of SEQ ID NOs: 1 or 253.

In some embodiments, a combination, mixture, or composition of the present disclosure comprises at least one KPV, wherein said KPV or agriculturally acceptable salt thereof comprises or consists of an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof.

In some embodiments, a combination, mixture, or composition of the present disclosure comprises at least one KPV, wherein the KPV, or an agriculturally acceptable salt thereof, comprises or consists of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof.

In some embodiments, a combination, mixture, or composition of the present disclosure comprises at least one KPV, wherein the KPV consists essentially of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof.

In some embodiments, a combination, mixture, or composition of the present disclosure comprises at least one KPV, wherein the KPV consists of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof.

In some embodiments, a combination, mixture, or composition of the present disclosure comprises, consists essentially of, or consists of, a KPV, or an agriculturally acceptable salt having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCAACCPCCPGTSCKTESNGVSYCRKD” (SEQ ID NO: 2); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAGVNGVAYCRKDEP” (SEQ ID NO: 3); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEANGVSRCRKDEP” (SEQ ID NO: 4); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAFSNGVSYCRKDEP” (SEQ ID NO: 5); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEWNGVSYCRKDEP” (SEQ ID NO: 6); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAICPCCPGTSCKAESNGVSYCRKDEP” (SEQ ID NO: 7); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGIDRPCAACCPCCPGTSCKAESNGVSYCRKDEP” (SEQ ID NO: 10); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSHCRKDEP” (SEQ ID NO: 11); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSSKAESNGVCYCRKDEP” (SEQ ID NO: 12); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTEADRPCHACCPCCPGTSCKAESNGVRYCRKDEP” (SEQ ID NO: 13); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAICCPCCPGTSCKAESNGVSYCRKDEP” (SEQ ID NO: 16); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICEGADRPCAACCPCCPGTSCKAESNGVSYCRKDEP” (SEQ ID NO: 17); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESRGVSYCRKDEP” (SEQ ID NO: 18); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYGRKDEP” (SEQ ID NO: 19); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKYESNGVSYCRKDEP” (SEQ ID NO: 23); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACNPCCPGTSCKAESNGVSYCRKMEP” (SEQ ID NO: 25); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPYAACCPCCPGTSCKAESNGVSYCRKDEP” (SEQ ID NO: 30); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPKAACCPCCPGTSCKAESNGVSYCRKDEP” (SEQ ID NO: 31); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKNEP” (SEQ ID NO: 35); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTKADRPCAACCPCCPGTSCKAASNGVSYCRKDEP” (SEQ ID NO: 39); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AVCTGADRPCAACCPCCPGTSCKAESNGVSYCRKDEP” (SEQ ID NO: 40); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGSSCKAEGNGVSYCRKDEP” (SEQ ID NO: 45); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEPNGVSYCRRDEP” (SEQ ID NO: 46); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKDVP” (SEQ ID NO: 48); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEPNGVSYCRKDEP” (SEQ ID NO: 50); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSFCRKDEP” (SEQ ID NO: 54); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTDADRPCAACCPCCPGTSCKAESNGVSYCRKDEP” (SEQ ID NO: 55); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AIYTGADRPCAACCPCCPGTSCKAESNGVSNCRKDEP” (SEQ ID NO: 56); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAGCPCCPGTSCKAESNGVSYCRKDEP” (SEQ ID NO: 57); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESVGVSYCRKDEP” (SEQ ID NO: 60); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICRGADRPCAACCPCCPGTSCKAESNGVSYCRKDEP” (SEQ ID NO: 66); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCWAESNGVSYCRKDEP” (SEQ ID NO: 68); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCYPGTSCKAESNGVSYCRKDEP” (SEQ ID NO: 70); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAECPCCPGTSCKAESNGVSYCRKDEP” (SEQ ID NO: 71); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADGPCAACCPCCPGTSCKAESNGVSYCRKDES” (SEQ ID NO: 73); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADSPCAACCPCCPGTTCKAESNGVSYCRKDEP” (SEQ ID NO: 74); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAETNGVSYCRKDEP” (SEQ ID NO: 76); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRGDEP” (SEQ ID NO: 77); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCSACCPCCPGHSCKAESNGVSYCRKDEP” (SEQ ID NO: 78); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPRAACCPCCPGTSCKAESNGVSYCRKDEP” (SEQ ID NO: 79); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRADEP” (SEQ ID NO: 85); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRNDEP” (SEQ ID NO: 102); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGEDRPCAACCPCCPGTSCKAESNGVSYCRKDEP” (SEQ ID NO: 110); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCFAESNGVSYCRKDEP” (SEQ ID NO: 114); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEANGVSYCRKDEP” (SEQ ID NO: 124); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRLDEP” (SEQ ID NO: 126); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPRTSCKAESNGVSYCRKDEP” (SEQ ID NO: 140); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRMDEP” (SEQ ID NO: 146); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGISCKAESNGVSYCRKDEP” (SEQ ID NO: 147); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEKNGVSYCRKDEP” (SEQ ID NO: 149); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “WICTGADRPCAACCPCCPGTSCKAESNGVSYCRKDEP” (SEQ ID NO: 151); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRACAACCPCCPGTSCKAESNGVSYCRKDEP” (SEQ ID NO: 152); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCREDEP” (SEQ ID NO: 157); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGQSCKAESNGVSYCRKDEP” (SEQ ID NO: 158); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRVDEP” (SEQ ID NO: 160); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAHSNGVSYCRKDEP” (SEQ ID NO: 161); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKDES” (SEQ ID NO: 162); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AVCTGADRPCAACCPCCPGTSCKAESNGVWYCRKDEP” (SEQ ID NO: 164); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVYYCRKDEP” (SEQ ID NO: 167); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESLGVSYCRKDEP” (SEQ ID NO: 168); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPPTSCKAESNGVSYCRKDEP” (SEQ ID NO: 170); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCMCCPGTSCKAESNGVSYCRKDEP” (SEQ ID NO: 173); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKWESNGVSYCRKDEP” (SEQ ID NO: 176); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEGNGVSYCRKDEP” (SEQ ID NO: 178); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAWSNGVSYCRKDEP” (SEQ ID NO: 180); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGANRPCAAGCPCCPGTSCKAESNGVSYCRKDEP” (SEQ ID NO: 183); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKDEQ” (SEQ ID NO: 184); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAAFCPCCPGTSCKAESNGVSYCRKDEP” (SEQ ID NO: 185); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESWGVSYCRKDEP” (SEQ ID NO: 186); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCQKDEP” (SEQ ID NO: 189); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTTCQAESNGVSYCRKDEP” (SEQ ID NO: 191); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGIDRPCAACCPCCPGTSCKAESNGVSYCRKDKP” (SEQ ID NO: 192); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGAGRPCAACCPCCPGTSCKAESNGVSYCRKDFP” (SEQ ID NO: 195); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AACTGADRPCAACCPCCPGTSCKAESNGVSYCRKDEP” (SEQ ID NO: 199); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AIHTGADRPCAACCPCCPGTSCDAESNGVSYCRKDEP” (SEQ ID NO: 204); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCAAESNGVSYCRKDEP” (SEQ ID NO: 205); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCDAESNGVSYCRKDEP” (SEQ ID NO: 206); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCIAESNGVSYCRKDEP” (SEQ ID NO: 209); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCLAESNGVSYCRKDEP” (SEQ ID NO: 210); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCMAESNGVSYCRKDEP” (SEQ ID NO: 211); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCNAESNGVSYCRKDEP” (SEQ ID NO: 212); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCRAESNGVSYCRKDEP” (SEQ ID NO: 215); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCVAESNGVSYCRKDEP” (SEQ ID NO: 216); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCYAESNGVSYCRKDEP” (SEQ ID NO: 217); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRDDEP” (SEQ ID NO: 218); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRHDEP” (SEQ ID NO: 220); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRIDEP” (SEQ ID NO: 221); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRPDEP” (SEQ ID NO: 222); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRQDEP” (SEQ ID NO: 223); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRSDEP” (SEQ ID NO: 224); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRTDEP” (SEQ ID NO: 225); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “VICTGADRPCAACCPCCPGTSCKAESNGVSYCRKDEP” (SEQ ID NO: 226); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “LICTGADRPCAACCPCCPGTSCKAESNGVSYCRKDEP” (SEQ ID NO: 227); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAGVNGVAYCRKD” (SEQ ID NO: 228); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEANGVSRCRKD” (SEQ ID NO: 229); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEVNGVSYCRKDEP” (SEQ ID NO: 230); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAESNGVSYCRKDEP” (SEQ ID NO: 231); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “DICTGADRPCAACCPCCPGTSCKAESNGVSYCRKDEP” (SEQ ID NO: 232); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSLCRKDEP” (SEQ ID NO: 233); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADKPCAACCPCCPGTSCKAESNGVSYCRKDEP” (SEQ ID NO: 235); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADHPCAACCPCCPGTSCKAESNGVSYCRKDEP” (SEQ ID NO: 236); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADYPCAACCPCCPGTSCKAESNGVSYCRKDEP” (SEQ ID NO: 237); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADLPCAACCPCCPGTSCKAESNGVSYCRKDEP” (SEQ ID NO: 238); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADMPCAACCPCCPGTSCKAESNGVSYCRKDEP” (SEQ ID NO: 239); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADSPCAACCPCCPGTSCKAESNGVSYCRKDEP” (SEQ ID NO: 240); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCGGCCPCCPGTSCKAESNGVSYCRKDEP” (SEQ ID NO: 243); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCGACCPCCPGTSCKAESNGVSYCRKDEP” (SEQ ID NO: 244); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAGCCPCCPGTSCKAESNGVSYCRKDEP” (SEQ ID NO: 245); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTKCKAESNGVSYCRKDEP” (SEQ ID NO: 246); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTRCKAESNGVSYCRKDEP” (SEQ ID NO: 247); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADGPCAACCPCCPGTSCKAESNGVSYCRKDEP” (SEQ ID NO: 248); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKTESNGVSYCRKD” (SEQ ID NO: 249); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “PICTGADRPCAACCPCCPGTSCKAESNGVSYCRKDEP” (SEQ ID NO: 250); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “EAAICTGADRPCAACCPCCPGTSCKAESNGVSYCRKD” (SEQ ID NO: 251); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “NSAICTGADRPCAACCPCCPGTSCKAESNGVSYCRKDEP” (SEQ ID NO: 252); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSAICTGADRPCAACCPCCPGTSCKAESNGVSYCRKDEP” (SEQ ID NO: 253); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSAICTGADRPCAACCPCCPGTSCKAESNGVSYCRKD” (SEQ ID NO: 254); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “EAAVCTGADRPCAACCPCCPGTSCKAESNGVSYCRKDEP” (SEQ ID NO: 255); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSQYCTGADRPCAACCPCCPGTSCKAESNGVSYCRKA” (SEQ ID NO: 256); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCAACCPCCPGTSCKAESNGVWYCRKDEP” (SEQ ID NO: 257); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCGACCPCCPGTSCKAESNGVSYCRA” (SEQ ID NO: 258); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCGACCPCCPGTSCKAESNGVSYCRKDEP” (SEQ ID NO: 259); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTPADRPCAACCPCCPGTSCKAESNGVSYCRKDEP” (SEQ ID NO: 260); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCSAESNGVSYCRKDEP” (SEQ ID NO: 263); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCQAASNGVSYCRKDEP” (SEQ ID NO: 264); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “NAICTGADRPCAACCPCCPGTSCYAESNGVSYCRKDEP” (SEQ ID NO: 265); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVWYCRKDEP” (SEQ ID NO: 269); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKATSNGVSYCRKDEP” (SEQ ID NO: 270); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GAICTGADRPCAACCPCCPGTSCKAESNGVSYCRKDEP” (SEQ ID NO: 274); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GAICTGADRPCAACCPCCPGTSCKAESNGVSYCRKD” (SEQ ID NO: 275); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GSICTGADRPCAACCPCCPGTSCKAESNGVSYCRKDEP” (SEQ ID NO: 276); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAFSNGVSYCRKD” (SEQ ID NO: 277); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCYAESNGVSYCRKDEP” (SEQ ID NO: 278); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCYAESNGVSYCRKD” (SEQ ID NO: 279); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKAESNGVSWCRKDEP” (SEQ ID NO: 280); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAMSNGVSYCRKDEP” (SEQ ID NO: 282); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “GICTGADRPCAACCPCCPGTSCKAESNGVSYCRKDEP” (SEQ ID NO: 283); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKASSNGVSYCRKDEP” (SEQ ID NO: 284); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “LICTGADRPCAACCPCCPGTSCYAESNGVSYCRKD” (SEQ ID NO: 285); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKAEP” (SEQ ID NO: 286); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKEEP” (SEQ ID NO: 287); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKGEP” (SEQ ID NO: 288); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKHEP” (SEQ ID NO: 289); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKIEP” (SEQ ID NO: 290); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKKEP” (SEQ ID NO: 291); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKMEP” (SEQ ID NO: 292); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKQEP” (SEQ ID NO: 294); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKREP” (SEQ ID NO: 295); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKSEP” (SEQ ID NO: 296); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKVEP” (SEQ ID NO: 297); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKYEP” (SEQ ID NO: 299); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKQEANGVSLCRKDEP” (SEQ ID NO: 300); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCQIWSNGVSYCRKDEP” (SEQ ID NO: 302); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKYESLGVSYCRKDEP” (SEQ ID NO: 303); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTARQNGVPYCRKDEP” (SEQ ID NO: 304); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKSTGNGAGYCRKDEP” (SEQ ID NO: 305); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAGSAGAFYCRKDEP” (SEQ ID NO: 306); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCPDESEGVYYCRKDEP” (SEQ ID NO: 308); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKADHWGVPYCRKDEP” (SEQ ID NO: 312); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCKEEIEGPSYCRKDEP” (SEQ ID NO: 313); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAEDNGGTYCRKDEP” (SEQ ID NO: 315); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCSFESGGVSYCRKDEP” (SEQ ID NO: 316); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKSESNGSYYCRKDEP” (SEQ ID NO: 317); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGANRPCAACCPCCPGTSCKAESNGVSYCRKDEP” (SEQ ID NO: 318); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGANRPCAACCPCCPGTSCVAESNGVSYCRKDEP” (SEQ ID NO: 319); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKTESNGVSYCRKDEP” (SEQ ID NO: 320); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRA” (SEQ ID NO: 321); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRKD” (SEQ ID NO: 322); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “ALCTGADRPCAACCPCCPGTSCKAESNGVSYCRKDEP” (SEQ ID NO: 323); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCQGPESNGVSYCRKDEP” (SEQ ID NO: 325); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “QACTPADQPCAACCPCCSGASCTEQLNENGHTVYYCRA” (SEQ ID NO: 327); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCQGPESNGVSYCRA” (SEQ ID NO: 328); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCAGPESNGVSYCRKDEP” (SEQ ID NO: 329); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKGPESNGVSYCRKDEP” (SEQ ID NO: 331); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCNGPESNGVSYCRKDEP” (SEQ ID NO: 332); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “NAICTGADRPCAACCPCCPGTSCQGPESNGVSYCRKDEP” (SEQ ID NO: 334); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “TICTGADRPCAACCPCCPGTSCTEPESNGVSYCRND” (SEQ ID NO: 343); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTEPESNGVSYCRND” (SEQ ID NO: 344); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCHKADSNGLSYCRKDEP” (SEQ ID NO: 346); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCIKRESNGVSYCRKDEP” (SEQ ID NO: 347); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTKRESNGVKYCRKDEP” (SEQ ID NO: 348); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “TICSGGDRPRAACCPCCPGTSSGKARVNGVFYCRKDEP” (SEQ ID NO: 350); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCTKAESNGISYCRKDEP” (SEQ ID NO: 354); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “SICTGADRPCAACCPCCPGTSCQKDDHNGVGYCRKDEP” (SEQ ID NO: 355); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a combination, mixture, or composition of the present disclosure comprises, consists essentially of, or consists of, a KPV having pesticidal activity against one or more pest species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AICTGADRPCAACCPCCPGTSCKAESNGVSYCRVDEP” (SEQ ID NO: 358); wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

### Excipients: Generally

In some embodiments, an engineered, non-naturally occurring pesticidal KPV of the present disclosure, or an agriculturally acceptable salt thereof, can further comprise one or more excipients to make an agriculturally useful composition.

For example, in some embodiments, the excipient can be any agriculturally acceptable additive, carrier, surfactant, emulsifier, thickener, preservative, solvent, disintegrant, glidant, lubricant, diluent, filler, bulking agent, binder, emollient, stiffening agent, stabilizer, solubilizing agents, dispersing agent, suspending agent, antioxidant, antiseptic, wetting agent, humectant, fragrant, suspending agents, pigments, colorants, isotonic agents, viscosity enhancing agents, mucoadhesive agents, and/or any combination thereof.

The use of excipients for the formulation of agricultural compositions is known to those having ordinary skill in the art.

In some embodiments, the excipient can be added to an pesticidal composition, preparation, and/or formulation of the present disclosure, which may aid in achieving a desired modification to the characteristics of the pesticidal composition, preparation, and/or formulation. Such modifications include, but are not limited to, physical stability, chemical stability, therapeutic efficacy, and/or any combination thereof.

In some embodiments, excipients can be formulated alongside an engineered, non-naturally occurring pesticidal KPV, or an agriculturally acceptable salt thereof, when preparing an pesticidal composition, e.g., for the purpose of bulking up compositions (thus often referred to as bulking agents, fillers or diluents).

In yet other embodiments, an excipient can be used to provide stability, or prevent contamination. In other embodiments, an excipient can be used to confer a physical property to a composition (e.g., a composition that is a dry granular, or dry flowable powder physical form). Reference to an excipient includes both one and more than one such excipients. Suitable excipients are described in Remington's Pharmaceutical Sciences, by E. W. Martin, the disclosure of which is incorporated herein by reference in its entirety.

In some embodiments, e.g., the excipient can be independently selected from thickeners, viscosity enhancing agents, bulking agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, emulsifiers, and diagnostic agents.

In some embodiments, an pesticidal composition of the present disclosure comprising an excipient may contain an amount of excipient ranging from about 0.005 wt % to about 99 wt %.

### Formulations: Generally

Any of the pesticidal compositions of the present disclosure can be formulated according to the methods described herein, and used to inhibit and/or kill the insects, and/or control the damage caused by the insect's actions, especially their damage to plants, for example, crops.

Pesticidal compositions of the present disclosure can be formulated for use as agrochemical compositions. For example, in some embodiments, agrochemical compositions can include, but is not limited to, aerosols and/or aerosolized products (e.g., sprays, fumigants, powders, dusts, and/or gases); granules, dusts, powders, or seed dressings.

In some embodiments, the pesticidal compositions may be formulated as a powder, dust, pellet, granule, spray, emulsion, colloid, solution, or such like, and may be prepared by such conventional means as desiccation, lyophilization, homogenization, extraction, filtration, centrifugation, or sedimentation. In all such compositions, the engineered, non-naturally occurring pesticidal KPV may be present in said pesticidal composition in a concentration of from about 0.0001% to about 99.999% by weight.

The compositions may be formulated prior to administration or application in the field, in an appropriate means such as lyophilized, freeze-dried, desiccated, or in an aqueous carrier, medium or suitable diluent, such as saline and/or other buffer. In some embodiments, the formulated compositions may be in the form of a dust or granular material, or a suspension in oil (vegetable or mineral), or water or oil/water emulsions, or as a wettable powder, or in combination with any other carrier material suitable for agricultural application. Suitable carriers can be solid or liquid and are well known in the art. In some embodiments, the compositions may be mixed with one or more solid or liquid adjuvants and prepared by various means, e.g., by homogeneously mixing, blending and/or grinding the pesticidal composition with suitable adjuvants using conventional compositions techniques. Suitable compositions and application methods are described in U.S. Pat. No. 6,468,523, the disclosure of which is incorporated by reference herein in its entirety.

In some embodiments, the active ingredients of the present disclosure can be applied in the form of compositions and can be applied to the crop area or plant to be treated, simultaneously or in succession, with other non-active compounds. These compounds can be fertilizers, weed killers, cryoprotectants, surfactants, detergents, soaps, dormant oils, polymers, and/or time-release or biodegradable carriers that permit long-term dosing of a target area following a single application of the composition. One or more of these non-active compounds can be prepared, if desired, together with further agriculturally acceptable carriers, surfactants or application-promoting adjuvants customarily employed in the art of composition. Suitable carriers and adjuvants can be solid or liquid and correspond to the substances ordinarily employed in composition technology, e.g. natural or regenerated mineral substances, solvents, dispersants, wetting agents, tackifiers, binders or fertilizers.

Methods of applying an active ingredients of the present disclosure or an agrochemical composition of the present disclosure that has an engineered, non-naturally occurring pesticidal KPV, as produced by the methods described herein of the present disclosure, include leaf application, seed coating and soil application. In some embodiments, the number of applications and the rate of application may depend on the intensity of infestation by the corresponding pest.

In some embodiments, pesticidal compositions of the present disclosure may be prophylactically applied to an environmental area to prevent infection by a plant pest, for example, an insect pest, which may be killed or reduced in numbers in a given area by the methods of the present disclosure. In some embodiments, the pest comes into contact with, an pesticidal-effective amount of the pesticidal compositions of the present disclosure as described herein.

In some embodiments, the pesticidal compositions of the present disclosure may be made by formulating the engineered, non-naturally occurring pesticidal KPV with the desired agriculturally-acceptable carrier. The combinations and/or compositions may be formulated prior to administration in an appropriate means such as lyophilized, freeze-dried, desiccated, or in an aqueous carrier, medium or suitable diluent, such as saline and/or other buffer. In some embodiments, the formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. In some embodiments, the composition may be mixed with one or more solid or liquid adjuvants and prepared by various means, e.g., by homogeneously mixing, blending and/or grinding the pesticidal composition with suitable adjuvants using conventional composition techniques. Suitable composition and application methods are described in U.S. Pat. No. 6,468,523, herein incorporated by reference in its entirety.

In some embodiments an pesticidal composition of the present disclosure can be formulated at a pH ranging from about 5 to about 11; from about 5.5 to about 11; from about 6 to about 11; from about 6.5 to about 11; from about 7 to about 11; from about 7.5 to about 11; from about 8 to about 11; from about 8.5 to about 11; from about 9 to about 11; from about 9.5 to about 11; from about 10 to about 11; or from about 10.5 to about 11.

In some embodiments an pesticidal composition of the present disclosure can be formulated at a pH ranging from about 5 to about 11; from about 5 to about 10.5; from about 5 to about 10; from about 5 to about 9.5; from about 5 to about 9; from about 5 to about 8.5; from about 5 to about 8; from about 5 to about 7.5; from about 5 to about 7; from about 5 to about 6.5; from about 5 to about 6; or from about 5 to about 5.5.

In some embodiments an pesticidal composition of the present disclosure can be formulated into a granule form (granular formulation). Methods of generating a granular formulation are well known in the art, and include: crystallization, precipitation, pan-coating, fluid bed coating, agglomeration (e.g., fluid bed agglomeration), rotary atomization, extrusion, prilling, spheronization, size reduction methods, drum granulation, and/or high shear granulation, and the like.

In some embodiments, the granular formulation can be generated via agglomeration, e.g., spray-drying agglomeration; rewet agglomeration; fluid bed agglomeration; and the like.

In some embodiments, the type of agglomeration can be fluid bed agglomeration. Exemplary methods of fluid bed agglomeration are provided in U.S. Pat. No. 7,582,147; the disclosure of which is incorporated herein by reference in its entirety.

In some embodiments, the granular formulation can be generated via fluid bed agglomeration.

In some embodiments, the granular formulation can be generated by spraying the active and inert ingredients onto a blank carrier in a fluid bed.

In some embodiments, the granular formulation can be generated by spraying the active and inert ingredients (excipients) onto a blank carrier and granulated in pan granulator.

In some embodiments, the granular formulation can be generated by mixing the active and inert powders (i.e., one or more excipients described herein) and water, and subsequently granulated by passing the ingredients through an extruder.

In some embodiments, the granular formulation can be generated by mixing the active and inert powders (i.e., one or more excipients described herein) with water, and granulated by roll compaction.

Pesticidal compositions comprising an engineered, non-naturally occurring pesticidal KPV, or an agriculturally acceptable salt thereof; and an excipient, said compositions can include, but are not limited to, aerosols and/or aerosolized products, e.g., sprays, fumigants, powders, dusts, and/or gases; and/or seed dressings.

In some embodiments, an pesticidal composition of the present disclosure comprises: an engineered, non-naturally occurring pesticidal KPV, or an agriculturally acceptable salt thereof; and an excipient.

In some embodiments, an pesticidal composition of the present disclosure comprises: an engineered, non-naturally occurring pesticidal KPV, or an agriculturally acceptable salt thereof; and an excipient.

In some embodiments, an pesticidal composition of the present disclosure comprises: an engineered, non-naturally occurring pesticidal KPV, or an agriculturally acceptable salt thereof, wherein the engineered, non-naturally occurring pesticidal KPV consists of an amino acid sequence set forth in any one of SEQ ID NOs: 1-5.

In some embodiments, an pesticidal composition of the present disclosure comprises: an engineered, non-naturally occurring pesticidal KPV, or an agriculturally acceptable salt thereof; and an excipient, wherein the engineered, non-naturally occurring pesticidal KPV consists of an amino acid sequence as set forth in any one of SEQ ID NOs: 1-5.

### Sprayable Compositions

Examples of spray products of the present disclosure can include field sprayable compositions for agricultural usage and indoor sprays for use in interior spaces in a residential or commercial space. In some embodiments, residual sprays or space sprays having an engineered, non-naturally occurring pesticidal KPV and an excipient can be used to reduce or eliminate insects in an interior space.

Surface spraying indoors (SSI) is the technique of applying a variable volume sprayable volume of a composition onto indoor surfaces where vectors rest, such as on walls, windows, floors and ceilings. The primary goal of variable volume sprayable volume is to reduce the lifespan of the insect and thereby reduce or interrupt disease transmission. As its name implies, SSI involves applying the composition onto the walls and other surfaces of a house with a residual pesticidal (e.g., an engineered, non-naturally occurring pesticidal KPV).

In one embodiment, the composition having an engineered, non-naturally occurring pesticidal KPV and an excipient will inhibit growth and/or proliferation of insects that come in contact with these surfaces.

In contrast to SSI, which requires that the an engineered, non-naturally occurring pesticidal KPV or an agriculturally acceptable salt thereof, be bound to surfaces of dwellings, such as walls or ceilings, as with a paint, for example, space spray products of the disclosure rely on the production of a large number of small droplets intended to be distributed through a volume of air over a given period of time. When these droplets impact on a target insect, they deliver an effective dose of an engineered, non-naturally occurring pesticidal KPV, effective to inhibit or kill the insect. The traditional methods for generating a space-spray include thermal fogging (whereby a dense cloud of a composition having an engineered, non-naturally occurring pesticidal KPV is produced giving the appearance of a thick fog) and Ultra Low Volume (ULV), whereby droplets are produced by a cold, mechanical aerosol-generating machine. Ready-to-use aerosols such as aerosol cans may also be used.

In some embodiments, a sprayable composition may contain an amount of an engineered, non-naturally occurring pesticidal KPV, or an agriculturally acceptable salt thereof, ranging in concentration from about 0.0001 wt % to about 99.9999 wt %, based on the total weight of the sprayable composition.

In some embodiments, a sprayable composition may contain an amount of an excipient, or a plurality of excipients in the aggregate, ranging from about 0.0001 wt % to about 99.9999 wt %.

### Foams

The pesticidal compositions of the present disclosure comprising an engineered, non-naturally occurring pesticidal KPV, or an agriculturally acceptable salt thereof, and an excipient may be made available in a spray product as an aerosol-based application, including aerosolized foam applications. Pressurized cans are the typical vehicle for the formation of aerosols. An aerosol propellant that is compatible with the composition having an engineered, non-naturally occurring pesticidal KPV, or an agriculturally acceptable salt thereof, and an excipient are used. Preferably, a liquefied-gas type propellant is used.

Suitable propellants include compressed air, carbon dioxide, butane and nitrogen. The concentration of the propellant in the active compound composition is from about 5 percent to about 40 percent by weight of the pyridine composition, preferably from about 15 percent to about 30 percent by weight of the composition an engineered, non-naturally occurring pesticidal KPV, or an agriculturally acceptable salt thereof, and an excipient.

In one embodiment, pesticidal compositions of the present disclosure comprising an engineered, non-naturally occurring pesticidal KPV, or an agriculturally acceptable salt thereof, and an excipient can also include one or more foaming agents. Foaming agents that can be used include sodium laureth sulfate, cocamide DEA, and cocamidopropyl betaine. Preferably, the sodium laureth sulfate, cocamide DEA and cocamidopropyl are used in combination. The concentration of the foaming agent(s) in the active compound composition is from about 10 percent to about 25 percent by weight, more preferably 15 percent to 20 percent by weight of the composition.

When such compositions are used in an aerosol application not containing foaming agents, the active compositions of the present disclosure can be used without the need for mixing directly prior to use. However, aerosol compositions containing the foaming agents do require mixing (i.e., shaking) immediately prior to use. In addition, if the compositions containing foaming agents are used for an extended time, they may require additional mixing at periodic intervals during use.

In some embodiments, a foam may contain an amount of an engineered, non-naturally occurring pesticidal KPV, or an agriculturally acceptable salt thereof, ranging in concentration from about 0.0001 wt % to about 99.9999 wt %.

In some embodiments, a foam may contain an amount of an excipient or a plurality of excipients, in the aggregate, ranging from about 0.005 wt % to about 99 wt %.

### Fabric Treatments

In some embodiments, fabrics and garments may be made that contain an pesticidal-effective composition comprising an engineered, non-naturally occurring pesticidal KPV, or an agriculturally acceptable salt thereof, and an excipient. In some embodiments, the concentration of the engineered, non-naturally occurring pesticidal KPV, or an agriculturally acceptable salt thereof in the polymeric material, fiber, yarn, weave, net, or substrate described herein, can be varied within a relatively wide concentration range from, for example, 0.05 to 15 percent by weight, preferably 0.2 to 10 percent by weight, more preferably 0.4 to 8 percent by weight, especially 0.5 to 5, such as 1 to 3, percent by weight.

Similarly, the concentration of the pesticidal composition of the present disclosure comprising an engineered, non-naturally occurring pesticidal KPV, or an agriculturally acceptable salt thereof, and an excipient (whether for treating surfaces or for coating a fiber, yarn, net, weave) can be varied within a relatively wide concentration range from, for example 0.1 to 70 percent by weight, such as 0.5 to 50 percent by weight, preferably 1 to 40 percent by weight, more preferably 5 to 30 percent by weight, especially 10 to 20 percent by weight.

The concentration of the engineered, non-naturally occurring pesticidal KPV, or an agriculturally acceptable salt thereof may be chosen according to the field of application such that the requirements concerning inhibitory or lethal efficacy, durability and toxicity are met. Adapting the properties of the material can also be accomplished and so custom-tailored textile fabrics are obtainable in this way.

Accordingly, an effective amount of an engineered, non-naturally occurring pesticidal KPV, or an agriculturally acceptable salt thereof can depend on the specific use pattern, the insect against which control is most desired and the environment in which the an engineered, non-naturally occurring pesticidal KPV, or an agriculturally acceptable salt thereof will be used. Therefore, an effective amount of an engineered, non-naturally occurring pesticidal KPV, or an agriculturally acceptable salt thereof is sufficient that inhibition or killing or one or more targeted insects are achieved.

In some embodiments, a fabric treatment may contain an amount of an engineered, non-naturally occurring pesticidal KPV, or an agriculturally acceptable salt thereof ranging in concentration from about 0.0001 wt % to about 99.9999 wt %.

In some embodiments, a fabric treatment may contain an amount of an excipient, or a plurality of excipients, in the aggregate ranging from about 0.0001 wt % to about 99.9999 wt %.

### Surface-Treatment Compositions

In some embodiments, the present disclosure provides compositions having an engineered, non-naturally occurring pesticidal KPV, or an agriculturally acceptable salt thereof, and an excipient, for coating walls, floors and ceilings inside of buildings, and for coating a substrate or non-living material. The inventive compositions comprising an engineered, non-naturally occurring pesticidal KPV, or an agriculturally acceptable salt thereof, and an excipient, can be prepared using known techniques for the purpose in mind. Preparations of pesticidal compositions of the present disclosure comprising an engineered, non-naturally occurring pesticidal KPV, or an agriculturally acceptable salt thereof, and an excipient could be so formulated to also contain a binder to facilitate the binding of the compound to the surface or other substrate. Agents useful for binding are known in the art and tend to be polymeric in form. The type of binder suitable for a compositions to be applied to a wall surface having particular porosities and/or binding characteristics would be different compared to a fiber, yarn, weave or net-thus, a skilled person, based on known teachings, would select a suitable binder based on the desired surface and/or substrate.

Typical binders include but are not limited to poly vinyl alcohol, modified starch, poly vinyl acrylate, polyacrylic, polyvinyl acetate co polymer, polyurethane, and modified vegetable oils. Suitable binders can include latex dispersions derived from a wide variety of polymers and co-polymers and combinations thereof. Suitable latexes for use as binders in the inventive compositions comprise polymers and copolymers of styrene, alkyl styrenes, isoprene, butadiene, acrylonitrile lower alkyl acrylates, vinyl chloride, vinylidene chloride, vinyl esters of lower carboxylic acids and alpha, beta-ethylenically unsaturated carboxylic acids, including polymers containing three or more different monomer species copolymerized therein, as well as post-dispersed suspensions of silicones or polyurethanes. Also suitable may be a polytetrafluoroethylene (PTFE) polymer for binding the active ingredient to other surfaces.

In some embodiments, a surface-treatment composition may contain an amount of an engineered, non-naturally occurring pesticidal KPV, or an agriculturally acceptable salt thereof ranging in concentration from about 0.0001 wt % to about 99.9999 wt %.

In some embodiments, a surface-treatment composition may contain an amount of an excipient, or a plurality of excipients, in the aggregate, ranging from about 0.0001 wt % to about 99.9999 wt %.

### Dispersants

In some exemplary embodiments, an pesticidal composition of the present disclosure comprises an engineered, non-naturally occurring pesticidal KPV, or an agriculturally acceptable salt thereof, and an excipient, wherein the excipient can be a diluent or carrier (e.g., such as water), a polymeric binder, and/or additional components such as a dispersing agent, a polymerizing agent, an emulsifying agent, a thickener, an alcohol, a fragrance, or any other inert excipients used in the preparation of sprayable actives known in the art.

In some embodiments, a dispersant may contain an amount of an engineered, non-naturally occurring pesticidal KPV, or an agriculturally acceptable salt thereof, ranging in concentration from about 0.0001 wt % to about 99.9999 wt %.

In some embodiments, a dispersant may contain an amount of an excipient, or a plurality of excipients, in the aggregate, ranging from about 0.0001 wt % to about 99.9999 wt %.

### Suspensions

In some embodiments, an pesticidal composition comprising an engineered, non-naturally occurring pesticidal KPV, or an agriculturally acceptable salt thereof, and an excipient, can be prepared in a number of different forms or formulation types, such as suspensions or capsules suspensions. And a person skilled in the art can prepare the relevant composition based on the properties of the engineered, non-naturally occurring pesticidal KPV, or an agriculturally acceptable salt thereof, its uses, and also its application type.

For example, in some embodiments, the engineered, non-naturally occurring pesticidal KPV, or an agriculturally acceptable salt thereof, used in the methods, embodiments, and other aspects of the present disclosure, may be encapsulated in a suspension or capsule suspension formulation. An encapsulated engineered, non-naturally occurring pesticidal KPV, or an agriculturally acceptable salt thereof, of the present disclosure can provide improved wash-fastness, and also a longer period of activity. The formulation can be organic based or aqueous based, preferably aqueous based.

In some embodiments, a suspension may contain an amount of an engineered, non-naturally occurring pesticidal KPV, or an agriculturally acceptable salt thereof, ranging in concentration from about 0.0001 wt % to about 99.9999 wt %.

In some embodiments, a suspension may contain an amount of an excipient, or a plurality of excipients, in the aggregate, ranging from about 0.0001 wt % to about 99.9999 wt %.

### Microencapsulation

Microencapsulated engineered, non-naturally occurring pesticidal KPV, or an agriculturally acceptable salt thereof, suitable for use in the compositions and methods according to the present disclosure may be prepared with any suitable technique known in the art. For example, various processes for microencapsulating material have been previously developed. These processes can be divided into three categories: physical methods, phase separation, and interfacial reaction.

In the physical methods category, microcapsule wall material and core particles are physically brought together and the wall material flows around the core particle to form the microcapsule. In the phase separation category, microcapsules are formed by emulsifying or dispersing the core material in an immiscible continuous phase in which the wall material is dissolved and caused to physically separate from the continuous phase, such as by coacervation, and deposit around the core particles. In the interfacial reaction category, microcapsules are formed by emulsifying or dispersing the core material in an immiscible continuous phase and then an interfacial polymerization reaction is caused to take place at the surface of the core particles. The concentration of the engineered, non-naturally occurring pesticidal KPV, or an agriculturally acceptable salt thereof present in the microcapsules can vary from about 0.0001% to about 99.9999% by weight of the microcapsule.

In some embodiments, a microencapsulation may contain an amount of an engineered, non-naturally occurring pesticidal KPV, or an agriculturally acceptable salt thereof ranging from about 0.0001 wt % to about 99.9999 wt %.

In some embodiments, a microencapsulation may contain an amount of an excipient, or a plurality of excipients, in the aggregate, ranging from about 0.0001 wt % to about 99.9999 wt %.

Any of the compositions or formulations described herein can be made using an engineered, non-naturally occurring pesticidal KPV, or an agriculturally acceptable salt thereof described herein (e.g., an engineered, non-naturally occurring pesticidal KPV, or an agriculturally acceptable salt thereof consisting of an amino acid sequence set forth in any one of SEQ ID NOs: 1-5), and any of the excipients described herein.

Any of the compositions or formulations described herein can additionally contain other known biologically active agents, such as, for example, a fungicide, herbicide, or insecticide.

### Kits, Compositions, Dispersants, and the Ingredients Thereof

The combinations used in the pesticidal compositions of the present disclosure comprising an engineered, non-naturally occurring pesticidal KPV, or an agriculturally acceptable salt thereof and an excipient, methods, embodiments and other aspects according to the present disclosure, may be formed by mixing all ingredients together, optionally with water, and optionally using suitable mixing and/or dispersing aggregates. In general, such a combination is formed at a temperature of from 10° C. to 70° C., preferably 15° C. to 50° C., more preferably 20° C. to 40° C.

Generally, an pesticidal composition of the present disclosure comprising one or more of (A1 and A2), (B), (C), and/or (D) is possible, wherein it is possible to use: an engineered, non-naturally occurring pesticidal KPV, or an agriculturally acceptable salt thereof (A1) and an excipient (A2); solid polymer (B); optional additional additives (D); and to disperse them in the aqueous component (C). If a binder is present in an pesticidal composition of the present disclosure, it is preferred to use dispersions of the polymeric binder (B) in water as well as aqueous compositions of the an engineered, non-naturally occurring pesticidal KPV, or an agriculturally acceptable salt thereof (A1) and excipient (A2) in water which have been separately prepared before. Such separate compositions may contain additional additives for stabilizing (A1 and A2) and/or (B) in the respective pesticidal compositions and are commercially available. In a second process step, such raw compositions and optionally additional water (component (C)) are added. Also, combinations of the abovementioned ingredients based on the foregoing scheme are likewise possible, e.g., using a pre-formed dispersion of (A1 and A2) and/or (B) and mixing it with solid (A1 and A2) and/or (B). A dispersion of the polymeric binder (B) may be a pre-manufactured dispersion already made by a chemicals manufacturer.

Moreover, it is also within the scope of the present disclosure to use “hand-made” dispersions, i.e., dispersions made in small-scale by an end-user. Such dispersions may be made by providing a mixture of about 20 percent of the binder (B) in water, heating the mixture to temperature of 90° C. to 100° C. and intensively stirring the mixture for several hours. It is possible to manufacture the pesticidal composition as a final product so that it can be readily used by the end-user for the process according to the present disclosure. It is of course similarly possible to manufacture a concentrate, which may be diluted by the end-user with additional water (C) to the desired concentration for use.

In an embodiment, a composition (having an engineered, non-naturally occurring pesticidal KPV, or an agriculturally acceptable salt thereof and an excipient) suitable for SSI application or a coating formulation (having an engineered, non-naturally occurring pesticidal KPV, or an agriculturally acceptable salt thereof, and an excipient), contains the active ingredient and a carrier, such as water, and may also one or more co-formulants selected from a dispersant, a wetter, an anti-freeze, a thickener, a preservative, an emulsifier and a binder or sticker.

In some embodiments, an exemplary solid combination of an engineered, non-naturally occurring pesticidal KPV, or an agriculturally acceptable salt thereof, is generally milled to a desired particle size, such as the particle size distribution d(0.5) is generally from 3 to 20, preferably 5 to 15, especially 7 to 12, μm.

Furthermore, it may be possible to ship an pesticidal composition to the end-user as a kit, e.g., comprising at least a first component of an engineered, non-naturally occurring pesticidal KPV, or an agriculturally acceptable salt thereof (A1) and an excipient (A2) (either separately or as a mixture); and a second component comprising at least one polymeric binder (B). Further additives (D) may be a third separate component of the kit, or may be already mixed with components (A1 and A2) and/or (B). The end-user may prepare the pesticidal composition for use by mixing (A1) and (A2) and mixing. Alternatively, the end-user may prepare the pesticidal composition for use by just adding water (C) to the components of the kit and mixing. The components of the kit may also be compositions in water. Of course it is possible to combine an aqueous composition of one of the components with a dry composition of the other component(s). As an example, the kit can consist of one composition of an engineered, non-naturally occurring pesticidal KPV, or an agriculturally acceptable salt thereof (A1) and an excipient (A2) and optionally water (C); and a second, separate composition of at least one polymeric binder (B), water as component (C) and optionally one or more components (D).

The concentrations of the components (A1 and A2), (B), (C) and optionally (D) will be selected by the skilled artisan depending of the technique to be used for coating/treating. In some embodiments, the amount of an engineered, non-naturally occurring pesticidal KPV, or an agriculturally acceptable salt thereof can be up to 50, preferably 1 to 50, such as 10 to 40, especially 15 to 30, percent by weight, based on weight of the composition. The amount of polymeric binder (B) may be in the range of 0.01 to 30, preferably 0.5 to 15, more preferably 1 to 10, especially 1 to 5, percent by weight, based on weight of the composition. If present, in general the amount of additional components (D) is from 0.1 to 20, preferably 0.5 to 15, percent by weight, based on weight of the composition. If present, suitable amounts of pigments and/or dyestuffs and/or fragrances are in general 0.01 to 5, preferably 0.1 to 3, more preferably 0.2 to 2, percent by weight, based on weight of the composition. In some embodiments, a composition ready for use comprises 0.1 to 40, preferably 1 to 30, percent of components (A1 and A2), (B), and optionally (D), the residual amount being water (C). In some embodiments, a concentration of a concentrate to be diluted by the end-user may comprise 5 to 70, preferably 10 to 60, percent of components (A1 and A2), (B), and optionally (D), the residual amount being water (C).

Ranges and Descriptions of Composition Components % v/v; % w/w; and % w/v

As used herein, “v/v” or “% v/v” or “volume per volume” refers to the volume concentration of a solution (“v/v” stands for volume per volume). Here, v/v can be used when both components of a solution are liquids. For example, when 50 mL of ingredient X is diluted with 50 mL of water, there will be 50 mL of ingredient X in a total volume of 100 mL; therefore, this can be expressed as “ingredient X 50% v/v.” Percent volume per volume (% v/v) is calculated as follows: (volume of solute (mL)/volume of solution (100 mL)); e.g., % v/v=mL of solute/100 mL of solution.

As used herein, “w/w” or “% w/w” or “weight per weight” or “wt/wt” or “% wt/wt” refers to the weight concentration of a composition or solution, i.e., percent weight in weight (“w/w” stands for weight per weight). Here, w/w expresses the number of grams (g) of a constituent in 100 g of solution or mixture. For example, a mixture consisting of 30 g of ingredient X, and 70 g of water would be expressed as “ingredient X 30% w/w.” Percent weight per weight (% w/w) is calculated as follows: (weight of solute (g)/weight of solution (g))×100; or (mass of solute (g)/mass of solution (g))×100.

As used herein, “w/v” or “% w/v” or “weight per volume” refers to the mass concentration of a solution, i.e., percent weight in volume (“w/v” stands for weight per volume). Here, w/v expresses the number of grams (g) of a constituent in 100 mL of solution. For example, if 1 g of ingredient X is used to make up a total volume of 100 mL, then a “1% w/v solution of ingredient X” has been made. Percent weight per volume (% w/v) is calculated as follows: (Mass of solute (g)/Volume of solution (mL))×100.

In some embodiments, a pesticidal composition of the present disclosure can comprise: a KPV, a KPV-pesticidal protein, or an agriculturally acceptable salt thereof, and an excipient; wherein the KPV, KPV-pesticidal protein, or agriculturally acceptable salt thereof is in an amount ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; from about 99.99 to about 0.01%; or from about 99.99999% to about 0.000001% w/w of the total composition.

In some embodiments, a pesticidal composition of the present disclosure comprises: a KPV, a KPV-pesticidal protein, or an agriculturally acceptable salt thereof; and an excipient, wherein the concentration of the KPV, KPV-pesticidal protein, or agriculturally acceptable salt thereof ranges from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; from about 99.99 to about 0.01%; or from about 99.99999% to about 0.000001% wt/wt of the total composition.

In some embodiments, a pesticidal composition of the present disclosure comprises: a KPV, a KPV-pesticidal protein, or an agriculturally acceptable salt thereof; and an excipient, wherein the concentration of the KPV, KPV-pesticidal protein, or agriculturally acceptable salt thereof ranges from about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% by weight of the total composition.

In some embodiments, a pesticidal composition of the present disclosure can comprise: a KPV, a KPV-pesticidal protein, or an agriculturally acceptable salt thereof, and an excipient; wherein the excipient is in an amount ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; from about 99.99 to about 0.01%; or from about 99.99999% to about 0.000001% w/w of the total composition.

In some embodiments, a pesticidal composition of the present disclosure comprises: a KPV, a KPV-pesticidal protein, or an agriculturally acceptable salt thereof; and an excipient, wherein the concentration of the excipient ranges from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; from about 99.99 to about 0.01%; or from about 99.99999% to about 0.000001% wt/wt of the total combination or composition.

In some embodiments, a pesticidal composition of the present disclosure comprises: a KPV, a KPV-pesticidal protein, or an agriculturally acceptable salt thereof; and an excipient, wherein the concentration of the excipient ranges from about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% by weight of the total composition.

### Agriculturally Acceptable Salts

As used herein, the term agriculturally acceptable salt, hydrates, solvates, crystal forms and individual isomers, enantiomers, tautomers, diastereomers and prodrugs of the engineered, non-naturally occurring pesticidal KPV described herein can be utilized.

In some embodiments, an agriculturally acceptable salt of the present disclosure possesses the desired pharmacological activity of the parent compound. Such salts include: acid addition salts, formed with inorganic acids; acid addition salts formed with organic acids; or salts formed when an acidic proton present in the parent compound is replaced by a metal ion, e.g., an alkali metal ion, aluminum ion; or coordinates with an organic base such as ethanolamine, and the like.

In some embodiments, agriculturally acceptable salts include conventional toxic or non-toxic salts. For example, in some embodiments, convention non-toxic salts include those such as fumarate, phosphate, citrate, chlorydrate, and the like. In some embodiments, the agriculturally acceptable salts of the present disclosure can be synthesized from a parent compound by conventional chemical methods. In some embodiments, such salts can be prepared by reacting the free acid or base forms of these compounds with a stoichiometric amount of the appropriate base or acid in water or in an organic solvent, or in a mixture of the two. In some embodiments, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is incorporated herein by reference in its entirety.

In some embodiments, an agriculturally acceptable salt can be one of the following: hydrochloride; sodium; sulfate; acetate; phosphate or diphosphate; chloride; potassium; maleate; calcium; citrate; mesylate; nitrate; tartrate; aluminum; or gluconate.

In some embodiments, a list of agriculturally acceptable acids that can be used to form salts can be: glycolic acid; hippuric acid; hydrobromic acid; hydrochloric acid; isobutyric acid; lactic acid (DL); lactobionic acid; lauric acid; maleic acid; malic acid (− L); malonic acid; mandelic acid (DL); methanesulfonic acid; naphthalene-1,5-disulfonic acid; naphthalene-2-sulfonic acid; nicotinic acid; nitric acid; oleic acid; oxalic acid; palmitic acid; pamoic acid; phosphoric acid; proprionic acid; pyroglutamic acid (− L); salicylic acid; sebacic acid; stearic acid; succinic acid; sulfuric acid; tartaric acid (+ L); thiocyanic acid; toluenesulfonic acid (p); undecylenic acid; a 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; or glycerophosphoric acid.

In some embodiments, agriculturally acceptable salt can be any organic or inorganic addition salt.

In some embodiments, the agriculturally acceptable salts may use an inorganic acid and an organic acid as a free acid. The inorganic acid may be hydrochloric acid, bromic acid, nitric acid, sulfuric acid, perchloric acid, phosphoric acid, etc. The organic acid may be citric acid, acetic acid, lactic acid, maleic acid, fumaric acid, gluconic acid, methane sulfonic acid, gluconic acid, succinic acid, tartaric acid, galacturonic acid, embonic acid, glutamic acid, aspartic acid, oxalic acid, (D) or (L) malic acid, maleic acid, methane sulfonic acid, ethane sulfonic acid, 4-toluene sulfonic acid, salicylic acid, citric acid, benzoic acid, malonic acid, etc.

In some embodiments, the agriculturally acceptable salts include alkali metal salts (sodium salts, potassium salts, etc.) and alkaline earth metal salts (calcium salts, magnesium salts, etc.). For example, the acid addition salt may include acetate, aspartate, benzoate, besylate, bicarbonate/carbonate, bisulfate/sulfate, borate, camsylate, citrate, edisilate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthalate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salt, etc., and among them, hydrochloride or trifluoroacetate may be used.

In yet other embodiments, the agriculturally acceptable salt can be a salt with an acid such as acetic acid, propionic acid, butyric acid, formic acid, trifluoroacetic acid, maleic acid, tartaric acid, citric acid, stearic acid, succinic acid, ethylsuccinic acid, lactobionic acid, gluconic acid, glucoheptonic acid, benzoic acid, methanesulfonic acid, ethanesulfonic acid, 2-hydroxyethanesulfonic acid, benzenesulfonic acid, p-toluenesulfonic acid, laurylsulfuric acid, malic acid, aspartic acid, glutaminic acid, adipic acid, cysteine, N-acetylcysteine, hydrochloric acid, hydrobromic acid, phosphoric acid, sulfuric acid, hydroiodic acid, nicotinic acid, oxalic acid, picric acid, thiocyanic acid, undecanoic acid, polyacrylate or carboxyvinyl polymer.

In some embodiments, the agriculturally acceptable salt can be prepared from either inorganic or organic bases. Salts derived from inorganic bases include, but are not limited to, the sodium, potassium, lithium, ammonium, calcium, magnesium, ferrous, zinc, copper, manganous, aluminum, ferric, manganic salts, and the like. Preferred inorganic salts are the ammonium, sodium, potassium, calcium, and magnesium salts. Salts derived from organic bases include, but are not limited to, salts of primary, secondary, and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. Preferred organic bases are isopropylamine, diethylamine, ethanolamine, piperidine, tromethamine, and choline.

In some embodiments, agriculturally acceptable salt refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66:1-19 (1977), the disclosure of which is incorporated herein by reference in its entirety.

In some embodiments, the salts of the present disclosure can be prepared in situ during the final isolation and purification of the compounds of the disclosure, or separately by reacting the free base function with a suitable organic acid. Examples of agriculturally acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other agriculturally acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further agriculturally acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Exemplary descriptions of pharmaceutically acceptable salts is provided in P. H. Stahl and C. G. Wermuth, (editors), Handbook of Pharmaceutical Salts: Properties, Selection and Use, John Wiley & Sons, August 23, (2002), the disclosure of which is incorporated herein by reference in its entirety.

Methods of Using the Pesticidal Kappa Peptide Variants (KPV)

Any of the methods described herein, e.g., methods of protecting plants, plant parts, and seeds; or methods of using mixtures and compositions; can be implemented using any one or more of the KPVs or KPV-pesticidal proteins as described herein. For example, any of the methods of using the present disclosure as described herein can be implemented using, e.g., one or more KPV having the amino acid sequence selected from any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, which are likewise described herein.

In some embodiments, the present disclosure provides a method for controlling an invertebrate pest in agronomic and/or nonagronomic applications, comprising contacting the invertebrate pest or its environment, a solid surface, including a plant surface, or part thereof, with a pesticidally effective amount of one or more of the KPVs of the invention, one or more KPV-pesticidal proteins, or an agriculturally acceptable salt thereof.

In some embodiments, the present disclosure provides a method for controlling an invertebrate pest in agronomic and/or nonagronomic applications, comprising contacting the invertebrate pest or its environment, a solid surface, including a plant surface or part thereof, with a pesticidally effective amount of a composition comprising at least one KPV of the invention and an excipient.

In some embodiments, the present disclosure provides a method for controlling an invertebrate pest in agronomic and/or nonagronomic applications, comprising contacting the invertebrate pest or its environment, a solid surface, including a plant surface or part thereof, with a pesticidally effective amount of a composition comprising at least one KPV-pesticidal protein of the invention and an excipient.

Examples of suitable compositions comprising: (1) at least one KPV of the invention; two or more of the KPVs of the present disclosure; a KPV-pesticidal protein; two or more KPV-pesticidal proteins; or an agriculturally acceptable salt thereof; and (2) an excipient; include said compositions formulated with inactive ingredients to be delivered in the form of: a liquid solution, an emulsion, a powder, a granule, a nanoparticle, a microparticle, or a combination thereof.

In some embodiments, to achieve contact with a compound, mixture, or composition of the invention to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a KPV, a KPV-pesticidal protein, or an agriculturally acceptable salt thereof, and an excipient, can be applied to the plant foliage or the soil. Compounds of this invention can also be effectively delivered through plant uptake by contacting the plant with a composition comprising a compound of this invention applied as a soil drench of a liquid formulation, a granular formulation to the soil, a nursery box treatment or a dip of transplants. Of note is a composition of the present disclosure in the form of a soil drench liquid formulation. Also of note is a method for controlling an invertebrate pest comprising contacting the invertebrate pest or its environment with a biologically effective amount of a KPV or KPV-pesticidal protein. Of further note, in some illustrative embodiments, the illustrative method contemplates a soil environment, wherein the composition is applied to the soil as a soil drench formulation. Of further note is that a KPV, a KPV-pesticidal protein, or an agriculturally acceptable salt thereof, is also effective by localized application to the locus of infestation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. The compounds of this invention can also be impregnated into materials for fabricating invertebrate control devices (e.g., insect netting, application onto clothing, application into candle formulations and the like).

In some embodiments, a KPV, a KPV-pesticidal protein, or an agriculturally acceptable salt thereof, is also useful in seed treatments for protecting seeds from invertebrate pests. In the context of the present disclosure and claims, treating a seed means contacting the seed with a biologically effective amount of a KPV, a KPV-pesticidal protein, or an agriculturally acceptable salt thereof, which is typically formulated as a composition of the invention. This seed treatment protects the seed from invertebrate soil pests and generally can also protect roots and other plant parts in contact with the soil of the seedling developing from the germinating seed. The seed treatment may also provide protection of foliage by translocation of the KPV or KPV-pesticidal protein within the developing plant. Seed treatments can be applied to all types of seeds, including those from which plants genetically transformed to express specialized traits will germinate. In addition, a KPV or a KPV-pesticidal protein can be transformed into a plant or part thereof, for example a plant cell, or plant seed, that is already transformed, e.g., those expressing herbicide resistance such as glyphosate acetyltransferase, which provides resistance to glyphosate.

One method of seed treatment is by spraying or dusting the seed with a KPV, a KPV-pesticidal protein, or an agriculturally acceptable salt thereof, (i.e. as a formulated composition or a mixture comprising a KPV, a KPV-pesticidal protein, or an agriculturally acceptable salt thereof and an excipient) before sowing the seeds. Compositions formulated for seed treatment generally consist of a KPV, a KPV-pesticidal protein, or an agriculturally acceptable salt thereof, and a film former or adhesive agent. Therefore, typically, a seed coating composition of the present disclosure consists of a biologically effective amount of a KPV, a KPV-pesticidal protein, or an agriculturally acceptable salt thereof, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Monograph No. 57, and references listed therein, the disclosures of which are incorporated herein by reference in their entireties.

The treated seed typically comprises a KPV, a KPV-pesticidal protein, or an agriculturally acceptable salt thereof, in an amount ranging from about 0.01 g to 1 kg per 100 kg of seed (i.e. from about 0.00001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

In some embodiments, the invention provides a method for controlling insects comprising, providing to said insect a transgenic plant that comprises in its genome a stably incorporated expression cassette, wherein said stably incorporated expression cassette comprises a polynucleotide operable to encode a KPV.

In some embodiments, the present disclosure provides a method for controlling insects and/or for protecting against a pest, wherein the pest is selected from the group consisting of: group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola); Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex (Various Leps.); Saddleback Caterpillar (Sibine stimulea); Saddle Prominent Caterpillar Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca, Chilo suppressalis; Culex pipiens; Culex quinquefasciatus; Diabrotica virgifera, Diatraea saccharalis; Helicoverpa armigera; Helicoverpa zea, Heliothis virescens; Leptinotarsa decemlineata; Ostrinia furnacalis; Ostrinia nubilalis; Pectinophora gossypiella, Plodia interpunctella, Plutella xylostella, Pseudoplusia includens; Spodoptera exigua; Spodoptera frugiperda; Spodoptera littoralis; Trichoplusia ni; and Xanthogaleruca luteola.

### Methods of Using Mixtures

In some embodiments, the invention provides a method of combating, controlling, or inhibiting a pest comprising, applying a pesticidally effective amount of the combination, mixture, or composition comprising, consisting essentially of, or consisting of a KPV, a KPV-pesticidal protein, and/or combinations thereof, to (i) the pest, a locus of the pest, a food supply of the pest, a habitat of the pest, or a breeding ground of the pest; (ii) a plant, a seed, a plant part, a locus of a plant, or an environment of a plant that is susceptible to an attack by the pest; (iii) an animal, a locus of an animal, or an environment of an animal susceptible to an attack by the pest; or (iv) a combination of any one of (i)-(iii).

Controlling pests of the Phyla Nematoda, Arthropoda, and/or Mollusca generally means that pest populations, pest activity, or both, are reduced in an locus. This can come about when: (a) pest populations are repulsed from a locus; (b) pests are incapacitated in, or around, a locus; or (c) pests are exterminated in, or around, a locus. Of course, a combination of these results can occur. Generally, pest populations, activity, or both are desirably reduced more than fifty percent, preferably more than 90 percent, and most preferably more than 98 percent. Generally, the locus is not in, or on, a human; consequently, the locus is generally a non-human locus. In another embodiment, the locus to which a KPV, or an agriculturally acceptable salt thereof, or a composition or formulation comprising any one or more KPVs or their agriculturally acceptable salt thereof, is applied can be any locus that is inhabited, or that may become inhabited, or that may be traversed, by a pest of Phyla Nematoda, Arthropoda, and/or Mollusca. For example, the locus can be: (a) where crops, trees, fruits, cereals, fodder species, vines, turf, and/or ornamental plants, are growing; (b) where domesticated animals are residing; (c) the interior or exterior surfaces of buildings (such as places where grains are stored); (d) the materials of construction used in buildings (such as impregnated wood); and (e) the soil around buildings.

In some embodiments, the present disclosure provides a method of using an pesticidal-effective amount of: (1) an engineered, non-naturally occurring pesticidal KPV, or an agriculturally acceptable salt thereof, and/or (2) an pesticidal composition comprising: an engineered, non-naturally occurring pesticidal KPV, or an agriculturally acceptable salt thereof and an excipient; to combat, control, inhibit, or kill an insect pest, a non-insect arthropod pest or a nematode pest (e.g., insects, nematodes, and a non-insect arthropod pests, which includes arachnids, for example in addition to insects, would include various species known to attack plants, for example, aphids, spider mites, whiteflies, mealybugs, corn earworm, and tomato hornworm).

In some embodiments, the present disclosure provides a method of combating, controlling, or inhibiting a pest (e.g. an insect, and an arachnid etc. that damage or destroy plants, plant parts, and seeds) comprising: applying an pesticidal-effective amount of an engineered, non-naturally occurring pesticidal KPV, or an agriculturally acceptable salt thereof; to the locus of the pest, or to a plant or animal susceptible to an attack by the pest.

In some embodiments, the present disclosure provides a method of combating, controlling, or inhibiting a pest comprising: applying an pesticidal-effective amount of an pesticidal composition comprising: an engineered, non-naturally occurring pesticidal KPV, or an agriculturally acceptable salt thereof; and an excipient; the locus of the pest, or to a plant or animal susceptible to an attack by the pest.

In some embodiments, the present disclosure provides a method of combating, controlling, or inhibiting a plant pest comprising: applying a pesticidal-effective amount of an engineered, non-naturally occurring pesticidal KPV, or an agriculturally acceptable salt thereof to the locus of the pest, or to a plant or animal susceptible to an attack by the plant pest.

In some embodiments, the present disclosure provides a method of combating, controlling, or inhibiting a plant pest comprising: applying an pesticidal-effective amount of an engineered, non-naturally occurring pesticidal KPV, or an agriculturally acceptable salt thereof and an excipient; the locus of the pest, or to a plant or animal susceptible to an attack by the plant pest.

In some embodiments, the present disclosure provides a method of using a mixture comprising: (1) two or more KPVs, wherein the amino acid sequence of each KPV is the same or different; (2) at least one KPV, and at least one KPV-pesticidal protein, wherein the amino acid sequence of each KPV is the same or different; and/or (3) two or more KPV-pesticidal protein, wherein the amino acid sequence of each KPV is the same or different; to control insects, wherein the KPV is selected from one or any combination of the KPVs described herein, e.g., a KPV having pesticidal activity against one or more insect species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein said method comprises, preparing the mixture and then applying said mixture to (i) the insect, a locus of the insect, a food supply of the insect, a habitat of the insect, or a breeding ground of the insect; (ii) a plant, a seed, a plant part, a locus of a plant, or an environment of a plant that is susceptible to an attack by the insect; (iii) an animal, a locus of an animal, or an environment of an animal susceptible to an attack by the insect; or (iv) a combination of any one of (i)-(iii).

In some embodiments, the present disclosure provides a method of using a mixture comprising: (1) two or more KPVs, wherein the amino acid sequence of each KPV is the same or different; (2) at least one KPV, and at least one KPV-pesticidal protein, wherein the amino acid sequence of each KPV is the same or different; and/or (3) two or more KPV-pesticidal protein, wherein the amino acid sequence of each KPV is the same or different; to control insects, wherein the KPV is selected from one or any combination of the KPVs described herein, e.g., a KPV having pesticidal activity against one or more insect species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; wherein the KPV comprises at least one amino acid substitution relative to the amino acid sequence set forth in SEQ ID NO: 1, or an agriculturally acceptable salt thereof, with the proviso that the KPV does not comprise the amino acid sequence of SEQ ID NOs: 1 or 253; and wherein said method comprises, preparing the mixture and then applying said mixture to the locus of an insect.

In some embodiments, the present disclosure provides a method of using a mixture to control insects, said mixture comprising: (1) two or more KPVs, wherein the amino acid sequence of each KPV is the same or different; (2) at least one KPV, and at least one KPV-pesticidal protein, wherein the amino acid sequence of each KPV is the same or different; and/or (3) two or more KPV-pesticidal protein, wherein the amino acid sequence of each KPV is the same or different;; wherein the insects are selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola; Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana) (ground only); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex; Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata; Ostrinia furnacalis; Ostrinia nubilalis; Pectinophora gossypiella, Plodia interpunctella, Plutella xylostella, Pseudoplusia includens; Spodoptera exigua; Spodoptera frugiperda; Spodoptera littoralis; Trichoplusia ni; and/or Xanthogaleruca luteola.

### Methods of Using Compositions

In some embodiments, the present disclosure provides a method of using a composition comprising: (1) a KPV, a KPV-pesticidal protein, or an agriculturally acceptable salt thereof; and (2) an excipient; to control insects, wherein the KPV is selected from one or any combination of the KPVs described herein, e.g., a KPV having pesticidal activity against one or more insect species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein said method comprises, preparing the composition and then applying said composition to (i) the insect, a locus of the insect, a food supply of the insect, a habitat of the insect, or a breeding ground of the insect; (ii) a plant, a seed, a plant part, a locus of a plant, or an environment of a plant that is susceptible to an attack by the insect; (iii) an animal, a locus of an animal, or an environment of an animal susceptible to an attack by the insect; or (iv) a combination of any one of (i)-(iii).

In some embodiments, the present disclosure provides a method of using a composition comprising: (1) a KPV, a KPV-pesticidal protein, or an agriculturally acceptable salt thereof; and (2) an excipient; to control insects, wherein the KPV is selected from one or any combination of the KPVs described herein, e.g., a KPV having pesticidal activity against one or more insect species, said KPV comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; wherein the KPV comprises at least one amino acid substitution relative to the amino acid sequence set forth in SEQ ID NO: 1, or an agriculturally acceptable salt thereof, with the proviso that the KPV does not comprise the amino acid sequence of SEQ ID NOs: 1 or 253; and wherein said method comprises, preparing the composition and then applying said composition to the locus of an insect.

In some embodiments, the present disclosure provides a method of using a composition to control insects, said mixture comprising: (1) a KPV, a KPV-pesticidal protein, or an agriculturally acceptable salt thereof, and (2) an excipient; wherein the insects are selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola; Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana) (ground only); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex; Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca, Chilo suppressalis; Culex pipiens; Culex quinquefasciatus; Diabrotica virgifera; Diatraea saccharalis; Helicoverpa armigera, Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua, Spodoptera frugiperda, Spodoptera littoralis; Trichoplusia ni; and/or Xanthogaleruca luteola.

In some embodiments, the present disclosure provides a method of protecting a plant from insects comprising, providing a plant which expresses one or more KPVs, one or more KPV-pesticidal proteins, or polynucleotides encoding the same.

### Methods of Protecting Plants

In some embodiments, a method of protecting a plant from insects comprises providing a combination of: (1) a first KPV, or polynucleotide encoding the same; and (2) one or more additional KPVs, or polynucleotides encoding the same; wherein the first KPV and one or more additional KPVs are the same or different.

In some embodiments, a method of protecting a plant from insects comprises providing a pesticidally effective combination of: (1) a first KPV, or polynucleotide encoding the same; and (2) one or more additional KPVs, or polynucleotides encoding the same; wherein the first KPV and one or more additional KPVs are the same or different; and wherein (1) is expressed in a transgenic plant, plant tissue, plant cell, plant seed, or part thereof; and (2) is applied to the transgenic plant, plant tissue, plant cell, plant seed, or part thereof, e.g., as a composition.

In some embodiments, a method of protecting a plant from insects comprises providing a pesticidally effective combination of: (1) a first KPV, or polynucleotide encoding the same; and (2) one or more additional KPVs, or polynucleotides encoding the same; wherein the first KPV and one or more additional KPVs are the same or different; and wherein (2) is expressed in a transgenic plant, plant tissue, plant cell, plant seed, or part thereof; and (1) is applied to the transgenic plant, plant tissue, plant cell, plant seed, or part thereof, e.g., as a composition.

In some embodiments, the present disclosure provides a method of protecting a plant from insects comprising, providing a plant that expresses a KPV, or polynucleotide encoding the same, wherein said KPV comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary polynucleotide sequence thereof, with the proviso that the KPV does not comprise the amino acid sequence of SEQ ID NOs: 1 or 253.

In some embodiments, the present disclosure provides a method of protecting a plant from insects comprising, providing a plant that expresses a KPV, or polynucleotide encoding the same, wherein the KPV has an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or a complementary polynucleotide sequence thereof, with the proviso that the KPV does not comprise the amino acid sequence of SEQ ID NOs: 1 or 253.

In some embodiments, the present disclosure provides a method of protecting a plant from insects comprising, providing a plant that expresses a KPV, or polynucleotide encoding the same, wherein the KPV further comprises a homopolymer or heteropolymer of two or more KPVs, wherein the amino acid sequence of each KPV is the same or different.

In some embodiments, the present disclosure provides a method of protecting a plant from insects comprising, providing a plant that expresses a KPV, or polynucleotide encoding the same, wherein the KPV is a fused protein comprising two or more KPVs separated by a cleavable or non-cleavable linker, and wherein the amino acid sequence of each KPV may be the same or different.

In some embodiments, the present disclosure provides a method of protecting a plant from insects comprising, providing a plant that expresses a KPV, or polynucleotide encoding the same, wherein the KPV is a fused protein comprising two or more KPVs separated by a cleavable linker. In some embodiments, the linker has an amino acid sequence as set forth in any one of SEQ ID NOs: 360-369.

In some embodiments, the present disclosure provides a method of protecting a plant from insects comprising, providing a plant that expresses a KPV, or polynucleotide encoding the same, wherein the KPV is a fused protein comprising two or more KPVs separated by a linker, wherein the linker is cleavable inside at least one of (i) the gut or hemolymph of an insect, and (ii) cleavable inside the gut of a mammal.

In some embodiments, the present disclosure provides a method for controlling insects comprising, providing to said insect a transgenic plant that comprises in its genome a stably incorporated expression cassette, wherein said stably incorporated expression cassette comprises polynucleotide operable to encode a KPV.

In some embodiments, the present disclosure provides a method of combating, controlling, or inhibiting a pest comprising, applying a pesticidally effective amount of a mixture comprising: (1) a KPV, a KPV-pesticidal protein, or an agriculturally acceptable salt thereof; and (2) an excipient; wherein the KPV is selected from one or any combination of the KPVs described herein, e.g., a KPV having an amino acid sequence set forth in in SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; wherein the mixture is applied to (i) the pest, a locus of the pest, a food supply of the pest, a habitat of the pest, or a breeding ground of the pest; (ii) a plant, a seed, a plant part, a locus of a plant, or an environment of a plant that is susceptible to an attack by the pest; (iii) an animal, a locus of an animal, or an environment of an animal susceptible to an attack by the pest; or (iv) a combination of any one of (i)-(iii).

In some embodiments, the present disclosure provides a method of combating, controlling, or inhibiting a pest comprising, applying a pesticidally effective amount of a mixture comprising: (1) a KPV, a KPV-pesticidal protein, or an agriculturally acceptable salt thereof; and (2) an excipient; to (i) the pest, a locus of the pest, a food supply of the pest, a habitat of the pest, or a breeding ground of the pest; (ii) a plant, a seed, a plant part, a locus of a plant, or an environment of a plant that is susceptible to an attack by the pest; (iii) an animal, a locus of an animal, or an environment of an animal susceptible to an attack by the pest; or (iv) a combination of any one of (i)-(iii), wherein the pest is selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola; Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana) (ground only); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex; Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera, Diatraea saccharalis; Helicoverpa armigera; Helicoverpa zea, Heliothis virescens; Leptinotarsa decemlineata, Ostrinia furnacalis; Ostrinia nubilalis; Pectinophora gossypiella, Plodia interpunctella, Plutella xylostella, Pseudoplusia includens; Spodoptera exigua, Spodoptera frugiperda, Spodoptera littoralis; Trichoplusia ni; and/or Xanthogaleruca luteola.

Any of the methods described herein can be practiced using an engineered, non-naturally occurring pesticidal KPV, or an agriculturally acceptable salt thereof described herein (e.g., an engineered, non-naturally occurring pesticidal KPV, or an agriculturally acceptable salt thereof consisting of an amino acid sequence set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358), and any of the excipients described herein.

### Crops and Pests

“Pest” refers to any pest that contributes or causes, or is associated with damage, consumption, or retardation of the growth of plants or plant parts, including seeds and the like, or pests that attack, or feed on animals, such as humans. In various embodiments, pests as used herein, may be herbivores, or lay their eggs on plants or plant parts that give rise to offspring that may damage or consume plants or plant parts, or pests that feed on animal tissue, for example, animal blood or animal food, for example, flies and mosquitoes. In general, pests may also include nematodes and arthropods, which may conveniently be sub-divided into insect arthropods or “insects” and non-insect arthropods.

Pests are generally defined as organisms such as ants, aphids, beetles, bristletails, cockroaches, crickets, earwigs, fleas, flies, grasshoppers, leafhoppers, lice, locusts, mites, moths, nematodes, scales, symphylans, termites, thrips, ticks, wasps, and whiteflies. Within the definition broadly of pests can include: the Phyla Nematoda and/or Arthropoda. For example, specific crop pests and insects that may be controlled by these methods include the following: Dictyoptera (cockroaches); Isoptera (termites); Orthoptera (locusts, grasshoppers and crickets); Diptera (house flies, mosquito, tsetse fly, crane-flies and fruit flies); Hymenoptera (ants, wasps, bees, saw-flies, ichneumon flies and gall-wasps); Anoplura (biting and sucking lice); Siphonaptera (fleas); and Hemiptera (bugs and aphids), as well as arachnids such as Acari (ticks and mites), and the parasites that each of these organisms harbor.

In another embodiment, the KPVs and compositions and formulations containing one or more KPVs may be used to combat, control or inhibit pests in the Subphyla Chelicerata, Myriapoda, and/or Hexapoda.

In another embodiment, the KPVs and compositions and formulations containing one or more KPVs may be used to control pests in the Classes of Arachnida, Symphyla, and/or Insecta.

In some embodiments, the present disclosure provides a method for combating, controlling, or inhibiting insects, nematodes and non-insect arthropods.

In some embodiments, the present disclosure provides a method for combating, controlling, or inhibiting insects, (also referred to herein as an insect pest) include, but are not limited to, insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, and the like. More particularly, insect pests include Coleoptera, Lepidoptera, and Diptera.

In some embodiments, the present disclosure provides a method of combating, controlling, or inhibiting a pest comprising: (1) applying an pesticidal-effective amount of an engineered, non-naturally occurring pesticidal KPV, or an agriculturally acceptable salt thereof to the locus of the pest, or to a plant or animal susceptible to an attack by the pest; or (2) applying an pesticidal-effective amount of an pesticidal composition comprising: an engineered, non-naturally occurring pesticidal KPV, or an agriculturally acceptable salt thereof and an excipient; the locus of the pest, or to a plant or animal susceptible to an attack by the pest; wherein the pest is an insect pest, which may include, but are not limited to, insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, and the like. More particularly, insect pests include Coleoptera, Lepidoptera, and Diptera.

Insects of suitable agricultural, household and/or medical/veterinary importance for treatment with the pesticidal polypeptides are described below.

Insect pests include, but are not limited to, insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, and the like. More particularly, insect pests include Coleoptera, Lepidoptera, and Diptera.

Insects of suitable agricultural, household and/or medical/veterinary importance for treatment with the pesticidal peptides described herein include, but are not limited to, members of the following classes and orders:

The order Coleoptera includes the suborders Adephaga and Polyphaga. Suborder Adephaga includes the superfamilies Caraboidea and Gyrinoidea. Suborder Polyphaga includes the superfamilies Hydrophiloidea, Staphylinoidea, Cantharoidea, Cleroidea, Elateroidea, Dascilloidea, Dryopoidea, Byrrhoidea, Cucujoidea, Meloidea, Mordelloidea, Tenebrionoidea, Bostrichoidea, Scarabaeoidea, Cerambycoidea, Chrysomeloidea, and Curculionoidea. Superfamily Caraboidea includes the families Cicindelidae, Carabidae, and Dytiscidae. Superfamily Gyrinoidea includes the family Gyrinidae. Superfamily Hydrophiloidea includes the family Hydrophilidae. Superfamily Staphylinoidea includes the families Silphidae and Staphylinidae. Superfamily Cantharoidea includes the families Cantharidae and Lampyridae. Superfamily Cleroidea includes the families Cleridae and Dermestidae. Superfamily Elateroidea includes the families Elateridae and Buprestidae. Superfamily Cucujoidea includes the family Coccinellidae. Superfamily Meloidea includes the family Meloidae. Superfamily Tenebrionoidea includes the family Tenebrionidae. Superfamily Scarabaeoidea includes the families Passalidae and Scarabaeidae. Superfamily Cerambycoidea includes the family Cerambycidae. Superfamily Chrysomeloidea includes the family Chrysomelidae. Superfamily Curculionoidea includes the families Curculionidae and Scolytidae.

Examples of Coleoptera include, but are not limited to: the American bean weevil Acanthoscelides obtectus, the leaf beetle Agelastica alni, click beetles (Agriotes lineatus, Agriotes obscurus, Agriotes bicolor), the grain beetle Ahasverus advena, the summer schafer Amphimallon solstitialis, the furniture beetle Anobium punctatum, Anthonomus spp. (weevils), the Pygmy mangold beetle Atomaria linearis, carpet beetles (Anthrenus spp., Attagenus spp.), the cowpea weevil Callosobruchus maculates, the fried fruit beetle Carpophilus hemipterus, the cabbage seedpod weevil Ceutorhynchus assimilis, the rape winter stem weevil Ceutorhynchus picitarsis, the wireworms Conoderus vespertinus and Conoderus falli, the banana weevil Cosmopolites sordidus, the New Zealand grass grub Costelytra zealandica, the June beetle Cotinis nitida, the sunflower stem weevil Cylindrocopturus adspersus, the larder beetle Dermestes lardarius, the corn rootworms Diabrotica virgifera, Diabrotica virgifera virgifera, and Diabrotica barberi, the Mexican bean beetle Epilachna varivestis, the old house borer Hylotropes bajulus, the lucerne weevil Hypera postica, the shiny spider beetle Gibbium psylloides, the cigarette beetle Lasioderma serricorne, the Colorado potato beetle Leptinotarsa decemlineata, Lyctus beetles (Lyctus spp.), the pollen beetle Meligethes aeneus, the common cockshafer Melolontha melolontha, the American spider beetle Mezium americanum, the golden spider beetle Niptus hololeucus, the grain beetles Oryzaephilus surinamensis and Oryzaephilus mercator, the black vine weevil Otiorhynchus sulcatus, the mustard beetle Phaedon cochleariae, the crucifer flea beetle Phyllotreta cruciferae, the striped flea beetle Phyllotreta striolata, the cabbage steam flea beetle Psylliodes chrysocephala, Ptinus spp. (spider beetles), the lesser grain borer Rhizopertha dominica, the pea and been weevil Sitona lineatus, the rice and granary beetles Sitophilus oryzae and Sitophilus granaries, the red sunflower seed weevil Smicronyx fulvus, the drugstore beetle Stegobium paniceum, the yellow mealworm beetle Tenebrio molitor, the flour beetles Tribolium castaneum and Tribolium confusum, warehouse and cabinet beetles (Trogoderma spp.), and the sunflower beetle Zygogramma exclamationis.

Examples of Dermaptera (earwigs) include, but are not limited to: the European earwig, Forficula auricularia, and the striped earwig, Labidura riparia.

Examples of Dictvontera include, but are not limited to: the oriental cockroach, Blatta orientalis, the German cockroach, Blatella germanica, the Madeira cockroach, Leucophaea maderae, the American cockroach, Periplaneta americana, and the smokybrown cockroach Periplaneta fuliginosa.

Examples of Diplonoda include, but are not limited to: the spotted snake millipede Blaniulus guttulatus, the flat-back millipede Brachydesmus superus, and the greenhouse millipede Oxidus gracilis.

The order Diptera includes the Suborders Nematocera, Brachycera, and Cyclorrhapha. Suborder Nematocera includes the families Tipulidae, Psychodidae, Culicidae, Ceratopogonidae, Chironomidae, Simuliidae, Bibionidae, and Cecidomyiidae. Suborder Brachycera includes the families Stratiomyidae, Tabanidae, Therevidae, Asilidae, Mydidae, Bombyliidae, and Dolichopodidae. Suborder Cyclorrhapha includes the Divisions Aschiza and Aschiza. Division Aschiza includes the families Phoridae, Syrphidae, and Conopidae. Division Aschiza includes the Sections Acalyptratae and Calyptratae. Section Acalyptratae includes the families Otitidae, Tephritidae, Agromyzidae, and Drosophilidae. Section Calyptratae includes the families Hippoboscidae, Oestridae, Tachinidae, Anthomyiidae, Muscidae, Calliphoridae, and Sarcophagidae.

Examples of Diptera include, but are not limited to: the house fly (Musca domestica), the African tumbu fly (Cordylobia anthropophaga), biting midges (Culicoides spp.), bee louse (Braula spp.), the beet fly Pegomyia betae, black flies (Cnephia spp., Eusimulium spp., Simulium spp.), bot flies (Cuterebra spp., Gastrophilus spp., Oestrus spp.), craneflies (Tipula spp.), eye gnats (Hippelates spp.), filth-breeding flies (Calliphora spp., Fannia spp., Hermetia spp., Lucilia spp., Musca spp., Muscina spp., Phaenicia spp., Phormia spp.), flesh flies (Sarcophaga spp., Wohlfahrtia spp.); the flit fly Oscinella frit, fruitflies (Dacus spp., Drosophila spp.), head and canon flies (Hydrotea spp.), the hessian fly Mayetiola destructor, horn and buffalo flies (Haematobia spp.), horse and deer flies (Chrysops spp., Haematopota spp., Tabanus spp.), louse flies (Lipoptena spp., Lynchia spp., and Pseudolynchia spp.), medflies (Ceratitus spp.), mosquitoes (Aedes spp., Anopheles spp., Culex spp., Psorophora spp.), sandflies (Phlebotomus spp., Lutzomyia spp.), screw-worm flies (Chtysomya bezziana and Cochliomyia hominivorax), sheep keds (Melophagus spp.); stable flies (Stomoxys spp.), tsetse flies (Glossina spp.), and warble flies (Hypoderma spp.).

Examples of Isontera (termites) include, but are not limited to: species from the familes Hodotennitidae, Kalotermitidae, Mastotermitidae, Rhinotennitidae, Serritermitidae, Termitidae, and Termopsidae.

Examples of Heteroptera include, but are not limited to: the bed bug Cimex lectularius, the cotton stainer Dysdercus intermedius, the Sunn pest Eurygaster integriceps, the tarnished plant bug Lygus lineolaris, the green stink bug Nezara antennata, the southern green stink bug Nezara viridula, and the triatomid bugs Panstrogylus megistus, Rhodnius ecuadoriensis, Rhodnius pallescans, Rhodnius prolixus, Rhodnius robustus, Triatoma dimidiata, Triatoma infestans, and Triatoma sordida.

Examples of Homoptera include, but are not limited to: the California red scale Aonidiella aurantii, the black bean aphid Aphis fabae, the cotton or melon aphid Aphis gossypii, the green apple aphid Aphis pomi, the citrus spiny whitefly Aleurocanthus spiniferus, the oleander scale Aspidiotus hederae, the sweet potato whitefly Bemesia tabaci, the cabbage aphid Brevicoryne brassicae, the pear psylla Cacopsylla pyricola, the currant aphid Cryptomyzus ribis, the grape phylloxera Daktulosphaira vitifoliae, the citrus psylla Diaphorina citri, the potato leafhopper Empoasca fabae, the bean leafhopper Empoasca solana, the vine leafhopper Empoasca vitis, the woolly aphid Eriosoma lanigerum, the European fruit scale Eulecanium corni, the mealy plum aphid Hyalopterus arundinis, the small brown planthopper Laodelphax striatellus, the potato aphid Macrosiphum euphorbiae, the green peach aphid Myzus persicae, the green rice leafhopper Nephotettix cinticeps, the brown planthopper Nilaparvata lugens, gall-forming aphids (Pemphigus spp.), the hop aphid Phorodon humuli, the bird-cherry aphid Rhopalosiphum padi, the black scale Saissetia oleae, the greenbug Schizaphis graminum, the grain aphid Sitobion avenae, and the greenhouse whitefly Trialeurodes vaporariorum.

Examples of Isopoda include, but are not limited to: the common pillbug Armadillidium vulgare and the common woodlouse Oniscus asellus.

The order Lepidoptera includes the families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, and Tineidae.

Examples of Lepidoptera include, but are not limited to: Adoxophyes orana (summer fruit tortrix moth), Agrotis ipsolon (black cutworm), Archips podana (fruit tree tortrix moth), Bucculatrix pyrivorella (pear leafminer), Bucculatrix thurberiella (cotton leaf perforator), Bupalus piniarius (pine looper), Carpocapsa pomonella (codling moth), Chilo suppressalis (striped rice borer), Choristoneura fumiferana (eastern spruce budworm), Cochylis hospes (banded sunflower moth), Diatraea grandiosella (southwestern corn borer), Earls insulana (Egyptian bollworm), Euphestia kuehniella (Mediterranean flour moth), Eupoecilia ambiguella (European grape berry moth), Euproctis chrysorrhoea (brown-tail moth), Euproctis subflava (oriental tussock moth), Galleria mellonella (greater wax moth), Helicoverpa armigera (cotton bollworm), Helicoverpa zea (cotton bollworm), Heliothis virescens (tobacco budworm), Hofmannophila pseudopretella (brown house moth), Homeosoma electellum (sunflower moth), Homona magnanima (oriental tea tree tortrix moth), Lithocolletis blancardella (spotted tentiform leafminer), Lymantria dispar (gypsy moth), Malacosoma neustria (tent caterpillar), Mamestra brassicae (cabbage armyworm), Mamestra configurata (Bertha armyworm), the hornworms Manduca sexta and Manuduca quinquemaculata, Operophtera brumata (winter moth), Ostrinia nubilalis (European corn borer), Panolis flammea (pine beauty moth), Pectinophora gossypiella (pink bollworm), Phyllocnistis citrella (citrus leafminer), Pieris brassicae (cabbage white butterfly), Plutella xylostella (diamondback moth), Rachiplusia ni (soybean looper), Spilosoma virginica (yellow bear moth), Spodoptera exigua (beet armyworm), Spodoptera frugiperda (fall armyworm), Spodoptera littoralis (cotton leafworin), Spodoptera litura (common cutworm), Spodoptera praefica (yellowstriped armyworm), Sylepta derogata (cotton leaf roller), Tineola bisselliella (webbing clothes moth), Tineola pellionella (case-making clothes moth), Tortrix viridana (European oak leafroller), Trichoplusia ni (cabbage looper), and Yponomeuta padella (small ermine moth).

Examples of Orthoptera include, but are not limited to: the common cricket Acheta domesticus, tree locusts (Anacridium spp.), the migratory locust Locusta migratoria, the twostriped grasshopper Melanoplus bivittatus, the differential grasshopper Melanoplus dfferentialis, the redlegged grasshopper Melanoplus femurrubrum, the migratory grasshopper Melanoplus sanguinipes, the northern mole cricket Neocurtilla hexadectyla, the red locust Nomadacris septemfasciata, the shortwinged mole cricket Scapteriscus abbreviatus, the southern mole cricket Scapteriscus borellii, the tawny mole cricket Scapteriscus vicinus, and the desert locust Schistocerca gregaria.

Examples of Phthiraptera include, but are not limited to: the cattle biting louse Bovicola bovis, biting lice (Damalinia spp.), the cat louse Felicola subrostrata, the shortnosed cattle louse Haematopinus eloysternus, the tail-switch louse Haematopinus quadriperiussus, the hog louse Haematopinus suis, the face louse Linognathus ovillus, the foot louse Linognathus pedalis, the dog sucking louse Linognathus setosus, the long-nosed cattle louse Linognathus vituli, the chicken body louse Menacanthus stramineus, the poultry shaft louse Menopon gallinae, the human body louse Pediculus humanus, the pubic louse Phthirus pubis, the little blue cattle louse Solenopotes capillatus, and the dog biting louse Trichodectes canis.

Examples of Psocoptera include, but are not limited to: the booklice Liposcelis bostrychophila, Liposcelis decolor, Liposcelis entomophila, and Trogium pulsatorium. Examples of Siphonaptera include, but are not limited to: the bird flea Ceratophyllus gallinae, the dog flea Ctenocephalides canis, the cat flea Ctenocephalides fells, the human flea Pulex irritans, and the oriental rat flea Xenopsylla cheopis.

Examples of Symphyla include, but are not limited to: the garden symphylan Scutigerella immaculate.

Examples of Thysamura include, but are not limited to: the gray silverfish Ctenolepisma longicaudata, the four-lined silverfish Ctenolepisma quadriseriata, the common silverfish Lepisma saccharina, and the firebrat Thennobia domestica;

Examples of Thysanoptera include, but are not limited to: the tobacco thrips Frankliniella fusca, the flower thrips Frankliniella intonsa, the western flower thrips Frankliniella occidentalis, the cotton bud thrips Frankliniella schultzei, the banded greenhouse thrips Hercinothrips femoralis, the soybean thrips Neohydatothrips variabilis, Kelly's citrus thrips Pezothrips kellyanus, the avocado thrips Scirtothrips perseae, the melon thrips, Thrips palmi, and the onion thrips, Thrips tabaci.

Examples of Nematodes include, but are not limited to: parasitic nematodes such as root-knot, cyst, and lesion nematodes, including Heterodera spp., Meloidogyne spp., and Globodera spp.; particularly members of the cyst nematodes, including, but not limited to: Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include, but are not limited to: Pratylenchus spp.

Other insect species susceptible to the present disclosure include: athropod pests that cause public and animal health concerns, for example, mosquitos for example, mosquitoes from the genera Aedes, Anopheles and Culex, from ticks, flea, and flies etc.

In one embodiment, a KPV, a KPV-pesticidal protein, or an agriculturally acceptable salt thereof can be employed to treat ectoparasites. Ectoparasites include, but are not limited to: fleas, ticks, mange, mites, mosquitoes, nuisance and biting flies, lice, and combinations comprising one or more of the foregoing ectoparasites. The term “fleas” includes the usual or accidental species of parasitic flea of the order Siphonaptera, and in particular the species Ctenocephalides, in particular C. fells and C. cams, rat fleas (Xenopsylla cheopis) and human fleas (Pulex irritans).

The present disclosure may be used to control, inhibit, and/or kill insect pests of major crops, e.g., in some embodiments, the major crops and corresponding insect pest include, but are not limited to: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis, corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, banded winged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus, chinch bug; Acrosternum hilare, green stink bug; Soybean: Pseudoplusia includens, soybean looper; Anticarsia gemmatalis, velvet bean caterpillar; Plathypena scabra, green clover worm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

In some embodiments, a KPV, a KPV-pesticidal protein, or an agriculturally acceptable salt thereof can be employed to treat any one or more of the foregoing insects, or any of the insects described herein.

The insects that are susceptible to present disclosure include but are not limited to the following: familes such as: Blattaria, Coleoptera, Collembola, Diptera, Echinostomida, Hemiptera, Hymenoptera, Isoptera, Lepidoptera, Neuroptera, Orthoptera, Rhabditida, Siphonoptera, and Thysanoptera. Genus Species are indicated as follows: Actebia fennica, Agrotis ipsilon, A. segetum, Anticarsia gemmatalis, Argyrotaenia citrana, Artogeia rapae, Bombyx mori, Busseola fusca, Cacyreus marshall, Chilo suppressalis, Christoneura fumiferana, C. occidentalis, C. pinus pinus, C. rosacena, Cnaphalocrocis medinalis, Conopomorpha cramerella, Ctenopsuestis obliquana, Cydia pomonella, Danaus plexippus, Diatraea saccharallis, D. grandiosella, Earias vittella, Elasmolpalpus lignoselius, Eldana saccharina, Ephestia kuehniella, Epinotia aporema, Epiphyas postvittana, Galleria mellonella, Genus-Species, Helicoverpa zea, H. punctigera, H. armigera, Heliothis virescens, Hyphantria cunea, Lambdina fiscellaria, Leguminivora glycinivorella, Lobesia botrana, Lymantria dispar, Malacosoma disstria, Mamestra brassicae, M. configurata, Manduca sexta, Marasmia patnalis, Maruca vitrata, Orgyia leucostigma, Ostrinia nubilalis, O. furnacalis, Pandemis pyrusana, Pectinophora gossypiella, Perileucoptera coffeella, Phthorimaea opercullela, Pianotortrix octo, Piatynota stultana, Pieris brassicae, Plodia interpunctala, Plutella xylostella, Pseudoplusia includens, Rachiplusia nu, Sciropophaga incertulas, Sesamia calamistis, Spilosoma virginica, Spodoptera exigua, Spodoptera frugiperda, Spodoptera littoralis, Spodoptera exempta, Spodoptera litura, Tecia solanivora, Thaumetopoea pityocampa, Trichoplusia ni, Wiseana cervinata, Wiseana copularis, Wiseana jocosa, Blattaria blattella, Collembola xenylla, Collembola folsomia, Folsomia candida, Echinostomida fasciola, Hemiptera oncopeltrus, Hemiptera bemisia, Hemiptera macrosiphum, Hemiptera rhopalosiphum, Hemiptera myzus, Hymenoptera diprion, Hymenoptera apis, Hymenoptera macrocentrus, Hymenoptera meteorus, Hymenoptera nasonia, Hymenoptera solenopsis, Isopoda porcellio, Isoptera reticulitermes, Orthoptera Achta, Prostigmata tetranychus, Rhabitida acrobeloides, Rhabitida caenorhabditis, Rhabitida distolabrellus, Rhabitida panagrellus, Rhabitida pristionchus, Rhabitida pratylenchus, Rhabitida ancylostoma, Rhabitida nippostrongylus, Rhabitida panagrellus, Rhabitida haemonchus, Rhabitida meloidogyne, and Siphonaptera ctenocephalides.

The present disclosure provides methods for plant transformation, which may be used for transformation of any plant species, including, but not limited to, monocots and dicots. Crops for which a transgenic approach would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

The present disclosure provides methods for plant transformation, which may be used for transformation of any plant species, including, but not limited to, monocots and dicots. Crops for which a transgenic approach or plaint incorporated protectants (PIP) would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

In some embodiments, the compositions, mixtures, and/or methods of the present disclosure can be applied to the locus of an insect and/or pest selected from the group consisting of: Loopers; Omnivorous Leafroller; Hornworms; Imported Cabbageworm; Diamondback Moth; Green Cloverworm; Webworm; Saltmarsh Caterpillar; Armyworms; Cutworms; Cross-Striped Cabbageworm; Podworms; Velvetbean Caterpillar; Soybean Looper; Tomato Fruitworm; Variegated Cutworm; Melonworms; Rindworm complex; Fruittree Leafroller; Citrus Cutworm; Heliothis; Orangedog; Citrus Cutworm; Redhumped Caterpillar; Tent Caterpillars; Fall Webworm; Walnut Caterpillar; Cankerworms; Gypsy Moth; Variegated Leafroller; Redbanded Leafroller; Tufted Apple Budmoth; Oriental Fruit Moth); Filbert Leafroller; Obliquebanded Leafroller; Codling Moth; Twig Borer; Grapeleaf Skeletonizer; Grape Leafroller; Achema Sphinx Moth (Hornworm); Orange Tortrix; Tobacco Budworm); Grape Berry Moth; Spanworm; Alfalfa Caterpillar; Cotton Bollworm; Head Moth; Amorbia Moth; Omnivorous Looper; Ello Moth (Hornworm); Io Moth; Oleander Moth; Azalea Caterpillar; Hornworm; Leafrollers; Banana Skipper; Batrachedra comosae (Hodges); Thecla Moth; Artichoke Plume Moth; Thistle Butterfly; Bagworm; Spring & Fall Cankerworm; Elm Spanworm; California Oakworm; Pine Butterfly; Spruce Budworms; Saddle Prominent Caterpillar; Douglas Fir Tussock Moth; Western Tussock Moth; Blackheaded Budworm; Mimosa Webworm; Jack Pine Budworm; Saddleback Caterpillar; Greenstriped Mapleworm; or Hemlock Looper.

In some embodiments, the peptides, proteins, compositions, mixtures, and/or methods of the present disclosure can be applied to the locus of an insect and/or pest selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola) (Essex Skipper); Fall Webworm (Melissopus latiferreanus); Filbert Leafroller (Archips rosanus); Fruittree Leafroller (Archips argyrospilia); Grape Berry Moth (Paralobesia viteana); Grape Leafroller (Platynota stultana); Grapeleaf Skeletonizer (Harrisina americana) (ground only); Green Cloverworm (Plathypena scabra); Greenstriped Mapleworm (Dryocampa rubicunda); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Redbanded Leafroller (Argyrotaenia velutinana); Redhumped Caterpillar (Schizura concinna); Rindworm Complex (Various Leps.); Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata; Ostrinia furnacalis; Ostrinia nubilalis; Pectinophora gossypiella; Plodia interpunctella, Plutella xylostella, Pseudoplusia includens; Spodoptera exigua, Spodoptera frugiperda; Spodoptera littoralis; Trichoplusia ni, and/or Xanthogaleruca luteola.

In some embodiments, the peptides, proteins, compositions, mixtures, and/or methods of the present disclosure can be applied to the locus of an adult beetle selected from the group consisting of: Asiatic garden beetle (Maladera castanea); Gold spotted oak borer (Agrilus coxalis auroguttatus); Green June beetle (Cotinis nitida); Japanese beetle (Popillia japonica); May or June beetle (Phyllophaga sp.); Oriental beetle (Anomala orientalis); and/or Soap berry-borer (Agrilus prionurus).

In some embodiments, the compositions, mixtures, and/or methods of the present disclosure can be applied to the locus of an insect and/or pest that is a larvae (annual white grub) selected from the group consisting of: Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); and Billbug (Curculionoidea).

Examples of Nematodes include, but are not limited to: parasitic nematodes such as root-knot, cyst, and lesion nematodes, including Aphelenchoides spp., Belonolaimus spp., Criconemella spp., Ditylenchus spp., Hirschmanniella spp., Hoplolaimus spp., Heterodera spp., Meloidogyne spp., Pratylenchus spp., Radopholus spp., and Globodera spp.; particularly members of the cyst nematodes, including, but not limited to: Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include, but are not limited to: Pratylenchus spp.

In some embodiments, the present disclosure provides a method for combating, controlling, or inhibiting plant pests of the Order Acarina. A non-exhaustive list of particular genera includes, but is not limited to, Acarus spp., Aculops spp., Boophilus spp., Demodex spp., Dermacentor spp., Epitrimerus spp., Eriophyes spp., Ixodes spp., Oligonychus spp., Panonychus spp., Rhizoglyphus spp., and Tetranychus spp. A non-exhaustive list of particular species includes, but is not limited to, Acarapis woodi, Acarus siro, Aceria mangiferae, Aculops lycopersici, Aculus pelekassi, Aculus schlechtendali, Amblyomma americanum, Brevipalpus obovatus, Brevipalpus phoenicis, Dermacentor variabilis, Dermatophagoides pteronyssinus, Eotetranychus carpini, Notoedres cati, Oligonychus coffee, Oligonychus ilicis, Panonychus citri, Panonychus ulmi, Phyllocoptruta oleivora, Polyphagotarsonemus latus, Rhipicephalus sanguineus, Sarcoptes scabiei, Tegolophus perseaflorae, Tetranychus urticae, and Varroa destructor.

In another embodiment, the present disclosure provides a method for combating, controlling, or inhibiting a plant pest of the Order Symphyla. A non-exhaustive list of particular sp. includes, but is not limited to, Scutigerella immaculata.

Other plant pests treated with the methods described herein may include non-insect arthropods, for example, aphids, mites, for example, spider mites, ticks, thrips, whiteflies, mealybugs, earworms, for example, corn earworm, and hornworms, for example, tomato hornworm. In some embodiments, the pest to be controlled, treated or killed with the compositions of the present disclosure may include, insects and/or other plant pests, including for example, but not limited to: thrips, aphids, whiteflies, mites, including spider mites and other arthropods pests that are known to defoliate and generally attack plants, for example, beet flea beetles, Chaetocnema concinna; beet leafhoppers, Circulifer tenellus; beet leafminers, Pegomya betae; sugarbeet root aphids, Pemphigus betae; garden symphylans, Scutigerella immaculata; lucerne fleas, Sminthurus viridis; sugarbeet root maggots, Tetanops myopaeformis, wireworms. corn rootworms (Diabrotica spp.), corn silk flies (Euxesta stigmatias), European corn borers (Ostrinia nubilalis), stalk borers (Diatraea spp.), aphids, billbugs (Sphenophorus spp.), fall armyworms (Spodoptera frugiperda), corn earworms (Helicoverpa zea), cutworms, wireworms, velvet bean caterpillars, Anticarsia gemmatalis Hübner; soybean loopers, Chrysodeixis includens Walker; leaf beetles such as Cerotoma spp. and Epilachna varivestis Mulsant, tortricid pod and stem borers, Crocidosema spp.; bollworm, Helicoverpa spp., armyworms, Spodoptera sp., pod-feeding stink bugs, soybean aphids, Aphis glycines, Matsumura; and the bean stem flies Melanagromyza soja Zehntner, thrips, and ticks.

In some embodiments, the present disclosure provides a method of combating, controlling, or inhibiting an insect pest comprising: (1) applying an pesticidal-effective amount of an engineered, non-naturally occurring pesticidal KPV, or an agriculturally acceptable salt thereof to the locus of the pest, or to a plant or animal susceptible to an attack by the pest; or (2) applying an pesticidal-effective amount of an pesticidal composition comprising: an engineered, non-naturally occurring pesticidal KPV, or an agriculturally acceptable salt thereof and an excipient; the locus of the pest, or to a plant or animal susceptible to an attack by the pest; wherein the pest is an insect pest belonging to the genera: Coleoptera, Diptera, Hymenoptera, or Lepidoptera.

In some embodiments, the present disclosure provides a method of combating, controlling, or inhibiting an insect comprising: (1) applying an pesticidal-effective amount of an engineered, non-naturally occurring pesticidal KPV, or an agriculturally acceptable salt thereof to the locus of the insect, or to a plant or animal susceptible to an attack by the insect; or (2) applying an pesticidal-effective amount of an pesticidal composition comprising: an engineered, non-naturally occurring pesticidal KPV, or an agriculturally acceptable salt thereof and an excipient; the locus of the insect, or to a plant or animal susceptible to an attack by the insect; wherein the insect is an insect from the order Coleoptera, Diptera, or Lepidoptera.

Any of the insects, nematodes and non-insect arthropods described herein can be combatted, controlled, or inhibited using an engineered, non-naturally occurring pesticidal KPV, or an agriculturally acceptable salt thereof consisting of an amino acid sequence set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, and any of the excipients described herein.

### Illustrative Embodiments

In some embodiments, a Kappa Peptide Variant (KPV) having pesticidal activity against one or more pests, comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a Kappa Peptide Variant (KPV) having pesticidal activity against one or more pests, comprises, consists essentially of, or consists of: an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; or an agriculturally acceptable salt thereof.

In some embodiments, an Kappa Peptide Variant (KPV) having pesticidal activity against one or more pest species, comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a homopolymer or heteropolymer of two or more KPVs, wherein the amino acid sequence of each KPV is the same or different.

In some embodiments, an Kappa Peptide Variant (KPV) having pesticidal activity against one or more pest species, comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable or non-cleavable linker, and wherein the amino acid sequence of each KPV may be the same or different.

In some embodiments, an Kappa Peptide Variant (KPV) having pesticidal activity against one or more pest species, comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different.

In some embodiments, an Kappa Peptide Variant (KPV) having pesticidal activity against one or more pest species, comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different; wherein the linker has an amino acid sequence as set forth in any one of SEQ ID NOs: 360-369.

In some embodiments, an Kappa Peptide Variant (KPV) having pesticidal activity against one or more pest species, comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different; wherein the linker is cleavable inside at least one of (i) the gut or hemolymph of an insect, and (ii) cleavable inside the gut of a mammal.

In some embodiments, a combination comprises, consists essentially of, or consists of: two or more Kappa Peptide Variant (KPVs) having pesticidal activity against one or more pest species, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a combination comprises, consists essentially of, or consists of: two or more Kappa Peptide Variant (KPVs) having pesticidal activity against one or more pest species, wherein each of the two or more KPVs independently comprises, consists essentially of, or consists of: an amino acid sequence that is at least 90%, 95%, 96%, 97%, 97%, 98%, 99%, or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein each of the KPVs independently comprises at least one amino acid substitution relative to the amino acid sequence set forth in SEQ ID NO: 1, or an agriculturally acceptable salt thereof, with the proviso that the KPV does not comprise the amino acid sequence of SEQ ID NOs: 1 or 253.

In some embodiments, a combination comprises, consists essentially of, or consists of: two or more Kappa Peptide Variant (KPVs) having pesticidal activity against one or more pest species, wherein each of the two or more KPVs independently comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a combination comprises, consists essentially of, or consists of: two or more Kappa Peptide Variant (KPVs) having pesticidal activity against one or more pest species, wherein each of the two or more KPVs independently consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a combination comprises, consists essentially of, or consists of: two or more Kappa Peptide Variant (KPVs) having pesticidal activity against one or more pest species, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a homopolymer or heteropolymer of two or more KPVs, wherein the amino acid sequence of each KPV is the same or different.

In some embodiments, a combination comprises, consists essentially of, or consists of: two or more Kappa Peptide Variant (KPVs) having pesticidal activity against one or more pest species, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable or non-cleavable linker, and wherein the amino acid sequence of each KPV may be the same or different.

In some embodiments, a combination comprises, consists essentially of, or consists of: two or more Kappa Peptide Variant (KPVs) having pesticidal activity against one or more pest species, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different.

In some embodiments, a combination comprises, consists essentially of, or consists of: two or more Kappa Peptide Variant (KPVs) having pesticidal activity against one or more pest species, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different; wherein the linker has an amino acid sequence as set forth in any one of SEQ ID NOs: 360-369.

In some embodiments, a combination comprises, consists essentially of, or consists of: two or more Kappa Peptide Variant (KPVs) having pesticidal activity against one or more pest species, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different; wherein the linker is cleavable inside at least one of (i) the gut or hemolymph of an insect, and (ii) cleavable inside the gut of a mammal.

In some embodiments, a composition comprises, consists essentially of, or consists of: an Kappa Peptide Variant (KPV) having pesticidal activity against one or more pest species, and an agriculturally acceptable excipient; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a composition comprises, consists essentially of, or consists of: an Kappa Peptide Variant (KPV) having pesticidal activity against one or more pest species, and an agriculturally acceptable excipient; wherein the KPV comprises, consists essentially of, or consists of: an amino acid sequence that is at least 90%, 95%, 96%, 97%, 97%, 98%, 99% or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a composition comprises, consists essentially of, or consists of: an Kappa Peptide Variant (KPV) having pesticidal activity against one or more pest species, and an agriculturally acceptable excipient; wherein the KPV comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a composition comprises, consists essentially of, or consists of: an Kappa Peptide Variant (KPV) having pesticidal activity against one or more pest species, and an agriculturally acceptable excipient; wherein the KPV consists of an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a composition comprises, consists essentially of, or consists of: an Kappa Peptide Variant (KPV) having pesticidal activity against one or more pest species, and an agriculturally acceptable excipient; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a homopolymer or heteropolymer of two or more KPVs, wherein the amino acid sequence of each KPV is the same or different.

In some embodiments, a composition comprises, consists essentially of, or consists of: an Kappa Peptide Variant (KPV) having pesticidal activity against one or more pest species, and an agriculturally acceptable excipient; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable or non-cleavable linker, and wherein the amino acid sequence of each KPV may be the same or different.

In some embodiments, a composition comprises, consists essentially of, or consists of: an Kappa Peptide Variant (KPV) having pesticidal activity against one or more pest species, and an agriculturally acceptable excipient; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different.

In some embodiments, a composition comprises, consists essentially of, or consists of: an Kappa Peptide Variant (KPV) having pesticidal activity against one or more pest species, and an agriculturally acceptable excipient; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different; wherein the linker has an amino acid sequence as set forth in any one of SEQ ID NOs: 360-369.

In some embodiments, a composition comprises, consists essentially of, or consists of: an Kappa Peptide Variant (KPV) having pesticidal activity against one or more pest species, and an agriculturally acceptable excipient; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different; wherein the linker is cleavable inside at least one of (i) the gut or hemolymph of an insect, and (ii) cleavable inside the gut of a mammal.

In some embodiments, a composition comprises, consists essentially of, or consists of: a combination of two or more Kappa Peptide Variant (KPVs), each having pesticidal activity against one or more pest species, and an agriculturally acceptable excipient; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a composition comprises, consists essentially of, or consists of: a combination of two or more Kappa Peptide Variant (KPVs), each having pesticidal activity against one or more pest species, and an agriculturally acceptable excipient; wherein each of the two or more KPVs independently comprises, consists essentially of, or consists of: an amino acid sequence that is at least 90%, 95%, 96%, 97%, 97%, 98%, 99%, or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein each of the KPVs independently comprises at least one amino acid substitution relative to the amino acid sequence set forth in SEQ ID NO: 1, or an agriculturally acceptable salt thereof, with the proviso that the KPV does not comprise the amino acid sequence of SEQ ID NOs: 1 or 253.

In some embodiments, a composition comprises, consists essentially of, or consists of: a combination of two or more Kappa Peptide Variant (KPVs), each having pesticidal activity against one or more pest species, and an agriculturally acceptable excipient; wherein each of the two or more KPVs independently comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a composition comprises, consists essentially of, or consists of: a combination of two or more Kappa Peptide Variant (KPVs), each having pesticidal activity against one or more pest species, and an agriculturally acceptable excipient; wherein each of the two or more KPVs independently consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a composition comprises, consists essentially of, or consists of: a combination of two or more Kappa Peptide Variant (KPVs), each having pesticidal activity against one or more pest species, and an agriculturally acceptable excipient; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a homopolymer or heteropolymer of two or more KPVs, wherein the amino acid sequence of each KPV is the same or different.

In some embodiments, a composition comprises, consists essentially of, or consists of: a combination of two or more Kappa Peptide Variant (KPVs), each having pesticidal activity against one or more pest species, and an agriculturally acceptable excipient; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable or non-cleavable linker, and wherein the amino acid sequence of each KPV may be the same or different.

In some embodiments, a composition comprises, consists essentially of, or consists of: a combination of two or more Kappa Peptide Variant (KPVs), each having pesticidal activity against one or more pest species, and an agriculturally acceptable excipient; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different.

In some embodiments, a composition comprises, consists essentially of, or consists of: a combination of two or more Kappa Peptide Variant (KPVs), each having pesticidal activity against one or more pest species, and an agriculturally acceptable excipient; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different; wherein the linker has an amino acid sequence as set forth in any one of SEQ ID NOs: 360-369.

In some embodiments, a composition comprises, consists essentially of, or consists of: a combination of two or more Kappa Peptide Variant (KPVs), each having pesticidal activity against one or more pest species, and an agriculturally acceptable excipient; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different; wherein the linker is cleavable inside at least one of (i) the gut or hemolymph of an insect, and (ii) cleavable inside the gut of a mammal.

In some embodiments, a polynucleotide of the present disclosure, or a complementary nucleotide sequence thereof, is operable to encode an Kappa Peptide Variant (KPV), said KPV comprising, consisting essentially of, or consisting of, an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253.

In some embodiments, a polynucleotide of the present disclosure, or a complementary nucleotide sequence thereof, is operable to encode an Kappa Peptide Variant (KPV), comprising, consisting essentially of, or consisting of, an amino acid sequence that is at least 95% identical to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a polynucleotide of the present disclosure, or a complementary nucleotide sequence thereof, is operable to encode an Kappa Peptide Variant (KPV), comprising an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a polynucleotide of the present disclosure, or a complementary nucleotide sequence thereof, is operable to encode an Kappa Peptide Variant (KPV), consisting of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a vector comprises a polynucleotide, or a complementary nucleotide sequence thereof, operable to encode an Kappa Peptide Variant (KPV), said KPV comprising, consisting essentially of, or consisting of, an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253.

In some embodiments, a vector comprises a polynucleotide, or a complementary nucleotide sequence thereof, operable to encode an Kappa Peptide Variant (KPV), comprising, consisting essentially of, or consisting of, an amino acid sequence that is at least 95% identical to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a vector comprises a polynucleotide, or a complementary nucleotide sequence thereof, operable to an Kappa Peptide Variant (KPV), comprising an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a vector comprises a polynucleotide, or a complementary nucleotide sequence thereof, operable to an Kappa Peptide Variant (KPV), consisting of an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a polynucleotide of the present disclosure comprises a polynucleotide operable to hybridize under stringent hybridization conditions with a polynucleotide segment encoding a KPV, said KPV comprising, consisting essentially of, or consisting of, an amino acid sequence that is at least 90%, 95%, 96%, 97%, 97%, 98%, 99% or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253.

In some embodiments, a polynucleotide of the present disclosure comprises a polynucleotide operable to hybridize under stringent hybridization conditions with a polynucleotide segment encoding a KPV, said KPV comprising, consisting essentially of, or consisting of, an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a yeast strain comprises: a first expression cassette comprising a polynucleotide operable to encode a KPV, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253.

In some embodiments, a yeast strain comprises: a first expression cassette comprising a polynucleotide operable to encode a KPV, said KPV comprising an amino acid sequence that is at least 95% identical to an amino acid sequence as set forth in any one of 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; wherein the KPV comprises at least one amino acid substitution relative to the amino acid sequence set forth in SEQ ID NO: 1; or a complementary nucleotide sequence thereof, with the proviso that the KPV does not comprise the amino acid sequence of SEQ ID NOs: 1 or 253.

In some embodiments, a yeast strain comprises: a first expression cassette comprising a polynucleotide operable to encode a KPV, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; and wherein the yeast strain is a yeast cell that is selected from any species belonging to the genera Saccharomyces, Pichia, Kluyveromyces, Hansenula, Yarrowia or Schizosaccharomyces.

In some embodiments, a yeast strain comprises: a first expression cassette comprising a polynucleotide operable to encode a KPV, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; and wherein the yeast strain is a yeast cell selected from the group consisting of Kluyveromyces lactis, Kluyveromyces marxianus, Saccharomyces cerevisiae, and Pichia pastoris.

In some embodiments, a yeast strain comprises: a first expression cassette comprising a polynucleotide operable to encode a KPV, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; and wherein the yeast strain is Kluyveromyces lactis or Kluyveromyces marxianus.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; wherein the KPV comprises, consists essentially of, or consists of, an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the KPV is a homopolymer or heteropolymer of two or more KPVs, wherein the amino acid sequence of each KPV is the same or different.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the KPV is a fused protein comprising two or more KPVs separated by a cleavable or non-cleavable linker, and wherein the amino acid sequence of each KPV may be the same or different.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the KPV is a fused protein comprising two or more KPVs separated by a cleavable, and wherein the amino acid sequence of each KPV may be the same or different.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the KPV is a fused protein comprising two or more KPVs separated by a cleavable or non-cleavable linker, and wherein the amino acid sequence of each KPV may be the same or different; wherein the linker has an amino acid sequence as set forth in any one of SEQ ID NOs: 360-369.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the KPV is a fused protein comprising two or more KPVs separated by a cleavable or non-cleavable linker, and wherein the amino acid sequence of each KPV may be the same or different; and wherein the linker is cleavable inside at least one of (i) the gut or hemolymph of an insect, and (ii) cleavable inside the gut of a mammal.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the vector is a plasmid comprising an alpha-MF signal.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the host cell is a yeast strain.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the host cell is a yeast strain is selected from any species belonging to the genera Saccharomyces, Pichia, Kluyveromyces, Hansenula, Yarrowia, or Schizosaccharomyces.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the host cell is a yeast strain is selected from the group consisting of Kluyveromyces lactis, Kluyveromyces marxianus, Saccharomyces cerevisiae, and Pichia pastoris.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the host cell is a yeast strain is Kluyveromyces lactis.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the KPV is secreted into the growth medium.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein expression of the KPV provides a yield of: at least 70 mg/L, at least 80 mg/L, at least 90 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, at least 190 mg/L, at least 200 mg/L, at least 500 mg/L, at least 750 mg/L, at least 1,000 mg/L, at least 1,250 mg/L, at least 1,500 mg/L, at least 1,750 mg/L, at least 2,000 mg/L, at least 2,500 mg/L, at least 3,000 mg/L, at least 3,500 mg/L, at least 4,000 mg/L, at least 4,500 mg/L, at least 5,000 mg/L, at least 5,500 mg/L, at least at least 6,000 mg/L, at least 6,500 mg/L, at least 7,000 mg/L, at least 7,500 mg/L, at least 8,000 mg/L, at least 8,500 mg/L, at least 9,000 mg/L, at least 9,500 mg/L, at least 10,000 mg/L, at least 11,000 mg/L, at least 12,000 mg/L, at least 12,500 mg/L, at least 13,000 mg/L, at least 14,000 mg/L, at least 15,000 mg/L, at least 16,000 mg/L, at least 17,000 mg/L, at least 17,500 mg/L, at least 18,000 mg/L, at least 19,000 mg/L, at least 20,000 mg/L, at least 25,000 mg/L, at least 30,000 mg/L, at least 40,000 mg/L, at least 50,000 mg/L, at least 60,000 mg/L, at least 70,000 mg/L, at least 80,000 mg/L, at least 90,000 mg/L, or at least 100,000 mg/L of KPV per liter of yeast culture medium.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein expression of the KPV in the medium results in the expression of a single KPV in the medium.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein expression of the KPV in the medium results in the expression of a KPV polymer comprising two or more KPV polypeptides in the medium.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the vector comprises two or three expression cassettes, each expression cassette operable to encode the KPV of the first expression cassette.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the vector comprises two or three expression cassettes, each expression cassette operable to encode the KPV of the first expression cassette, or a KPV of a different expression cassette.

In some embodiments, a method of producing a KPV comprises: (a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof, with the proviso that the KPV does not comprise the amino acid sequence of SEQ ID NOs: 1 or 253; (b) introducing the vector into a host cell; and (c) growing the host cell in a growth medium under conditions operable to enable expression of the KPV and secretion into the growth medium; and wherein the vector comprises two or three expression cassettes, each expression cassette operable to encode the KPV of the first expression cassette, or a KPV of a different expression cassette; and wherein the expression cassette is operable to encode a KPV as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99% or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a homopolymer or heteropolymer of two or more KPVs, wherein the amino acid sequence of each KPV is the same or different.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable or non-cleavable linker, and wherein the amino acid sequence of each KPV may be the same or different.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different; wherein the linker has an amino acid sequence as set forth in any one of SEQ ID NOs: 360-369.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different; wherein the linker is cleavable inside at least one of (i) the gut or hemolymph of an insect, and (ii) cleavable inside the gut of a mammal.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is an insect.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is a Lepidopteran insect.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is a Dipteran insect.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is a Coleopteran insect.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is a non-insect arthropod pest.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is selected from the group comprising Acariformes sp.; Acleris gloverana, Acrobasis vaccinii; Adoxophyes orana, Aedes aegypti; Agrotis ipsilon; Aleyrodidae sp.; Alsophila pometaria, Ambylomma americanum; Amblyomma maculatum; Amorbia humerosana; Anarsia lineatella, Anomala orientalis; Anthonomus eugenii; Anticarsia gemmatalis; Aphidoidea sp.; Aphis glycines Matsumura; Archips argyrospilia, Archips podana; Archips rosana; Argyrotaenia citrana; Argyrotaenia ljungiana; Argyrotaenia velutinana; Automeris io; Batrachedra comosae; Busseola fusca; Caudra cautella, Cerotoma spp.; Chaetocnema concinna; Chilo auricilius; Chilo partellus; Chilo suppressalis; Choristoneura fumiferana, Choristoneura pinus; Choristoneura rosaceana, Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida, Cydia funebrana, Cydia molesta, Cydia nigricana, Cydia pomonella; Cydia splendana; Cylas formicarius; Datana integerrima, Datana major; Dermacentor albipictus; Dermacentor andersoni; Dermacentor occidentalis; Dermacentor variabilis; Diabrotica spp.; Diabrotica virgifera, Diaphania hyalinata, Diaprepes abbreviatus; Diatraea crambidoides; Diatraea saccharalis; Diatraea spp.; Dorysthenes buqueti; Dryocampa rubicunda, Earias insulana; Ennomos subsignaria; Ephestia elutella, Epilachna varivestis Mulsant, Epiphyas postvittana, Erinnyis ello; Erionota thrax; Estigmene acrea; Euetheola humilis; Eumorpha achemon; Euwallacea fornicatus; Euxesta stigmatias; Evergestis rimosalis; Grapholita delineana; Grapholita molesta, Grapholita packardi; Halyomorpha halys; Harrisina americana, Helicoverpa armigera; Helicoverpa spp.; Helicoverpa zea, Heliothis virescens; Heterocampa guttivitta; Homadaula anisocentra; Hypera postica; Hypera zoilus; Hypercompe scribonia, Hyphantria cunea, Hypothenemus hampei, Ixodes cookei, Ixodes pacificus; Ixodes scapularis; Lambdina fiscellaria, Lasiocampidae sp.; Leptinotarsa decemlineata, Lissorhoptrus oryzophilus; Listronotus maculicollis; Lobesia botrana, Lymantria dispar; Maladera castanea, Maliarpha separatella; Manduca quinquemaculata, Manduca sexta; Melanagromyza soja Zehntner; Melissopus latiferreanus; Neophasia menapia, Noctua pronuba, Noctuid sp.; Nymphula stagnata; Orgyia pseudotsugata; Orgyia vetusta, Ostrinia furnacalis; Ostrinia nubilalis; Otiorhynchus ovatus; Paleacrita merriccata; Pandemis cerasana, Papilio cresphontes; Paralobesia viteana, Pectinophora gossypiella, Pegomya betae; Pemphigus betae; Peridroma saucia; Phryganidia californica; Phyllophaga sp.; Pieris rapae; Plathypena scabra; Platynota flavedana, Platynota idaeusalis; Platynota stultana; Platyptilia carduidactyla; Playnota stultana; Plodia interpunctella, Plutella xylostella, Popillia japonica; Pseudaletia unipuncta, Pseudoplusia includens; Rhipicephalus sangunineus; Rhizotroqus majalis; Sabulodes aegrotata; Schizura concinna; Scirpophaga incertulas; Scirpophaga innotata, Scutigerella immaculata; Sesamia calamistis; Sibine stimulea; Sminthurus viridis; Sparganothis pilleriana, Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua, Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni, Xanthogaleruca luteola, Xylomyges curialis; or Xylotrechus quadripes.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is selected from the group consisting of: Acariformes sp.; Acleris gloverana, Acrobasis vaccinii; Adoxophyes orana; Aedes aegypti, Agrotis ipsilon; Aleyrodidae sp.; Alsophila pometaria, Ambylomma americanum; Amblyomma maculatum; Amorbia humerosana; Anarsia lineatella; Anomala orientalis; Anthonomus eugenii; Anticarsia gemmatalis; Aphidoidea sp.; Aphis glycines Matsumura; Archips argyrospilia, Archips podana; Archips rosana, Argyrotaenia citrana, Argyrotaenia ljungiana, Argyrotaenia velutinana; Automeris io; Batrachedra comosae; Busseola fusca; Caudra cautella, Cerotoma spp.; Chaetocnema concinna; Chilo auricilius; Chilo partellus; Chilo suppressalis; Choristoneura fumiferana, Choristoneura pinus; Choristoneura rosaceana, Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida; Cydia funebrana; Cydia molesta; Cydia nigricana; Cydia pomonella; Cydia splendana, Cylas formicarius; Datana integerrima, Datana major; Dermacentor albipictus; Dermacentor andersoni; Dermacentor occidentalis; Dermacentor variabilis; Diabrotica spp.; Diabrotica virgifera; Diaphania hyalinata; Diaprepes abbreviatus; Diatraea crambidoides; Diatraea saccharalis; Diatraea spp.; Dorysthenes buqueti; Dryocampa rubicunda; Earias insulana; Ennomos subsignaria; Ephestia elutella, Epilachna varivestis Mulsant, Epiphyas postvittana, Erinnyis ello; Erionota thrax; Estigmene acrea, Euetheola humilis; Eumorpha achemon; Euwallacea fornicatus; Euxesta stigmatias; Evergestis rimosalis; Grapholita delineana; Grapholita molesta; Grapholita packardi; Halyomorpha halys; Harrisina americana, Helicoverpa armigera, Helicoverpa spp.; Helicoverpa zea, Heliothis virescens; Heterocampa guttivitta; Homadaula anisocentra; Hypera postica, Hypera zoilus; Hypercompe scribonia, Hyphantria cunea; Hypothenemus hampei; Ixodes cookei; Ixodes pacificus; Ixodes scapularis; Lambdina fiscellaria; Lasiocampidae sp.; Leptinotarsa decemlineata, Lissorhoptrus oryzophilus; Listronotus maculicollis; Lobesia botrana, Lymantria dispar; Maladera castanea; Maliarpha separatella, Manduca quinquemaculata, Manduca sexta, Melanagromyza soja Zehntner; Melissopus latiferreanus; Neophasia menapia; Noctua pronuba; Noctuid sp.; Nymphula stagnata; Orgyia pseudotsugata; Orgyia vetusta; Ostrinia furnacalis; Ostrinia nubilalis; Otiorhynchus ovatus; Paleacrita merriccata; Pandemis cerasana, Papilio cresphontes; Paralobesia viteana, Pectinophora gossypiella, Pegomya betae; Pemphigus betae; Peridroma saucia, Phryganidia californica, Phyllophaga sp.; Pieris rapae; Plathypena scabra; Platynota flavedana; Platynota idaeusalis; Platynota stultana, Platyptilia carduidactyla, Playnota stultana; Plodia interpunctella, Plutella xylostella, Popillia japonica, Pseudaletia unipuncta, Pseudoplusia includens; Rhipicephalus sangunineus; Rhizotroqus majalis; Sabulodes aegrotata; Schizura concinna; Scirpophaga incertulas; Scirpophaga innotata, Scutigerella immaculata; Sesamia calamistis; Sibine stimulea, Sminthurus viridis; Sparganothis pilleriana, Sphenophorus apicalis; Sphenophorus cariosus; Sphenophorus inaequalis; Sphenophorus minimus; Sphenophorus parvulus; Sphenophorus spp.; Spilonota ocellana; Spodoptera exigua; Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola; Thyridopteryx ephemeraeformis; Thysanoptera sp.; Trichoplusia ni, Xanthogaleruca luteola, Xylomyges curialis; and Xylotrechus quadripes.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99% or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein the KPV comprises at least one amino acid substitution relative to the amino acid sequence set forth in SEQ ID NO: 1; or an agriculturally acceptable salt thereof; and wherein the pest is selected from the group comprising Acariformes sp.; Acleris gloverana, Acrobasis vaccinii; Adoxophyes orana; Aedes aegypti, Agrotis ipsilon, Aleyrodidae sp.; Alsophila pometaria; Ambylomma americanum; Amblyomma maculatum; Amorbia humerosana, Anarsia lineatella; Anomala orientalis; Anthonomus eugenii, Anticarsia gemmatalis; Aphidoidea sp.; Aphis glycines Matsumura; Archips argyrospilia; Archips podana; Archips rosana; Argyrotaenia citrana; Argyrotaenia ljungiana; Argyrotaenia velutinana, Automeris io; Batrachedra comosae; Busseola fusca; Caudra cautella; Cerotoma spp.; Chaetocnema concinna; Chilo auricilius; Chilo partellus; Chilo suppressalis; Choristoneura fumiferana; Choristoneura pinus; Choristoneura rosaceana; Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida, Cydia funebrana; Cydia molesta; Cydia nigricana; Cydia pomonella; Cydia splendana; Cylas formicarius; Datana integerrima; Datana major; Dermacentor albipictus; Dermacentor andersoni; Dermacentor occidentalis; Dermacentor variabilis; Diabrotica spp.; Diabrotica virgifera; Diaphania hyalinata; Diaprepes abbreviatus; Diatraea crambidoides; Diatraea saccharalis; Diatraea spp.; Dorysthenes buqueti, Dryocampa rubicunda, Earias insulana, Ennomos subsignaria; Ephestia elutella, Epilachna varivestis Mulsant; Epiphyas postvittana, Erinnyis ello; Erionota thrax; Estigmene acrea; Euetheola humilis; Eumorpha achemon; Euwallacea fornicatus; Euxesta stigmatias; Evergestis rimosalis; Grapholita delineana, Grapholita molesta; Grapholita packardi; Halyomorpha halys; Harrisina americana, Helicoverpa armigera, Helicoverpa spp.; Helicoverpa zea; Heliothis virescens; Heterocampa guttivitta, Homadaula anisocentra; Hypera postica; Hypera zoilus; Hypercompe scribonia; Hyphantria cunea; Hypothenemus hampei; Ixodes cookei; Ixodes pacificus; Ixodes scapularis; Lambdina fiscellaria, Lasiocampidae sp.; Leptinotarsa decemlineata, Lissorhoptrus oryzophilus; Listronotus maculicollis; Lobesia botrana, Lymantria dispar; Maladera castanea; Maliarpha separatella, Manduca quinquemaculata; Manduca sexta; Melanagromyza soja Zehntner; Melissopus latiferreanus; Neophasia menapia; Noctua pronuba, Noctuid sp.; Nymphula stagnata; Orgyia pseudotsugata; Orgyia vetusta; Ostrinia furnacalis; Ostrinia nubilalis; Otiorhynchus ovatus; Paleacrita merriccata, Pandemis cerasana, Papilio cresphontes; Paralobesia viteana, Pectinophora gossypiella, Pegomya betae; Pemphigus betae; Peridroma saucia, Phryganidia californica, Phyllophaga sp.; Pieris rapae; Plathypena scabra, Platynota flavedana; Platynota idaeusalis; Platynota stultana, Platyptilia carduidactyla, Playnota stultana; Plodia interpunctella, Plutella xylostella, Popillia japonica; Pseudaletia unipuncta, Pseudoplusia includens; Rhipicephalus sangunineus; Rhizotroqus majalis; Sabulodes aegrotata, Schizura concinna; Scirpophaga incertulas; Scirpophaga innotata; Scutigerella immaculata, Sesamia calamistis; Sibine stimulea; Sminthurus viridis; Sparganothis pilleriana; Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua, Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni; Xanthogaleruca luteola; Xylomyges curialis; or Xylotrechus quadripes.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group comprising Acariformes sp.; Acleris gloverana, Acrobasis vaccinii; Adoxophyes orana; Aedes aegypti; Agrotis ipsilon; Aleyrodidae sp.; Alsophila pometaria, Ambylomma americanum; Amblyomma maculatum; Amorbia humerosana, Anarsia lineatella, Anomala orientalis; Anthonomus eugenii; Anticarsia gemmatalis; Aphidoidea sp.; Aphis glycines Matsumura; Archips argyrospilia, Archips podana, Archips rosana, Argyrotaenia citrana; Argyrotaenia ljungiana; Argyrotaenia velutinana; Automeris io; Batrachedra comosae; Busseola fusca; Caudra cautella, Cerotoma spp.; Chaetocnema concinna; Chilo auricilius; Chilo partellus; Chilo suppressalis; Choristoneura fumiferana, Choristoneura pinus; Choristoneura rosaceana; Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida; Cydia funebrana, Cydia molesta; Cydia nigricana, Cydia pomonella; Cydia splendana, Cylas formicarius; Datana integerrima, Datana major; Dermacentor albipictus; Dermacentor andersoni; Dermacentor occidentalis; Dermacentor variabilis; Diabrotica spp.; Diabrotica virgifera, Diaphania hyalinata; Diaprepes abbreviatus; Diatraea crambidoides; Diatraea saccharalis; Diatraea spp.; Dorysthenes buqueti; Dryocampa rubicunda; Earias insulana; Ennomos subsignaria; Ephestia elutella; Epilachna varivestis Mulsant, Epiphyas postvittana, Erinnyis ello; Erionota thrax; Estigmene acrea; Euetheola humilis; Eumorpha achemon; Euwallacea fornicatus; Euxesta stigmatias; Evergestis rimosalis; Grapholita delineana; Grapholita molesta, Grapholita packardi, Halyomorpha halys; Harrisina americana, Helicoverpa armigera, Helicoverpa spp.; Helicoverpa zea, Heliothis virescens; Heterocampa guttivitta, Homadaula anisocentra; Hypera postica; Hypera zoilus; Hypercompe scribonia, Hyphantria cunea, Hypothenemus hampei, Ixodes cookei; Ixodes pacificus; Ixodes scapularis; Lambdina fiscellaria, Lasiocampidae sp.; Leptinotarsa decemlineata, Lissorhoptrus oryzophilus; Listronotus maculicollis; Lobesia botrana; Lymantria dispar; Maladera castanea; Maliarpha separatella, Manduca quinquemaculata, Manduca sexta; Melanagromyza soja Zehntner; Melissopus latiferreanus; Neophasia menapia, Noctua pronuba, Noctuid sp.; Nymphula stagnata; Orgyia pseudotsugata; Orgyia vetusta, Ostrinia furnacalis; Ostrinia nubilalis; Otiorhynchus ovatus; Paleacrita merriccata; Pandemis cerasana, Papilio cresphontes; Paralobesia viteana; Pectinophora gossypiella, Pegomya betae; Pemphigus betae; Peridroma saucia, Phryganidia californica; Phyllophaga sp.; Pieris rapae; Plathypena scabra, Platynota flavedana; Platynota idaeusalis; Platynota stultana; Platyptilia carduidactyla, Playnota stultana; Plodia interpunctella, Plutella xylostella, Popillia japonica, Pseudaletia unipuncta; Pseudoplusia includens; Rhipicephalus sangunineus; Rhizotroqus majalis; Sabulodes aegrotata, Schizura concinna; Scirpophaga incertulas; Scirpophaga innotata, Scutigerella immaculata; Sesamia calamistis; Sibine stimulea; Sminthurus viridis; Sparganothis pilleriana, Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua, Spodoptera frugiperda, Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni, Xanthogaleruca luteola, Xylomyges curialis; or Xylotrechus quadripes.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group comprising Acariformes sp.; Acleris gloverana, Acrobasis vaccinii; Adoxophyes orana, Aedes aegypti; Agrotis ipsilon, Aleyrodidae sp.; Alsophila pometaria, Ambylomma americanum; Amblyomma maculatum; Amorbia humerosana, Anarsia lineatella; Anomala orientalis; Anthonomus eugenii; Anticarsia gemmatalis; Aphidoidea sp.; Aphis glycines Matsumura; Archips argyrospilia, Archips podana; Archips rosana; Argyrotaenia citrana; Argyrotaenia ljungiana; Argyrotaenia velutinana; Automeris io; Batrachedra comosae; Busseola fusca, Caudra cautella, Cerotoma spp.; Chaetocnema concinna, Chilo auricilius; Chilo partellus; Chilo suppressalis; Choristoneura fumiferana, Choristoneura pinus; Choristoneura rosaceana; Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida, Cydia funebrana; Cydia molesta; Cydia nigricana, Cydia pomonella; Cydia splendana, Cylas formicarius; Datana integerrima; Datana major; Dermacentor albipictus; Dermacentor andersoni; Dermacentor occidentalis; Dermacentor variabilis; Diabrotica spp.; Diabrotica virgifera, Diaphania hyalinata, Diaprepes abbreviatus; Diatraea crambidoides; Diatraea saccharalis; Diatraea spp.; Dorysthenes buqueti, Dryocampa rubicunda, Earias insulana; Ennomos subsignaria; Ephestia elutella, Epilachna varivestis Mulsant, Epiphyas postvittana, Erinnyis ello; Erionota thrax; Estigmene acrea; Euetheola humilis; Eumorpha achemon; Euwallacea fornicatus; Euxesta stigmatias; Evergestis rimosalis; Grapholita delineana; Grapholita molesta, Grapholita packardi; Halyomorpha halys; Harrisina americana, Helicoverpa armigera; Helicoverpa spp.; Helicoverpa zea, Heliothis virescens; Heterocampa guttivitta, Homadaula anisocentra; Hypera postica, Hypera zoilus; Hypercompe scribonia, Hyphantria cunea; Hypothenemus hampei; Ixodes cookei; Ixodes pacificus; Ixodes scapularis; Lambdina fiscellaria, Lasiocampidae sp.; Leptinotarsa decemlineata; Lissorhoptrus oryzophilus; Listronotus maculicollis; Lobesia botrana, Lymantria dispar; Maladera castanea, Maliarpha separatella, Manduca quinquemaculata, Manduca sexta, Melanagromyza soja Zehntner; Melissopus latiferreanus; Neophasia menapia; Noctua pronuba, Noctuid sp.; Nymphula stagnata; Orgyia pseudotsugata; Orgyia vetusta; Ostrinia furnacalis; Ostrinia nubilalis; Otiorhynchus ovatus; Paleacrita merriccata; Pandemis cerasana, Papilio cresphontes; Paralobesia viteana; Pectinophora gossypiella, Pegomya betae; Pemphigus betae; Peridroma saucia; Phryganidia californica; Phyllophaga sp.; Pieris rapae; Plathypena scabra; Platynota flavedana; Platynota idaeusalis; Platynota stultana; Platyptilia carduidactyla; Playnota stultana; Plodia interpunctella, Plutella xylostella, Popillia japonica; Pseudaletia unipuncta; Pseudoplusia includens; Rhipicephalus sangunineus; Rhizotroqus majalis, Sabulodes aegrotata, Schizura concinna; Scirpophaga incertulas; Scirpophaga innotata, Scutigerella immaculata; Sesamia calamistis; Sibine stimulea, Sminthurus viridis; Sparganothis pilleriana, Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua; Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta; Thecla basilides; Thymelicus lineola; Thyridopteryx ephemeraeformis; Thysanoptera sp.; Trichoplusia ni, Xanthogaleruca luteola, Xylomyges curialis; or Xylotrechus quadripes.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is selected from the group consisting of: Aedes aegypti; Busseola fusca; Chilo suppressalis; Culex pipiens; Culex quinquefasciatus; Diabrotica virgifera; Diatraea saccharalis; Helicoverpa armigera; Helicoverpa zea, Heliothis virescens; Leptinotarsa decemlineata, Ostrinia furnacalis; Ostrinia nubilalis; Pectinophora gossypiella; Plodia interpunctella; Plutella xylostella, Pseudoplusia includens; Spodoptera exigua, Spodoptera frugiperda; Spodoptera littoralis; Trichoplusia ni; and Xanthogaleruca luteola.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence that is at least 90%, 95%, 96%, 97%, 97%, 98%, 99% or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein the KPV comprises at least one amino acid substitution relative to the amino acid sequence set forth in SEQ ID NO: 1; or an agriculturally acceptable salt thereof; and wherein the pest is selected from the group consisting of: Aedes aegypti; Busseola fusca, Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata; Ostrinia furnacalis; Ostrinia nubilalis; Pectinophora gossypiella; Plodia interpunctella, Plutella xylostella, Pseudoplusia includens; Spodoptera exigua, Spodoptera frugiperda, Spodoptera littoralis; Trichoplusia ni; and Xanthogaleruca luteola.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group consisting of: Aedes aegypti; Busseola fusca; Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata; Ostrinia furnacalis; Ostrinia nubilalis; Pectinophora gossypiella, Plodia interpunctella, Plutella xylostella, Pseudoplusia includens; Spodoptera exigua, Spodoptera frugiperda, Spodoptera littoralis; Trichoplusia ni, and Xanthogaleruca luteola.

In some embodiments, a method of combating, controlling, or inhibiting a pest comprises: applying a pesticidally effective amount of an Kappa Peptide Variant (KPV) to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV has pesticidal activity against one or more pest species, and comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group consisting of: Aedes aegypti; Busseola fusca; Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata; Ostrinia furnacalis; Ostrinia nubilalis; Pectinophora gossypiella; Plodia interpunctella, Plutella xylostella, Pseudoplusia includens; Spodoptera exigua; Spodoptera frugiperda; Spodoptera littoralis; Trichoplusia ni; and Xanthogaleruca luteola.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein each of the KPVs independently comprises at least one amino acid substitution relative to the amino acid sequence set forth in SEQ ID NO: 1, with the proviso that the KPVs do not comprise the amino acid sequence of SEQ ID NOs: 1 or 253.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein each of the two or more KPVs independently consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a homopolymer or heteropolymer of two or more KPVs, wherein the amino acid sequence of each KPV is the same or different.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable or non-cleavable linker, and wherein the amino acid sequence of each KPV may be the same or different.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇, wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different; wherein the linker has an amino acid sequence as set forth in any one of SEQ ID NOs: 360-369.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different; wherein the linker is cleavable inside at least one of (i) the gut or hemolymph of an insect, and (ii) cleavable inside the gut of a mammal.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is selected from the group comprising Acariformes sp.; Acleris gloverana, Acrobasis vaccinii; Adoxophyes orana, Aedes aegypti; Agrotis ipsilon, Aleyrodidae sp.; Alsophila pometaria; Ambylomma americanum; Amblyomma maculatum; Amorbia humerosana, Anarsia lineatella; Anomala orientalis; Anthonomus eugenii; Anticarsia gemmatalis; Aphidoidea sp.; Aphis glycines Matsumura; Archips argyrospilia, Archips podana, Archips rosana, Argyrotaenia citrana, Argyrotaenia ljungiana; Argyrotaenia velutinana, Automeris io; Batrachedra comosae; Busseola fusca; Caudra cautella, Cerotoma spp.; Chaetocnema concinna, Chilo auricilius; Chilo partellus; Chilo suppressalis; Choristoneura fumiferana; Choristoneura pinus; Choristoneura rosaceana, Chrysodeixis includens Walker; Circulifer tenellus; Colias eurytheme; Coniesta ignefusalis; Conoderus rudis; Cosmopolites sordidus; Cotinis nitida, Crambus sp.; Crocidosema spp.; Culex pipiens; Culex quinquefasciatus; Curculio caryae; Curculio occidentis; Cyclocephala borealis; Cyclocephala lurida; Cydia funebrana; Cydia molesta, Cydia nigricana, Cydia pomonella; Cydia splendana; Cylas formicarius; Datana integerrima; Datana major; Dermacentor albipictus; Dermacentor andersoni, Dermacentor occidentalis; Dermacentor variabilis; Diabrotica spp.; Diabrotica virgifera, Diaphania hyalinata, Diaprepes abbreviatus; Diatraea crambidoides; Diatraea saccharalis; Diatraea spp.; Dorysthenes buqueti, Dryocampa rubicunda, Earias insulana; Ennomos subsignaria, Ephestia elutella, Epilachna varivestis Mulsant, Epiphyas postvittana, Erinnyis ello; Erionota thrax; Estigmene acrea, Euetheola humilis; Eumorpha achemon; Euwallacea fornicatus; Euxesta stigmatias; Evergestis rimosalis; Grapholita delineana; Grapholita molesta; Grapholita packardi; Halyomorpha halys; Harrisina americana, Helicoverpa armigera, Helicoverpa spp.; Helicoverpa zea, Heliothis virescens; Heterocampa guttivitta, Homadaula anisocentra; Hypera postica; Hypera zoilus; Hypercompe scribonia, Hyphantria cunea, Hypothenemus hampei; Ixodes cookei, Ixodes pacificus; Ixodes scapularis; Lambdina fiscellaria; Lasiocampidae sp.; Leptinotarsa decemlineata, Lissorhoptrus oryzophilus; Listronotus maculicollis; Lobesia botrana; Lymantria dispar; Maladera castanea, Maliarpha separatella, Manduca quinquemaculata; Manduca sexta; Melanagromyza soja Zehntner; Melissopus latiferreanus; Neophasia menapia, Noctua pronuba, Noctuid sp.; Nymphula stagnata; Orgyia pseudotsugata; Orgyia vetusta; Ostrinia furnacalis; Ostrinia nubilalis; Otiorhynchus ovatus; Paleacrita merriccata; Pandemis cerasana, Papilio cresphontes; Paralobesia viteana, Pectinophora gossypiella, Pegomya betae; Pemphigus betae; Peridroma saucia; Phryganidia californica; Phyllophaga sp.; Pieris rapae; Plathypena scabra; Platynota flavedana, Platynota idaeusalis; Platynota stultana, Platyptilia carduidactyla, Playnota stultana; Plodia interpunctella, Plutella xylostella, Popillia japonica; Pseudaletia unipuncta, Pseudoplusia includens; Rhipicephalus sangunineus; Rhizotroqus majalis; Sabulodes aegrotata, Schizura concinna, Scirpophaga incertulas; Scirpophaga innotata, Scutigerella immaculata; Sesamia calamistis; Sibine stimulea, Sminthurus viridis; Sparganothis pilleriana; Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua, Spodoptera frugiperda, Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta; Thecla basilides; Thymelicus lineola, Thyridopteryx ephemeraeformis; Thysanoptera sp.; Trichoplusia ni, Xanthogaleruca luteola, Xylomyges curialis; or Xylotrechus quadripes.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein each of the KPVs independently comprises at least one amino acid substitution relative to the amino acid sequence set forth in SEQ ID NO: 1, with the proviso that the KPVs do not comprise the amino acid sequence of SEQ ID NOs: 1 or 253; or an agriculturally acceptable salt thereof; and wherein the pest is selected from the group comprising Acariformes sp.; Acleris gloverana, Acrobasis vaccinii; Adoxophyes orana; Aedes aegypti; Agrotis ipsilon; Aleyrodidae sp.; Alsophila pometaria; Ambylomma americanum; Amblyomma maculatum; Amorbia humerosana; Anarsia lineatella, Anomala orientalis; Anthonomus eugenii; Anticarsia gemmatalis; Aphidoidea sp.; Aphis glycines Matsumura, Archips argyrospilia, Archips podana, Archips rosana, Argyrotaenia citrana, Argyrotaenia ljungiana; Argyrotaenia velutinana, Automeris io; Batrachedra comosae; Busseola fusca; Caudra cautella, Cerotoma spp.; Chaetocnema concinna; Chilo auricilius; Chilo partellus; Chilo suppressalis; Choristoneura fumiferana, Choristoneura pinus; Choristoneura rosaceana; Chrysodeixis includens Walker, Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida, Cydia funebrana, Cydia molesta; Cydia nigricana; Cydia pomonella; Cydia splendana, Cylas formicarius; Datana integerrima, Datana major; Dermacentor albipictus; Dermacentor andersoni; Dermacentor occidentalis; Dermacentor variabilis; Diabrotica spp.; Diabrotica virgifera, Diaphania hyalinata; Diaprepes abbreviatus; Diatraea crambidoides; Diatraea saccharalis; Diatraea spp.; Dorysthenes buqueti, Dryocampa rubicunda; Earias insulana, Ennomos subsignaria; Ephestia elutella, Epilachna varivestis Mulsant, Epiphyas postvittana, Erinnyis ello; Erionota thrax; Estigmene acrea, Euetheola humilis; Eumorpha achemon; Euwallacea fornicatus; Euxesta stigmatias; Evergestis rimosalis; Grapholita delineana; Grapholita molesta; Grapholita packardi; Halyomorpha halys; Harrisina americana, Helicoverpa armigera; Helicoverpa spp.; Helicoverpa zea, Heliothis virescens; Heterocampa guttivitta, Homadaula anisocentra, Hypera postica, Hypera zoilus; Hypercompe scribonia, Hyphantria cunea, Hypothenemus hampei; Ixodes cookei, Ixodes pacificus; Ixodes scapularis; Lambdina fiscellaria; Lasiocampidae sp.; Leptinotarsa decemlineata, Lissorhoptrus oryzophilus; Listronotus maculicollis; Lobesia botrana; Lymantria dispar; Maladera castanea, Maliarpha separatella, Manduca quinquemaculata, Manduca sexta, Melanagromyza soja Zehntner; Melissopus latiferreanus; Neophasia menapia, Noctua pronuba, Noctuid sp.; Nymphula stagnata; Orgyia pseudotsugata; Orgyia vetusta, Ostrinia furnacalis; Ostrinia nubilalis; Otiorhynchus ovatus; Paleacrita merriccata, Pandemis cerasana; Papilio cresphontes; Paralobesia viteana, Pectinophora gossypiella, Pegomya betae; Pemphigus betae; Peridroma saucia; Phryganidia californica, Phyllophaga sp.; Pieris rapae; Plathypena scabra; Platynota flavedana, Platynota idaeusalis; Platynota stultana, Platyptilia carduidactyla, Playnota stultana; Plodia interpunctella, Plutella xylostella, Popillia japonica, Pseudaletia unipuncta; Pseudoplusia includens; Rhipicephalus sangunineus; Rhizotroqus majalis; Sabulodes aegrotata; Schizura concinna; Scirpophaga incertulas; Scirpophaga innotata, Scutigerella immaculata; Sesamia calamistis; Sibine stimulea, Sminthurus viridis; Sparganothis pilleriana, Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua, Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni; Xanthogaleruca luteola, Xylomyges curialis; or Xylotrechus quadripes.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein each of the two or more KPVs independently comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group comprising Acariformes sp.; Acleris gloverana, Acrobasis vaccinii; Adoxophyes orana; Aedes aegypti, Agrotis ipsilon, Aleyrodidae sp.; Alsophila pometaria; Ambylomma americanum; Amblyomma maculatum; Amorbia humerosana; Anarsia lineatella, Anomala orientalis; Anthonomus eugenii, Anticarsia gemmatalis; Aphidoidea sp.; Aphis glycines Matsumura; Archips argyrospilia; Archips podana, Archips rosana, Argyrotaenia citrana; Argyrotaenia ljungiana; Argyrotaenia velutinana, Automeris io; Batrachedra comosae; Busseola fusca, Caudra cautella, Cerotoma spp.; Chaetocnema concinna; Chilo auricilius; Chilo partellus; Chilo suppressalis; Choristoneura fumiferana, Choristoneura pinus; Choristoneura rosaceana, Chrysodeixis includens Walker, Circulifer tenellus; Colias eurytheme; Coniesta ignefusalis; Conoderus rudis; Cosmopolites sordidus; Cotinis nitida, Crambus sp.; Crocidosema spp.; Culex pipiens; Culex quinquefasciatus; Curculio caryae; Curculio occidentis; Cyclocephala borealis; Cyclocephala lurida; Cydia funebrana; Cydia molesta; Cydia nigricana; Cydia pomonella, Cydia splendana, Cylas formicarius; Datana integerrima, Datana major; Dermacentor albipictus; Dermacentor andersoni, Dermacentor occidentalis; Dermacentor variabilis; Diabrotica spp.; Diabrotica virgifera, Diaphania hyalinata; Diaprepes abbreviatus; Diatraea crambidoides; Diatraea saccharalis; Diatraea spp.; Dorysthenes buqueti; Dryocampa rubicunda, Earias insulana, Ennomos subsignaria; Ephestia elutella, Epilachna varivestis Mulsant, Epiphyas postvittana; Erinnyis ello; Erionota thrax; Estigmene acrea; Euetheola humilis; Eumorpha achemon; Euwallacea fornicatus; Euxesta stigmatias; Evergestis rimosalis; Grapholita delineana, Grapholita molesta; Grapholita packardi; Halyomorpha halys; Harrisina americana, Helicoverpa armigera; Helicoverpa spp.; Helicoverpa zea, Heliothis virescens; Heterocampa guttivitta, Homadaula anisocentra; Hypera postica; Hypera zoilus; Hypercompe scribonia, Hyphantria cunea, Hypothenemus hampei; Ixodes cookei, Ixodes pacificus; Ixodes scapularis; Lambdina fiscellaria, Lasiocampidae sp.; Leptinotarsa decemlineata, Lissorhoptrus oryzophilus; Listronotus maculicollis; Lobesia botrana, Lymantria dispar; Maladera castanea, Maliarpha separatella, Manduca quinquemaculata, Manduca sexta; Melanagromyza soja Zehntner; Melissopus latiferreanus; Neophasia menapia, Noctua pronuba, Noctuid sp.; Nymphula stagnata; Orgyia pseudotsugata; Orgyia vetusta, Ostrinia furnacalis; Ostrinia nubilalis; Otiorhynchus ovatus; Paleacrita merriccata, Pandemis cerasana, Papilio cresphontes; Paralobesia viteana, Pectinophora gossypiella, Pegomya betae; Pemphigus betae; Peridroma saucia; Phryganidia californica; Phyllophaga sp.; Pieris rapae; Plathypena scabra, Platynota flavedana; Platynota idaeusalis; Platynota stultana, Platyptilia carduidactyla, Playnota stultana; Plodia interpunctella, Plutella xylostella, Popillia japonica; Pseudaletia unipuncta; Pseudoplusia includens; Rhipicephalus sangunineus; Rhizotroqus majalis; Sabulodes aegrotata; Schizura concinna; Scirpophaga incertulas; Scirpophaga innotata, Scutigerella immaculata; Sesamia calamistis; Sibine stimulea, Sminthurus viridis; Sparganothis pilleriana; Sphenophorus apicalis; Sphenophorus cariosus; Sphenophorus inaequalis; Sphenophorus minimus; Sphenophorus parvulus; Sphenophorus spp.; Spilonota ocellana; Spodoptera exigua, Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni; Xanthogaleruca luteola, Xylomyges curialis; or Xylotrechus quadripes.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group comprising Acariformes sp.; Acleris gloverana, Acrobasis vaccinii, Adoxophyes orana; Aedes aegypti; Agrotis ipsilon, Aleyrodidae sp.; Alsophila pometaria, Ambylomma americanum; Amblyomma maculatum; Amorbia humerosana, Anarsia lineatella; Anomala orientalis; Anthonomus eugenii; Anticarsia gemmatalis; Aphidoidea sp.; Aphis glycines Matsumura; Archips argyrospilia, Archips podana; Archips rosana, Argyrotaenia citrana; Argyrotaenia ljungiana; Argyrotaenia velutinana, Automeris io; Batrachedra comosae; Busseola fusca; Caudra cautella; Cerotoma spp.; Chaetocnema concinna; Chilo auricilius; Chilo partellus; Chilo suppressalis; Choristoneura fumiferana; Choristoneura pinus; Choristoneura rosaceana, Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida, Cydia funebrana; Cydia molesta, Cydia nigricana; Cydia pomonella; Cydia splendana; Cylas formicarius; Datana integerrima; Datana major; Dermacentor albipictus; Dermacentor andersoni; Dermacentor occidentalis; Dermacentor variabilis; Diabrotica spp.; Diabrotica virgifera, Diaphania hyalinata, Diaprepes abbreviatus; Diatraea crambidoides; Diatraea saccharalis; Diatraea spp.; Dorysthenes buqueti; Dryocampa rubicunda, Earias insulana; Ennomos subsignaria; Ephestia elutella; Epilachna varivestis Mulsant, Epiphyas postvittana, Erinnyis ello; Erionota thrax; Estigmene acrea; Euetheola humilis; Eumorpha achemon; Euwallacea fornicatus; Euxesta stigmatias; Evergestis rimosalis; Grapholita delineana, Grapholita molesta, Grapholita packardi, Halyomorpha halys; Harrisina americana; Helicoverpa armigera, Helicoverpa spp.; Helicoverpa zea, Heliothis virescens; Heterocampa guttivitta; Homadaula anisocentra; Hypera postica; Hypera zoilus; Hypercompe scribonia; Hyphantria cunea, Hypothenemus hampei; Ixodes cookei; Ixodes pacificus; Ixodes scapularis; Lambdina fiscellaria, Lasiocampidae sp.; Leptinotarsa decemlineata; Lissorhoptrus oryzophilus; Listronotus maculicollis; Lobesia botrana, Lymantria dispar; Maladera castanea; Maliarpha separatella; Manduca quinquemaculata, Manduca sexta, Melanagromyza soja Zehntner; Melissopus latiferreanus; Neophasia menapia, Noctua pronuba, Noctuid sp.; Nymphula stagnata; Orgyia pseudotsugata; Orgyia vetusta; Ostrinia furnacalis; Ostrinia nubilalis; Otiorhynchus ovatus; Paleacrita merriccata, Pandemis cerasana, Papilio cresphontes; Paralobesia viteana, Pectinophora gossypiella, Pegomya betae; Pemphigus betae; Peridroma saucia, Phryganidia californica, Phyllophaga sp.; Pieris rapae; Plathypena scabra; Platynota flavedana, Platynota idaeusalis; Platynota stultana; Platyptilia carduidactyla; Playnota stultana; Plodia interpunctella, Plutella xylostella; Popillia japonica, Pseudaletia unipuncta; Pseudoplusia includens; Rhipicephalus sangunineus; Rhizotroqus majalis; Sabulodes aegrotata, Schizura concinna; Scirpophaga incertulas; Scirpophaga innotata, Scutigerella immaculata; Sesamia calamistis; Sibine stimulea, Sminthurus viridis; Sparganothis pilleriana, Sphenophorus apicalis; Sphenophorus cariosus; Sphenophorus inaequalis; Sphenophorus minimus; Sphenophorus parvulus; Sphenophorus spp.; Spilonota ocellana; Spodoptera exigua, Spodoptera frugiperda, Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni; Xanthogaleruca luteola, Xylomyges curialis; or Xylotrechus quadripes.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is selected from the group consisting of: Aedes aegypti; Busseola fusca, Chilo suppressalis; Culex pipiens; Culex quinquefasciatus; Diabrotica virgifera, Diatraea saccharalis; Helicoverpa armigera; Helicoverpa zea, Heliothis virescens; Leptinotarsa decemlineata, Ostrinia furnacalis; Ostrinia nubilalis; Pectinophora gossypiella, Plodia interpunctella, Plutella xylostella; Pseudoplusia includens; Spodoptera exigua; Spodoptera frugiperda, Spodoptera littoralis; Trichoplusia ni; and Xanthogaleruca luteola.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein each of the KPVs independently comprises at least one amino acid substitution relative to the amino acid sequence set forth in SEQ ID NO: 1, with the proviso that the KPVs do not comprise the amino acid sequence of SEQ ID NOs: 1 or 253; or an agriculturally acceptable salt thereof; and wherein the pest is selected from the group consisting of: Aedes aegypti, Busseola fusca, Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata, Ostrinia furnacalis; Ostrinia nubilalis; Pectinophora gossypiella, Plodia interpunctella, Plutella xylostella; Pseudoplusia includens; Spodoptera exigua, Spodoptera frugiperda, Spodoptera littoralis; Trichoplusia ni; and Xanthogaleruca luteola.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein each of the two or more KPVs independently comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group consisting of: Aedes aegypti, Busseola fusca, Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata, Ostrinia furnacalis; Ostrinia nubilalis; Pectinophora gossypiella, Plodia interpunctella, Plutella xylostella; Pseudoplusia includens; Spodoptera exigua; Spodoptera frugiperda; Spodoptera littoralis; Trichoplusia ni; and Xanthogaleruca luteola.

In some embodiments, a method of combating, controlling, or inhibiting a pest comprises: applying a pesticidally effective amount of a combination two or more Kappa Peptide Variant (KPVs) having pesticidal activity against one or more pest species, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein each of the two or more KPVs independently consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group consisting of: Aedes aegypti; Busseola fusca; Chilo suppressalis; Culex pipiens; Culex quinquefasciatus; Diabrotica virgifera, Diatraea saccharalis; Helicoverpa armigera, Helicoverpa zea; Heliothis virescens; Leptinotarsa decemlineata, Ostrinia furnacalis; Ostrinia nubilalis; Pectinophora gossypiella; Plodia interpunctella, Plutella xylostella, Pseudoplusia includens; Spodoptera exigua; Spodoptera frugiperda, Spodoptera littoralis; Trichoplusia ni; and Xanthogaleruca luteola.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence that is at least 90%, 95%, 96%, 97%, 97%, 98%, 99% or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein the KPV comprises at least one amino acid substitution relative to the amino acid sequence set forth in SEQ ID NO: 1; or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV consists of an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a homopolymer or heteropolymer of two or more KPVs, wherein the amino acid sequence of each KPV is the same or different.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable or non-cleavable linker, and wherein the amino acid sequence of each KPV may be the same or different.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different; wherein the linker has an amino acid sequence as set forth in any one of SEQ ID NOs: 360-369.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable linker, and wherein the amino acid sequence of each KPV may be the same or different; wherein the linker is cleavable inside at least one of (i) the gut or hemolymph of an insect, and (ii) cleavable inside the gut of a mammal.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is selected from the group comprising Acariformes sp.; Acleris gloverana; Acrobasis vaccinii; Adoxophyes orana; Aedes aegypti; Agrotis ipsilon, Aleyrodidae sp.; Alsophila pometaria, Ambylomma americanum; Amblyomma maculatum; Amorbia humerosana; Anarsia lineatella; Anomala orientalis; Anthonomus eugenii, Anticarsia gemmatalis; Aphidoidea sp.; Aphis glycines Matsumura, Archips argyrospilia, Archips podana, Archips rosana; Argyrotaenia citrana; Argyrotaenia ljungiana; Argyrotaenia velutinana, Automeris io; Batrachedra comosae; Busseola fusca; Caudra cautella, Cerotoma spp.; Chaetocnema concinna; Chilo auricilius; Chilo partellus; Chilo suppressalis; Choristoneura fumiferana, Choristoneura pinus; Choristoneura rosaceana, Chrysodeixis includens Walker; Circulifer tenellus; Colias eurytheme; Coniesta ignefusalis; Conoderus rudis; Cosmopolites sordidus; Cotinis nitida, Crambus sp.; Crocidosema spp.; Culex pipiens; Culex quinquefasciatus; Curculio caryae; Curculio occidentis; Cyclocephala borealis; Cyclocephala lurida, Cydia funebrana, Cydia molesta; Cydia nigricana, Cydia pomonella, Cydia splendana, Cylas formicarius; Datana integerrima, Datana major; Dermacentor albipictus; Dermacentor andersoni; Dermacentor occidentalis; Dermacentor variabilis; Diabrotica spp.; Diabrotica virgifera, Diaphania hyalinata; Diaprepes abbreviatus; Diatraea crambidoides; Diatraea saccharalis; Diatraea spp.; Dorysthenes buqueti, Dryocampa rubicunda, Earias insulana, Ennomos subsignaria; Ephestia elutella; Epilachna varivestis Mulsant; Epiphyas postvittana, Erinnyis ello; Erionota thrax; Estigmene acrea, Euetheola humilis; Eumorpha achemon; Euwallacea fornicatus; Euxesta stigmatias; Evergestis rimosalis; Grapholita delineana, Grapholita molesta; Grapholita packardi; Halyomorpha halys; Harrisina americana; Helicoverpa armigera; Helicoverpa spp.; Helicoverpa zea, Heliothis virescens; Heterocampa guttivitta, Homadaula anisocentra, Hypera postica; Hypera zoilus; Hypercompe scribonia; Hyphantria cunea; Hypothenemus hampei; Ixodes cookei; Ixodes pacificus; Ixodes scapularis; Lambdina fiscellaria, Lasiocampidae sp.; Leptinotarsa decemlineata; Lissorhoptrus oryzophilus; Listronotus maculicollis; Lobesia botrana; Lymantria dispar; Maladera castanea, Maliarpha separatella; Manduca quinquemaculata, Manduca sexta; Melanagromyza soja Zehntner; Melissopus latiferreanus; Neophasia menapia, Noctua pronuba, Noctuid sp.; Nymphula stagnata; Orgyia pseudotsugata; Orgyia vetusta; Ostrinia furnacalis; Ostrinia nubilalis; Otiorhynchus ovatus; Paleacrita merriccata, Pandemis cerasana, Papilio cresphontes; Paralobesia viteana, Pectinophora gossypiella; Pegomya betae; Pemphigus betae; Peridroma saucia; Phryganidia californica, Phyllophaga sp.; Pieris rapae; Plathypena scabra; Platynota flavedana, Platynota idaeusalis; Platynota stultana; Platyptilia carduidactyla, Playnota stultana, Plodia interpunctella; Plutella xylostella; Popillia japonica; Pseudaletia unipuncta; Pseudoplusia includens; Rhipicephalus sangunineus; Rhizotroqus majalis; Sabulodes aegrotata; Schizura concinna; Scirpophaga incertulas; Scirpophaga innotata; Scutigerella immaculata; Sesamia calamistis; Sibine stimulea, Sminthurus viridis; Sparganothis pilleriana, Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua, Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni; Xanthogaleruca luteola, Xylomyges curialis; or Xylotrechus quadripes.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence that is at least 90%, 95%, 96%, 97%, 97%, 98%, 99% or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein the KPV comprises at least one amino acid substitution relative to the amino acid sequence set forth in SEQ ID NO: 1; or an agriculturally acceptable salt thereof; and wherein the pest is selected from the group comprising Acariformes sp.; Acleris gloverana, Acrobasis vaccinii; Adoxophyes orana; Aedes aegypti; Agrotis ipsilon, Aleyrodidae sp.; Alsophila pometaria; Ambylomma americanum; Amblyomma maculatum; Amorbia humerosana, Anarsia lineatella, Anomala orientalis; Anthonomus eugenii, Anticarsia gemmatalis; Aphidoidea sp.; Aphis glycines Matsumura; Archips argyrospilia; Archips podana, Archips rosana; Argyrotaenia citrana; Argyrotaenia ljungiana; Argyrotaenia velutinana; Automeris io; Batrachedra comosae; Busseola fusca; Caudra cautella; Cerotoma spp.; Chaetocnema concinna; Chilo auricilius; Chilo partellus; Chilo suppressalis; Choristoneura fumiferana; Choristoneura pinus; Choristoneura rosaceana, Chrysodeixis includens Walker; Circulifer tenellus; Colias eurytheme; Coniesta ignefusalis; Conoderus rudis; Cosmopolites sordidus; Cotinis nitida, Crambus sp.; Crocidosema spp.; Culex pipiens; Culex quinquefasciatus; Curculio caryae; Curculio occidentis; Cyclocephala borealis; Cyclocephala lurida; Cydia funebrana; Cydia molesta; Cydia nigricana; Cydia pomonella; Cydia splendana, Cylas formicarius; Datana integerrima; Datana major; Dermacentor albipictus; Dermacentor andersoni, Dermacentor occidentalis; Dermacentor variabilis; Diabrotica spp.; Diabrotica virgifera, Diaphania hyalinata, Diaprepes abbreviatus; Diatraea crambidoides; Diatraea saccharalis; Diatraea spp.; Dorysthenes buqueti, Dryocampa rubicunda, Earias insulana, Ennomos subsignaria; Ephestia elutella, Epilachna varivestis Mulsant, Epiphyas postvittana, Erinnyis ello; Erionota thrax; Estigmene acrea, Euetheola humilis; Eumorpha achemon; Euwallacea fornicatus; Euxesta stigmatias; Evergestis rimosalis; Grapholita delineana; Grapholita molesta; Grapholita packardi; Halyomorpha halys; Harrisina americana, Helicoverpa armigera; Helicoverpa spp.; Helicoverpa zea, Heliothis virescens; Heterocampa guttivitta, Homadaula anisocentra; Hypera postica; Hypera zoilus; Hypercompe scribonia; Hyphantria cunea, Hypothenemus hampei; Ixodes cookei, Ixodes pacificus; Ixodes scapularis; Lambdina fiscellaria; Lasiocampidae sp.; Leptinotarsa decemlineata; Lissorhoptrus oryzophilus; Listronotus maculicollis; Lobesia botrana; Lymantria dispar; Maladera castanea, Maliarpha separatella, Manduca quinquemaculata, Manduca sexta, Melanagromyza soja Zehntner; Melissopus latiferreanus; Neophasia menapia, Noctua pronuba, Noctuid sp.; Nymphula stagnata; Orgyia pseudotsugata; Orgyia vetusta; Ostrinia furnacalis; Ostrinia nubilalis; Otiorhynchus ovatus; Paleacrita merriccata, Pandemis cerasana, Papilio cresphontes; Paralobesia viteana; Pectinophora gossypiella, Pegomya betae; Pemphigus betae; Peridroma saucia, Phryganidia californica, Phyllophaga sp.; Pieris rapae; Plathypena scabra; Platynota flavedana; Platynota idaeusalis; Platynota stultana; Platyptilia carduidactyla, Playnota stultana; Plodia interpunctella, Plutella xylostella; Popillia japonica, Pseudaletia unipuncta; Pseudoplusia includens; Rhipicephalus sangunineus; Rhizotroqus majalis; Sabulodes aegrotata, Schizura concinna; Scirpophaga incertulas; Scirpophaga innotata, Scutigerella immaculata; Sesamia calamistis; Sibine stimulea, Sminthurus viridis; Sparganothis pilleriana, Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua; Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola; Thyridopteryx ephemeraeformis; Thysanoptera sp.; Trichoplusia ni, Xanthogaleruca luteola, Xylomyges curialis; or Xylotrechus quadripes.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein the KPV comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group comprising Acariformes sp.; Acleris gloverana; Acrobasis vaccinii; Adoxophyes orana; Aedes aegypti; Agrotis ipsilon; Aleyrodidae sp.; Alsophila pometaria; Ambylomma americanum; Amblyomma maculatum; Amorbia humerosana; Anarsia lineatella, Anomala orientalis; Anthonomus eugenii, Anticarsia gemmatalis; Aphidoidea sp.; Aphis glycines Matsumura; Archips argyrospilia, Archips podana; Archips rosana; Argyrotaenia citrana; Argyrotaenia ljungiana; Argyrotaenia velutinana; Automeris io; Batrachedra comosae; Busseola fusca; Caudra cautella, Cerotoma spp.; Chaetocnema concinna, Chilo auricilius; Chilo partellus; Chilo suppressalis; Choristoneura fumiferana, Choristoneura pinus; Choristoneura rosaceana; Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida; Cydia funebrana, Cydia molesta, Cydia nigricana, Cydia pomonella; Cydia splendana; Cylas formicarius; Datana integerrima; Datana major; Dermacentor albipictus; Dermacentor andersoni; Dermacentor occidentalis; Dermacentor variabilis; Diabrotica spp.; Diabrotica virgifera, Diaphania hyalinata, Diaprepes abbreviatus; Diatraea crambidoides; Diatraea saccharalis; Diatraea spp.; Dorysthenes buqueti; Dryocampa rubicunda, Earias insulana; Ennomos subsignaria; Ephestia elutella; Epilachna varivestis Mulsant, Epiphyas postvittana, Erinnyis ello; Erionota thrax; Estigmene acrea, Euetheola humilis; Eumorpha achemon; Euwallacea fornicatus; Euxesta stigmatias; Evergestis rimosalis; Grapholita delineana; Grapholita molesta, Grapholita packardi; Halyomorpha halys; Harrisina americana; Helicoverpa armigera, Helicoverpa spp.; Helicoverpa zea, Heliothis virescens; Heterocampa guttivitta; Homadaula anisocentra; Hypera postica; Hypera zoilus; Hypercompe scribonia, Hyphantria cunea, Hypothenemus hampei; Ixodes cookei, Ixodes pacificus; Ixodes scapularis; Lambdina fiscellaria, Lasiocampidae sp.; Leptinotarsa decemlineata; Lissorhoptrus oryzophilus; Listronotus maculicollis; Lobesia botrana, Lymantria dispar; Maladera castanea, Maliarpha separatella; Manduca quinquemaculata, Manduca sexta, Melanagromyza soja Zehntner; Melissopus latiferreanus; Neophasia menapia, Noctua pronuba; Noctuid sp.; Nymphula stagnata, Orgyia pseudotsugata; Orgyia vetusta; Ostrinia furnacalis; Ostrinia nubilalis; Otiorhynchus ovatus; Paleacrita merriccata, Pandemis cerasana; Papilio cresphontes; Paralobesia viteana; Pectinophora gossypiella, Pegomya betae; Pemphigus betae; Peridroma saucia; Phryganidia californica; Phyllophaga sp.; Pieris rapae; Plathypena scabra, Platynota flavedana, Platynota idaeusalis; Platynota stultana; Platyptilia carduidactyla; Playnota stultana; Plodia interpunctella, Plutella xylostella, Popillia japonica; Pseudaletia unipuncta; Pseudoplusia includens; Rhipicephalus sangunineus; Rhizotroqus majalis; Sabulodes aegrotata; Schizura concinna; Scirpophaga incertulas; Scirpophaga innotata, Scutigerella immaculata; Sesamia calamistis; Sibine stimulea, Sminthurus viridis; Sparganothis pilleriana; Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua, Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta; Thecla basilides; Thymelicus lineola, Thyridopteryx ephemeraeformis; Thysanoptera sp.; Trichoplusia ni, Xanthogaleruca luteola, Xylomyges curialis; or Xylotrechus quadripes.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV consists of an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group comprising Acariformes sp.; Acleris gloverana, Acrobasis vaccinii, Adoxophyes orana; Aedes aegypti; Agrotis ipsilon; Aleyrodidae sp.; Alsophila pometaria, Ambylomma americanum; Amblyomma maculatum; Amorbia humerosana, Anarsia lineatella, Anomala orientalis; Anthonomus eugenii; Anticarsia gemmatalis; Aphidoidea sp.; Aphis glycines Matsumura; Archips argyrospilia, Archips podana, Archips rosana, Argyrotaenia citrana, Argyrotaenia ljungiana; Argyrotaenia velutinana, Automeris io; Batrachedra comosae; Busseola fusca; Caudra cautella; Cerotoma spp.; Chaetocnema concinna, Chilo auricilius; Chilo partellus; Chilo suppressalis; Choristoneura fumiferana, Choristoneura pinus; Choristoneura rosaceana; Chrysodeixis includens Walker; Circulifer tenellus; Colias eurytheme; Coniesta ignefusalis; Conoderus rudis; Cosmopolites sordidus; Cotinis nitida, Crambus sp.; Crocidosema spp.; Culex pipiens; Culex quinquefasciatus; Curculio caryae; Curculio occidentis; Cyclocephala borealis; Cyclocephala lurida; Cydia funebrana; Cydia molesta; Cydia nigricana; Cydia pomonella; Cydia splendana; Cylas formicarius; Datana integerrima; Datana major; Dermacentor albipictus; Dermacentor andersoni; Dermacentor occidentalis; Dermacentor variabilis; Diabrotica spp.; Diabrotica virgifera, Diaphania hyalinata, Diaprepes abbreviatus; Diatraea crambidoides; Diatraea saccharalis; Diatraea spp.; Dorysthenes buqueti; Dryocampa rubicunda, Earias insulana; Ennomos subsignaria, Ephestia elutella, Epilachna varivestis Mulsant, Epiphyas postvittana; Erinnyis ello; Erionota thrax; Estigmene acrea; Euetheola humilis; Eumorpha achemon; Euwallacea fornicatus; Euxesta stigmatias; Evergestis rimosalis; Grapholita delineana; Grapholita molesta, Grapholita packardi; Halyomorpha halys; Harrisina americana, Helicoverpa armigera; Helicoverpa spp.; Helicoverpa zea, Heliothis virescens; Heterocampa guttivitta; Homadaula anisocentra; Hypera postica, Hypera zoilus; Hypercompe scribonia; Hyphantria cunea, Hypothenemus hampei; Ixodes cookei, Ixodes pacificus; Ixodes scapularis; Lambdina fiscellaria, Lasiocampidae sp.; Leptinotarsa decemlineata, Lissorhoptrus oryzophilus; Listronotus maculicollis; Lobesia botrana, Lymantria dispar; Maladera castanea, Maliarpha separatella, Manduca quinquemaculata; Manduca sexta, Melanagromyza soja Zehntner; Melissopus latiferreanus; Neophasia menapia, Noctua pronuba; Noctuid sp.; Nymphula stagnata; Orgyia pseudotsugata; Orgyia vetusta; Ostrinia furnacalis; Ostrinia nubilalis; Otiorhynchus ovatus; Paleacrita merriccata, Pandemis cerasana; Papilio cresphontes; Paralobesia viteana, Pectinophora gossypiella, Pegomya betae; Pemphigus betae; Peridroma saucia, Phryganidia californica; Phyllophaga sp.; Pieris rapae; Plathypena scabra; Platynota flavedana; Platynota idaeusalis; Platynota stultana; Platyptilia carduidactyla, Playnota stultana; Plodia interpunctella, Plutella xylostella, Popillia japonica; Pseudaletia unipuncta; Pseudoplusia includens; Rhipicephalus sangunineus; Rhizotroqus majalis; Sabulodes aegrotata; Schizura concinna; Scirpophaga incertulas; Scirpophaga innotata, Scutigerella immaculata; Sesamia calamistis; Sibine stimulea; Sminthurus viridis; Sparganothis pilleriana; Sphenophorus apicalis; Sphenophorus cariosus; Sphenophorus inaequalis; Sphenophorus minimus; Sphenophorus parvulus; Sphenophorus spp.; Spilonota ocellana; Spodoptera exigua, Spodoptera frugiperda, Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni, Xanthogaleruca luteola, Xylomyges curialis; or Xylotrechus quadripes.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is selected from the group consisting of: group consisting of: Aedes aegypti; Busseola fusca; Chilo suppressalis; Culex pipiens; Culex quinquefasciatus; Diabrotica virgifera; Diatraea saccharalis; Helicoverpa armigera, Helicoverpa zea, Heliothis virescens; Leptinotarsa decemlineata, Ostrinia furnacalis; Ostrinia nubilalis; Pectinophora gossypiella, Plodia interpunctella; Plutella xylostella, Pseudoplusia includens; Spodoptera exigua, Spodoptera frugiperda, Spodoptera littoralis; Trichoplusia ni; and Xanthogaleruca luteola.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence that is at least 90%, 95%, 96%, 97%, 97%, 98%, 99% or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein the KPV comprises at least one amino acid substitution relative to the amino acid sequence set forth in SEQ ID NO: 1; or an agriculturally acceptable salt thereof; and wherein the pest is selected from the group consisting of: Aedes aegypti, Busseola fusca; Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata, Ostrinia furnacalis; Ostrinia nubilalis; Pectinophora gossypiella, Plodia interpunctella, Plutella xylostella; Pseudoplusia includens; Spodoptera exigua; Spodoptera frugiperda, Spodoptera littoralis; Trichoplusia ni; and Xanthogaleruca luteola.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group consisting of: Aedes aegypti, Busseola fusca, Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata, Ostrinia furnacalis; Ostrinia nubilalis; Pectinophora gossypiella, Plodia interpunctella; Plutella xylostella, Pseudoplusia includens; Spodoptera exigua, Spodoptera frugiperda, Spodoptera littoralis; Trichoplusia ni; and Xanthogaleruca luteola.

In some embodiments, a method of combating, controlling, or inhibiting a pest comprises: applying a pesticidally effective amount of a composition comprising an Kappa Peptide Variant (KPV) having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the KPV consists of an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group consisting of: Aedes aegypti; Busseola fusca; Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata, Ostrinia furnacalis; Ostrinia nubilalis; Pectinophora gossypiella; Plodia interpunctella; Plutella xylostella; Pseudoplusia includens; Spodoptera exigua, Spodoptera frugiperda, Spodoptera littoralis; Trichoplusia ni; and Xanthogaleruca luteola.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein each of the KPVs independently comprises at least one amino acid substitution relative to the amino acid sequence set forth in SEQ ID NO: 1, or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof, with the proviso that the KPV does not comprise the amino acid sequence of SEQ ID NOs: 1 or 253.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a homopolymer or heteropolymer of two or more KPVs, wherein the amino acid sequence of each KPV is the same or different.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the KPV is a fused protein comprising two or more KPVs separated by a cleavable or non-cleavable linker, and wherein the amino acid sequence of each KPV may be the same or different.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different; wherein the linker has an amino acid sequence as set forth in any one of SEQ ID NOs: 360-369.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; wherein the two or more KPVs are a fused protein separated by a cleavable linker, and wherein the amino acid sequence of each KPV may be the same or different; wherein the linker is cleavable inside at least one of (i) the gut or hemolymph of an insect, and (ii) cleavable inside the gut of a mammal.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is selected from the group comprising Acariformes sp.; Acleris gloverana, Acrobasis vaccinii; Adoxophyes orana, Aedes aegypti, Agrotis ipsilon; Aleyrodidae sp.; Alsophila pometaria, Ambylomma americanum; Amblyomma maculatum; Amorbia humerosana; Anarsia lineatella, Anomala orientalis; Anthonomus eugenii; Anticarsia gemmatalis; Aphidoidea sp.; Aphis glycines Matsumura; Archips argyrospilia, Archips podana, Archips rosana, Argyrotaenia citrana, Argyrotaenia ljungiana; Argyrotaenia velutinana, Automeris io; Batrachedra comosae; Busseola fusca, Caudra cautella, Cerotoma spp.; Chaetocnema concinna; Chilo auricilius; Chilo partellus; Chilo suppressalis; Choristoneura fumiferana, Choristoneura pinus; Choristoneura rosaceana, Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida; Cydia funebrana; Cydia molesta, Cydia nigricana; Cydia pomonella, Cydia splendana; Cylas formicarius; Datana integerrima; Datana major; Dermacentor albipictus; Dermacentor andersoni, Dermacentor occidentalis; Dermacentor variabilis; Diabrotica spp.; Diabrotica virgifera, Diaphania hyalinata; Diaprepes abbreviatus; Diatraea crambidoides; Diatraea saccharalis; Diatraea spp.; Dorysthenes buqueti; Dryocampa rubicunda, Earias insulana; Ennomos subsignaria; Ephestia elutella, Epilachna varivestis Mulsant, Epiphyas postvittana, Erinnyis ello; Erionota thrax; Estigmene acrea; Euetheola humilis; Eumorpha achemon; Euwallacea fornicatus; Euxesta stigmatias; Evergestis rimosalis; Grapholita delineana; Grapholita molesta; Grapholita packardi; Halyomorpha halys; Harrisina americana, Helicoverpa armigera; Helicoverpa spp.; Helicoverpa zea, Heliothis virescens; Heterocampa guttivitta; Homadaula anisocentra; Hypera postica, Hypera zoilus; Hypercompe scribonia, Hyphantria cunea, Hypothenemus hampei; Ixodes cookei; Ixodes pacificus; Ixodes scapularis; Lambdina fiscellaria, Lasiocampidae sp.; Leptinotarsa decemlineata, Lissorhoptrus oryzophilus; Listronotus maculicollis; Lobesia botrana, Lymantria dispar; Maladera castanea, Maliarpha separatella, Manduca quinquemaculata, Manduca sexta, Melanagromyza soja Zehntner; Melissopus latiferreanus; Neophasia menapia, Noctua pronuba; Noctuid sp.; Nymphula stagnata; Orgyia pseudotsugata; Orgyia vetusta; Ostrinia furnacalis; Ostrinia nubilalis; Otiorhynchus ovatus; Paleacrita merriccata, Pandemis cerasana, Papilio cresphontes; Paralobesia viteana; Pectinophora gossypiella, Pegomya betae; Pemphigus betae; Peridroma saucia; Phryganidia californica; Phyllophaga sp.; Pieris rapae; Plathypena scabra, Platynota flavedana, Platynota idaeusalis; Platynota stultana; Platyptilia carduidactyla, Playnota stultana; Plodia interpunctella, Plutella xylostella, Popillia japonica, Pseudaletia unipuncta; Pseudoplusia includens; Rhipicephalus sangunineus; Rhizotroqus majalis; Sabulodes aegrotata; Schizura concinna; Scirpophaga incertulas; Scirpophaga innotata, Scutigerella immaculata; Sesamia calamistis; Sibine stimulea, Sminthurus viridis; Sparganothis pilleriana; Sphenophorus apicalis; Sphenophorus cariosus; Sphenophorus inaequalis; Sphenophorus minimus; Sphenophorus parvulus; Sphenophorus spp.; Spilonota ocellana; Spodoptera exigua; Spodoptera frugiperda, Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni; Xanthogaleruca luteola, Xylomyges curialis; or Xylotrechus quadripes.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein each of the KPVs independently comprises at least one amino acid substitution relative to the amino acid sequence set forth in SEQ ID NO: 1, with the proviso that the KPVs do not comprise the amino acid sequence of SEQ ID NOs: 1 or 253; or an agriculturally acceptable salt thereof; and wherein the pest is selected from the group comprising Acariformes sp.; Acleris gloverana, Acrobasis vaccinii; Adoxophyes orana; Aedes aegypti; Agrotis ipsilon, Aleyrodidae sp.; Alsophila pometaria, Ambylomma americanum; Amblyomma maculatum; Amorbia humerosana; Anarsia lineatella, Anomala orientalis; Anthonomus eugenii, Anticarsia gemmatalis; Aphidoidea sp.; Aphis glycines Matsumura; Archips argyrospilia, Archips podana, Archips rosana, Argyrotaenia citrana, Argyrotaenia ljungiana; Argyrotaenia velutinana, Automeris io; Batrachedra comosae; Busseola fusca, Caudra cautella, Cerotoma spp.; Chaetocnema concinna; Chilo auricilius; Chilo partellus; Chilo suppressalis; Choristoneura fumiferana, Choristoneura pinus; Choristoneura rosaceana, Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida; Cydia funebrana; Cydia molesta, Cydia nigricana; Cydia pomonella, Cydia splendana; Cylas formicarius; Datana integerrima; Datana major; Dermacentor albipictus; Dermacentor andersoni, Dermacentor occidentalis; Dermacentor variabilis; Diabrotica spp.; Diabrotica virgifera, Diaphania hyalinata; Diaprepes abbreviatus; Diatraea crambidoides; Diatraea saccharalis; Diatraea spp.; Dorysthenes buqueti; Dryocampa rubicunda, Earias insulana; Ennomos subsignaria; Ephestia elutella, Epilachna varivestis Mulsant, Epiphyas postvittana, Erinnyis ello; Erionota thrax; Estigmene acrea; Euetheola humilis; Eumorpha achemon; Euwallacea fornicatus; Euxesta stigmatias; Evergestis rimosalis; Grapholita delineana; Grapholita molesta; Grapholita packardi; Halyomorpha halys; Harrisina americana, Helicoverpa armigera; Helicoverpa spp.; Helicoverpa zea, Heliothis virescens; Heterocampa guttivitta; Homadaula anisocentra; Hypera postica, Hypera zoilus; Hypercompe scribonia, Hyphantria cunea, Hypothenemus hampei; Ixodes cookei; Ixodes pacificus; Ixodes scapularis; Lambdina fiscellaria, Lasiocampidae sp.; Leptinotarsa decemlineata, Lissorhoptrus oryzophilus; Listronotus maculicollis; Lobesia botrana, Lymantria dispar; Maladera castanea, Maliarpha separatella, Manduca quinquemaculata, Manduca sexta, Melanagromyza soja Zehntner; Melissopus latiferreanus; Neophasia menapia, Noctua pronuba; Noctuid sp.; Nymphula stagnata; Orgyia pseudotsugata; Orgyia vetusta; Ostrinia furnacalis; Ostrinia nubilalis; Otiorhynchus ovatus; Paleacrita merriccata, Pandemis cerasana, Papilio cresphontes; Paralobesia viteana; Pectinophora gossypiella, Pegomya betae; Pemphigus betae; Peridroma saucia; Phryganidia californica; Phyllophaga sp.; Pieris rapae; Plathypena scabra, Platynota flavedana, Platynota idaeusalis; Platynota stultana; Platyptilia carduidactyla, Playnota stultana; Plodia interpunctella, Plutella xylostella, Popillia japonica, Pseudaletia unipuncta; Pseudoplusia includens; Rhipicephalus sangunineus; Rhizotroqus majalis; Sabulodes aegrotata; Schizura concinna; Scirpophaga incertulas; Scirpophaga innotata, Scutigerella immaculata; Sesamia calamistis; Sibine stimulea, Sminthurus viridis; Sparganothis pilleriana; Sphenophorus apicalis; Sphenophorus cariosus; Sphenophorus inaequalis; Sphenophorus minimus; Sphenophorus parvulus; Sphenophorus spp.; Spilonota ocellana; Spodoptera exigua; Spodoptera frugiperda, Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni; Xanthogaleruca luteola, Xylomyges curialis; or Xylotrechus quadripes.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group comprising Acariformes sp.; Acleris gloverana, Acrobasis vaccinii, Adoxophyes orana, Aedes aegypti; Agrotis ipsilon, Aleyrodidae sp.; Alsophila pometaria; Ambylomma americanum; Amblyomma maculatum; Amorbia humerosana, Anarsia lineatella, Anomala orientalis; Anthonomus eugenii; Anticarsia gemmatalis; Aphidoidea sp.; Aphis glycines Matsumura; Archips argyrospilia, Archips podana, Archips rosana, Argyrotaenia citrana, Argyrotaenia ljungiana; Argyrotaenia velutinana, Automeris io; Batrachedra comosae; Busseola fusca; Caudra cautella, Cerotoma spp.; Chaetocnema concinna, Chilo auricilius; Chilo partellus; Chilo suppressalis; Choristoneura fumiferana, Choristoneura pinus; Choristoneura rosaceana; Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida; Cydia funebrana, Cydia molesta, Cydia nigricana; Cydia pomonella; Cydia splendana; Cylas formicarius; Datana integerrima, Datana major; Dermacentor albipictus; Dermacentor andersoni; Dermacentor occidentalis; Dermacentor variabilis; Diabrotica spp.; Diabrotica virgifera; Diaphania hyalinata; Diaprepes abbreviatus; Diatraea crambidoides; Diatraea saccharalis; Diatraea spp.; Dorysthenes buqueti; Dryocampa rubicunda, Earias insulana, Ennomos subsignaria; Ephestia elutella, Epilachna varivestis Mulsant, Epiphyas postvittana; Erinnyis ello; Erionota thrax; Estigmene acrea, Euetheola humilis; Eumorpha achemon; Euwallacea fornicatus; Euxesta stigmatias; Evergestis rimosalis; Grapholita delineana, Grapholita molesta; Grapholita packardi; Halyomorpha halys; Harrisina americana, Helicoverpa armigera; Helicoverpa spp.; Helicoverpa zea, Heliothis virescens; Heterocampa guttivitta; Homadaula anisocentra; Hypera postica, Hypera zoilus; Hypercompe scribonia; Hyphantria cunea, Hypothenemus hampei; Ixodes cookei, Ixodes pacificus; Ixodes scapularis; Lambdina fiscellaria; Lasiocampidae sp.; Leptinotarsa decemlineata; Lissorhoptrus oryzophilus; Listronotus maculicollis; Lobesia botrana; Lymantria dispar; Maladera castanea, Maliarpha separatella, Manduca quinquemaculata; Manduca sexta, Melanagromyza soja Zehntner; Melissopus latiferreanus; Neophasia menapia; Noctua pronuba; Noctuid sp.; Nymphula stagnata; Orgyia pseudotsugata; Orgyia vetusta; Ostrinia furnacalis; Ostrinia nubilalis; Otiorhynchus ovatus; Paleacrita merriccata, Pandemis cerasana, Papilio cresphontes; Paralobesia viteana; Pectinophora gossypiella, Pegomya betae; Pemphigus betae; Peridroma saucia; Phryganidia californica; Phyllophaga sp.; Pieris rapae; Plathypena scabra; Platynota flavedana; Platynota idaeusalis; Platynota stultana; Platyptilia carduidactyla; Playnota stultana; Plodia interpunctella, Plutella xylostella, Popillia japonica, Pseudaletia unipuncta; Pseudoplusia includens; Rhipicephalus sangunineus; Rhizotroqus majalis; Sabulodes aegrotata, Schizura concinna; Scirpophaga incertulas; Scirpophaga innotata, Scutigerella immaculata; Sesamia calamistis; Sibine stimulea, Sminthurus viridis; Sparganothis pilleriana, Sphenophorus apicalis; Sphenophorus cariosus; Sphenophorus inaequalis; Sphenophorus minimus; Sphenophorus parvulus; Sphenophorus spp.; Spilonota ocellana; Spodoptera exigua, Spodoptera frugiperda, Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni; Xanthogaleruca luteola, Xylomyges curialis; or Xylotrechus quadripes.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group comprising Acariformes sp.; Acleris gloverana, Acrobasis vaccinii; Adoxophyes orana; Aedes aegypti; Agrotis ipsilon; Aleyrodidae sp.; Alsophila pometaria; Ambylomma americanum; Amblyomma maculatum; Amorbia humerosana, Anarsia lineatella, Anomala orientalis; Anthonomus eugenii, Anticarsia gemmatalis; Aphidoidea sp.; Aphis glycines Matsumura; Archips argyrospilia, Archips podana, Archips rosana; Argyrotaenia citrana; Argyrotaenia ljungiana; Argyrotaenia velutinana; Automeris io; Batrachedra comosae; Busseola fusca; Caudra cautella, Cerotoma spp.; Chaetocnema concinna; Chilo auricilius; Chilo partellus; Chilo suppressalis; Choristoneura fumiferana, Choristoneura pinus; Choristoneura rosaceana, Chrysodeixis includens Walker; Circulifer tenellus; Colias eurytheme; Coniesta ignefusalis; Conoderus rudis; Cosmopolites sordidus; Cotinis nitida; Crambus sp.; Crocidosema spp.; Culex pipiens; Culex quinquefasciatus; Curculio caryae; Curculio occidentis; Cyclocephala borealis; Cyclocephala lurida, Cydia funebrana; Cydia molesta; Cydia nigricana; Cydia pomonella; Cydia splendana, Cylas formicarius; Datana integerrima, Datana major; Dermacentor albipictus; Dermacentor andersoni; Dermacentor occidentalis; Dermacentor variabilis; Diabrotica spp.; Diabrotica virgifera, Diaphania hyalinata, Diaprepes abbreviatus; Diatraea crambidoides; Diatraea saccharalis; Diatraea spp.; Dorysthenes buqueti; Dryocampa rubicunda; Earias insulana; Ennomos subsignaria; Ephestia elutella; Epilachna varivestis Mulsant, Epiphyas postvittana, Erinnyis ello; Erionota thrax; Estigmene acrea; Euetheola humilis; Eumorpha achemon; Euwallacea fornicatus; Euxesta stigmatias; Evergestis rimosalis; Grapholita delineana; Grapholita molesta, Grapholita packardi; Halyomorpha halys; Harrisina americana, Helicoverpa armigera, Helicoverpa spp.; Helicoverpa zea, Heliothis virescens; Heterocampa guttivitta, Homadaula anisocentra; Hypera postica, Hypera zoilus; Hypercompe scribonia, Hyphantria cunea, Hypothenemus hampei; Ixodes cookei, Ixodes pacificus; Ixodes scapularis; Lambdina fiscellaria; Lasiocampidae sp.; Leptinotarsa decemlineata, Lissorhoptrus oryzophilus; Listronotus maculicollis; Lobesia botrana; Lymantria dispar; Maladera castanea, Maliarpha separatella, Manduca quinquemaculata, Manduca sexta, Melanagromyza soja Zehntner; Melissopus latiferreanus; Neophasia menapia; Noctua pronuba, Noctuid sp.; Nymphula stagnata; Orgyia pseudotsugata; Orgyia vetusta, Ostrinia furnacalis; Ostrinia nubilalis; Otiorhynchus ovatus; Paleacrita merriccata, Pandemis cerasana, Papilio cresphontes; Paralobesia viteana; Pectinophora gossypiella, Pegomya betae; Pemphigus betae; Peridroma saucia, Phryganidia californica, Phyllophaga sp.; Pieris rapae; Plathypena scabra, Platynota flavedana, Platynota idaeusalis; Platynota stultana; Platyptilia carduidactyla, Playnota stultana; Plodia interpunctella, Plutella xylostella, Popillia japonica, Pseudaletia unipuncta; Pseudoplusia includens; Rhipicephalus sangunineus; Rhizotroqus majalis; Sabulodes aegrotata; Schizura concinna, Scirpophaga incertulas; Scirpophaga innotata, Scutigerella immaculata; Sesamia calamistis; Sibine stimulea, Sminthurus viridis; Sparganothis pilleriana; Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua, Spodoptera frugiperda, Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola, Thyridopteryx ephemeraeformis; Thysanoptera sp.; Trichoplusia ni; Xanthogaleruca luteola, Xylomyges curialis; or Xylotrechus quadripes.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253; and wherein the pest is selected from the group consisting of: group consisting of: Aedes aegypti, Busseola fusca; Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua, Spodoptera frugiperda; Spodoptera littoralis; Trichoplusia ni; and Xanthogaleruca luteola.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence that is at least 90%, 95%, 96%, 97%, 97%, 98%, 99%, or 100% identical to the amino acid sequence according to an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, wherein each of the KPVs independently comprises at least one amino acid substitution relative to the amino acid sequence set forth in SEQ ID NO: 1, with the proviso that the KPVs do not comprise the amino acid sequence of SEQ ID NOs: 1 or 253; or an agriculturally acceptable salt thereof; and wherein the pest is selected from the group consisting of: group consisting of: Aedes aegypti; Busseola fusca, Chilo suppressalis; Culex pipiens; Culex quinquefasciatus; Diabrotica virgifera, Diatraea saccharalis; Helicoverpa armigera; Helicoverpa zea, Heliothis virescens; Leptinotarsa decemlineata; Ostrinia furnacalis; Ostrinia nubilalis; Pectinophora gossypiella, Plodia interpunctella, Plutella xylostella, Pseudoplusia includens; Spodoptera exigua, Spodoptera frugiperda; Spodoptera littoralis; Trichoplusia ni; and Xanthogaleruca luteola.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein each of the two or more KPVs independently comprises, consists essentially of, or consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group consisting of: group consisting of: Aedes aegypti, Busseola fusca; Chilo suppressalis; Culex pipiens; Culex quinquefasciatus; Diabrotica virgifera, Diatraea saccharalis; Helicoverpa armigera, Helicoverpa zea, Heliothis virescens; Leptinotarsa decemlineata, Ostrinia furnacalis; Ostrinia nubilalis; Pectinophora gossypiella, Plodia interpunctella; Plutella xylostella; Pseudoplusia includens; Spodoptera exigua, Spodoptera frugiperda; Spodoptera littoralis; Trichoplusia ni; and Xanthogaleruca luteola.

In some embodiments, a method of combating, controlling, or inhibiting a pest comprises: applying a pesticidally effective amount of a composition comprising a combination of two or more Kappa Peptide Variant (KPVs), each having pesticidal activity against one or more pest species, and an agriculturally acceptable excipient, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof, wherein each of the two or more KPVs independently consists of: an amino acid sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358, or an agriculturally acceptable salt thereof; and wherein the pest is selected from the group consisting of: group consisting of: Aedes aegypti; Busseola fusca; Chilo suppressalis; Culex pipiens; Culex quinquefasciatus; Diabrotica virgifera; Diatraea saccharalis; Helicoverpa armigera, Helicoverpa zea, Heliothis virescens; Leptinotarsa decemlineata, Ostrinia furnacalis; Ostrinia nubilalis; Pectinophora gossypiella, Plodia interpunctella; Plutella xylostella, Pseudoplusia includens; Spodoptera exigua, Spodoptera frugiperda; Spodoptera littoralis; Trichoplusia ni; and Xanthogaleruca luteola.

In some embodiments, a Kappa Peptide Variant (KPV), having pesticidal activity against one or more pests, comprises, consists essentially of, or consists of, an amino acid sequence that is at least 90%, 95%, 96%, 97%, 97%, 98%, 99%, or 100% identical to the amino acid sequence set forth in any one of SEQ ID NOs: 2, 229, 264, or 325, or an agriculturally acceptable salt thereof, with the proviso that the KPV does not comprise the amino acid sequence of SEQ ID NOs: 1 or 253.

In some embodiments, a Kappa Peptide Variant (KPV), having pesticidal activity against one or more pests, comprises, consists essentially of, or consists of, an amino acid sequence that is at least 90%, 95%, 96%, 97%, 97%, 98%, 99%, or 100% identical to the amino acid sequence set forth in SEQ ID NO: 2, or an agriculturally acceptable salt thereof, with the proviso that the KPV does not comprise the amino acid sequence of SEQ ID NOs: 1 or 253.

In some embodiments, a Kappa Peptide Variant (KPV), having pesticidal activity against one or more pests, comprises, consists essentially of, or consists of, an amino acid sequence that is at least 90%, 95%, 96%, 97%, 97%, 98%, 99%, or 100% identical to the amino acid sequence set forth in SEQ ID NO: 229, or an agriculturally acceptable salt thereof, with the proviso that the KPV does not comprise the amino acid sequence of SEQ ID NOs: 1 or 253.

In some embodiments, a Kappa Peptide Variant (KPV), having pesticidal activity against one or more pests, comprises, consists essentially of, or consists of, an amino acid sequence that is at least 90%, 95%, 96%, 97%, 97%, 98%, 99%, or 100% identical to the amino acid sequence set forth in SEQ ID NO: 264, or an agriculturally acceptable salt thereof, with the proviso that the KPV does not comprise the amino acid sequence of SEQ ID NOs: 1 or 253.

In some embodiments, a Kappa Peptide Variant (KPV), having pesticidal activity against one or more pests, comprises, consists essentially of, or consists of, an amino acid sequence that is at least 90%, 95%, 96%, 97%, 97%, 98%, 99%, or 100% identical to the amino acid sequence set forth in SEQ ID NO: 325, or an agriculturally acceptable salt thereof, with the proviso that the KPV does not comprise the amino acid sequence of SEQ ID NOs: 1 or 253.

In some embodiments, a Kappa Peptide Variant (KPV), having pesticidal activity against one or more pests, consists of an amino acid sequence as set forth in SEQ ID NO: 2, or an agriculturally acceptable salt thereof.

In some embodiments, a Kappa Peptide Variant (KPV), having pesticidal activity against one or more pests, consists of an amino acid sequence as set forth in SEQ ID NO: 229, or an agriculturally acceptable salt thereof.

In some embodiments, a Kappa Peptide Variant (KPV), having pesticidal activity against one or more pests, consists of an amino acid sequence as set forth in SEQ ID NO: 264, or an agriculturally acceptable salt thereof.

In some embodiments, a Kappa Peptide Variant (KPV), having pesticidal activity against one or more pests, consists of an amino acid sequence as set forth in SEQ ID NO: 325, or an agriculturally acceptable salt thereof.

In some embodiments, a combination comprises, consists essentially of, or consists of, two or more Kappa Peptide Variants (KPVs) having pesticidal activity against one or more pests, wherein each of the two or more KPVs independently comprise, consist essentially of, or consist of, an amino acid sequence that is at least 90%, 95%, 96%, 97%, 97%, 98%, 99%, or 100% identical to the amino acid sequence set forth in any one of SEQ ID NOs: 2, 229, 264, or 325, or an agriculturally acceptable salt thereof, with the proviso that each of the KPVs independently does not comprise the amino acid sequence of SEQ ID NOs: 1 or 253.

In some embodiments, a composition comprises, consists essentially of, or consists of, one or more Kappa Peptide Variants (KPVs) having pesticidal activity against one or more pests, and an agriculturally acceptable excipient; wherein each of the one or more KPVs independently comprise, consist essentially of, or consist of, an amino acid sequence that is at least 90%, 95%, 96%, 97%, 97%, 98%, 99%, or 100% identical to the amino acid sequence set forth in any one of SEQ ID NOs: 2, 229, 264, or 325, or an agriculturally acceptable salt thereof, with the proviso that each of the KPVs independently does not comprise the amino acid sequence of SEQ ID NOs: 1 or 253.

### Examples

### Example 1. Overview of Kappa Peptide Variants (KPVs)

The wild-type Kappa peptide (WT Kappa or Kappa or Native Kappa) is an excitatory toxin that affects the nicotinic acetylcholine receptor. Kappa peptides are produced and can be isolated from the Australian Blue Mountains Funnel-web Spider, Hadronyche versuta. An exemplary WT Kappa peptide amino acid sequence is provided herein, having the amino acid sequence of: AICTGADRPCAACCPCCPGTSCKAESNGVSYCRKDEP (SEQ ID NO: 1) (NCBI Accession No. P82228).

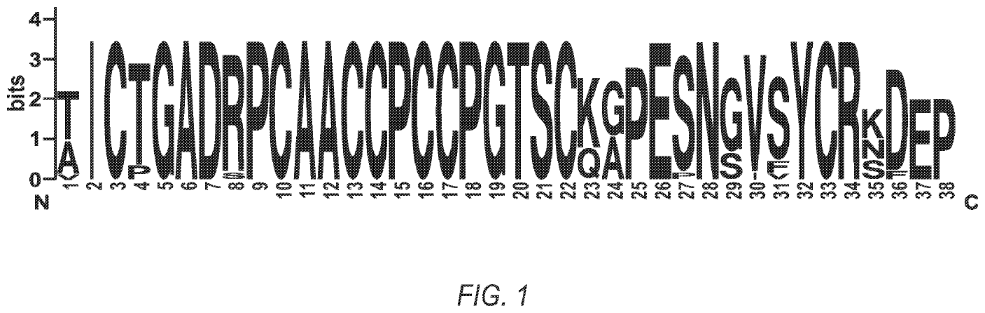

To develop Kappa Peptide Variants (KPVs) possessing desirable characteristics, known wild-type peptides having shared sequence identity to that of Kappa (“Kappa-like” peptides) were evaluated to identify the degree of sequence conservation, and highly conserved residues. Next, residue positions in the known WT peptides that were less conserved, were identified as potential targets for mutation. FIG. 1 shows a sequence logo demonstrating amino acid sequence conservation in all known Kappa peptide variants sequences.

### Example 2. Expression and Activity Analysis

Generation of the KPVs was performed as follows: Individual ORFs were constructed containing either a polynucleotide operable to encode a wild-type Kappa, or a polynucleotide operable to encode each of the KPVs. These ORFs were then inserted into a pKlac1 vector (Catalog No. N3740; New England Biolabs®; 240 County Road, Ipswich, MA 01938-2723). The pKlac1 vector contains the Kluyveromyces lactis P_{LAC4-PBI} promoter; DNA encoding the K. lactis α-mating factor (α-MF) secretion domain (for secreted expression), a multiple cloning site (MCS), the Kluyveromyces lactis LAC4 transcription terminator (TT), and a fungal acetamidase selectable marker gene (amdS) expressed from the yeast ADH2 promoter (P_{ADH2}). In addition, an E. coli replication origin (ORI) and ampicillin resistance gene (Ap^{R}) are present for propagation of pKLAC1 in E. coli.

The resulting vectors, i.e., pKlac1-WT-Kappa, and the various pKlac1-KPV vectors, were then linearized, and transformed into electrocompetent Kluyveromyces lactis host cells, for stable integration of multiple copies of the linearized vectors into the Kluyveromyces lactis host genome at the LAC4 loci. The transformed Kluyveromyces lactis were then plated on selection agar containing acetamide as the sole nitrogen source to identify strains containing multiple insertions of the expression cassette and its acetamidase selection. The expression vector was linearized by the digestion with the restriction enzyme SacII; the resulting linear plasmid was then transformed into K. lactis cell by electroporation. Positive transformation colonies were picked for yield analysis.

Next, positively identified transformants were used to inoculate the culture wells of a 48-well deep-well culture plate. The deep-well culture plate was sealed with a sterile breathable seal and cultured at 27° C., 250 rpm for 4 days. After six days in culture, the deep-well culture plate was spun at 4000 rpm for 10 minute to collect the cell pellets and supernatants, the cell pellets were resuspended into 1 mL of 50% glycerol for long-term storage of the strain at −80° C. freezer. The supernatants containing the peptides were subsequently subjected to rpHPLC evaluation to determine peptide yield.

### Yield Analysis

Briefly, to run HPLC, the supernatants were filtered through 0.2 μm filter membrane; then, 200 μL of filtered supernatant was then transferred to a 300 μL 96-well plate for rpHPLC analysis using Ultramate 3000 HPLC system (ThermoFisher®) controlled by Chromeleon 7 software. The HPLC was performed using monolithic C18 columns using water with 0.1% Trifloroacetic acid, and acetonitrile as the mobile phase. An elution protocol using 16-30% acetonitrile was used for peptide purification, in which the peptides were eluted between a range of 22-26% acetonitrile. The corresponding peptide peak area from the HPLC chromatograph was used to calculate the peptide yield. Yield comparisons were based on peak area (mAU) as determined by dividing the average area for the KPV by the average area for WT. Any relative values greater than 1 are considered an improvement on expression.

For some of the peaks observed in the HPLC analysis, the peptides were purified by rpHPLC and subjected to LC/MS identification using a Waters/Micromass ESI-TOF mass spectrometer on-line with an Agilent HPLC system. The LC/MS results confirmed the correct amino acid sequences.

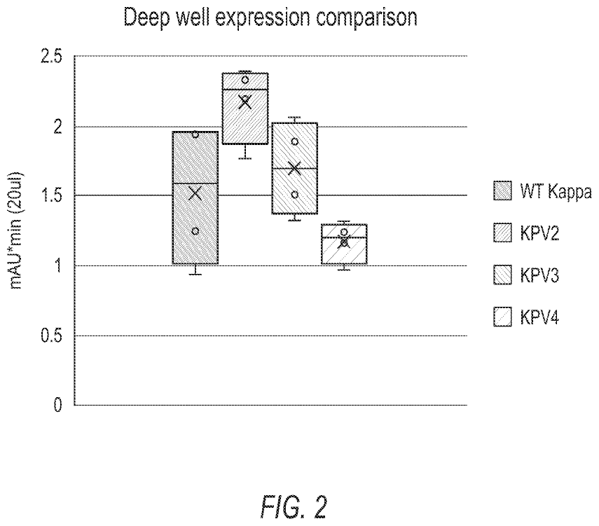

For example, as shown in FIG. 2, KPV2 had a 45% increase in expression relative to WT Kappa; and KPV3 had a 30% improvement in expression relative to WT Kappa.

A summary of illustrative KPVs and their expression is provided in Example 11 below.

### Fly Injection Assay

A housefly injection assay was performed to determine the activity of KPVs as compared to WT Kappa. Adult houseflies were immobilized with CO₂ for 10 minutes, and then transferred to a CO₂ pan to keep immobilized. Flies with weight between 12-20 mg were picked for injection.

Each of the individual KPVs and WT Kappa were diluted in water to proper doses for injection: here, a dose of 10 ng/μL was used for a single dose comparison. Next, a 0.5 μL peptide solution was injected into each housefly at the dorsal thorax using a hand-microapplicator with 1 cc all-glass syringe with 30 gauge straight needle. The injected flies were then transferred to a 2 oz. transparent portion container with a wet #4 filter paper.

Activity of the KPVs was determined based on percent knock-down (% KD), i.e., the percentage of flies dead, unmoving, or unable to hold on to the side of a container when said container is up-ended. Activity of the KPVs was assessed at 24-hours post-injection, and compared to the activity WT Kappa. WT Kappa, when injected at a dose of 10 ng/μL, has a LD₅₀ of 80%. The activity comparison between the KPVs and WT Kappa was determined by comparing the % KD of a given KPV, relative to WT, by dividing the % KD for KPVs by the percent mortality for WT.

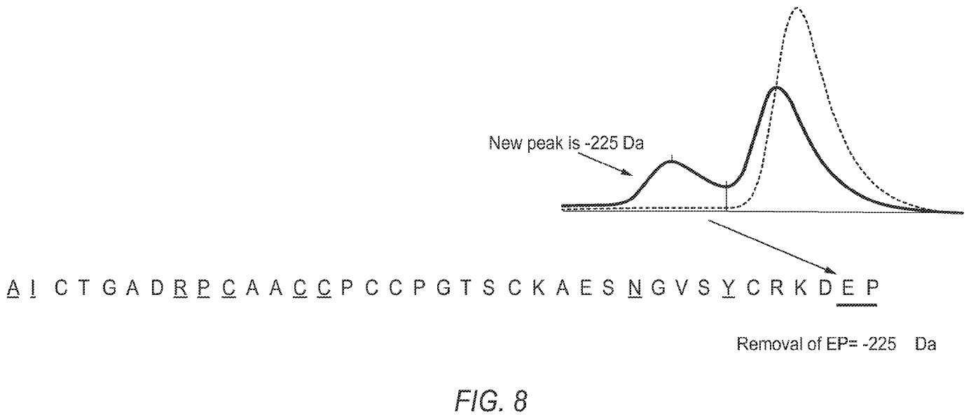

For example, as shown in FIG. 8, KPV2 has similar activity to WT Kappa in a fly injection assay. The LD₅₀ of WT Kappa was 8.5 ng/μL, whereas the LD₅₀ for KPV2 was 8.7 ng/μL.

A summary of illustrative KPVs and their activity is provided in Example 11 below.

### Example 3. Point Mutations

A point mutation strategy was initiated to identify KPVs, i.e., peptides having a mutation relative to WT Kappa (SEQ ID NO: 1) that result in improvements to expression and/or activity. Briefly, KPV ORFs were created according to the methods described in Example 2.

To evaluate the impact of a single point mutation in a KPV, the expression and activity for each KPV was compared relative to WT Kappa.

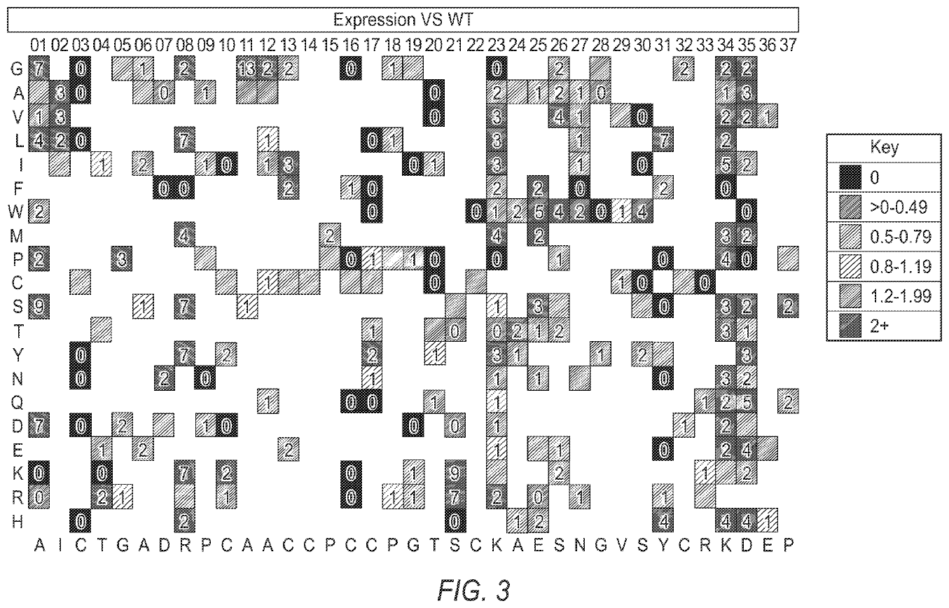

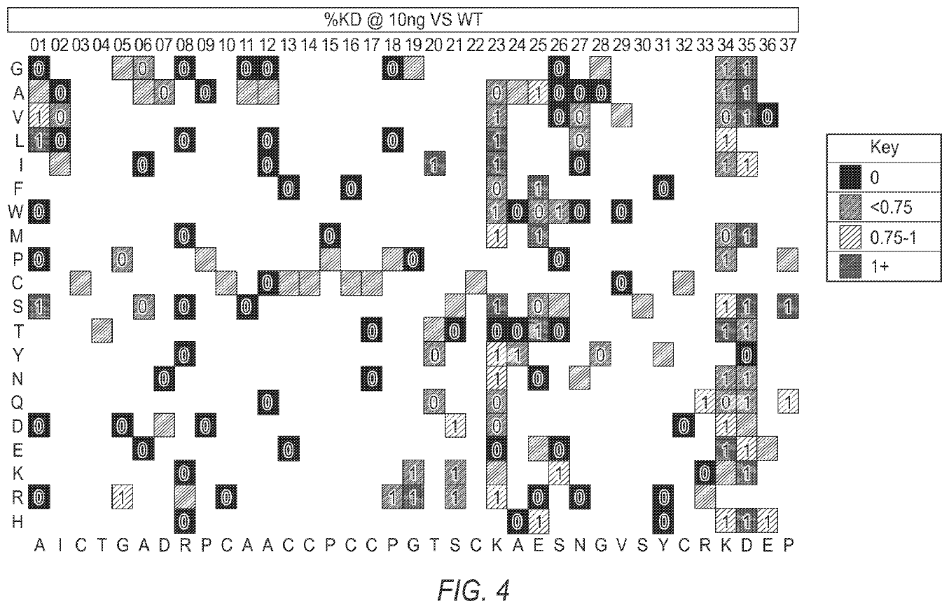

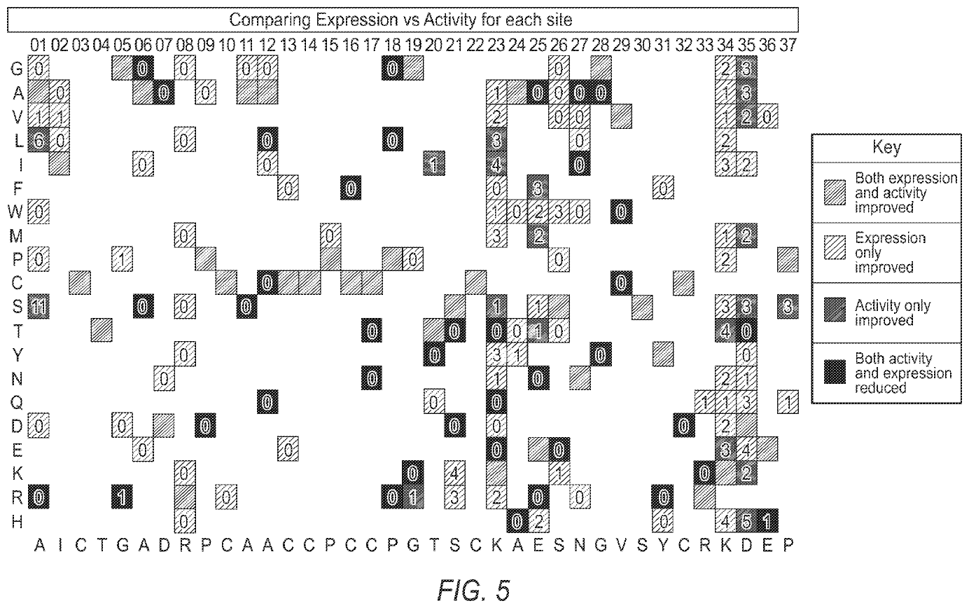

FIGS. 3-5 show the impact each mutation has on the expression and/or activity of a KPV as compared to WT Kappa. From this analysis, positions where both activity and expression are improved were identified. The table below shows illustrative KPVs having a single point mutation that provides an improvement in expression and/or activity relative to WT Kappa.

The tables below provide a list of illustrative KPVs having a single point mutation, and their corresponding expression and activity relative to WT Kappa.

TABLE 2
Point mutations that provide either an improvement in expression or
activity. Here, “Mut.” refers to the point mutation relative to WT Kappa, e.g.,
E25F refers to a substitution of E with F at position 25 of the WT Kappa
sequence (SEQ ID NO: 1). “Exp.” refers to the expression of a corresponding KPV
relative to the expression of WT Kappa. “Act.” refers to the activity of a
corresponding KPV relative to the expression of WT Kappa. Here, activity was
assessed based on the %KD of flies dosed with 10 ng/μL of a given KPV, relative
to the %KD of flies dosed with 10 ng/μL of WT Kappa. KPVs having an N-terminus
addition to the WT-Kappa sequence are indicated as “1+” (e.g., “1+G” indicates
the addition of a G residue appended to the N-terminus of the WT Kappa peptide).
Mutations where an amino acid has been deleted are indicated as “-”

| SEQ ID NO | Mut. | Exp. | Act. | Amino Acid Sequence |
|---|---|---|---|---|
| 5 | E25F | 2.23 | 1.25 | AICTGADRPCAACCPCCPGTSCKAFSNGVSYCRKDEP |
| 6 | S26W | 4.46 | 0.63 | AICTGADRPCAACCPCCPGTSCKAEWNGVSYCRKDEP |
| 7 | C13I | 3.08 | N/A | AICTGADRPCAAICPCCPGTSCKAESNGVSYCRKDEP |
| 10 | A6I | 1.52 | 0.00 | AICTGIDRPCAACCPCCPGTSCKAESNGVSYCRKDEP |
| 11 | Y31H | 3.70 | 0.00 | AICTGADRPCAACCPCCPGTSCKAESNGVSHCRKDEP |
| 16 | A12I | 1.10 | 0.00 | AICTGADRPCAICCPCCPGTSCKAESNGVSYCRKDEP |
| 17 | T4E | 1.12 | N/A | AICEGADRPCAACCPCCPGTSCKAESNGVSYCRKDEP |
| 18 | N27R | 1.33 | 0.00 | AICTGADRPCAACCPCCPGTSCKAESRGVSYCRKDEP |
| 19 | C32G | 1.61 | N/A | AICTGADRPCAACCPCCPGTSCKAESNGVSYGRKDEP |
| 23 | A24Y | 1.16 | 0.50 | AICTGADRPCAACCPCCPGTSCKYESNGVSYCRKDEP |
| 30 | C10Y | 1.68 | N/A | AICTGADRPYAACCPCCPGTSCKAESNGVSYCRKDEP |
| 31 | C10K | 2.42 | N/A | AICTGADRPKAACCPCCPGTSCKAESNGVSYCRKDEP |
| 35 | D35N | 1.63 | 0.56 | AICTGADRPCAACCPCCPGTSCKAESNGVSYCRKNEP |
| 40 | I2V | 2.29 | 0.38 | AVCTGADRPCAACCPCCPGTSCKAESNGVSYCRKDEP |
| 48 | E36V | 1.28 | 0.00 | AICTGADRPCAACCPCCPGTSCKAESNGVSYCRKDVP |
| 50 | S26P | 1.31 | 0.00 | AICTGADRPCAACCPCCPGTSCKAEPNGVSYCRKDEP |
| 54 | Y31F | 2.85 | 0.63 | AICTGADRPCAACCPCCPGTSCKAESNGVSFCRKDEP |
| 55 | G5D | 1.78 | 0.00 | AICTDADRPCAACCPCCPGTSCKAESNGVSYCRKDEP |
| 57 | C13G | 1.50 | N/A | AICTGADRPCAAGCPCCPGTSCKAESNGVSYCRKDEP |
| 60 | N27V | 1.44 | 0.25 | AICTGADRPCAACCPCCPGTSCKAESVGVSYCRKDEP |
| 66 | T4R | 2.03 | N/A | AICRGADRPCAACCPCCPGTSCKAESNGVSYCRKDEP |
| 68 | K23W | 1.14 | 0.56 | AICTGADRPCAACCPCCPGTSCWAESNGVSYCRKDEP |
| 70 | C17Y | 2.03 | N/A | AICTGADRPCAACCPCYPGTSCKAESNGVSYCRKDEP |
| 71 | C13E | 1.61 | 0.00 | AICTGADRPCAAECPCCPGTSCKAESNGVSYCRKDEP |
| 76 | S26T | 1.62 | 0.00 | AICTGADRPCAACCPCCPGTSCKAETNGVSYCRKDEP |
| 77 | K34G | 2.41 | 0.69 | AICTGADRPCAACCPCCPGTSCKAESNGVSYCRGDEP |
| 79 | C10R | 1.17 | 0.00 | AICTGADRPRAACCPCCPGTSCKAESNGVSYCRKDEP |
| 85 | K34A | 1.31 | 0.50 | AICTGADRPCAACCPCCPGTSCKAESNGVSYCRADEP |
| 102 | K34N | 2.71 | 0.56 | AICTGADRPCAACCPCCPGTSCKAESNGVSYCRNDEP |
| 110 | A6E | 1.51 | 0.00 | AICTGEDRPCAACCPCCPGTSCKAESNGVSYCRKDEP |
| 114 | K23F | 1.77 | 0.13 | AICTGADRPCAACCPCCPGTSCFAESNGVSYCRKDEP |
| 124 | S26A | 1.51 | 0.00 | AICTGADRPCAACCPCCPGTSCKAEANGVSYCRKDEP |
| 126 | K34L | 2.13 | 0.75 | AICTGADRPCAACCPCCPGTSCKAESNGVSYCRLDEP |
| 140 | G19R | 0.66 | 1.25 | AICTGADRPCAACCPCCPRTSCKAESNGVSYCRKDEP |
| 146 | K34M | 2.77 | 0.25 | AICTGADRPCAACCPCCPGTSCKAESNGVSYCRMDEP |
| 147 | T20I | 0.74 | 1.25 | AICTGADRPCAACCPCCPGISCKAESNGVSYCRKDEP |
| 149 | S26K | 1.73 | 0.75 | AICTGADRPCAACCPCCPGTSCKAEKNGVSYCRKDEP |
| 151 | A1W | 1.51 | 0.00 | WICTGADRPCAACCPCCPGTSCKAESNGVSYCRKDEP |
| 152 | P9A | 1.20 | 0.00 | AICTGADRACAACCPCCPGTSCKAESNGVSYCRKDEP |
| 157 | K34E | 2.25 | 1.13 | AICTGADRPCAACCPCCPGTSCKAESNGVSYCREDEP |
| 158 | T20Q | 1.02 | 0.25 | AICTGADRPCAACCPCCPGQSCKAESNGVSYCRKDEP |
| 160 | K34V | 2.44 | 0.56 | AICTGADRPCAACCPCCPGTSCKAESNGVSYCRVDEP |
| 161 | E25H | 1.64 | 1.00 | AICTGADRPCAACCPCCPGTSCKAHSNGVSYCRKDEP |
| 162 | P37S | 2.48 | 1.25 | AICTGADRPCAACCPCCPGTSCKAESNGVSYCRKDES |
| 167 | S30Y | 1.50 | N/A | AICTGADRPCAACCPCCPGTSCKAESNGVYYCRKDEP |
| 168 | N27L | 1.09 | 0.25 | AICTGADRPCAACCPCCPGTSCKAESLGVSYCRKDEP |
| 170 | G19P | 1.07 | 0.00 | AICTGADRPCAACCPCCPPTSCKAESNGVSYCRKDEP |
| 173 | P15M | 1.50 | 0.00 | AICTGADRPCAACCMCCPGTSCKAESNGVSYCRKDEP |
| 176 | A24W | 1.61 | 0.00 | AICTGADRPCAACCPCCPGTSCKWESNGVSYCRKDEP |
| 178 | S26G | 1.81 | 0.00 | AICTGADRPCAACCPCCPGTSCKAEGNGVSYCRKDEP |
| 180 | E25W | 5.10 | 0.38 | AICTGADRPCAACCPCCPGTSCKAWSNGVSYCRKDEP |
| 184 | P37Q | 1.50 | 0.75 | AICTGADRPCAACCPCCPGTSCKAESNGVSYCRKDEQ |
| 185 | C13F | 2.25 | 0.00 | AICTGADRPCAAFCPCCPGTSCKAESNGVSYCRKDEP |
| 186 | N27W | 2.18 | 0.00 | AICTGADRPCAACCPCCPGTSCKAESWGVSYCRKDEP |
| 189 | R33Q | 1.34 | 1.00 | AICTGADRPCAACCPCCPGTSCKAESNGVSYCQKDEP |
| 199 | I2A | 2.50 | 0.00 | AACTGADRPCAACCPCCPGTSCKAESNGVSYCRKDEP |
| 205 | K23A | 1.59 | 0.38 | AICTGADRPCAACCPCCPGTSCAAESNGVSYCRKDEP |
| 206 | K23D | 1.06 | 0.13 | AICTGADRPCAACCPCCPGTSCDAESNGVSYCRKDEP |
| 209 | K23I | 3.03 | 1.25 | AICTGADRPCAACCPCCPGTSCIAESNGVSYCRKDEP |
| 210 | K23L | 2.53 | 1.12 | AICTGADRPCAACCPCCPGTSCLAESNGVSYCRKDEP |
| 211 | K23M | 3.95 | 0.88 | AICTGADRPCAACCPCCPGTSCMAESNGVSYCRKDEP |
| 212 | K23N | 1.37 | 0.75 | AICTGADRPCAACCPCCPGTSCNAESNGVSYCRKDEP |
| 215 | K23R | 2.14 | 0.88 | AICTGADRPCAACCPCCPGTSCRAESNGVSYCRKDEP |
| 216 | K23V | 2.88 | 0.63 | AICTGADRPCAACCPCCPGTSCVAESNGVSYCRKDEP |
| 217 | K23Y | 3.25 | 0.94 | AICTGADRPCAACCPCCPGTSCYAESNGVSYCRKDEP |
| 218 | K34D | 2.33 | 0.75 | AICTGADRPCAACCPCCPGTSCKAESNGVSYCRDDEP |
| 220 | K34H | 4.26 | 1.00 | AICTGADRPCAACCPCCPGTSCKAESNGVSYCRHDEP |
| 221 | K34I | 4.97 | 0.63 | AICTGADRPCAACCPCCPGTSCKAESNGVSYCRIDEP |
| 222 | K34P | 3.68 | 0.25 | AICTGADRPCAACCPCCPGTSCKAESNGVSYCRPDEP |
| 223 | K34Q | 2.02 | 0.38 | AICTGADRPCAACCPCCPGTSCKAESNGVSYCRQDEP |
| 224 | K34S | 2.71 | 1.00 | AICTGADRPCAACCPCCPGTSCKAESNGVSYCRSDEP |
| 225 | K34T | 2.87 | 1.25 | AICTGADRPCAACCPCCPGTSCKAESNGVSYCRTDEP |
| 226 | A1V | 2.23 | 1.22 | VICTGADRPCAACCPCCPGTSCKAESNGVSYCRKDEP |
| 227 | A1L | 4.46 | 1.25 | LICTGADRPCAACCPCCPGTSCKAESNGVSYCRKDEP |
| 230 | S26V | 3.79 | 0.00 | AICTGADRPCAACCPCCPGTSCKAEVNGVSYCRKDEP |
| 231 | A1S | 8.92 | 1.25 | SICTGADRPCAACCPCCPGTSCKAESNGVSYCRKDEP |
| 232 | A1D | 6.69 | N/A | DICTGADRPCAACCPCCPGTSCKAESNGVSYCRKDEP |
| 233 | Y31L | 6.69 | N/A | AICTGADRPCAACCPCCPGTSCKAESNGVSLCRKDEP |
| 235 | R8K | 6.69 | 0.00 | AICTGADKPCAACCPCCPGTSCKAESNGVSYCRKDEP |
| 236 | R8H | 4.46 | 0.00 | AICTGADHPCAACCPCCPGTSCKAESNGVSYCRKDEP |
| 237 | R8Y | 6.69 | 0.00 | AICTGADYPCAACCPCCPGTSCKAESNGVSYCRKDEP |
| 238 | R8L | 6.69 | 0.00 | AICTGADLPCAACCPCCPGTSCKAESNGVSYCRKDEP |
| 239 | R8M | 4.46 | 0.00 | AICTGADMPCAACCPCCPGTSCKAESNGVSYCRKDEP |
| 240 | R8S | 6.69 | 0.00 | AICTGADSPCAACCPCCPGTSCKAESNGVSYCRKDEP |
| 244 | A11G | 13.37 | 0.00 | AICTGADRPCGACCPCCPGTSCKAESNGVSYCRKDEP |
| 245 | A12G | 2.23 | 0.00 | AICTGADRPCAGCCPCCPGTSCKAESNGVSYCRKDEP |
| 246 | S21K | 8.92 | 0.50 | AICTGADRPCAACCPCCPGTKCKAESNGVSYCRKDEP |
| 247 | S21R | 6.69 | 0.50 | AICTGADRPCAACCPCCPGTRCKAESNGVSYCRKDEP |
| 248 | R8G | 2.23 | 0.00 | AICTGADGPCAACCPCCPGTSCKAESNGVSYCRKDEP |
| 250 | A1P | 2.23 | 0.00 | PICTGADRPCAACCPCCPGTSCKAESNGVSYCRKDEP |
| 260 | G5P | 3.12 | 0.34 | AICTPADRPCAACCPCCPGTSCKAESNGVSYCRKDEP |
| 263 | K23S | 0.89 | 1.02 | AICTGADRPCAACCPCCPGTSCSAESNGVSYCRKDEP |
| 269 | S30W | 4.46 | 1.25 | AICTGADRPCAACCPCCPGTSCKAESNGVWYCRKDEP |
| 270 | E25T | 1.78 | 0.79 | AICTGADRPCAACCPCCPGTSCKATSNGVSYCRKDEP |
| 274 | 1+G | 2.67 | 0.07 | GAICTGADRPCAACCPCCPGTSCKAESNGVSYCRKDEP |
| 282 | E25M | 2.01 | 1.24 | AICTGADRPCAACCPCCPGTSCKAMSNGVSYCRKDEP |
| 283 | A1G | 6.69 | 0.00 | GICTGADRPCAACCPCCPGTSCKAESNGVSYCRKDEP |
| 284 | E25S | 2.90 | 0.43 | AICTGADRPCAACCPCCPGTSCKASSNGVSYCRKDEP |
| 286 | D35A | 2.79 | 1.13 | AICTGADRPCAACCPCCPGTSCKAESNGVSYCRKAEP |
| 287 | D35E | 4.28 | 1.00 | AICTGADRPCAACCPCCPGTSCKAESNGVSYCRKEEP |
| 288 | D35G | 2.30 | 1.13 | AICTGADRPCAACCPCCPGTSCKAESNGVSYCRKGEP |
| 289 | D35H | 3.66 | 1.25 | AICTGADRPCAACCPCCPGTSCKAESNGVSYCRKHEP |
| 290 | D35I | 1.74 | 0.88 | AICTGADRPCAACCPCCPGTSCKAESNGVSYCRKIEP |
| 291 | D35K | 1.99 | 1.25 | AICTGADRPCAACCPCCPGTSCKAESNGVSYCRKKEP |
| 292 | D35M | 2.10 | 1.13 | AICTGADRPCAACCPCCPGTSCKAESNGVSYCRKMEP |
| 294 | D35Q | 3.26 | 1.25 | AICTGADRPCAACCPCCPGTSCKAESNGVSYCRKQEP |
| 296 | D35S | 2.00 | 1.25 | AICTGADRPCAACCPCCPGTSCKAESNGVSYCRKSEP |
| 297 | D35V | 2.07 | 1.13 | AICTGADRPCAACCPCCPGTSCKAESNGVSYCRKVEP |
| 299 | D35Y | 3.47 | 0.00 | AICTGADRPCAACCPCCPGTSCKAESNGVSYCRKYEP |
| 318 | D7N | 2.23 | 0.99 | AICTGANRPCAACCPCCPGTSCKAESNGVSYCRKDEP |
| 320 | A24T | 2.23 | 0.20 | AICTGADRPCAACCPCCPGTSCKTESNGVSYCRKDEP |
| 323 | I2L | 2.23 | 0.00 | ALCTGADRPCAACCPCCPGTSCKAESNGVSYCRKDEP |

TABLE 3
Point mutations providing an improvement in expression and activity.
Here, “Mut.” refers to the point mutation relative to WT Kappa, e.g., E25F refers
to a substitution of E with F at position 25 of the WT Kappa sequence (SEQ ID NO: 1).
“Exp.” refers to the expression of a corresponding KPV relative to the expression of
WT Kappa. “Act.” refers to the activity of a corresponding KPV relative to the
expression of WT Kappa. Here, activity was assessed based on the %KD of flies dosed
with 10 ng/μL of a given KPV, relative to the %KD of flies dosed with 10 ng/μL of
WT Kappa. KPVs having an N-terminus addition to the WT-Kappa sequence are indicated
as “1+” (e.g., “1+G” indicates the addition of a G residue appended to the N-terminus
of the WT Kappa peptide). Mutations where an amino acid has been deleted are
indicated as “-”

| SEQ ID NO | Mut. | Exp. | Act. | Amino Acid Sequence |
|---|---|---|---|---|
| 5 | E25F | 2.23 | 1.25 | AICTGADRPCAACCPCCPGTSCKAFSNGVSYCRKDEP |
| 157 | K34E | 2.25 | 1.13 | AICTGADRPCAACCPCCPGTSCKAESNGVSYCREDEP |
| 162 | P37S | 2.48 | 1.25 | AICTGADRPCAACCPCCPGTSCKAESNGVSYCRKDES |
| 209 | K23I | 3.03 | 1.25 | AICTGADRPCAACCPCCPGTSCIAESNGVSYCRKDEP |
| 210 | K23L | 2.53 | 1.12 | AICTGADRPCAACCPCCPGTSCLAESNGVSYCRKDEP |
| 225 | K34T | 2.87 | 1.25 | AICTGADRPCAACCPCCPGTSCKAESNGVSYCRTDEP |
| 226 | A1V | 2.23 | 1.22 | VICTGADRPCAACCPCCPGTSCKAESNGVSYCRKDEP |
| 227 | A1L | 4.46 | 1.25 | LICTGADRPCAACCPCCPGTSCKAESNGVSYCRKDEP |
| 231 | A1S | 8.92 | 1.25 | SICTGADRPCAACCPCCPGTSCKAESNGVSYCRKDEP |
| 269 | S30W | 4.46 | 1.25 | AICTGADRPCAACCPCCPGTSCKAESNGVWYCRKDEP |
| 282 | E25M | 2.01 | 1.24 | AICTGADRPCAACCPCCPGTSCKAMSNGVSYCRKDEP |
| 286 | D35A | 2.79 | 1.13 | AICTGADRPCAACCPCCPGTSCKAESNGVSYCRKAEP |
| 288 | D35G | 2.30 | 1.13 | AICTGADRPCAACCPCCPGTSCKAESNGVSYCRKGEP |
| 289 | D35H | 3.66 | 1.25 | AICTGADRPCAACCPCCPGTSCKAESNGVSYCRKHEP |
| 291 | D35K | 1.99 | 1.25 | AICTGADRPCAACCPCCPGTSCKAESNGVSYCRKKEP |
| 292 | D35M | 2.10 | 1.13 | AICTGADRPCAACCPCCPGTSCKAESNGVSYCRKMEP |
| 294 | D35Q | 3.26 | 1.25 | AICTGADRPCAACCPCCPGTSCKAESNGVSYCRKQEP |
| 296 | D35S | 2.00 | 1.25 | AICTGADRPCAACCPCCPGTSCKAESNGVSYCRKSEP |
| 297 | D35V | 2.07 | 1.13 | AICTGADRPCAACCPCCPGTSCKAESNGVSYCRKVEP |

### Example 4. Multiple Mutations

A multiple mutation strategy was initiated to identify KPVs having two or more mutations that result in improvement to expression and/or activity relative to WT Kappa (SEQ ID NO: 1).

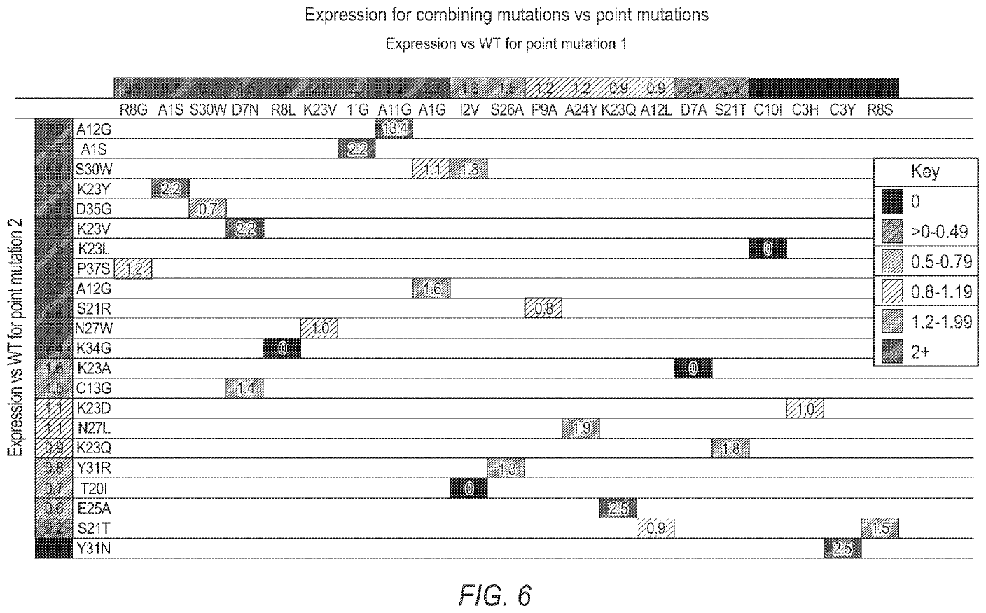

FIG. 6 shows a graph illustrating the effect multiple mutations have on expression, as compared to a single point mutation. As shown in FIG. 6, each axis of the graph shows the effect a single mutation has on the expression of a KPV relative to WT Kappa. The points along the graph where the X- and Y-axis intersect, shows the expression of a KPV having both of the mutations (i.e., the mutation corresponding to the X- and the Y-axis), relative to WT Kappa.

As demonstrated in FIG. 6, the influence of each individual mutation is not always predictive of expression for a KPV with both of those mutations. For example, each of the single point mutations, C3Y and Y31N, have result in no expression for a KPV having each of those individual mutations; however, when C3Y and Y31N are both present in a KPV, it results in an improvement in expression.

The tables below provide a list of illustrative KPVs having a multiple mutations, and their corresponding expression and activity relative to WT Kappa.

TABLE 4
Multiple mutations providing an improvement in expression or activity. Here, “Mut.” refers to
the point mutation relative to WT Kappa, e.g., E25F refers to a substitution of E with F at
position 25 of the WT Kappa sequence (SEQ ID NO: 1). “Exp.” refers to the expression of a
corresponding KPV relative to the expression of WT Kappa. ″Act.″ refers to the activity of a
corresponding KPV relative to the expression of WT Kappa. Here, activity was assessed based on
the %KD of flies dosed with 10 ng/μL of a given KPV, relative to the %KD of flies dosed with
10 ng/μL of WT Kappa. KPVs having an N-terminus addition to theWT-Kappa sequence are indicated
as “1+” (e.g., “1+G” indicates the addition of a G residue appended to the N-terminus of the WT
Kappa peptide). Mutations where an amino acid has been deleted are indicated as “-”

| SEQ | | | | | |
|---|---|---|---|---|---|
| ID | No. of | | | | |
| NO | mutations | Mut. | Exp. | Act. | Amino Acid Sequence |
| 2 | 4 | A1G A24T E36- P37- | 2.90 | 1.10 | GICTGADRPCAACCPCCPGTSCKTESNGVSYCRKD |
| 3 | 4 | A1S E25G S26V S30A | 1.90 | 0.75 | SICTGADRPCAACCPCCPGTSCKAGVNGVAYCRKDEP |
| 4 | 2 | S26A Y31R | 1.32 | 1.25 | AICTGADRPCAACCPCCPGTSCKAEANGVSRCRKDEP |
| 12 | 2 | C22S S30C | 1.35 | 0.50 | AICTGADRPCAACCPCCPGTSSKAESNGVCYCRKDEP |
| 13 | 3 | G5E A11H S30R | 1.77 | N/A | AICTEADRPCHACCPCCPGTSCKAESNGVRYCRKDEP |
| 25 | 2 | C14N D35M | 1.25 | N/A | AICTGADRPCAACNPCCPGTSCKAESNGVSYCRKMEP |
| 39 | 2 | G5K E25A | 1.51 | 0.00 | AICTKADRPCAACCPCCPGTSCKAASNGVSYCRKDEP |
| 45 | 2 | T20S S26G | 1.60 | 0.00 | AICTGADRPCAACCPCCPGSSCKAEGNGVSYCRKDEP |
| 46 | 2 | S26P K34R | 1.48 | 0.00 | AICTGADRPCAACCPCCPGTSCKAEPNGVSYCRRDEP |
| 56 | 2 | C3Y Y31N | 2.51 | N/A | AIYTGADRPCAACCPCCPGTSCKAESNGVSNCRKDEP |
| 73 | 2 | R8G P37S | 1.20 | N/A | AICTGADGPCAACCPCCPGTSCKAESNGVSYCRKDES |
| 74 | 2 | R8S S21T | 1.50 | 0.00 | AICTGADSPCAACCPCCPGTTCKAESNGVSYCRKDEP |
| 78 | 2 | A11S T20H | 1.19 | 0.25 | AICTGADRPCSACCPCCPGHSCKAESNGVSYCRKDEP |
| 164 | 2 | I2V S30W | 1.77 | 0.00 | AVCTGADRPCAACCPCCPGTSCKAESNGVWYCRKDEP |
| 183 | 2 | D7N C13G | 1.36 | 0.00 | AICTGANRPCAAGCPCCPGTSCKAESNGVSYCRKDEP |
| 191 | 2 | S21T K23Q | 1.82 | 0.00 | AICTGADRPCAACCPCCPGTTCQAESNGVSYCRKDEP |
| 192 | 2 | A6I E36K | 1.74 | 0.00 | AICTGIDRPCAACCPCCPGTSCKAESNGVSYCRKDKP |
| 195 | 2 | D7G E36F | 1.01 | 1.25 | AICTGAGRPCAACCPCCPGTSCKAESNGVSYCRKDEP |
| 204 | 2 | C3H K23D | 1.04 | 0.00 | AIHTGADRPCAACCPCCPGTSCDAESNGVSYCRKDEP |
| 228 | 6 | A1S E25G S26V S30A | 3.12 | 0.23 | SICTGADRPCAACCPCCPGTSCKAGVNGVAYCRKD |
| | | E36- P37- | | | |
| 229 | 4 | S26A Y31R E36- P37- | 1.56 | 1.25 | AICTGADRPCAACCPCCPGTSCKAEANGVSRCRKD |
| 243 | 2 | A11G A12G | 4.46 | 0.00 | AICTGADRPCGGCCPCCPGTSCKAESNGVSYCRKDEP |
| 249 | 3 | A24T E36- P37- | 8.92 | 1.00 | AICTGADRPCAACCPCCPGTSCKTESNGVSYCRKD |
| 251 | 4 | 2′E 1′A E36- P37- | 6.69 | 0.00 | EAAICTGADRPCAACCPCCPGTSCKAESNGVSYCRKD |
| 252 | 2 | 2′N 1′S | 1.56 | 0.06 | NSAICTGADRPCAACCPCCPGTSCKAESNGVSYCRKDEP |
| 253 | 2 | 2′G 1′S | 6.69 | 0.00 | GSAICTGADRPCAACCPCCPGTSCKAESNGVSYCRKDEP |
| 254 | 4 | 2′G 1′S E36- P37- | 4.46 | 0.03 | GSAICTGADRPCAACCPCCPGTSCKAESNGVSYCRKD |
| 255 | 3 | 2′E 1′A I2V | 8.92 | 0.00 | EAAVCTGADRPCAACCPCCPGTSCKAESNGVSYCRKDEP |
| 256 | 7 | 2′G 1′S A1Q 12Y | 3.34 | 0.00 | GSQYCTGADRPCAACCPCCPGTSCKAESNGVSYCRKA |
| | | D35- E36- P37A | | | |
| 257 | 2 | A1G S30W | 1.11 | 0.00 | GICTGADRPCAACCPCCPGTSCKAESNGVWYCRKDEP |
| 258 | 5 | A11G K34A D35- E36- | 1.34 | 0.00 | AICTGADRPCGACCPCCPGTSCKAESNGVSYCRA |
| | | P37- | | | |
| 259 | 2 | A1G A11G | 1.56 | 0.00 | GICTGADRPCGACCPCCPGTSCKAESNGVSYCRKDEP |
| 264 | 2 | K23Q E25A | 2.45 | 1.25 | AICTGADRPCAACCPCCPGTSCQAASNGVSYCRKDEP |
| 265 | 2 | 1′N K23Y | 1.78 | 0.00 | NAICTGADRPCAACCPCCPGTSCYAESNGVSYCRKDEP |
| 275 | 3 | 1′G E36- P37- | 4.01 | 0.06 | GAICTGADRPCAACCPCCPGTSCKAESNGVSYCRKD |
| 276 | 2 | 1′G A1S | 2.23 | 0.06 | GSICTGADRPCAACCPCCPGTSCKAESNGVSYCRKDEP |
| 277 | 4 | A1S E25F E36- P37- | 1.11 | 0.07 | SICTGADRPCAACCPCCPGTSCKAFSNGVSYCRKD |
| 278 | 2 | A1S K23Y | 2.23 | 0.22 | SICTGADRPCAACCPCCPGTSCYAESNGVSYCRKDEP |
| 279 | 3 | K23Y E36- P37- | 2.67 | 1.25 | AICTGADRPCAACCPCCPGTSCYAESNGVSYCRKD |
| 280 | 2 | A1S Y31W | 2.23 | 0.00 | SICTGADRPCAACCPCCPGTSCKAESNGVSWCRKDEP |
| 285 | 4 | A1L K23Y E36- P37- | 1.78 | 0.00 | LICTGADRPCAACCPCCPGTSCYAESNGVSYCRKD |
| 300 | 3 | A24Q S26A Y31L | 1.84 | 0.06 | AICTGADRPCAACCPCCPGTSCKQEANGVSLCRKDEP |
| 302 | 4 | A1S K23Q A24I E25W | 1.93 | 0.00 | SICTGADRPCAACCPCCPGTSCQIWSNGVSYCRKDEP |
| 303 | 2 | A24Y N27L | 1.87 | 0.00 | AICTGADRPCAACCPCCPGTSCKYESLGVSYCRKDEP |
| 304 | 4 | K23T E25R S26Q S30P | 1.38 | 0.00 | AICTGADRPCAACCPCCPGTSCTARQNGVPYCRKDEP |
| 305 | 5 | A24S E25T S26G V29A | 1.19 | 0.00 | AICTGADRPCAACCPCCPGTSCKSTGNGAGYCRKDEP |
| | | S30G | | | |
| 306 | 4 | E25G N27A V29A S30F | 1.59 | 0.00 | AICTGADRPCAACCPCCPGTSCKAGSAGAFYCRKDEP |
| 308 | 4 | K23P A24D N27E S30Y | 1.71 | 0.00 | AICTGADRPCAACCPCCPGTSCPDESEGVYYCRKDEP |
| 312 | 4 | E25D S26H N27W S30P | 3.62 | 0.00 | AICTGADRPCAACCPCCPGTSCKADHWGVPYCRKDEP |
| 313 | 5 | A1S A24E S26I N27E | 2.09 | 0.00 | SICTGADRPCAACCPCCPGTSCKEEIEGPSYCRKDEP |
| | | V29P | | | |
| 315 | 3 | S26D V29G S30T | 1.82 | 0.00 | AICTGADRPCAACCPCCPGTSCKAEDNGGTYCRKDEP |
| 316 | 4 | A1S K23S A24F N27G | 1.90 | 0.00 | SICTGADRPCAACCPCCPGTSCSFESGGVSYCRKDEP |
| 317 | 3 | A24S V29S S30Y | 1.58 | 0.00 | AICTGADRPCAACCPCCPGTSCKSESNGSYYCRKDEP |
| 319 | 2 | D7N K23V | 4.46 | 0.75 | AICTGANRPCAACCPCCPGTSCVAESNGVSYCRKDEP |
| 321 | 4 | K34A D35- E36-P37- | 4.46 | 1.23 | AICTGADRPCAACCPCCPGTSCKAESNGVSYCRA |
| 322 | 2 | E36- P37- | 2.23 | 0.59 | AICTGADRPCAACCPCCPGTSCKAESNGVSYCRKD |

TABLE 5
Multiple mutations providing an improvement in expression and activity. Here, “Mut.” refers
to the point mutation relative to WT Kappa, e.g., E25F refers to a substitution of E with F
at position 25 of the WT Kappa sequence (SEQ ID NO: 1). “Exp.” refers to the expression of a
corresponding KPV relative to the expression of WT Kappa. “Act.” refers to the activity of a
corresponding KPV relative to the expression of WT Kappa. Here, activity was assessed based
on the %KD of flies dosed with 10 ng/uL of a given KPV, relative to the %KD of flies dosed
with 10 ng/uL of WT Kappa. KPVs having an N-terminus addition to the WT-Kappa sequence are
indicated as “1+” (e.g., “1+G” indicates the addition of a G residue appended to the N-
terminus of the WT Kappa peptide). Mutations where an amino acid has been deleted are
indicated as “-”

| SEQ ID | No. of | | | | |
|---|---|---|---|---|---|
| NO | mutations | Mut. | Exp. | Act. | Amino Acid Sequence |
| 2 | 4 | A1G A24T E36- P37- | 2.90 | 1.10 | GICTGADRPCAACCPCCPGTSCKTESNGVSYCRKD |
| 4 | 2 | S26A Y31R | 1.32 | 1.25 | AICTGADRPCAACCPCCPGTSCKAEANGVSRCRKDEP |
| 195 | 2 | D7G E36F | 1.01 | 1.25 | AICTGAGRPCAACCPCCPGTSCKAESNGVSYCRKDFP |
| 229 | 4 | S26A Y31R E36- | 1.56 | 1.25 | AICTGADRPCAACCPCCPGTSCKAEANGVSRCRKD |
| | | P37- | | | |
| 264 | 2 | K23Q E25A | 2.45 | 1.25 | AICTGADRPCAACCPCCPGTSCQAASNGVSYCRKDEP |
| 279 | 3 | K23Y E36- P37- | 2.67 | 1.25 | AICTGADRPCAACCPCCPGTSCYAESNGVSYCRKD |
| 321 | 4 | K34A D35- E36- P37- | 4.46 | 1.23 | AICTGADRPCAACCPCCPGTSCKAESNGVSYCRA |

### Example 5. Insertion Mutants

KPVs comprising amino acid insertions were also evaluated, wherein the insertion of one or more amino acids resulted in an elongation of the KPV amino acid sequence.

The tables below provide a list of illustrative KPVs having insertion mutations, and their corresponding expression and activity relative to WT Kappa.

Tables 6-8 below shows the effect of insertion mutations on KPV expression and activity. Here, “Mut.” refers to the point mutation relative to WT Kappa, e.g., E25F refers to a substitution of E with F at position 25 of the WT Kappa sequence (SEQ ID NO: 1). “Exp.” refers to the expression of a corresponding KPV relative to the expression of WT Kappa. “Act.” refers to the activity of a corresponding KPV relative to the expression of WT Kappa. Here, activity was assessed based on the % KD of flies dosed with 10 ng/μL of a given KPV, relative to the % KD of flies dosed with 10 ng/μL of WT Kappa. KPVs having an N-terminus addition to the WT-Kappa sequence are indicated as “1+” (e.g., “1+G” indicates the addition of a G residue appended to the N-terminus of the WT Kappa peptide).

In the tables 6-8 below, the sequence alignment begins at position 1 (aligned to position 1 of WT Kappa) and ends at position 47. The total length of the sequence alignment was based on the longest sequence analyzed; accordingly, as shown in the wild type Kappa sequence (SEQ ID NO: 1), there are positions indicated with a dash “-” where an amino acid is not present relative to the longest sequence. The numbering for the amino acids of the KPVs starts with position 1 of WT Kappa (i.e., A1), and is then aligned with the longest sequence. Each space is considered 1, e.g., the sequence “AIC-TG” corresponds to position A1, I2, C3, −4, T5, and so on, wherein there is no amino acid present at position 4 relative to the longest sequence. Any amino acids that come before A1 are named +1 or +2 to indicate an addition to the N-terminus.

TABLE 6
Effect of insertion mutations on KPV expression and activity.

| | | No | | | |
|---|---|---|---|---|---|
| SEQ ID | Sequence Alignment | of | | | |
| NO. | 1 5 10 15 20 25 30 35 40 45 | mut. | Mut. | Exp. | Act. |
| 1 | --AICTGADRPCAA--CCPCCPGTSC-KAESNG---VSYCRKDEP---- | 0 | (WT sequence; NA) | NA | NA |
| 324 | --AICTGADRPCAA--CCPCCPGTSWKQRVMVF---LIAGRMNRKRPLN | 18 | C24W 25K K26Q A27R E28V | 0 | N/A |
| | | | S29M N30V G31F V35L S36I | | |
| | | | Y37A C38G K40M D41N | | |
| | | | E42R P43K 44R 45P | | |
| 325 | --AICTGADRPCAA--CCPCCPGTSCQGPESNG---VSYCRKDEP---- | 3 | 25Q K26G A27P | 2.2 | 1.2 |
| 326 | --TICTGADRPCAA--CCPCCPGTSCTEPESNGGHTVYYCRND------ | 11 | A1T 25T K26E A27P 32G 33H | 0.0 | N/A |
| | | | 34T S36Y K40N E42 P43 | | |
| 327 | --QACTPADQPCAA--CCPCCSGASCTEQLNENGHTVYYCRA------- | 21 | A1Q I2A G5P R8Q P20S T22A | 4.5 | 0 |
| | | | 25T K26E A27Q E28L S29N | | |
| | | | N30E G31N 32G 33H 34T | | |
| | | | S36Y K40A D4I E42 P43 | | |
| 328 | --AICTGADRPCAA--CCPCCPGTSCQGPESNG---VSYCRA------- | 7 | 25Q K26G A27P K40A D4I | 1.6 | 1.2 |
| | | | E42 P43 | | |
| 329 | --AICTGADRPCAA--CCPCCPGTSCAGPESNG---VSYCRKDEP---- | 3 | 25A K26G A27P | 1.8 | 1.2 |
| 330 | --AICTGADRPCAA--CCPCCPGTSCQGAESNG---VSYCRKDEP---- | 2 | 25Q K26G | 0.4 | 0.6 |
| 331 | --AICTGADRPCAA--CCPCCPGTSCKGPESNG---VSYCRKDEP---- | 3 | 25K K26G A27P | 2.0 | 1.3 |
| 332 | --AICTGADRPCAA--CCPCCPGTSCNGPESNG---VSYCRKDEP---- | 3 | 25N K26G A27P | 1.6 | 1.0 |
| 333 | -QAICTGADRPCAA--CCPCCPGTSCQGPESNG---VSYCRKDEP---- | 3 | +1Q 25Q K26G A27P | 0.0 | N/A |
| 334 | -NAICTGADRPCAA--CCPCCPGTSCQGPESNG---VSYCRKDEP---- | 3 | +1N 25Q K26G A27P | 2.7 | 0 |
| 335 | --AICTGADRPCAASQCCPCCPGTSCTEPESNGGHTVYYCRND------ | 12 | 13S 14Q 25T K26E A27P 32G | 0.0 | N/A |
| | | | 33H 34T S36Y K40N E42 P43 | | |
| 336 | --AICTGADRPCAA--CCPCCPGTSCTEPESNGGHTVYYCRND------ | 10 | 25T K26E A27P 32G 33H 34T | 0.0 | N/A |
| | | | S36Y K40N E42 P43 | | |
| 337 | --QACTGADRPCAA--CCPCCPGTSCTEPESNGGHTVYYCRND------ | 12 | A1Q I2A 25T K26E A27P 32G | 0.0 | N/A |
| | | | 33H 34T S36Y K40N E42 P43 | | |
| 338 | QATICTGADRPCAA--CCPCCPGTSCTEPESNGGHTVYYCRND------ | 11 | +2Q +1A A1T 25T K26E A27P | 0.0 | N/A |
| | | | 32G 33H 34T S36Y K40N E42 | | |
| | | | P43 | | |
| 339 | --AICTGADRPCAA--CCPCCSGASCGGPESNG---VSYCRKDEP---- | 5 | P20S T22A 25G K26G A27P | 0.0 | N/A |
| 340 | --AICTGADRPCAA--CCPCCSGASCGGPESNG---VSYCRA------- | 9 | P20S T22A 25G K26G A27P | 0.0 | N/A |
| | | | K40A D4 E42 P43 | | |
| 341 | --AICTGADRPCGA--CCPCCSGASCGGPESNG---VSYCRKDEP---- | 6 | A11G P20S T22A 25G K26G | 0.0 | N/A |
| | | | A27P | | |
| 342 | --AICTGADRPCGA--CCPCCSGASCGGPESNG---VSYCRA------- | 10 | A11G P20S T22A 25G K26G | 0.0 | N/A |
| | | | A27P K40A D41 E42 P43 | | |
| 343 | --TICTGADRPCAA--CCPCCPGTSCTEPESNG---VSYCRND------ | 7 | A1T 25T K26E A27P K40N | 0.4 | 1.3 |
| | | | E42 P43 | | |
| 344 | --AICTGADRPCAA--CCPCCPGTSCTEPESNG---VSYCRND------ | 6 | 25T K26E A27P K40N E42 | 0.9 | 1.3 |
| | | | P43 | | |
| 345 | --SICTGADRPCAA--CCPCCPGTSCQQDEFQN---VWNCRKDEP---- | 9 | A1S 25Q K26Q A27D S29F | 0.4 | 0.0 |
| | | | N30Q G31N S36W Y37N | | |
| 346 | --SICTGADRPCAA--CCPCCPGTSCHKADSNG---LSYCRKDEP---- | 4 | A1S 25H E28D V35L | 1.3 | 0.2 |
| 347 | --SICTGADRPCAA--CCPCCPGTSCIKRESNG---VSYCRKDEP---- | 3 | A1S 25I A27R | 0.4 | 1.3 |
| 348 | --AICTGADRPCAA--CCPCCPGTSCTKRESNG---VKYCRKDEP---- | 3 | 25T A27R S36K | 0.6 | 1.3 |
| 349 | --AICTGADRPCAA--CCPCCPGTSCQKAESNG---VLYCRKDEP---- | 2 | 25Q S36L | 0.6 | 0.1 |
| 350 | --TICSGGDRPRAA--CCPCCPGTSSGKARVNG---VFYCRKDEP---- | 9 | A1T T4S A6G C10R C24S | 1.0 | 0.7 |
| | | | 25G E28R S29V S36F | | |
| 351 | --SICTGADRPCAA--CCPCCPGTSCRKADNGG---VDYCRKDEP---- | 6 | A1S 25R E28D S29N N30G | 0.7 | 0.0 |
| | | | S36D | | |
| 352 | --AICTGADRPCAA--CCPCCPGTSCVKARVNG---VFYCRKDEP---- | 4 | 25V E28R S29V S36F | 0.4 | 0.9 |
| 353 | --SICTGADSPCAA--CCPSCPGTSCKGASWWG---VCNCRKDEP---- | 10 | A1S R8S C18S 25K K26G | 0.4 | 0.5 |
| | | | E28S S29W N30W S36C | | |
| | | | Y37N | | |
| 354 | --AICTGADRPCAA--CCPCCPGTSCTKAESNG---ISYCRKDEP---- | 2 | 25T V35I | 1.6 | 1.3 |
| 355 | --SICTGADRPCAA--CCPCCPGTSCQKDDHNG---VGYCRKDEP---- | 6 | A1S 25Q A27D E28D S29H | 1.9 | 0.0 |
| | | | S36G | | |
| 356 | --AICTGADRPCAA--CCPCCPGTSCYKAPDNG---LPYCRKDEP---- | 5 | 25Y E28P S29D V35L S36P | 0.6 | 0.0 |
| 357 | --AICTGADRPCAA--YCPCCPGTSCQKALSNG---SSYCRKDEP---- | 4 | C15Y 25Q E28L V35S | 0.6 | 0.0 |
| 358 | --AICTGADRPCAA--CCPCCPGTSC-KAESNG---VSYCRVDEP---- | 1 | K40V | 2.8 | 0.6 |

TABLE 7
KPVs having improved expression or activity.

| | | No. | | | |
|---|---|---|---|---|---|
| SEQ ID | Sequence Alignment | of | | | |
| NO. | 1 5 10 15 20 25 30 35 40 45 | mut. | Mut. | Exp. | Act. |
| 325 | --AICTGADRPCAA--CCPCCPGTSCQGPESNG---VSYCRKDEP---- | 3 | 25Q K26G A27P | 2.23 | 1.24 |
| 327 | --QACTPADQPCAA--CCPCCSGASCTEQLNENGHTVYYCRA------- | 21 | A1Q I2A G5P R8Q P20S T22A | 4.46 | 0.00 |
| | | | 25T K26E A27Q E28L S29N | | |
| | | | N30E G31N 32G 33H 34T | | |
| | | | S36Y K40A D41 E42 P43 | | |
| 328 | --AICTGADRPCAA--CCPCCPGTSCQGPESNG---VSYCRA------- | 7 | 25Q K26G A27P K40A D41 | 1.56 | 1.25 |
| | | | E42 P43 | | |
| 329 | --AICTGADRPCAA--CCPCCPGTSCAGPESNG---VSYCRKDEP---- | 3 | 25A K26G A27P | 1.78 | 1.25 |
| 331 | --AICTGADRPCAA--CCPCCPGTSCKGPESNG---VSYCRKDEP---- | 3 | 25K K26G A27P | 2.01 | 1.25 |
| 332 | --AICTGADRPCAA--CCPCCPGTSCNGPESNG---VSYCRKDEP---- | 3 | 25N K26G A27P | 1.56 | 1.00 |
| 334 | -NAICTGADRPCAA--CCPCCPGTSCQGPESNG---VSYCRKDEP---- | 3 | +1N 25Q K26G A27P | 2.67 | 0.00 |
| 343 | --TICTGADRPCAA--CCPCCPGTSCTEPESNG---VSYCRND------ | 7 | A1T 25T K26E A27P K40N | 0.45 | 1.25 |
| | | | E42 P43 | | |
| 344 | --AICTGADRPCAA--CCPCCPGTSCTEPESNG---VSYCRND------ | 6 | 25T K26E A27P K40N E42 | 0.89 | 1.25 |
| | | | P43 | | |
| 346 | --SICTGADRPCAA--CCPCCPGTSCHKADSNG---LSYCRKDEP---- | 4 | A1S 25H E28D V35L | 1.29 | 0.20 |
| 347 | --SICTGADRPCAA--CCPCCPGTSCIKRESNG---VSYCRKDEP---- | 3 | A1S 25I A27R | 0.39 | 1.25 |
| 348 | --AICTGADRPCAA--CCPCCPGTSCTKRESNG---VKYCRKDEP---- | 3 | 25T A27R S36K | 0.60 | 1.25 |
| 350 | --TICSGGDRPRAA--CCPCCPGTSSGKARVNG---VFYCRKDEP---- | 9 | A1T T4S A6G C10R C24S | 1.03 | 0.74 |
| | | | 25G E28R S29V S36F | | |
| 354 | --AICTGADRPCAA--CCPCCPGTSCTKAESNG---ISYCRKDEP---- | 2 | 25T V35I | 1.63 | 1.25 |
| 355 | --SICTGADRPCAA--CCPCCPGTSCQKDDHNG---VGYCRKDEP---- | 6 | A1S 25Q A27D E28D S29H | 1.86 | 0.00 |
| | | | S36G | | |
| 358 | --AICTGADRPCAA--CCPCCPGTSC-KAESNG---VSYCRVDEP---- | 1 | K40V | 2.82 | 0.63 |

TABLE 8
KPVs having improved expression and activity.

| SEQ | | No. | | | |
|---|---|---|---|---|---|
| ID | Sequence Alignment | of | | | |
| NO. | 1 5 10 15 20 25 30 35 40 45 | mut. | Mut. | Exp. | Act. |
| 325 | --AICTGADRPCAA--CCPCCPGTSCQGPESNG---VSYCRKDEP---- | 3 | 25Q K26G | 2.23 | 1.24 |
| | | | A27P | | |
| 328 | --AICTGADRPCAA--CCPCCPGTSCQGPESNG---VSYCRA------- | 7 | 25Q K26G | 1.56 | 1.25 |
| | | | A27P K40A D41 | | |
| | | | E42 P43 | | |
| 329 | --AICTGADRPCAA--CCPCCPGTSCAGPESNG---VSYCRKDEP---- | 3 | 25A K26G A27P | 1.78 | 1.25 |
| 331 | --AICTGADRPCAA--CCPCCPGTSCKGPESNG---VSYCRKDEP---- | 3 | 25K K26G A27P | 2.01 | 1.25 |
| 354 | --AICTGADRPCAA--CCPCCPGTSCTKAESNG---ISYCRKDEP---- | 2 | 25T V35I | 1.63 | 1.25 |

### Example 6. Yield Per Copy Number

To evaluate the relationship between copy number and yield, four recombinant yeast cell strains were made:

(1) a first recombinant yeast cell strain transformed with a vector comprising a dual expression cassette comprising two nucleotide sequences operable to encode the WT Kappa peptide:

(SEQ ID NO: 1)
“AICTGADRPCAACCPCCPGTSCKAESNGVSYCRKDEP”.

(2) a second recombinant yeast cell strain transformed with a vector comprising a dual expression cassette comprising two nucleotide sequences operable to encode the KPV2 peptide:

(SEQ ID NO: 2)
″GICTGADRPCAACCPCCPGTSCKTESNGVSYCRKD”.

(3) a third recombinant yeast cell strain transformed with a vector comprising a dual expression cassette comprising two nucleotide sequences operable to encode the KPV3 peptide:

(SEQ ID NO: 3)
“SICTGADRPCAACCPCCPGTSCKAGVNGVAYCRKDEP”.

(4) a fourth recombinant yeast cell strain transformed with a vector comprising a dual expression cassette comprising two nucleotide sequences operable to encode the KPV4 peptide:

(SEQ ID NO: 4)
“AICTGADRPCAACCPCCPGTSCKAEANGVSRCRKDEP”.

Briefly, the vectors comprised a 5′-homology arm and a 3′-homology arm corresponding to loci in the LAC4 promoter (pLAC4) (allowing integration of the expression cassettes into the endogenous pLAC4 loci), and the actual polynucleotide comprising the expression cassette. Here, the expression cassettes, i.e., cassette #1 comprised following:
(1) Cassette #1: an intact heterologous pLAC4, and a nucleotide sequence operable to encode a heterologous polypeptide (WT Kappa, KPV2, KPV3, or KPV4);
(2) an alpha-MF signal sequence.

For each of the expression cassettes, there was a single copy of an amdS transgene (note: the relationship between copy number and yield is independent of the selection marker used in the vector). In addition, the vectors comprised the following elements: a Kex2 cleavage site; a multiple cloning site; a LAC4 terminator (cassette #1); and ADH1 promoter; a β-lactamase (bla) gene; and an origin of replication site.

These aforementioned vectors were transformed into the K. lactis strain, YCT306, (New England Biolabs, Ipswich, MA, USA), resulting in recombinant yeast cell strains possessing a varied number of copies. Individual recombinant yeast cells, each with a potentially different number of copies of a heterologous polynucleotide, were screened for expression of the heterologous polypeptide after being grown in culture.

After 6 days, the supernatant of the deep well cultures were assessed for the level of expression of either WT Kappa, KPV2, KPV2, or KPV4, was determined via HPLC. The recombinant yeast cells then had gDNA extracted using an Invitrogen yeast gDNA extraction kit (ThermoFisher; Catalog No. 78870) and qPCR was used to estimate the number of copies of the nucleotide sequence operable to encode the peptides that were integrated.

Here, qPCR primers for actin were used as an endogenous control, and copies of pLAC4 were used to estimate the number of copies of heterologous polynucleotide. pLAC4 primers amplify the pLAC4 copy driving expression cassette #1. Number of copies of the nucleotide sequences operable to encode the peptides that had been integrated into the host cell genome, were determined according to the following formula:

$$KVP Copies = ( RQ of pLAC 4 - 1 ) \qquad Formula ( a )$$

Wherein RQ is relative quantification. Here, each transformant has one native pLAC4 sequence and one KPV sequence for each additional pLAC4 sequence beyond the native sequence.

### Results

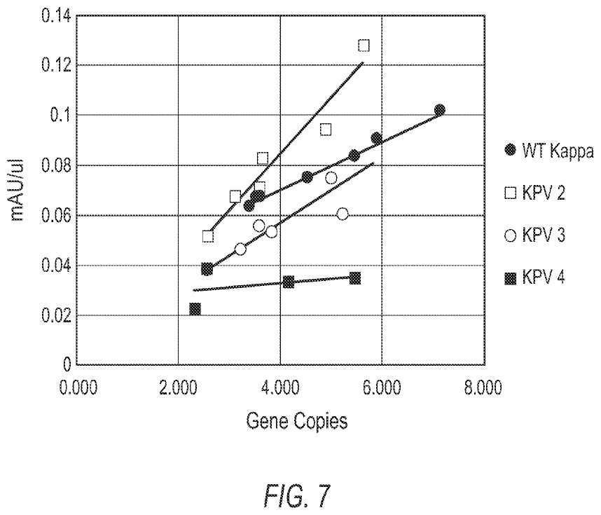

The results of the yield-per-copy-number assay is shown in FIG. 7. The average area per gene is shown in the table below; the amount of peptide (area) per gene insertion (gene) illustrates that some variant peptides have better rates of expression, when the number of gene copies are equal.

TABLE 9
Average area per gene mean. Here, the amount of peptide
(area) per gene insertion (gene) is show. The percent
change relative to WT Kappa (Native) is also shown.

| Name | Average area/gene | % WT |
|---|---|---|
| WT Kappa | 1.68 | — |
| KPV2 | 2.1 | 125 |
| KPV3 | 1.43 | 85.11905 |
| KPV4 | 0.98 | 58.33333 |

The results of the yield-per-gene-copy analysis revealed that KPV2 had a 25% improvement in expression relative to WT Kappa when controlling for the number of gene copies.

Example 7. Stability in Acidic and/or Heated Conditions

KPVs of the present disclosure were evaluated after incubation in acidic and/or heated conditions. Briefly, WT Kappa, KPV1, KPV2, KPV3, or KPV4 were added to either pure water or buffers, and then subjected to heated conditions at 54° C., or heat and acidic conditions (pH 3.5), for 14 days. Pre-incubation and post-incubation samples were then evaluated via HPLC as described above.

As shown in FIG. 8, incubation of WT Kappa, KPV1, KPV3, and KPV4 under heat and acidic conditions causes a peak shift which correlates to the cleavage of the residues E and P from the C-terminus. The cleavage phenomenon was observed with KPV1, KPV3, and KPV4, but not KPV2 as it does not have a C-terminus “EP.”

### Example 8. KPV2 Stability Assay

The stability of KPV2 was evaluated after incubation in acidic and/or heated conditions. Briefly, KPV2 was added to either pure water or Tris pH 7.2, and incubated at 54° C. for 7 days. The treated KPV2 was then evaluated via HPLC as described above, and compared to pre-incubation samples.

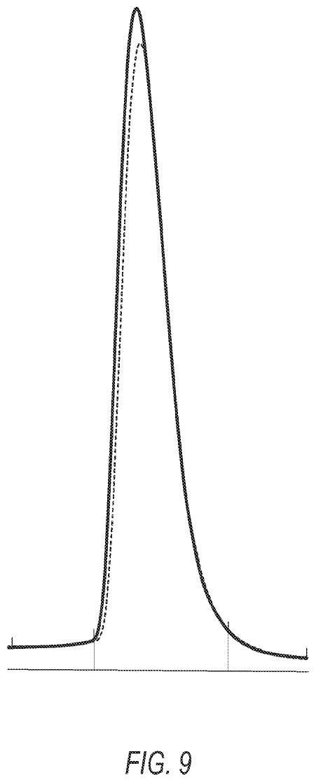

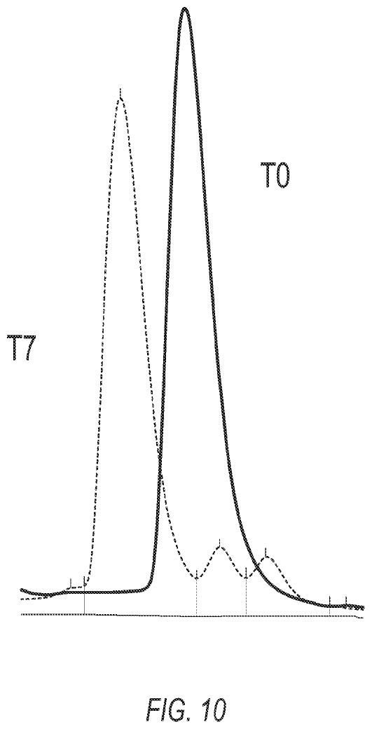

As shown in FIG. 9, incubation of KPV2 in water and acid (pH 3.0) for 7 days did not result in any new peaks. As shown in FIG. 10, incubation of KPV2 in Tris buffer (pH 7.2) for 7 days resulted in new peaks forming.

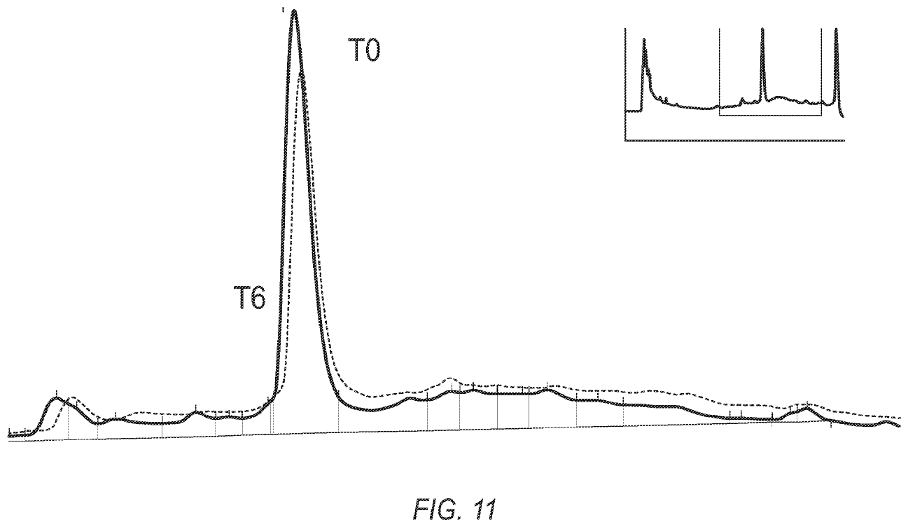

Next, KPV2 beer was analyzed after incubation at 54° C. for 6 days. As shown in FIG. 11, there was a 17% loss in peak area, but no new peaks formed.

### Example 9. Fly Injection Assay

A housefly injection assay was performed to determine the activity of KPV2 as compared to WT Kappa. Adult houseflies were immobilized with CO₂ for 10 minutes, and then transferred to a CO₂ pan to keep immobilized. Flies with weight between 12-20 mg were picked for injection. The KPV2 or WT Kappa were diluted in water to proper doses for injection. Next, a 0.5 μL peptide solution was injected into each housefly at the dorsal thorax using a hand-microapplicator with 1 cc all-glass syringe with 30 gauge straight needle. The injected flies were then transferred to a 2 oz. transparent portion container with a wet #4 filter paper. Fly mortality was assessed at 24-hours post-injection.

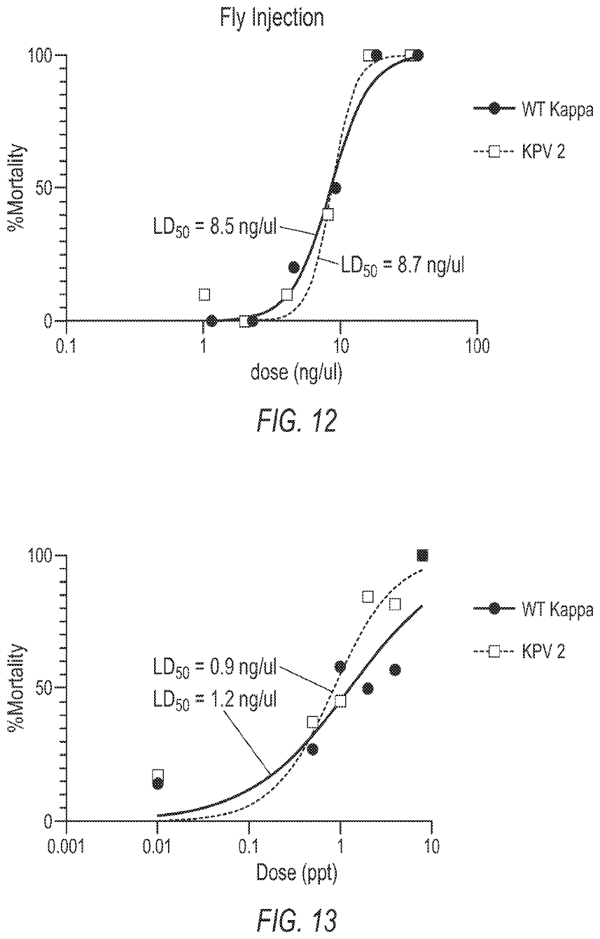

As shown in FIG. 12, KPV2 has similar activity to WT Kappa in a fly injection assay. The LD₅₀ of WT Kappa was 8.5 ng/μL, whereas the LD₅₀ for KPV2 was 8.7 ng/μL.

### Example 10. Lepidopteran Diet Assay

A diet incorporation assay was performed to determine the concentration of: WT Kappa, or concentrated KPV2 fermentation beer, that was required to kill 50% of a neonate corn earworm (CEW) population (LD₅₀). Briefly, a general lepidopteran diet was prepared in 96-well plates at 100 μL of diet per well. The plates containing the diet were placed under fan on a benchtop overnight, to allow the diet to dry. The next day, the now desiccated diet was incubated with 60 μL of a given treatment solution for about 1 hour at 24° C. The treatments consisted of WT Kappa, or KPV2, at the concentrations ranging from 0.01 parts per thousand (ppT) to 9 ppT. Additional water was added to account for evaporation loss during the hour incubation period.

At each concentration of either WT Kappa or KPV2, the treatment solution was applied to 8 wells within a 96-well plate. A single neonate CEW larva was then placed in each well after the treatment solution had been absorbed by the diet. LD₅₀ was scored after 4 days.

As shown in FIG. 13, the LD₅₀ for WT Kappa was 1.2 ppT, whereas the LD₅₀ for KPV2 was 0.9 ppT. This shows that WT Kappa and KPV2 have similar activity levels in a lepidopteran diet assay, although, KPV2 shows an improvement in bioactivity compared with wild-type Kappa peptide of SEQ ID NO: 1.

### Example 11. Summary of Illustrative KPVs.

A summary of the mutants evaluated is presented in the table 10 below.

TABLE 10
Summary of KPVs evaluated. Here, “Mut.” refers to the point mutation relative to WT Kappa, e.g., E25F
refers to a substitution of E with F at position 25 of the WT Kappa sequence (SEQ ID NO: 1). “Exp.” refers to the expression of a
corresponding KPV relative to the expression of WT Kappa. “Act.” refers to the activity of a corresponding KPV relative to the
expression of WT Kappa. Here, activity was assessed based on the %KD of flies dosed with 10 ng/uL of a given KPV, relative to the
%KD of flies dosed with 10 ng/uL of WT Kappa. KPVs having an N-terminus addition to the WT-Kappa sequence are indicated as
“1+” (e.g., “1+G” indicates the addition of a G residue appended to the N-terminus of the WT Kappa peptide). Mutations where an
amino acid has been deleted are indicated as “-”

| SEQ ID | | | No. of | | | |
|---|---|---|---|---|---|---|
| NO. | Name | Amino acid sequence | mutations | Mut. | Exp. | Act. |
| 1 | WT | AICTGADRPCAACCPCCPGTSCKAESNGVSYCRKDEP | 0 | | 1 | 1 |
| | Kappa | | | | | |
| 2 | KPV2 | GICTGADRPCAACCPCCPGTSCKTESNGVSYCRKD | 4 | A1G A24T E36- | 2.902201 | 1.1 |
| | | | | P37- | | |
| 3 | KPV3 | SICTGADRPCAACCPCCPGTSCKAGVNGVAYCRKDEP | 4 | AIS E25G S26V | 1.8983 | 0.75 |
| | | | | S30A | | |
| 4 | KPV4 | AICTGADRPCAACCPCCPGTSCKAEANGVSRCRKDEP | 2 | S26A Y31R | 1.320145 | 1.25 |
| 5 | KPV5 | AICTGADRPCAACCPCCPGTSCKAFSNGVSYCRKDEP | 1 | E25F | 2.229033 | 1.25 |
| 6 | KPV1 | AICTGADRPCAACCPCCPGTSCKAEWNGVSYCRKDEP | 1 | S26W | 4.458066 | 0.625 |
| 7 | KPV7 | AICTGADRPCAAICPCCPGTSCKAESNGVSYCRKDEP | 1 | C13I | 3.077208 | N/A |
| 8 | KPV8 | AICTGADRPCALCCPCCPGTSCKAESNGVSYCRKDEP | 1 | A12L | 0.931736 | 0 |
| 9 | KPV9 | AINTGADRPCAACCPCCPGTSCKAESNGVSYCRKDEP | 1 | C3N | 0 | N/A |
| 10 | KPV10 | AICTGIDRPCAACCPCCPGTSCKAESNGVSYCRKDEP | 1 | A6I | 1.524101 | 0 |
| 11 | KPV11 | AICTGADRPCAACCPCCPGTSCKAESNGVSHCRKDEP | 1 | Y31H | 3.70131 | 0 |
| 12 | KPV12 | AICTGADRPCAACCPCCPGTSSKAESNGVCYCRKDEP | 2 | C22S S30C | 1.354138 | 0.5 |
| 13 | KPV13 | AICTEADRPCHACCPCCPGTSCKAESNGVRYCRKDEP | 3 | G5E A11H S30R | 1.773475 | N/A |
| 14 | KPV14 | AICTGADRPCAACCPCCPGTSCKAESNGVSPCRKDEP | 1 | Y31P | 0 | N/A |
| 15 | KPV15 | AICTGADLPCAACCPCCPGTSCKAESNGVSYCRGDEP | 2 | R8L K34G | 0 | N/A |
| 16 | KPV16 | AICTGADRPCAICCPCCPGTSCKAESNGVSYCRKDEP | 1 | A12I | 1.100306 | 0 |
| 17 | KPV17 | AICEGADRPCAACCPCCPGTSCKAESNGVSYCRKDEP | 1 | T4E | 1.124826 | N/A |
| 18 | KPV18 | AICTGADRPCAACCPCCPGTSCKAESRGVSYCRKDEP | 1 | N27R | 1.332683 | 0 |
| 19 | KPV19 | AICTGADRPCAACCPCCPGTSCKAESNGVSYGRKDEP | 1 | C32G | 1.611312 | N/A |
| 20 | KPV20 | AICTGADRPCAACCPCCPGTSCKAESNAVSYCRKDEP | 1 | G28A | 0.438841 | 0 |
| 21 | KPV21 | AICTGADRPCAACCPCCLGTSCKAESNGVSYCRKDEP | 1 | P18L | 0.62608 | 0 |
| 22 | KPV22 | AIYTGADRPCAACCPCCPGTSCKAESNGVSYCRKDEP | 1 | C3Y | 0 | N/A |
| 23 | KPV23 | AICTGADRPCAACCPCCPGTSCKYESNGVSYCRKDEP | 1 | A24Y | 1.155754 | 0.5 |
| 24 | KPV24 | AICTGADRPCAACCPCCPGTSCKAESFGVSYCRKDEP | 1 | N27F | 0 | N/A |
| 25 | KPV25 | AICTGADRPCAACNPCCPGTSCKAESNGVSYCRKMEP | 2 | C14N D35M | 1.246587 | N/A |
| 26 | KPV26 | AICIGADRPCAACCPCCPGTSCKAESNGVSYCRKDEP | 1 | T4I | 0.941209 | N/A |
| 27 | KPV27 | AICTGADRPCAACCPCCPGTSCKAESNGCSYCRKDEP | 1 | V29C | 0.609919 | 0 |
| 28 | KPV28 | AICTGADRPCAACCPCCPGVSCKAESNGVSYCRKDEP | 1 | T20V | 0 | N/A |
| 29 | KPV29 | AICTGADRPCAACCPCPPGTSCKAESNGVSYCRKDEP | 1 | C17P | 0.957927 | N/A |
| 30 | KPV30 | AICTGADRPYAACCPCCPGTSCKAESNGVSYCRKDEP | 1 | C10Y | 1.684592 | N/A |
| 31 | KPV31 | AICTGADRPKAACCPCCPGTSCKAESNGVSYCRKDEP | 1 | C10K | 2.415436 | N/A |
| 32 | KPV32 | AICTGADRPDAACCPCCPGTSCKAESNGVSYCRKDEP | 1 | C10D | 0 | N/A |
| 33 | KPV33 | AICTGADRPCAACCPCCRGTSCKAESNGVSYCRKDEP | 1 | P18R | 0.874338 | 0.5 |
| 34 | KPV34 | AICTGGDRPCAACCPCCPGTSCKAESNGVSYCRKDEP | 1 | A6G | 0.546237 | 0.083333 |
| 35 | KPV35 | AICTGADRPCAACCPCCPGTSCKAESNGVSYCRKNEP | 1 | D35N | 1.63 | 0.56 |
| 36 | KPV36 | AICTGADRPCAACCPCCPGTTCKAESNGVSYCRKDEP | 1 | S21T | 0.241293 | 0 |
| 37 | KPV37 | AICTGADRPCAACCPQCPGTSCKAESNGVSYCRKDEP | 1 | C16Q | 0 | N/A |
| 38 | KPV38 | AICTGADRPCAACCPCCPGTSCKAESNYVSYCRKDEP | 1 | G28Y | 0.659237 | 0.25 |
| 39 | KPV39 | AICTKADRPCAACCPCCPGTSCKAASNGVSYCRKDEP | 2 | G5K E25A | 1.505991 | 0 |
| 40 | KPV40 | AVCTGADRPCAACCPCCPGTSCKAESNGVSYCRKDEP | 1 | I2V | 2.29 | 0.375 |
| 41 | KPV41 | AICTGADRPCAACCPCCPGTSCKANSNGVSYCRKDEP | 1 | E25N | 0.771524 | 0 |
| 42 | KPV42 | AICTGADEPCAACCPCCPGTSCKAESNGVSYCRKDEP | 1 | R8F | 0 | N/A |
| 43 | KPV43 | AICTGADRPIAACCPCCPGTSCKAESNGVSYCRKDEP | 1 | C10I | 0 | N/A |
| 44 | KPV44 | AICTLADRPCAACCPCCPGQSCKAESNGVSYCRTDEP | 3 | G5L T20Q K34T | 0 | N/A |
| 45 | KPV45 | AICTGADRPCAACCPCCPGSSCKAEGNGVSYCRKDEP | 2 | T20S S26G | 1.604904 | 0 |
| 46 | KPV46 | AICTGADRPCAACCPCCPGTSCKAEPNGVSYCRRDEP | 2 | S26P K34R | 1.484536 | 0 |
| 47 | KPV47 | AICTGADRPCAACCPCCPGTSCKAESNGVSYCRKEKAKR | 5 | D35E E36K P37A- | 0.922262 | 0 |
| | | PLN | | 38K -39R | | |
| 48 | KPV48 | AICTGADRPCAACCPCCPGTSCKAESNGVSYCRKDVP | 1 | E36V | 1.28058 | 0 |
| 49 | KPV49 | AICTGADRPCAACCPCCPGTSCKAESNGVSYCKKDEP | 1 | R33K | 0.904152 | 0 |
| 50 | KPV50 | AICTGADRPCAACCPCCPGTSCKAEPNGVSYCRKDEP | 1 | S26P | 1.305099 | 0 |
| 51 | KPV51 | AICTGADRPCAACCPCCPGTSCKAESNGVCYCRKDEP | 1 | S30C | 0 | N/A |
| 52 | KPV52 | AICTGADRICAACCPCCPGTSCKAESNGVSYCRKDEP | 1 | P9I | 0.555029 | N/A |
| 53 | KPV53 | AICTGADRPCAACCPCWPGTSCKAESNGVSYCRKDEP | 1 | C17W | 0 | N/A |
| 54 | KPV54 | AICTGADRPCAACCPCCPGTSCKAESNGVSFCRKDEP | 1 | Y31F | 2.85 | 0.625 |
| 55 | KPV55 | AICTDADRPCAACCPCCPGTSCKAESNGVSYCRKDEP | 1 | G5D | 1.782948 | 0 |
| 56 | KPV56 | AIYTGADRPCAACCPCCPGTSCKAESNGVSNCRKDEP | 2 | C3Y Y31N | 2.506269 | N/A |
| 57 | KPV57 | AICTGADRPCAAGCPCCPGTSCKAESNGVSYCRKDEP | 1 | C13G | 1.503808 | N/A |
| 58 | KPV58 | AICTRADRPQAACCPCCPGTSCKAESNGVSYCRKDEP | 2 | G5R C10Q | 0.972973 | 0 |
| 59 | KPV59 | AICTGADRPCSACCPCCPGTSCKAESNGVSYCRKDEP | 1 | A11S | 0.91864 | 0 |
| 60 | KPV60 | AICTGADRPCAACCPCCPGTSCKAESVGVSYCRKDEP | 1 | N27V | 1.435358 | 0.25 |
| 61 | KPV61 | AIPTGADRPCAACCPCCPGTNCKAESNGVSYCRKDEP | 2 | C3P S21N | 0 | N/A |
| 62 | KPV62 | AICTGADRNCAACCPCCPGTSCKAESNGVSYCRKDEP | 1 | P9N | 0 | N/A |
| 63 | KPV63 | AICTGADRPCAACCPCCPGTSCKAESNGVSNCRKDEP | 1 | Y31N | 0 | N/A |
| 64 | KPV64 | AICTIADRPCAACCPCFGGTSCKAESNGVSYCRKDEP | 3 | G5I C17F P18G | 0 | N/A |
| 65 | KPV65 | AICTGADRPCAACCPCCPGTSCKAPSNGVSYCRKDDP | 2 | E25P E36D | 0 | N/A |
| 66 | KPV66 | AICRGADRPCAACCPCCPGTSCKAESNGVSYCRKDEP | 1 | T4R | 2.025913 | N/A |
| 67 | KPV67 | AICTGADRPCAACCPCCPITSCKAESNGVSYCRKDEP | 1 | G19I | 0 | N/A |
| 68 | KPV68 | AICTGADRPCAACCPCCPGTSCWAESNGVSYCRKDEP | 1 | K23W | 1.140847 | 0.5625 |
| 69 | KPV69 | AICTGADRPCAACCPCCPGTSWKAESNGVSYCRKDEP | 1 | C22W | 0 | N/A |
| 70 | KPV70 | AICTGADRPCAACCPCYPGTSCKAESNGVSYCRKDEP | 1 | C17Y | 2.032042 | N/A |
| 71 | KPV71 | AICTGADRPCAAECPCCPGTSCKAESNGVSYCRKDEP | 1 | C13E | 1.607412 | 0 |
| 72 | KPV72 | AICTGADRPCAACCPYCPGTSCKAESNGVSYCRKDCP | 2 | C16Y E36C | 0.976317 | N/A |
| 73 | KPV73 | AICTGADGPCAACCPCCPGTSCKAESNGVSYCRKDES | 2 | R8G P37S | 1.198105 | N/A |
| 74 | KPV74 | AICTGADSPCAACCPCCPGTTCKAESNGVSYCRKDEP | 2 | R8S S21T | 1.499025 | 0 |
| 75 | KPV75 | AICTGADRPCAACCPCCPGCSCKAESNGVSYCRKDEP | 1 | T20C | 0 | N/A |
| 76 | KPV76 | AICTGADRPCAACCPCCPGTSCKAETNGVSYCRKDEP | 1 | S26T | 1.624408 | 0 |
| 77 | KPV77 | AICTGADRPCAACCPCCPGTSCKAESNGVSYCRGDEP | 1 | K34G | 2.411953 | 0.6875 |
| 78 | KPV78 | AICTGADRPCSACCPCCPGHSCKAESNGVSYCRKDEP | 2 | A11S T20H | 1.190861 | 0.25 |
| 79 | KPV79 | AICTGADRPRAACCPCCPGTSCKAESNGVSYCRKDEP | 1 | C10R | 1.167456 | 0 |
| 80 | KPV80 | AICTGAARPCAACCPCCPGTSCAAESNGVSYCRKDEP | 2 | D7A K23A | 0 | N/A |
| 81 | KPV81 | AICTGADRPCAACCPCCPGTDCKAESNGVSYCRKDEP | 1 | S21D | 0.393424 | 0.75 |
| 82 | KPV82 | KICTGADRPCAACCPCCPGTSCKAESNGVSYCRKDEP | 1 | A1K | 0 | N/A |
| 83 | KPV83 | AICTGADRPCAACCPCCPGTSCKAESNGVVYCRKDEP | 1 | S30V | 0 | N/A |
| 84 | KPV84 | AICTGADRPCAACCPCCPGTHCKAESNGVSYCRKDEP | 1 | S21H | 0 | N/A |
| 85 | KPV85 | AICTGADRPCAACCPCCPGTSCKAESNGVSYCRADEP | 1 | K34A | 1.306794 | 0.5 |
| 86 | KPV86 | AICTHADRPCAACCPCCPGTGCKAESNGVSYCRKDEP | 2 | G5H S21G | 0 | N/A |
| 87 | KPV87 | AICTGADRPCAACCPCCPGTTCKAERNGVSYCRKDEP | 2 | S21T S26R | 0.68097 | 0 |
| 88 | KPV88 | AICTGADRPCAACCPCCPGTSCKAASNGVSYCRKDEP | 1 | E25A | 0.559766 | 0.75 |
| 89 | KPV89 | AICTGADRPCAACCPGCPGTSCKAESNGVSYCRKDEP | 1 | C16G | 0 | N/A |
| 90 | KPV90 | AICTGADRPECCPCCPGTSCKAESNGVSYCRPDEP | 4 | C10- A11- A12E | 0 | N/A |
| | | | | K34P | | |
| 91 | KPV91 | AVCTGADRPCAACCPCCPGISCKAESNGVSYCRKDEP | 2 | I2V T20I | 0 | N/A |
| 92 | KPV92 | AICTGADRPCAACCPCCPGASCKAESNGVSYCRKDEP | 1 | T20A | 0 | N/A |
| 93 | KPV93 | AICTGADRPCAACCPCCPGTSCKAERNGVCYCRKDEP | 2 | S26R S30C | 0.824185 | 0 |
| 94 | KPV94 | AICTGVDRPCAACCPCCPGTSCKAESKGVSYCRKDEP | 2 | A6V N27K | 0.423516 | 0.5 |
| 95 | KPV95 | AICTGADRPCAACCPCCPGTSCKAESAGVSYCRKDEP | 1 | N27A | 0.748955 | 0 |
| 96 | KPV96 | AICTGADRPCAACCPCTPGTSCKAESNGVSYCRKDEP | 1 | C17T | 0.507662 | 0 |
| 97 | KPV97 | AICTRADRPCAACCPCCPGTSCKAESNGVSYCRKDEP | 1 | G5R | 0.925327 | 0.75 |
| 98 | KPV98 | AICTGADRPCAACCPCCPGTSCKAESNGVSYCRKDHP | 1 | E36H | 0.883812 | 0.75 |
| 99 | KPV99 | AIDTGADRPCAACCPCCPGTSCKAESNGVSYCRKDEP | 1 | C3D | 0 | N/A |
| 100 | KPV100 | AICTGVDRPCAACCPCCPGTSCNAESNGVSLQER | 9 | A6V K23N Y31L | 0 | N/A |
| | | | | C32Q R33E K34R | | |
| | | | | D35- E36- P37- | | |
| 101 | KPV101 | AICTGADRPIAACCPCCPGTSCLAESNGVSYCRKDEP | 2 | C10I K23L | 0 | N/A |
| 102 | KPV102 | AICTGADRPCAACCPCCPGTSCKAESNGVSYCRNDEP | 1 | K34N | 2.712176 | 0.5625 |
| 103 | KPV103 | AICTGADRPCAACCPCCPGTSCKAESNGVWYCRKGEP | 2 | S30W D35G | 0.679576 | 0 |
| 104 | KPV104 | IICTGADRPCAACCPCCPGTTCKAESNGVSYCRKDEP | 2 | A1I S21T | 0 | N/A |
| 105 | KPV105 | AICTGADRPCAACCPCFPGTSCKAESNGVSYCRKDEP | 1 | C17F | 0 | N/A |
| 106 | KPV106 | AICWGPDRPCAACCPCCPGCSCKAESNGVSYCRKDEP | 3 | T4W A6P T20C | 0 | N/A |
| 107 | KPV107 | AICTGADRPCAACCPCCPGTSCKAESNGVSYCCKDEP | 1 | R33C | 0 | N/A |
| 108 | KPV108 | AICTGADRPCAACCGCCPGTSCKAESNGVSYCRPDEP | 2 | P15G K34P | 0 | N/A |
| 109 | KPV109 | AICTGADRPCAACCPCQPGTSCKAESNGVSYCRKDEP | 1 | C17Q | 0 | N/A |
| 110 | KPV110 | AICTGEDRPCAACCPCCPGTSCKAESNGVSYCRKDEP | 1 | A6E | 1.510449 | 0 |
| 111 | KPV111 | AICTGADRPCAAFCPCCPGTSCKAESNGVSYSRKDEP | 2 | C13F C32S | 0 | N/A |
| 112 | KPV112 | AICTGADRPCAACCPCCGGTSCKAESNGVSYCRKDEP | 1 | P18G | 0.71134 | 0 |
| 113 | KPV113 | AICTGADRPCAACCPCCPRTSCKAESNGVSYCYKDEP | 2 | G19R R33Y | 0 | N/A |
| 114 | KPV114 | AICTGADRPCAACCPCCPGTSCFAESNGVSYCRKDEP | 1 | K23F | 1.773382 | 0.125 |
| 115 | KPV115 | AICTGADKPCAACCPCCPGTTCKAERNGVSYCRKDEP | 3 | R8K S21T S26R | 0.852048 | 0 |
| 116 | KPV116 | AICTGADRPCAACCPCCPGTSCKAESNGVSYCRKTEP | 1 | D35T | 0.597752 | 0.5 |
| 117 | KPV117 | AIGTGADRPCAACCPCCPGTSCKAESNGVSYCRKDEP | 1 | C3G | 0 | N/A |
| 118 | KPV118 | AICTGADRPCAACCPCCPGTSCTAESNGVSYCRKDEP | 1 | K23T | 0.420451 | 0 |
| 119 | KPV119 | AICTGADRPCAACCPCCPGYSCKAESNGVSYCRKDEP | 1 | T20Y | 0.82307 | 0.25 |
| 120 | KPV120 | AICTGADRDCAACCPCCPGTSCKAESNGVSYCRKDEP | 1 | P9D | 0.550014 | 0 |
| 121 | KPV121 | AICTGADRPCAACCPCCPGTSCKAESIGVSYCRKDEP | 1 | N27I | 0.562831 | 0 |
| 122 | KPV122 | AICTGADRPCAACCPCLPGTSCKAESNGVSYCRKDEP | 1 | C17L | 0 | N/A |
| 123 | KPV123 | AICKGADRPCAACCPCCPGTSCKAESNGVSYCRKDEP | 1 | T4K | 0 | N/A |
| 124 | KPV124 | AICTGADRPCAACCPCCPGTSCKAEANGVSYCRKDEP | 1 | S26A | 1.509241 | 0 |
| 125 | KPV125 | RICTGADRPCAACCPCCPGTSCKAESNGVSYCRKDEP | 1 | AIR | 0.499582 | 0 |
| 126 | KPV126 | AICTGADRPCAACCPCCPGTSCKAESNGVSYCRLDEP | 1 | K34L | 2.13299 | 0.75 |
| 127 | KPV127 | AICTGADRPCAACCPGTSCKAESNGVSYCRKDEP | 3 | C13- C14- P15- | 0 | N/A |
| 128 | KPV128 | AICTGADRPEAACCPCCPGTSCWAESNGVSYCRKDEP | 2 | C10E K23W | 0 | N/A |
| 129 | KPV129 | AICTGADRPCAACCPCCPGTSCKAESNGVIYCRKDEP | 1 | S30I | 0 | N/A |
| 130 | KPV130 | AICTGADRPCAACCPCCPGTSCKAESNGWSYCRKDEP | 1 | V29W | 0.859292 | 0 |
| 131 | KPV131 | AICTGADRPCAACCPCCPGTSCKAECNGVSYCRKDEL | 2 | S26C P37L | 0 | N/A |
| 132 | KPV132 | AICTGADRPCYACCPCCPGTSCKAESNGVSYRRKDEP | 2 | A11Y C32R | 0 | N/A |
| 133 | KPV133 | AICMGADRPCAACCPCCPGTSMKAESNGVSYCRKDEP | 2 | T4M C22M | 0 | N/A |
| 134 | KPV134 | AIHTGADRPCAACCPCCPGTSCKAESNGVSYCRKDEP | 1 | C3H | 0 | N/A |
| 135 | KPV135 | AICSGADRPCAACCPCCPGFSCKAESNGVSYCRKDEP | 2 | T4S T20F | 0.805795 | 0 |
| 136 | KPV136 | AICTGADRPCAACCPCCPGPSCKAESNGVSYCRKDEP | 1 | T20P | 0 | N/A |
| 137 | KPV137 | AIATGADRPCAACCPCCPGTSCKAESNGVSYCRKDEP | 1 | C3A | 0 | N/A |
| 138 | KPV138 | GICTGADRPCAACCPCCPGTSSKAESNGVSYCRKDEP | 2 | A1G C22S | 0.278908 | 0 |
| 139 | KPV139 | AICTGADRPCAACCPCCPGTSCKHESNGVSYCRKDEP | 1 | A24H | 0.742547 | 0 |
| 140 | KPV140 | AICTGADRPCAACCPCCPRTSCKAESNGVSYCRKDEP | 1 | G19R | 0.658679 | 1.25 |
| 141 | KPV141 | AICTGADRPCAACCPPCPGTSCKAESNGVSYCRKDEP | 1 | C16P | 0 | N/A |
| 142 | KPV142 | PICTGADRPCAACCPCCPGTTCKAERNGVSYCRKDEP | 3 | A1P S21T S26R | 0 | N/A |
| 143 | KPV143 | AICTGADRPCAACCPCCPGTTCKAGSNGVSYCRKDEP | 2 | S21T E25G | 0.90796 | 0 |
| 144 | KPV144 | AICTGADRPTAACCPCCPGTTCKAERNGVSYCRKDEG | 4 | C10T S21T S26R | 0.66871 | 0 |
| | | | | P37G | | |
| 145 | KPV145 | AICTGADRPCAACCPCCPGAEDNGVSYCRKDEP | 5 | T20- S21- C22- K23- | 0 | N/A |
| | | | | S26D | | |
| 146 | KPV146 | AICTGADRPCAACCPCCPGTSCKAESNGVSYCRMDEP | 1 | K34M | 2.774492 | 0.25 |
| 147 | KPV147 | AICTGADRPCAACCPCCPGISCKAESNGVSYCRKDEP | 1 | T20I | 0.744776 | 1.25 |
| 148 | KPV148 | AICTGADRPCAACCPCCPGTSCKAESNGVSYDRKDEP | 1 | C32D | 0.798737 | 0 |
| 149 | KPV149 | AICTGADRPCAACCPCCPGTSCKAEKNGVSYCRKDEP | 1 | S26K | 1.72973 | 0.75 |
| 150 | KPV150 | AICTGAARPCAACWPCCPGTSCKAESNGVSYCRKDEP | 2 | D7A C14W | 0.710783 | 0 |
| 151 | KPV151 | WICTGADRPCAACCPCCPGTSCKAESNGVSYCRKDEP | 1 | A1W | 1.506826 | 0 |
| 152 | KPV152 | AICTGADRACAACCPCCPGTSCKAESNGVSYCRKDEP | 1 | P9A | 1.19504 | 0 |
| 153 | KPV153 | AICTGADRPCAACCPCCPGTSNKAETNGVSYCRKDEP | 2 | C22N S26T | 0 | N/A |
| 154 | KPV154 | AICTGADRPCAACCPCCPKTSCKAESNGVSYCRKDEP | 1 | G19K | 0.64224 | 0.5 |
| 155 | KPV155 | AICSGADRPCAACCPGCPGTTCKAESNGVSYCRKDEP | 3 | T4S C16G S21T | 0.818055 | 0 |
| 156 | KPV156 | AICTGAFRPCAACCPCCPGTSCKAESNGVSYCRKDEP | 1 | D7F | 0 | N/A |
| 157 | KPV157 | AICTGADRPCAACCPCCPGTSCKAESNGVSYCREDEP | 1 | K34E | 2.252577 | 1.125 |
| 158 | KPV158 | AICTGADRPCAACCPCCPGQSCKAESNGVSYCRKDEP | 1 | T20Q | 1.024519 | 0.25 |
| 159 | KPV159 | AICTGADPPCAACCPCCPGTSCKAESNGVSYCYKDEP | 2 | R8P R33Y | 0 | N/A |
| 160 | KPV160 | AICTGADRPCAACCPCCPGTSCKAESNGVSYCRVDEP | 1 | K34V | 2.438841 | 0.5625 |
| 161 | KPV161 | AICTGADRPCAACCPCCPGTSCKAHSNGVSYCRKDEP | 1 | E25H | 1.641126 | 1 |
| 162 | KPV162 | AICTGADRPCAACCPCCPGTSCKAESNGVSYCRKDES | 1 | P37S | 2.481193 | 1.25 |
| 163 | KPV163 | AILTGADRPCAACCPCCPGTSCKAESNGVSYCRKDEP | 1 | C3L | 0 | N/A |
| 164 | KPV164 | AVCTGADRPCAACCPCCPGTSCKAESNGVWYCRKDEP | 2 | I2V S30W | 1.767902 | 0 |
| 165 | KPV165 | AICTGADRPCACCCPCCPGTSCKAESNGVSYCRKDEP | 1 | A12C | 0.889384 | 0 |
| 166 | KPV166 | AICTGADRPCAACCSCCPGTSCKAESNGVSECRKDEP | 2 | P15S Y31E | 0.833937 | 0 |
| 167 | KPV167 | AICTGADRPCAACCPCCPGTSCKAESNGVYYCRKDEP | 1 | S30Y | 1.504969 | N/A |
| 168 | KPV168 | AICTGADRPCAACCPCCPGTSCKAESLGVSYCRKDEP | 1 | N27L | 1.08814 | 0.25 |
| 169 | KPV169 | AICTGADRPCAACCPHCPGTSCKAESNGVSYCVKDEP | 2 | C16H R33V | 0.890777 | N/A |
| 170 | KPV170 | AICTGADRPCAACCPCCPPTSCKAESNGVSYCRKDEP | 1 | G19P | 1.069936 | 0 |
| 171 | KPV171 | AICTGADRPCAQCCPCCPGTSCKAESNGVSYCRKDEP | 1 | A12Q | 0.760379 | 0 |
| 172 | KPV172 | AICTGADRPCAACCPCCPDTSCKAESNGVSYCRKDEP | 1 | G19D | 0 | N/A |
| 173 | KPV173 | AICTGADRPCAACCMCCPGTSCKAESNGVSYCRKDEP | 1 | P15M | 1.504597 | 0 |
| 174 | KPV174 | AECTGADRPCDACCPCCPGTSCKAESNGVSYCRKDEP | 2 | I2E A11D | 0.705117 | 0.75 |
| 175 | KPV175 | AICTGADRPCAACCPCNPGTSCKAESNGVSYCRKDEP | 1 | C17N | 0.804124 | 0 |
| 176 | KPV176 | AICTGADRPCAACCPCCPGTSCKWESNGVSYCRKDEP | 1 | A24W | 1.614377 | 0 |
| 177 | KPV177 | AICTGADRPCAACCPCCPGTSCKAESNGVSSCRKDEP | 1 | Y31S | 0 | N/A |
| 178 | KPV178 | AICTGADRPCAACCPCCPGTSCKAEGNGVSYCRKDEP | 1 | S26G | 1.813597 | 0 |
| 179 | KPV179 | AICTGADRPCAACCPCCPGTSCKAESNGVSRCRKDEP | 1 | Y31R | 0.789496 | 0 |
| 180 | KPV180 | AICTGADRPCAACCPCCPGTSCKAWSNGVSYCRKDEP | 1 | E25W | 5.1 | 0.375 |
| 181 | KPV181 | AICTGADRPCAACCPCCPGTSCKAEENGVSYCRKDEP | 1 | S26E | 0.657008 | 0 |
| 182 | KPV182 | ATCTGADRPCAACCPCCPGTSCKAESNGVSYCRKDHP | 2 | I2T E36H | 0.748955 | 1.25 |
| 183 | KPV183 | AICTGANRPCAAGCPCCPGTSCKAESNGVSYCRKDEP | 2 | D7N C13G | 1.358596 | 0 |
| 184 | KPV184 | AICTGADRPCAACCPCCPGTSCKAESNGVSYCRKDEQ | 1 | P37Q | 1.501254 | 0.75 |
| 185 | KPV185 | AICTGADRPCAAFCPCCPGTSCKAESNGVSYCRKDEP | 1 | C13F | 2.250488 | 0 |
| 186 | KPV186 | AICTGADRPCAACCPCCPGTSCKAESWGVSYCRKDEP | 1 | N27W | 2.182223 | 0 |
| 187 | KPV187 | AICTGAARPCAACCPCCPGTSCKAETNGVSYCRKDEM | 3 | D7A S26T P37M | 0.939537 | 0 |
| 188 | KPV188 | AICTGSDRPCAACCPCCPGTSCKAESNGVSYCRKDEP | 1 | A6S | 0.898579 | 0.25 |
| 189 | KPV189 | AICTGADRPCAACCPCCPGTSCKAESNGVSYCQKDEP | 1 | R33Q | 1.339649 | 1 |
| 190 | KPV190 | AICTGADRACAACCPCCPGTRCKAESNGVSYCRKDEP | 2 | P9A S21R | 0.809511 | 0 |
| 191 | KPV191 | AICTGADRPCAACCPCCPGTTCQAESNGVSYCRKDEP | 2 | S21T K23Q | 1.823349 | 0 |
| 192 | KPV192 | AICTGIDRPCAACCPCCPGTSCKAESNGVSYCRKDKP | 2 | A6I E36K | 1.741711 | 0 |
| 193 | KPV193 | AICTGADRPCAACCPRCPGTSCKAESNGVSYCRKDEP | 1 | C16R | 0 | N/A |
| 194 | KPV194 | AICTGADRPCAACCPFCPGTSCKAESNGVSYCRKDEP | 1 | C16F | 0.757871 | 0 |
| 195 | KPV195 | AICTGAGRPCAACCPCCPGTSCKAESNGVSYCRKDEP | 2 | D7G E36F | 1.011981 | 1.25 |
| 196 | KPV196 | AICTGADRPCAACCPGCPGTSCNAESNHVSYCRKDEP | 3 | C16G K23N G28H | 0.801059 | N/A |
| 197 | KPV197 | AICTGADRPCAACCPCCPGTSCKAESNWVSYCRKDEP | 1 | G28W | 0 | N/A |
| 198 | KPV198 | AICTGADRPCALCCPCCPGTTCKAESNGVSYCRKDEP | 2 | A12L S21T | 0.857621 | 0.25 |
| 199 | KPV199 | AACTGADRPCAACCPCCPGTSCKAESNGVSYCRKDEP | 1 | I2A | 2.501811 | 0 |
| 200 | KPV200 | AICTGADRPCAACCPCCPGTTCKAESNGVTYCRKDEP | 2 | S21T S30T | 0.943717 | 1.25 |
| 201 | KPV201 | AICTGAARPCAACCPCCPGTSCKAESNGVSYCRKDEP | 1 | D7A | 0.32321 | 0.25 |
| 202 | KPV202 | AICTGADRPCAACCPKCPGTSCKAESNGVSYCRKDEP | 1 | C16K | 0 | N/A |
| 203 | KPV203 | AICTGADLPCAACCPCCPGTSCKDESNGVSYCRKDEP | 2 | R8L A24D | 0.912789 | 0 |
| 204 | KPV204 | AIHTGADRPCAACCPCCPGTSCDAESNGVSYCRKDEP | 2 | C3H K23D | 1.039287 | 0 |
| 205 | KPV205 | AICTGADRPCAACCPCCPGTSCAAESNGVSYCRKDEP | 1 | K23A | 1.593759 | 0.375 |
| 206 | KPV206 | AICTGADRPCAACCPCCPGTSCDAESNGVSYCRKDEP | 1 | K23D | 1.057676 | 0.125 |
| 207 | KPV207 | AICTGADRPCAACCPCCPGTSCEAESNGVSYCRKDEP | 1 | K23E | 0.835516 | 0 |
| 208 | KPV208 | AICTGADRPCAACCPCCPGTSCGAESNGVSYCRKDEP | 1 | K23G | 0 | N/A |
| 209 | KPV209 | AICTGADRPCAACCPCCPGTSCIAESNGVSYCRKDEP | 1 | K23I | 3.026191 | 1.25 |
| 210 | KPV210 | AICTGADRPCAACCPCCPGTSCLAESNGVSYCRKDEP | 1 | K23L | 2.527724 | 1.124294 |
| 211 | KPV211 | AICTGADRPCAACCPCCPGTSCMAESNGVSYCRKDEP | 1 | K23M | 3.954583 | 0.875 |
| 212 | KPV212 | AICTGADRPCAACCPCCPGTSCNAESNGVSYCRKDEP | 1 | K23N | 1.366397 | 0.75 |
| 213 | KPV213 | AICTGADRPCAACCPCCPGTSCPAESNGVSYCRKDEP | 1 | K23P | 0 | N/A |
| 214 | KPV214 | AICTGADRPCAACCPCCPGTSCQAESNGVSYCRKDEP | 1 | K23Q | 0.895886 | 0.9375 |
| 215 | KPV215 | AICTGADRPCAACCPCCPGTSCRAESNGVSYCRKDEP | 1 | K23R | 2.1395 | 0.875 |
| 216 | KPV216 | AICTGADRPCAACCPCCPGTSCVAESNGVSYCRKDEP | 1 | K23V | 2.87796 | 0.625 |
| 217 | KPV217 | AICTGADRPCAACCPCCPGTSCYAESNGVSYCRKDEP | 1 | K23Y | 3.25 | 0.9375 |
| 218 | KPV218 | AICTGADRPCAACCPCCPGTSCKAESNGVSYCRDDEP | 1 | K34D | 2.32934 | 0.75 |
| 219 | KPV219 | AICTGADRPCAACCPCCPGTSCKAESNGVSYCREDEP | 1 | K34F | 0 | N/A |
| 220 | KPV220 | AICTGADRPCAACCPCCPGTSCKAESNGVSYCRHDEP | 1 | K34H | 4.262747 | 1 |
| 221 | KPV221 | AICTGADRPCAACCPCCPGTSCKAESNGVSYCRIDEP | 1 | K34I | 4.96506 | 0.625 |
| 222 | KPV222 | AICTGADRPCAACCPCCPGTSCKAESNGVSYCRPDEP | 1 | K34P | 3.675 | 0.25 |
| 223 | KPV223 | AICTGADRPCAACCPCCPGTSCKAESNGVSYCRQDEP | 1 | K34Q | 2.016532 | 0.375 |
| 224 | KPV224 | AICTGADRPCAACCPCCPGTSCKAESNGVSYCRSDEP | 1 | K34S | 2.710226 | 1 |
| 225 | KPV225 | AICTGADRPCAACCPCCPGTSCKAESNGVSYCRTDEP | 1 | K34T | 2.872388 | 1.25 |
| 226 | KPV226 | VICTGADRPCAACCPCCPGTSCKAESNGVSYCRKDEP | 1 | A1V | 2.229033 | 1.217973 |
| 227 | KPV227 | LICTGADRPCAACCPCCPGTSCKAESNGVSYCRKDEP | 1 | A1L | 4.458066 | 1.25 |
| 228 | KPV228 | SICTGADRPCAACCPCCPGTSCKAGVNGVAYCRKD | 6 | A1S E25G S26V | 3.120646 | 0.230768 |
| | | | | S30A E36- P37- | | |
| 229 | KPV229 | AICTGADRPCAACCPCCPGTSCKAEANGVSRCRKD | 4 | S26A Y31R E36- | 1.560323 | 1.249961 |
| | | | | P37- | | |
| 230 | KPV230 | AICTGADRPCAACCPCCPGTSCKAEVNGVSYCRKDEP | 1 | S26V | 3.789356 | 0 |
| 231 | KPV231 | SICTGADRPCAACCPCCPGTSCKAESNGVSYCRKDEP | 1 | A1S | 8.916133 | 1.249975 |
| 232 | KPV232 | DICTGADRPCAACCPCCPGTSCKAESNGVSYCRKDEP | 1 | A1D | 6.687099 | N/A |
| 233 | KPV233 | AICTGADRPCAACCPCCPGTSCKAESNGVSLCRKDEP | 1 | Y31L | 6.687099 | N/A |
| 234 | KPV234 | AICTGADRPCAACCPCCPGTSCKAESNGVSECRKDEP | 1 | Y31E | 0 | N/A |
| 235 | KPV235 | AICTGADKPCAACCPCCPGTSCKAESNGVSYCRKDEP | 1 | R8K | 6.687099 | 0 |
| 236 | KPV236 | AICTGADHPCAACCPCCPGTSCKAESNGVSYCRKDEP | 1 | R8H | 4.458066 | 0 |
| 237 | KPV237 | AICTGADYPCAACCPCCPGTSCKAESNGVSYCRKDEP | 1 | R8Y | 6.687099 | 0 |
| 238 | KPV238 | AICTGADLPCAACCPCCPGTSCKAESNGVSYCRKDEP | 1 | R8L | 6.687099 | 0 |
| 239 | KPV239 | AICTGADMPCAACCPCCPGTSCKAESNGVSYCRKDEP | 1 | R8M | 4.458066 | 0 |
| 240 | KPV240 | AICTGADSPCAACCPCCPGTSCKAESNGVSYCRKDEP | 1 | R8S | 6.687099 | 0 |
| 241 | KPV241 | AICTGADRPCAACCPCCPGTSCKAESNGVSYCHH | 5 | R33H K34H D35- | 0 | N/A |
| | | | | E36- P37- | | |
| 242 | KPV242 | AICTGADRPCAACCPCCPGTSCKAESNGVSYCH | 5 | R33H K34- D35- | 0 | N/A |
| | | | | E36- P37- | | |
| 243 | KPV243 | AICTGADRPCGGCCPCCPGTSCKAESNGVSYCRKDEP | 2 | A11G A12G | 4.458066 | 0 |
| 244 | KPV244 | AICTGADRPCGACCPCCPGTSCKAESNGVSYCRKDEP | 1 | A11G | 13.3742 | 0 |
| 245 | KPV245 | AICTGADRPCAGCCPCCPGTSCKAESNGVSYCRKDEP | 1 | A12G | 2.229033 | 0 |
| 246 | KPV246 | AICTGADRPCAACCPCCPGTKCKAESNGVSYCRKDEP | 1 | S21K | 8.916133 | 0.500538 |
| 247 | KPV247 | AICTGADRPCAACCPCCPGTRCKAESNGVSYCRKDEP | 1 | S21R | 6.687099 | 0.5 |
| 248 | KPV248 | AICTGADGPCAACCPCCPGTSCKAESNGVSYCRKDEP | 1 | R8G | 2.229033 | 0 |
| 249 | KPV249 | AICTGADRPCAACCPCCPGTSCKTESNGVSYCRKD | 3 | A24T E36- P37- | 8.916133 | 1 |
| 250 | KPV250 | PICTGADRPCAACCPCCPGTSCKAESNGVSYCRKDEP | 1 | A1P | 2.229033 | 0 |
| 251 | KPV251 | EAAICTGADRPCAACCPCCPGTSCKAESNGVSYCRKD | 4 | 2′E 1′A E36- P37- | 6.687099 | 0 |
| 252 | KPV252 | NSAICTGADRPCAACCPCCPGTSCKAESNGVSYCRKDEP | 2 | 2′N 1′S | 1.560323 | 0.064768 |
| 253 | KPV253 | GSAICTGADRPCAACCPCCPGTSCKAESNGVSYCRKDEP | 2 | 2′G 1′S | 6.687099 | 0 |
| 254 | KPV254 | GSAICTGADRPCAACCPCCPGTSCKAESNGVSYCRKD | 4 | 2′G 1′S E36- P37- | 4.458066 | 0.032813 |
| 255 | KPV255 | EAAVCTGADRPCAACCPCCPGTSCKAESNGVSYCRKDEP | 3 | 2′E 1′A I2V | 8.916133 | 0 |
| 256 | KPV256 | GSQYCTGADRPCAACCPCCPGTSCKAESNGVSYCRKA | 7 | 2′G 1′S AIQ 12Y | 3.34355 | 0 |
| | | | | D35- E36- P37A | | |
| 257 | KPV257 | GICTGADRPCAACCPCCPGTSCKAESNGVWYCRKDEP | 2 | A1G S30W | 1.114517 | 0 |
| 258 | KPV258 | AICTGADRPCGACCPCCPGTSCKAESNGVSYCRA | 5 | A11G K34A D35- | 1.33742 | 0 |
| | | | | E36- P37- | | |
| 259 | KPV259 | GICTGADRPCGACCPCCPGTSCKAESNGVSYCRKDEP | 2 | A1G A11G | 1.560323 | 0 |
| 260 | KPV260 | AICTPADRPCAACCPCCPGTSCKAESNGVSYCRKDEP | 1 | G5P | 3.120646 | 0.339713 |
| 261 | KPV261 | AICTGADRPCAACCPCCSGASCKAESNGVSYCRKDEP | 2 | P18S T20A | 0.445807 | 1.25 |
| 262 | KPV262 | GICTGADRPCAACCPCCPGASCKAESNGVSYCRA | 6 | A1G T20A K34A | 0 | N/A |
| | | | | D35- E36- P37- | | |
| 263 | KPV263 | AICTGADRPCAACCPCCPGTSCSAESNGVSYCRKDEP | 1 | K23S | 0.891613 | 1.024675 |
| 264 | KPV264 | AICTGADRPCAACCPCCPGTSCQAASNGVSYCRKDEP | 2 | K23Q E25A | 2.451936 | 1.249938 |
| 265 | KPV265 | NAICTGADRPCAACCPCCPGTSCYAESNGVSYCRKDEP | 2 | 1′N K23Y | 1.783227 | 0 |
| 266 | KPV266 | QAICTGADRPCAACCPCCPGTSCYAESNGVSYCRKDEP | 2 | 1′Q K23Y | 0.445807 | 0.1125 |
| 267 | KPV267 | AICTGADRPCAACCPCCPGTSCVAESNGVSYCRA | 5 | K23V K34A D35- | 0.891613 | 0.690319 |
| | | | | E36- P37- | | |
| 268 | KPV268 | GICTGADRPCAACCPCCPGTSCVAESNGVSYCRA | 6 | A1G K23V K34A | 0.891613 | 0.07028 |
| | | | | D35- E36- P37- | | |
| 269 | KPV269 | AICTGADRPCAACCPCCPGTSCKAESNGVWYCRKDEP | 1 | S30W | 4.458066 | 1.25 |
| 270 | KPV270 | AICTGADRPCAACCPCCPGTSCKATSNGVSYCRKDEP | 1 | E25T | 1.783227 | 0.794625 |
| 271 | KPV271 | AICTGADRPCAACCPCCSGASCTEQSNGVSYCRKDEP | 5 | P18S T20A K23T | 0 | N/A |
| | | | | A24E E25Q | | |
| 272 | KPV272 | AICTGADRPCAACCPCCSGASCTEQSNGVSYCRA | 9 | P18S T20A K23T | 0 | N/A |
| | | | | A24E E25Q K34A | | |
| | | | | D35- E36- P37- | | |
| 273 | KPV273 | AICTGADRPCAACCPCCPGTSCKAFSNGVSYCRKD | 3 | E25F E36- P37- | 0.891613 | 0 |
| 274 | KPV274 | GAICTGADRPCAACCPCCPGTSCKAESNGVSYCRKDEP | 1 | 1′G | 2.67484 | 0.069159 |
| 275 | KPV275 | GAICTGADRPCAACCPCCPGTSCKAESNGVSYCRKD | 3 | 1′G E36- P37- | 4.01226 | 0.057994 |
| 276 | KPV276 | GSICTGADRPCAACCPCCPGTSCKAESNGVSYCRKDEP | 2 | 1′G A1S | 2.229033 | 0.063155 |
| 277 | KPV277 | SICTGADRPCAACCPCCPGTSCKAFSNGVSYCRKD | 4 | A1S E25F E36- P37- | 1.114517 | 0.069966 |
| 278 | KPV278 | SICTGADRPCAACCPCCPGTSCYAESNGVSYCRKDEP | 2 | A1S K23Y | 2.229033 | 0.218151 |
| 279 | KPV279 | AICTGADRPCAACCPCCPGTSCYAESNGVSYCRKD | 3 | K23Y E36- P37- | 2.67484 | 1.249961 |
| 280 | KPV280 | SICTGADRPCAACCPCCPGTSCKAESNGVSWCRKDEP | 2 | A1S Y31W | 2.229033 | 0 |
| 281 | KPV281 | AICTGADRPCAACCPCCPGTSCKARSNGVSYCRKDEP | 1 | E25R | 0.445807 | 0 |
| 282 | KPV282 | AICTGADRPCAACCPCCPGTSCKAMSNGVSYCRKDEP | 1 | E25M | 2.00613 | 1.244823 |
| 283 | KPV283 | GICTGADRPCAACCPCCPGTSCKAESNGVSYCRKDEP | 1 | A1G | 6.687099 | 0 |
| 284 | KPV284 | AICTGADRPCAACCPCCPGTSCKASSNGVSYCRKDEP | 1 | E25S | 2.897743 | 0.428971 |
| 285 | KPV285 | LICTGADRPCAACCPCCPGTSCYAESNGVSYCRKD | 4 | A1L K23Y E36- | 1.783227 | 0 |
| | | | | P37- | | |
| 286 | KPV286 | AICTGADRPCAACCPCCPGTSCKAESNGVSYCRKAEP | 1 | D35A | 2.792143 | 1.125 |
| 287 | KPV287 | AICTGADRPCAACCPCCPGTSCKAESNGVSYCRKEEP | 1 | D35E | 4.284759 | 1 |
| 288 | KPV288 | AICTGADRPCAACCPCCPGTSCKAESNGVSYCRKGEP | 1 | D35G | 2.298412 | 1.125 |
| 289 | KPV289 | AICTGADRPCAACCPCCPGTSCKAESNGVSYCRKHEP | 1 | D35H | 3.661744 | 1.25 |
| 290 | KPV290 | AICTGADRPCAACCPCCPGTSCKAESNGVSYCRKIEP | 1 | D35I | 1.73976 | 0.875 |
| 291 | KPV291 | AICTGADRPCAACCPCCPGTSCKAESNGVSYCRKKEP | 1 | D35K | 1.986626 | 1.25 |
| 292 | KPV292 | AICTGADRPCAACCPCCPGTSCKAESNGVSYCRKMEP | 1 | D35M | 2.104579 | 1.125 |
| 293 | KPV293 | AICTGADRPCAACCPCCPGTSCKAESNGVSYCRKPEP | 1 | D35P | 0 | N/A |
| 294 | KPV294 | AICTGADRPCAACCPCCPGTSCKAESNGVSYCRKQEP | 1 | D35Q | 3.261447 | 1.25 |
| 295 | KPV295 | AICTGADRPCAACCPCCPGTSCKAESNGVSYCRKREP | 1 | D35R | N/A | N/A |
| 296 | KPV296 | AICTGADRPCAACCPCCPGTSCKAESNGVSYCRKSEP | 1 | D35S | 2.00483 | 1.25 |
| 297 | KPV297 | AICTGADRPCAACCPCCPGTSCKAESNGVSYCRKVEP | 1 | D35V | 2.072444 | 1.125 |
| 298 | KPV298 | AICTGADRPCAACCPCCPGTSCKAESNGVSYCRKWEP | 1 | D35W | 0 | N/A |
| 299 | KPV299 | AICTGADRPCAACCPCCPGTSCKAESNGVSYCRKYEP | 1 | D35Y | 3.470902 | 0 |
| 300 | KPV300 | AICTGADRPCAACCPCCPGTSCKQEANGVSLCRKDEP | 3 | A24Q S26A Y31L | 1.83533 | 0.06212 |
| 301 | KPV301 | SICTGADRPCAACCPCCPGTSCKIGCNGLHYCRKDEP | 6 | A1S A24I E25G | 0.962942 | 0 |
| | | | | S26C V29L S30H | | |
| 302 | KPV302 | SICTGADRPCAACCPCCPGTSCQIWSNGVSYCRKDEP | 4 | AIS K23Q A24I | 1.926442 | 0 |
| | | | | E25W | | |
| 303 | KPV303 | AICTGADRPCAACCPCCPGTSCKYESLGVSYCRKDEP | 2 | A24Y N27L | 1.867373 | 0 |
| 304 | KPV304 | AICTGADRPCAACCPCCPGTSCTARQNGVPYCRKDEP | 4 | K23T E25R S26Q | 1.380886 | 0 |
| | | | | S30P | | |
| 305 | KPV305 | AICTGADRPCAACCPCCPGTSCKSTGNGAGYCRKDEP | 5 | A24S E25T S26G | 1.186124 | 0 |
| | | | | V29A S30G | | |
| 306 | KPV306 | AICTGADRPCAACCPCCPGTSCKAGSAGAFYCRKDEP | 4 | E25G N27A V29A | 1.588465 | 0 |
| | | | | S30F | | |
| 307 | KPV307 | AICTGADRPCAACCPCCPGTSCKATLNGVSYCRKDEP | 2 | E25T S26L | 0.90861 | 1.25 |
| 308 | KPV308 | AICTGADRPCAACCPCCPGTSCPDESEGVYYCRKDEP | 4 | K23P A24D N27E | 1.714498 | 0 |
| | | | | S30Y | | |
| 309 | KPV309 | TICGGDDTPCAACCPCCPGTSCKPESNGESLCRKDEP | 7 | A1T T4G A6D R8T | 0.682084 | 1.25 |
| | | | | A24P V29F Y31L | | |
| 310 | KPV310 | SICTGADRPCAACCPCCPGTSCKWESNVESYCRKDEP | 4 | A1S A24W G28V | 0.358094 | 0 |
| | | | | V29E | | |
| 311 | KPV311 | AICTGADRPCAACCPCCPGTSCVAESWGVSYCRKDEP | 2 | K23V N27W | 0.989635 | 0.0625 |
| 312 | KPV312 | AICTGADRPCAACCPCCPGTSCKADHWGVPYCRKDEP | 4 | E25D S26H N27W | 3.617721 | 0 |
| | | | | S30P | | |
| 313 | KPV313 | SICTGADRPCAACCPCCPGTSCKEEIEGPSYCRKDEP | 5 | A1S A24E S26I | 2.091112 | 0 |
| | | | | N27E V29P | | |
| 314 | KPV314 | IICTGADRPCAACCPCCPGTSCKKQSNGVSYCRKDEP | 3 | A1I A24K E25Q | 0.468933 | 1.25 |
| 315 | KPV315 | AICTGADRPCAACCPCCPGTSCKAEDNGGTYCRKDEP | 3 | S26D V29G S30T | 1.820284 | 0 |
| 316 | KPV316 | SICTGADRPCAACCPCCPGTSCSFESGGVSYCRKDEP | 4 | A1S K23S A24F | 1.898579 | 0 |
| | | | | N27G | | |
| 317 | KPV317 | AICTGADRPCAACCPCCPGTSCKSESNGSYYCRKDEP | 3 | A24S V29S S30Y | 1.576484 | 0 |
| 318 | KPV318 | AICTGANRPCAACCPCCPGTSCKAESNGVSYCRKDEP | 1 | D7N | 2.229033 | 0.991985 |
| 319 | KPV319 | AICTGANRPCAACCPCCPGTSCVAESNGVSYCRKDEP | 2 | D7N K23V | 4.458066 | 0.749544 |
| 320 | KPV320 | AICTGADRPCAACCPCCPGTSCKTESNGVSYCRKDEP | 1 | A24T | 2.229033 | 0.200598 |
| 321 | KPV321 | AICTGADRPCAACCPCCPGTSCKAESNGVSYCRA | 4 | K34A D35- E36- | 4.458066 | 1.233738 |
| | | | | P37- | | |
| 322 | KPV322 | AICTGADRPCAACCPCCPGTSCKAESNGVSYCRKD | 2 | E36- P37- | 2.229033 | 0.588068 |
| 323 | KPV323 | ALCTGADRPCAACCPCCPGTSCKAESNGVSYCRKDEP | 1 | I2L | 2.229033 | 0 |
| 324 | KPV324 | AICTGADRPCAACCPCCPGTSWKQRVMVELIAGRMNRKR | 18 | C24W 25K K26Q | 0 | N/A |
| | | PLN | | A27R E28V S29M | | |
| | | | | N30V G31F V35L | | |
| | | | | S36I Y37A C38G | | |
| | | | | K40M D41N E42R | | |
| | | | | P43K 44R 45P | | |
| 325 | KPV325 | AICTGADRPCAACCPCCPGTSCQGPESNGVSYCRKDEP | 3 | 25Q K26G A27P | 2.229033 | 1.23915 |
| 326 | KPV326 | TICTGADRPCAACCPCCPGTSCTEPESNGGHTVYYCRND | 11 | A1T 25T K26E A27P | 0 | N/A |
| | | | | 32G 33H 34T S36Y | | |
| | | | | K40N E42 P43 | | |
| 327 | KPV327 | QACTPADQPCAACCPCCSGASCTEQLNENGHTVYYCRA | 21 | A1Q 12A G5P R8Q | 4.458066 | 0 |
| | | | | P20S T22A 25T | | |
| | | | | K26E A27Q E28L | | |
| | | | | S29N N30E G31N | | |
| | | | | 32G 33H 34T S36Y | | |
| | | | | K40A D41 E42 P43 | | |
| 328 | KPV328 | AICTGADRPCAACCPCCPGTSCQGPESNGVSYCRA | 7 | 25Q K26G A27P | 1.560323 | 1.249981 |
| | | | | K40A D41 E42 P43 | | |
| 329 | KPV329 | AICTGADRPCAACCPCCPGTSCAGPESNGVSYCRKDEP | 3 | 25A K26G A27P | 1.783227 | 1.249963 |
| 330 | KPV330 | AICTGADRPCAACCPCCPGTSCQGAESNGVSYCRKDEP | 2 | 25Q K26G | 0.445807 | 0.625 |
| 331 | KPV331 | AICTGADRPCAACCPCCPGTSCKGPESNGVSYCRKDEP | 3 | 25K K26G A27P | 2.00613 | 1.25 |
| 332 | KPV332 | AICTGADRPCAACCPCCPGTSCNGPESNGVSYCRKDEP | 3 | 25N K26G A27P | 1.560323 | 0.99935 |
| 333 | KPV333 | QAICTGADRPCAACCPCCPGTSCQGPESNGVSYCRKDEP | 3 | +1Q 25Q K26G | 0 | N/A |
| | | | | A27P | | |
| 334 | KPV334 | NAICTGADRPCAACCPCCPGTSCQGPESNGVSYCRKDEP | 3 | +IN 25Q K26G | 2.67484 | 0 |
| | | | | A27P | | |
| 335 | KPV335 | AICTGADRPCAASQCCPCCPGTSCTEPESNGGHTVYYCR | 12 | 13S 14Q 25T K26E | 0 | N/A |
| | | ND | | A27P 32G 33H 34T | | |
| | | | | S36Y K40N E42 P43 | | |
| 336 | KPV336 | AICTGADRPCAACCPCCPGTSCTEPESNGGHTVYYCRND | 10 | 25T K26E A27P 32G | 0 | N/A |
| | | | | 33H 34T S36Y K40N | | |
| | | | | E42 P43 | | |
| 337 | KPV337 | QACTGADRPCAACCPCCPGTSCTEPESNGGHTVYYCRND | 12 | A1Q I2A 25T K26E | 0 | N/A |
| | | | | A27P 32G 33H 34T | | |
| | | | | S36Y K40N E42 P43 | | |
| 338 | KPV338 | QATICTGADRPCAACCPCCPGTSCTEPESNGGHTVYYCR | 11 | +2Q +1A A1T 25T | 0 | N/A |
| | | ND | | K26E A27P 32G 33H | | |
| | | | | 34T S36Y K40N E42 | | |
| | | | | P43 | | |
| 339 | KPV339 | AICTGADRPCAACCPCCSGASCGGPESNGVSYCRKDEP | 5 | P20S T22A 25G | 0 | N/A |
| | | | | K26G A27P | | |
| 340 | KPV340 | AICTGADRPCAACCPCCSGASCGGPESNGVSYCRA | 9 | P20S T22A 25G | 0 | N/A |
| | | | | K26G A27P K40A | | |
| | | | | D41 E42 P43 | | |
| 341 | KPV341 | AICTGADRPCGACCPCCSGASCGGPESNGVSYCRKDEP | 6 | A11G P20S T22A | 0 | N/A |
| | | | | 25G K26G A27P | | |
| 342 | KPV342 | AICTGADRPCGACCPCCSGASCGGPESNGVSYCRA | 10 | A11G P20S T22A | 0 | N/A |
| | | | | 25G K26G A27P | | |
| | | | | K40A D41 E42 P43 | | |
| 343 | KPV343 | TICTGADRPCAACCPCCPGTSCTEPESNGVSYCRND | 7 | A1T 25T K26E A27P | 0.445807 | 1.25 |
| | | | | K40N E42 P43 | | |
| 344 | KPV344 | AICTGADRPCAACCPCCPGTSCTEPESNGVSYCRND | 6 | 25T K26E A27P | 0.891613 | 1.25 |
| | | | | K40N E42 P43 | | |
| 345 | KPV345 | SICTGADRPCAACCPCCPGTSCQQDEFQNVWNCRKDEP | 9 | A1S 25Q K26Q | 0.446364 | 0 |
| | | | | A27D S29F N30Q | | |
| | | | | G31N S36W Y37N | | |
| 346 | KPV346 | SICTGADRPCAACCPCCPGTSCHKADSNGLSYCRKDEP | 4 | A1S 25H E28D | 1.293118 | 0.199105 |
| | | | | V35L | | |
| 347 | KPV347 | SICTGADRPCAACCPCCPGTSCIKRESNGVSYCRKDEP | 3 | A1S 25I A27R | 0.385065 | 1.25 |
| 348 | KPV348 | AICTGADRPCAACCPCCPGTSCTKRESNGVKYCRKDEP | 3 | 25T A27R S36K | 0.603789 | 1.25 |
| 349 | KPV349 | AICTGADRPCAACCPCCPGTSCQKAESNGVLYCRKDEP | 2 | 25Q S36L | 0.575369 | 0.114038 |
| 350 | KPV350 | TICSGGDRPRAACCPCCPGTSSGKARVNGVFYCRKDEP | 9 | A1T T4S A6G C10R | 1.027027 | 0.740746 |
| | | | | C24S 25G E28R | | |
| | | | | S29V S36F | | |
| 351 | KPV351 | SICTGADRPCAACCPCCPGTSCRKADNGGVDYCRKDEP | 6 | A1S 25R E28D S29N | 0.704374 | 0 |
| | | | | N30G S36D | | |
| 352 | KPV352 | AICTGADRPCAACCPCCPGTSCVKARVNGVFYCRKDEP | 4 | 25V E28R S29V | 0.360825 | 0.938155 |
| | | | | S36F | | |
| 353 | KPV353 | SICTGADSPCAACCPSCPGTSCKGASWWGVCNCRKDEP | 10 | A1S R8S C18S 25K | 0.420544 | 0.488713 |
| | | | | K26G E28S S29W | | |
| | | | | N30W S36C Y37N | | |
| 354 | KPV354 | AICTGADRPCAACCPCCPGTSCTKAESNGISYCRKDEP | 2 | 25T V35I | 1.634717 | 1.25 |
| 355 | KPV355 | SICTGADRPCAACCPCCPGTSCQKDDHNGVGYCRKDEP | 6 | A1S 25Q A27D | 1.858178 | 0 |
| | | | | E28D S29H S36G | | |
| 356 | KPV356 | AICTGADRPCAACCPCCPGTSCYKAPDNGLPYCRKDEP | 5 | 25Y E28P S29D | 0.589579 | 0 |
| | | | | V35L S36P | | |
| 357 | KPV357 | AICTGADRPCAAYCPCCPGTSCQKALSNGSSYCRKDEP | 4 | C15Y 25Q E28L | 0.644943 | 0 |
| | | | | V35S | | |
| 358 | KPV358 | AICTGADRPCAACCPCCPGTSCKAESNGVSYCRVDEP | 1 | K40V | 2.816383 | 0.625 |

Embodiments and implementations have been described. Nevertheless, it will be understood that various modifications may be made without departing from the spirit and scope of the disclosure. Accordingly, other embodiments and implementations are within the scope of the following claims.

1. A Kappa Peptide Variant (KPV), having pesticidal activity against one or more pests, said KPV comprising an amino acid sequence that is at least 90%, 95%, 96%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or an agriculturally acceptable salt thereof, with the proviso that the KPV does not comprise the amino acid sequence of SEQ ID NOs: 1 or 253.

2. The KPV of claim 1, wherein the KPV comprises, consists essentially of, or consists of, an amino sequence that is at least 90%, 95%, 96%, 97%, 98%, 99%, or 100% identical to a sequence set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358; or an agriculturally acceptable salt thereof.

3. A combination comprising, consisting essentially of, or consisting of, two or more KPVs of claim 1.

4. A composition comprising, consisting essentially of, or consisting of, one or more KPVs of claim 1, and an agriculturally acceptable excipient.

5. A polynucleotide operable to encode an Kappa Peptide Variant (KPV), said KPV comprising an amino acid sequence that is at least 90%, 95%, 96%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary polynucleotide sequence thereof, with the proviso that the polynucleotide does not encode an KPV comprising or consisting of the amino acid sequence of SEQ ID NOs: 1 or 253.

6. The polynucleotide of claim 5, wherein the polynucleotide encodes an KPV comprising, consisting essentially of, or consisting of, an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

7. A vector comprising the polynucleotide of claim 5.

8. (canceled)

9. A yeast strain comprising: a first expression cassette comprising a polynucleotide operable to encode an KPV, said KPV comprising an amino acid sequence that is at least 90%, 95%, 96%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; or a complementary nucleotide sequence thereof; with the proviso that the KPV does not comprise the amino acid sequence of SEQ ID NOs: 1 or 253.

10. The yeast strain of claim 9, wherein the KPV comprises an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

11. The yeast strain of claim 9, wherein the yeast strain is selected from any species belonging to the genera Saccharomyces, Pichia, Kluyveromyces, Hansenula, Yarrowia or Schizosaccharomyces.

12. The yeast strain of claim 11, wherein the yeast strain is selected from the group consisting of Kluyveromyces lactis, Kluyveromyces marxianus, Saccharomyces cerevisiae, and Pichia pastoris.

13. (canceled)

14. A method of producing a KPV, the method comprising:
(a) preparing a vector comprising a first expression cassette comprising, consisting essentially of, or consisting of, a polynucleotide operable to encode a KPV, or a complementary nucleotide sequence thereof, said KPV comprising an amino acid sequence that is at least 90%, 95%, 96%, 97%, 98%, 99%, or 100% identical to the amino acid sequence according to Formula (I): X₁—X₂—X₃—X₄—X₅—X₆—X₇—X₈—X₉—X₁₀—X₁₁—X₁₂—X₁₃—X₁₄—X₁₅—X₁₆—X₁₇—C—X₁₈—P—X₁₉—X₂₀—X₂₁—X₂₂—X₂₃—X₂₄—X₂₅—X₂₆—X₂₇—X₂₈-G-X₂₉—X₃₀—X₃₁—X₃₂—X₃₃—X₃₄—X₃₅—X₃₆—X₃₇; wherein the KPV comprises at least one amino acid substitution relative to the amino acid sequence set forth in SEQ ID NO: 1; wherein X₁ is G, E, N, or absent; X₂ is N, S, A, G, or absent; X₃ is A, T, S, Q, G, L, W, V, D, or P; X₄ is I, Y, V, A, or L; X₅ is C, H, or Y; X₆ is T, S, E, or R; X₇ is G, E, K, D, or P; X₈ is A, G, I, or E; X₉ is D, N, or G; X₁₀ is R, G, S, K, H, Y, L, or M; X₁₁ is P or A; X₁₂ is C, R, N, S, A, G, Y, or K; X₁₃ is A, H, G, or S; X₁₄ is A, G, or I; X₁₅ is C, G, I, E, or F; X₁₆ is C or N; X₁₇ is P or M; X₁₈ is C or Y; X₁₉ is G, R, or P; X₂₀ is T, G, H, S, E, N, I, or Q; X₂₁ is S, T, K, or R; X₂₂ is C or S; X₂₃ is G, Q, T, A, K, N, H, I, or absent; X₂₄ is K, E, G, T, P, Q, S, D, V, Y, R, N, M, L, I, A, F, or W; X₂₅ is A, D, P, R, S, E, I, F, Q, Y, T, or W; X₂₆ is E, R, D, T, G, W, A, F, H, M, or S; X₂₇ is S, V, H, G, Q, I, D, P, A, W, T, or K; X₂₈ is N, A, W, E, G, L, R, or V; X₂₉ is V, L, I, A, P, S, or G; X₃₀ is S, F, G, K, P, A, Y, T, R, C, or W; X₃₁ is Y, N, L, R, W, H, or F; X₃₂ is C or G; X₃₃ is R or Q; X₃₄ is K, N, A, R, V, G, L, M, E, D, H, I, P, Q, S, or T; X₃₅ is D, A, M, N, E, G, H, I, K, Q, R, S, V, Y, or absent; X₃₆ is E, K, F, V, or absent; X₃₇ is P, S, Q, or absent; with the proviso that the KPV does not comprise the amino acid sequence of SEQ ID NO: 1 OR 253;
(b) introducing the vector into a host cell; and
(c) growing the host cell in a growth medium under conditions operable to enable expression of the HVP and secretion into the growth medium.

15. The method of claim 14, wherein the KPV comprises, consists essentially of, or consists of, an amino sequence as set forth in any one of SEQ ID NOs: 2-7, 10-13, 16-19, 23, 25, 30-31, 35, 39-40, 45-46, 48, 50, 54-57, 60, 66, 68, 70-71, 73-74, 76-79, 85, 102, 110, 114, 124, 126, 140, 146-147, 149, 151-152, 157-158, 160-162, 164, 167-168, 170, 173, 176, 178, 180, 183-186, 189, 191-192, 195, 199, 204-206, 209-212, 215-218, 220-233, 235-240, 243-260, 263-265, 269-270, 274-280, 282-292, 294, 296-297, 299-300, 302-306, 308, 312-313, 315-323, 325, 327-329, 331-332, 334, 343-344, 346-348, 350, 354-355, or 358.

16. The method of claim 14, wherein the vector is a plasmid comprising an alpha-MF signal.

17. (canceled)

18. The method of claim 14, wherein the yeast strain is selected from any species belonging to the genera Saccharomyces, Pichia, Kluyveromyces, Hansenula, Yarrowia, or Schizosaccharomyces.

19-24. (canceled)

25. The method of claim 14, wherein the vector comprises two or three expression cassettes, each expression cassette operable to encode the KPV of the first expression cassette, or a KPV of a different expression cassette.

26. (canceled)

27. A method of combating, controlling, or inhibiting a pest comprising, applying a pesticidally effective amount of the Kappa Peptide Variant (KPV) of claim 1, the combination of claim 3, or the composition of claim 4, to: the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof.

28. The method of claim 27, wherein the pest is an insect.

29. The method of claim 27, wherein the pest is a lepidopteran insect, a dipteran insect, a coleopteran insect, or a non-insect arthropod pest.

30-32. (canceled)

33. The method of claim 27, wherein the pest is selected from the group comprising: Acariformes sp.; Acleris gloverana, Acrobasis vaccinii; Adoxophyes orana, Aedes aegypti; Agrotis ipsilon; Aleyrodidae sp.; Alsophila pometaria, Ambylomma americanum; Amblyomma maculatum; Amorbia humerosana; Anarsia lineatella, Anomala orientalis; Anthonomus eugenii; Anticarsia gemmatalis; Aphidoidea sp.; Aphis glycines Matsumura; Archips argyrospilia, Archips podana, Archips rosana, Argyrotaenia citrana, Argyrotaenia ljungiana; Argyrotaenia velutinana, Automeris io; Batrachedra comosae; Busseola fusca; Caudra cautella, Cerotoma spp.; Chaetocnema concinna, Chilo auricilius; Chilo partellus; Chilo suppressalis; Choristoneura fumiferana; Choristoneura pinus; Choristoneura rosaceana; Chrysodeixis includens Walker; Circulifer tenellus; Colias eurytheme; Coniesta ignefusalis; Conoderus rudis; Cosmopolites sordidus; Cotinis nitida, Crambus sp.; Crocidosema spp.; Culex pipiens; Culex quinquefasciatus; Curculio caryae; Curculio occidentis; Cyclocephala borealis; Cyclocephala lurida; Cydia funebrana; Cydia molesta; Cydia nigricana, Cydia pomonella; Cydia splendana; Cylas formicarius; Datana integerrima, Datana major; Dermacentor albipictus; Dermacentor andersoni; Dermacentor occidentalis; Dermacentor variabilis; Diabrotica spp.; Diabrotica virgifera; Diaphania hyalinata; Diaprepes abbreviatus; Diatraea crambidoides; Diatraea saccharalis; Diatraea spp.; Dorysthenes buqueti, Dryocampa rubicunda, Earias insulana; Ennomos subsignaria, Ephestia elutella, Epilachna varivestis Mulsant, Epiphyas postvittana; Erinnyis ello; Erionota thrax; Estigmene acrea, Euetheola humilis; Eumorpha achemon; Euwallacea fornicatus; Euxesta stigmatias; Evergestis rimosalis; Grapholita delineana; Grapholita molesta; Grapholita packardi; Halyomorpha halys; Harrisina americana, Helicoverpa armigera, Helicoverpa spp.; Helicoverpa zea, Heliothis virescens; Heterocampa guttivitta; Homadaula anisocentra; Hypera postica, Hypera zoilus; Hypercompe scribonia, Hyphantria cunea; Hypothenemus hampei; Ixodes cookei, Ixodes pacificus; Ixodes scapularis; Lambdina fiscellaria; Lasiocampidae sp.; Leptinotarsa decemlineata; Lissorhoptrus oryzophilus; Listronotus maculicollis; Lobesia botrana, Lymantria dispar; Maladera castanea, Maliarpha separatella, Manduca quinquemaculata, Manduca sexta, Melanagromyza soja Zehntner; Melissopus latiferreanus; Neophasia menapia, Noctua pronuba; Noctuid sp.; Nymphula stagnata; Orgyia pseudotsugata; Orgyia vetusta; Ostrinia furnacalis; Ostrinia nubilalis; Otiorhynchus ovatus; Paleacrita merriccata; Pandemis cerasana, Papilio cresphontes; Paralobesia viteana; Pectinophora gossypiella, Pegomya betae; Pemphigus betae; Peridroma saucia; Phryganidia californica; Phyllophaga sp.; Pieris rapae; Plathypena scabra; Platynota flavedana; Platynota idaeusalis; Platynota stultana, Platyptilia carduidactyla, Playnota stultana; Plodia interpunctella, Plutella xylostella, Popillia japonica; Pseudaletia unipuncta, Pseudoplusia includens; Rhipicephalus sangunineus; Rhizotroqus majalis; Sabulodes aegrotata; Schizura concinna; Scirpophaga incertulas; Scirpophaga innotata, Scutigerella immaculata; Sesamia calamistis; Sibine stimulea, Sminthurus viridis; Sparganothis pilleriana; Sphenophorus apicalis; Sphenophorus cariosus; Sphenophorus inaequalis; Sphenophorus minimus; Sphenophorus parvulus; Sphenophorus spp.; Spilonota ocellana, Spodoptera exigua; Spodoptera frugiperda; Spodoptera littoralis; Spodoptera sp.; Symmetrischema tangolias; Syntomeida epilais; Telchin licus; Tetanops myopaeformis; Tetranychidae sp.; Thaumatotibia (Cryptophlebia) leucotreta, Thecla basilides; Thymelicus lineola; Thyridopteryx ephemeraeformis; Thysanoptera sp.; Trichoplusia ni, Xanthogaleruca luteola, Xylomyges curialis; or Xylotrechus quadripes.

34. (canceled)

35. A Kappa Peptide Variant (KPV), having pesticidal activity against one or more pests, said KPV comprising, consisting essentially of, or consisting of, an amino acid sequence that is at least 90%, 95%, 96%, 97%, 98%, 99%, or 100% identical to the amino acid sequence set forth in any one of SEQ ID NOs: 2, 229, 264, or 325, or an agriculturally acceptable salt thereof, with the proviso that the KPV does not comprise the amino acid sequence of SEQ ID NOs: 1 or 253.

36-39. (canceled)

40. A Kappa Peptide Variant (KPV), having pesticidal activity against one or more pests, said KPV consisting of an amino acid sequence as set forth in SEQ ID NOS: 2, 229, 264, 325, or an agriculturally acceptable salt thereof.

41-43. (canceled)

44. (canceled)

45-48. (canceled)


SEQUENCE LISTING (379 sequences)

SEQ ID NO: 1 - protein, 37 aa, Hadronyche versuta
AICTGADRPCAACCPCCPGTSCKAESNGVSYCRKDEP

SEQ ID NO: 2 - protein, 35 aa, Synthetic construct
GICTGADRPCAACCPCCPGTSCKTESNGVSYCRKD

SEQ ID NO: 3 - protein, 37 aa, Synthetic construct
SICTGADRPCAACCPCCPGTSCKAGVNGVAYCRKDEP

SEQ ID NO: 4 - protein, 37 aa, Synthetic construct
AICTGADRPCAACCPCCPGTSCKAEANGVSRCRKDEP

SEQ ID NO: 5 - protein, 37 aa, Synthetic construct
AICTGADRPCAACCPCCPGTSCKAFSNGVSYCRKDEP

SEQ ID NO: 6 - protein, 37 aa, Synthetic construct
AICTGADRPCAACCPCCPGTSCKAEWNGVSYCRKDEP

SEQ ID NO: 7 - protein, 37 aa, Synthetic construct
AICTGADRPCAAICPCCPGTSCKAESNGVSYCRKDEP

SEQ ID NO: 8 - protein, 37 aa, Synthetic construct
AICTGADRPCALCCPCCPGTSCKAESNGVSYCRKDEP

SEQ ID NO: 9 - protein, 37 aa, Synthetic construct
AINTGADRPCAACCPCCPGTSCKAESNGVSYCRKDEP

SEQ ID NO: 10 - protein, 37 aa, Synthetic construct
AICTGIDRPCAACCPCCPGTSCKAESNGVSYCRKDEP

SEQ ID NO: 11 - protein, 37 aa, Synthetic construct
AICTGADRPCAACCPCCPGTSCKAESNGVSHCRKDEP

SEQ ID NO: 12 - protein, 37 aa, Synthetic construct
AICTGADRPCAACCPCCPGTSSKAESNGVCYCRKDEP

SEQ ID NO: 13 - protein, 37 aa, Synthetic construct
AICTEADRPCHACCPCCPGTSCKAESNGVRYCRKDEP

SEQ ID NO: 14 - protein, 37 aa, Synthetic construct
AICTGADRPCAACCPCCPGTSCKAESNGVSPCRKDEP

SEQ ID NO: 15 - protein, 37 aa, Synthetic construct
AICTGADLPCAACCPCCPGTSCKAESNGVSYCRGDEP

SEQ ID NO: 16 - protein, 37 aa, Synthetic construct
AICTGADRPCAICCPCCPGTSCKAESNGVSYCRKDEP

SEQ ID NO: 17 - protein, 37 aa, Synthetic construct
AICEGADRPCAACCPCCPGTSCKAESNGVSYCRKDEP

SEQ ID NO: 18 - protein, 37 aa, Synthetic construct
AICTGADRPCAACCPCCPGTSCKAESRGVSYCRKDEP

SEQ ID NO: 19 - protein, 37 aa, Synthetic construct
AICTGADRPCAACCPCCPGTSCKAESNGVSYGRKDEP

SEQ ID NO: 20 - protein, 37 aa, Synthetic construct
AICTGADRPCAACCPCCPGTSCKAESNAVSYCRKDEP

SEQ ID NO: 21 - protein, 37 aa, Synthetic construct
AICTGADRPCAACCPCCLGTSCKAESNGVSYCRKDEP

SEQ ID NO: 22 - protein, 37 aa, Synthetic construct
AIYTGADRPCAACCPCCPGTSCKAESNGVSYCRKDEP

SEQ ID NO: 23 - protein, 37 aa, Synthetic construct
AICTGADRPCAACCPCCPGTSCKYESNGVSYCRKDEP

SEQ ID NO: 24 - protein, 37 aa, Synthetic construct
AICTGADRPCAACCPCCPGTSCKAESFGVSYCRKDEP

SEQ ID NO: 25 - protein, 37 aa, Synthetic construct
AICTGADRPCAACNPCCPGTSCKAESNGVSYCRKMEP

SEQ ID NO: 26 - protein, 37 aa, Synthetic construct
AICIGADRPCAACCPCCPGTSCKAESNGVSYCRKDEP

SEQ ID NO: 27 - protein, 37 aa, Synthetic construct
AICTGADRPCAACCPCCPGTSCKAESNGCSYCRKDEP

SEQ ID NO: 28 - protein, 37 aa, Synthetic construct
AICTGADRPCAACCPCCPGVSCKAESNGVSYCRKDEP

SEQ ID NO: 29 - protein, 37 aa, Synthetic construct
AICTGADRPCAACCPCPPGTSCKAESNGVSYCRKDEP

SEQ ID NO: 30 - protein, 37 aa, Synthetic construct
AICTGADRPYAACCPCCPGTSCKAESNGVSYCRKDEP

SEQ ID NO: 31 - protein, 37 aa, Synthetic construct
AICTGADRPKAACCPCCPGTSCKAESNGVSYCRKDEP

SEQ ID NO: 32 - protein, 37 aa, Synthetic construct
AICTGADRPDAACCPCCPGTSCKAESNGVSYCRKDEP

SEQ ID NO: 33 - protein, 37 aa, Synthetic construct
AICTGADRPCAACCPCCRGTSCKAESNGVSYCRKDEP

SEQ ID NO: 34 - protein, 37 aa, Synthetic construct
AICTGGDRPCAACCPCCPGTSCKAESNGVSYCRKDEP

SEQ ID NO: 35 - protein, 37 aa, Synthetic construct
AICTGADRPCAACCPCCPGTSCKAESNGVSYCRKNEP

SEQ ID NO: 36 - protein, 37 aa, Synthetic construct
AICTGADRPCAACCPCCPGTTCKAESNGVSYCRKDEP

SEQ ID NO: 37 - protein, 37 aa, Synthetic construct
AICTGADRPCAACCPQCPGTSCKAESNGVSYCRKDEP

SEQ ID NO: 38 - protein, 37 aa, Synthetic construct
AICTGADRPCAACCPCCPGTSCKAESNYVSYCRKDEP

SEQ ID NO: 39 - protein, 37 aa, Synthetic construct
AICTKADRPCAACCPCCPGTSCKAASNGVSYCRKDEP

SEQ ID NO: 40 - protein, 37 aa, Synthetic construct
AVCTGADRPCAACCPCCPGTSCKAESNGVSYCRKDEP

SEQ ID NO: 41 - protein, 37 aa, Synthetic construct
AICTGADRPCAACCPCCPGTSCKANSNGVSYCRKDEP

SEQ ID NO: 42 - protein, 37 aa, Synthetic construct
AICTGADFPCAACCPCCPGTSCKAESNGVSYCRKDEP

SEQ ID NO: 43 - protein, 37 aa, Synthetic construct
AICTGADRPIAACCPCCPGTSCKAESNGVSYCRKDEP

SEQ ID NO: 44 - protein, 37 aa, Synthetic construct
AICTLADRPCAACCPCCPGQSCKAESNGVSYCRTDEP

SEQ ID NO: 45 - protein, 37 aa, Synthetic construct
AICTGADRPCAACCPCCPGSSCKAEGNGVSYCRKDEP

SEQ ID NO: 46 - protein, 37 aa, Synthetic construct
AICTGADRPCAACCPCCPGTSCKAEPNGVSYCRRDEP

SEQ ID NO: 47 - protein, 42 aa, Synthetic construct
AICTGADRPCAACCPCCPGTSCKAESNGVSYCRKEKAKRPLN

SEQ ID NO: 48 - protein, 37 aa, Synthetic construct
AICTGADRPCAACCPCCPGTSCKAESNGVSYCRKDVP

SEQ ID NO: 49 - protein, 37 aa, Synthetic construct
AICTGADRPCAACCPCCPGTSCKAESNGVSYCKKDEP

SEQ ID NO: 50 - protein, 37 aa, Synthetic construct
AICTGADRPCAACCPCCPGTSCKAEPNGVSYCRKDEP

SEQ ID NO: 51 - protein, 37 aa, Synthetic construct
AICTGADRPCAACCPCCPGTSCKAESNGVCYCRKDEP

SEQ ID NO: 52 - protein, 37 aa, Synthetic construct
AICTGADRICAACCPCCPGTSCKAESNGVSYCRKDEP

SEQ ID NO: 53 - protein, 37 aa, Synthetic construct
AICTGADRPCAACCPCWPGTSCKAESNGVSYCRKDEP

SEQ ID NO: 54 - protein, 37 aa, Synthetic construct
AICTGADRPCAACCPCCPGTSCKAESNGVSFCRKDEP

SEQ ID NO: 55 - protein, 37 aa, Synthetic construct
AICTDADRPCAACCPCCPGTSCKAESNGVSYCRKDEP

SEQ ID NO: 56 - protein, 37 aa, Synthetic construct
AIYTGADRPCAACCPCCPGTSCKAESNGVSNCRKDEP

SEQ ID NO: 57 - protein, 37 aa, Synthetic construct
AICTGADRPCAAGCPCCPGTSCKAESNGVSYCRKDEP

SEQ ID NO: 58 - protein, 37 aa, Synthetic construct
AICTRADRPQAACCPCCPGTSCKAESNGVSYCRKDEP

SEQ ID NO: 59 - protein, 37 aa, Synthetic construct
AICTGADRPCSACCPCCPGTSCKAESNGVSYCRKDEP

SEQ ID NO: 60 - protein, 37 aa, Synthetic construct
AICTGADRPCAACCPCCPGTSCKAESVGVSYCRKDEP

SEQ ID NO: 61 - protein, 37 aa, Synthetic construct
AIPTGADRPCAACCPCCPGTNCKAESNGVSYCRKDEP

SEQ ID NO: 62 - protein, 37 aa, Synthetic construct
AICTGADRNCAACCPCCPGTSCKAESNGVSYCRKDEP

SEQ ID NO: 63 - protein, 37 aa, Synthetic construct
AICTGADRPCAACCPCCPGTSCKAESNGVSNCRKDEP

SEQ ID NO: 64 - protein, 37 aa, Synthetic construct
AICTIADRPCAACCPCFGGTSCKAESNGVSYCRKDEP

SEQ ID NO: 65 - protein, 37 aa, Synthetic construct
AICTGADRPCAACCPCCPGTSCKAPSNGVSYCRKDDP

SEQ ID NO: 66 - protein, 37 aa, Synthetic construct
AICRGADRPCAACCPCCPGTSCKAESNGVSYCRKDEP

SEQ ID NO: 67 - protein, 37 aa, Synthetic construct
AICTGADRPCAACCPCCPITSCKAESNGVSYCRKDEP

SEQ ID NO: 68 - protein, 37 aa, Synthetic construct
AICTGADRPCAACCPCCPGTSCWAESNGVSYCRKDEP

SEQ ID NO: 69 - protein, 37 aa, Synthetic construct
AICTGADRPCAACCPCCPGTSWKAESNGVSYCRKDEP

SEQ ID NO: 70 - protein, 37 aa, Synthetic construct
AICTGADRPCAACCPCYPGTSCKAESNGVSYCRKDEP

SEQ ID NO: 71 - protein, 37 aa, Synthetic construct
AICTGADRPCAAECPCCPGTSCKAESNGVSYCRKDEP

SEQ ID NO: 72 - protein, 37 aa, Synthetic construct
AICTGADRPCAACCPYCPGTSCKAESNGVSYCRKDCP

SEQ ID NO: 73 - protein, 37 aa, Synthetic construct
AICTGADGPCAACCPCCPGTSCKAESNGVSYCRKDES

SEQ ID NO: 74 - protein, 37 aa, Synthetic construct
AICTGADSPCAACCPCCPGTTCKAESNGVSYCRKDEP

SEQ ID NO: 75 - protein, 37 aa, Synthetic construct
AICTGADRPCAACCPCCPGCSCKAESNGVSYCRKDEP

SEQ ID NO: 76 - protein, 37 aa, Synthetic construct
AICTGADRPCAACCPCCPGTSCKAETNGVSYCRKDEP

SEQ ID NO: 77 - protein, 37 aa, Synthetic construct
AICTGADRPCAACCPCCPGTSCKAESNGVSYCRGDEP

SEQ ID NO: 78 - protein, 37 aa, Synthetic construct
AICTGADRPCSACCPCCPGHSCKAESNGVSYCRKDEP

SEQ ID NO: 79 - protein, 37 aa, Synthetic construct
AICTGADRPRAACCPCCPGTSCKAESNGVSYCRKDEP

SEQ ID NO: 80 - protein, 37 aa, Synthetic construct
AICTGAARPCAACCPCCPGTSCAAESNGVSYCRKDEP

SEQ ID NO: 81 - protein, 37 aa, Synthetic construct
AICTGADRPCAACCPCCPGTDCKAESNGVSYCRKDEP

SEQ ID NO: 82 - protein, 37 aa, Synthetic construct
KICTGADRPCAACCPCCPGTSCKAESNGVSYCRKDEP

SEQ ID NO: 83 - protein, 37 aa, Synthetic construct
AICTGADRPCAACCPCCPGTSCKAESNGVVYCRKDEP

SEQ ID NO: 84 - protein, 37 aa, Synthetic construct
AICTGADRPCAACCPCCPGTHCKAESNGVSYCRKDEP

SEQ ID NO: 85 - protein, 37 aa, Synthetic construct
AICTGADRPCAACCPCCPGTSCKAESNGVSYCRADEP

SEQ ID NO: 86 - protein, 37 aa, Synthetic construct
AICTHADRPCAACCPCCPGTGCKAESNGVSYCRKDEP

SEQ ID NO: 87 - protein, 37 aa, Synthetic construct
AICTGADRPCAACCPCCPGTTCKAERNGVSYCRKDEP

SEQ ID NO: 88 - protein, 37 aa, Synthetic construct
AICTGADRPCAACCPCCPGTSCKAASNGVSYCRKDEP

SEQ ID NO: 89 - protein, 37 aa, Synthetic construct
AICTGADRPCAACCPGCPGTSCKAESNGVSYCRKDEP

SEQ ID NO: 90 - protein, 35 aa, Synthetic construct
AICTGADRPECCPCCPGTSCKAESNGVSYCRPDEP

SEQ ID NO: 91 - protein, 37 aa, Synthetic construct
AVCTGADRPCAACCPCCPGISCKAESNGVSYCRKDEP

SEQ ID NO: 92 - protein, 37 aa, Synthetic construct
AICTGADRPCAACCPCCPGASCKAESNGVSYCRKDEP

SEQ ID NO: 93 - protein, 37 aa, Synthetic construct
AICTGADRPCAACCPCCPGTSCKAERNGVCYCRKDEP

SEQ ID NO: 94 - protein, 37 aa, Synthetic construct
AICTGVDRPCAACCPCCPGTSCKAESKGVSYCRKDEP

SEQ ID NO: 95 - protein, 37 aa, Synthetic construct
AICTGADRPCAACCPCCPGTSCKAESAGVSYCRKDEP

SEQ ID NO: 96 - protein, 37 aa, Synthetic construct
AICTGADRPCAACCPCTPGTSCKAESNGVSYCRKDEP

SEQ ID NO: 97 - protein, 37 aa, Synthetic construct
AICTRADRPCAACCPCCPGTSCKAESNGVSYCRKDEP

SEQ ID NO: 98 - protein, 37 aa, Synthetic construct
AICTGADRPCAACCPCCPGTSCKAESNGVSYCRKDHP

SEQ ID NO: 99 - protein, 37 aa, Synthetic construct
AIDTGADRPCAACCPCCPGTSCKAESNGVSYCRKDEP

SEQ ID NO: 100 - protein, 34 aa, Synthetic construct
AICTGVDRPCAACCPCCPGTSCNAESNGVSLQER

SEQ ID NO: 101 - protein, 37 aa, Synthetic construct
AICTGADRPIAACCPCCPGTSCLAESNGVSYCRKDEP

SEQ ID NO: 102 - protein, 37 aa, Synthetic construct
AICTGADRPCAACCPCCPGTSCKAESNGVSYCRNDEP

SEQ ID NO: 103 - protein, 37 aa, Synthetic construct
AICTGADRPCAACCPCCPGTSCKAESNGVWYCRKGEP

SEQ ID NO: 104 - protein, 37 aa, Synthetic construct
IICTGADRPCAACCPCCPGTTCKAESNGVSYCRKDEP

SEQ ID NO: 105 - protein, 37 aa, Synthetic construct
AICTGADRPCAACCPCFPGTSCKAESNGVSYCRKDEP

SEQ ID NO: 106 - protein, 37 aa, Synthetic construct
AICWGPDRPCAACCPCCPGCSCKAESNGVSYCRKDEP

SEQ ID NO: 107 - protein, 37 aa, Synthetic construct
AICTGADRPCAACCPCCPGTSCKAESNGVSYCCKDEP

SEQ ID NO: 108 - protein, 37 aa, Synthetic construct
AICTGADRPCAACCGCCPGTSCKAESNGVSYCRPDEP

SEQ ID NO: 109 - protein, 37 aa, Synthetic construct
AICTGADRPCAACCPCQPGTSCKAESNGVSYCRKDEP

SEQ ID NO: 110 - protein, 37 aa, Synthetic construct
AICTGEDRPCAACCPCCPGTSCKAESNGVSYCRKDEP

SEQ ID NO: 111 - protein, 37 aa, Synthetic construct
AICTGADRPCAAFCPCCPGTSCKAESNGVSYSRKDEP

SEQ ID NO: 112 - protein, 37 aa, Synthetic construct
AICTGADRPCAACCPCCGGTSCKAESNGVSYCRKDEP

SEQ ID NO: 113 - protein, 37 aa, Synthetic construct
AICTGADRPCAACCPCCPRTSCKAESNGVSYCYKDEP

SEQ ID NO: 114 - protein, 37 aa, Synthetic construct
AICTGADRPCAACCPCCPGTSCFAESNGVSYCRKDEP

SEQ ID NO: 115 - protein, 37 aa, Synthetic construct
AICTGADKPCAACCPCCPGTTCKAERNGVSYCRKDEP

SEQ ID NO: 116 - protein, 37 aa, Synthetic construct
AICTGADRPCAACCPCCPGTSCKAESNGVSYCRKTEP

SEQ ID NO: 117 - protein, 37 aa, Synthetic construct
AIGTGADRPCAACCPCCPGTSCKAESNGVSYCRKDEP

SEQ ID NO: 118 - protein, 37 aa, Synthetic construct
AICTGADRPCAACCPCCPGTSCTAESNGVSYCRKDEP

SEQ ID NO: 119 - protein, 37 aa, Synthetic construct
AICTGADRPCAACCPCCPGYSCKAESNGVSYCRKDEP

SEQ ID NO: 120 - protein, 37 aa, Synthetic construct
AICTGADRDCAACCPCCPGTSCKAESNGVSYCRKDEP

SEQ ID NO: 121 - protein, 37 aa, Synthetic construct
AICTGADRPCAACCPCCPGTSCKAESIGVSYCRKDEP

SEQ ID NO: 122 - protein, 37 aa, Synthetic construct
AICTGADRPCAACCPCLPGTSCKAESNGVSYCRKDEP

SEQ ID NO: 123 - protein, 37 aa, Synthetic construct
AICKGADRPCAACCPCCPGTSCKAESNGVSYCRKDEP

SEQ ID NO: 124 - protein, 37 aa, Synthetic construct
AICTGADRPCAACCPCCPGTSCKAEANGVSYCRKDEP

SEQ ID NO: 125 - protein, 37 aa, Synthetic construct
RICTGADRPCAACCPCCPGTSCKAESNGVSYCRKDEP

SEQ ID NO: 126 - protein, 37 aa, Synthetic construct
AICTGADRPCAACCPCCPGTSCKAESNGVSYCRLDEP

SEQ ID NO: 127 - protein, 34 aa, Synthetic construct
AICTGADRPCAACCPGTSCKAESNGVSYCRKDEP

SEQ ID NO: 128 - protein, 37 aa, Synthetic construct
AICTGADRPEAACCPCCPGTSCWAESNGVSYCRKDEP

SEQ ID NO: 129 - protein, 37 aa, Synthetic construct
AICTGADRPCAACCPCCPGTSCKAESNGVIYCRKDEP

SEQ ID NO: 130 - protein, 37 aa, Synthetic construct
AICTGADRPCAACCPCCPGTSCKAESNGWSYCRKDEP

SEQ ID NO: 131 - protein, 37 aa, Synthetic construct
AICTGADRPCAACCPCCPGTSCKAECNGVSYCRKDEL

SEQ ID NO: 132 - protein, 37 aa, Synthetic construct
AICTGADRPCYACCPCCPGTSCKAESNGVSYRRKDEP

SEQ ID NO: 133 - protein, 37 aa, Synthetic construct
AICMGADRPCAACCPCCPGTSMKAESNGVSYCRKDEP

SEQ ID NO: 134 - protein, 37 aa, Synthetic construct
AIHTGADRPCAACCPCCPGTSCKAESNGVSYCRKDEP

SEQ ID NO: 135 - protein, 37 aa, Synthetic construct
AICSGADRPCAACCPCCPGFSCKAESNGVSYCRKDEP

SEQ ID NO: 136 - protein, 37 aa, Synthetic construct
AICTGADRPCAACCPCCPGPSCKAESNGVSYCRKDEP

SEQ ID NO: 137 - protein, 37 aa, Synthetic construct
AIATGADRPCAACCPCCPGTSCKAESNGVSYCRKDEP

SEQ ID NO: 138 - protein, 37 aa, Synthetic construct
GICTGADRPCAACCPCCPGTSSKAESNGVSYCRKDEP

SEQ ID NO: 139 - protein, 37 aa, Synthetic construct
AICTGADRPCAACCPCCPGTSCKHESNGVSYCRKDEP

SEQ ID NO: 140 - protein, 37 aa, Synthetic construct
AICTGADRPCAACCPCCPRTSCKAESNGVSYCRKDEP

SEQ ID NO: 141 - protein, 37 aa, Synthetic construct
AICTGADRPCAACCPPCPGTSCKAESNGVSYCRKDEP

SEQ ID NO: 142 - protein, 37 aa, Synthetic construct
PICTGADRPCAACCPCCPGTTCKAERNGVSYCRKDEP

SEQ ID NO: 143 - protein, 37 aa, Synthetic construct
AICTGADRPCAACCPCCPGTTCKAGSNGVSYCRKDEP

SEQ ID NO: 144 - protein, 37 aa, Synthetic construct
AICTGADRPTAACCPCCPGTTCKAERNGVSYCRKDEG

SEQ ID NO: 145 - protein, 33 aa, Synthetic construct
AICTGADRPCAACCPCCPGAEDNGVSYCRKDEP

SEQ ID NO: 146 - protein, 37 aa, Synthetic construct
AICTGADRPCAACCPCCPGTSCKAESNGVSYCRMDEP

SEQ ID NO: 147 - protein, 37 aa, Synthetic construct
AICTGADRPCAACCPCCPGISCKAESNGVSYCRKDEP

SEQ ID NO: 148 - protein, 37 aa, Synthetic construct
AICTGADRPCAACCPCCPGTSCKAESNGVSYDRKDEP

SEQ ID NO: 149 - protein, 37 aa, Synthetic construct
AICTGADRPCAACCPCCPGTSCKAEKNGVSYCRKDEP

SEQ ID NO: 150 - protein, 37 aa, Synthetic construct
AICTGAARPCAACWPCCPGTSCKAESNGVSYCRKDEP

SEQ ID NO: 151 - protein, 37 aa, Synthetic construct
WICTGADRPCAACCPCCPGTSCKAESNGVSYCRKDEP

SEQ ID NO: 152 - protein, 37 aa, Synthetic construct
AICTGADRACAACCPCCPGTSCKAESNGVSYCRKDEP

SEQ ID NO: 153 - protein, 37 aa, Synthetic construct
AICTGADRPCAACCPCCPGTSNKAETNGVSYCRKDEP

SEQ ID NO: 154 - protein, 37 aa, Synthetic construct
AICTGADRPCAACCPCCPKTSCKAESNGVSYCRKDEP

SEQ ID NO: 155 - protein, 37 aa, Synthetic construct
AICSGADRPCAACCPGCPGTTCKAESNGVSYCRKDEP

SEQ ID NO: 156 - protein, 37 aa, Synthetic construct
AICTGAFRPCAACCPCCPGTSCKAESNGVSYCRKDEP

SEQ ID NO: 157 - protein, 37 aa, Synthetic construct
AICTGADRPCAACCPCCPGTSCKAESNGVSYCREDEP

SEQ ID NO: 158 - protein, 37 aa, Synthetic construct
AICTGADRPCAACCPCCPGQSCKAESNGVSYCRKDEP

SEQ ID NO: 159 - protein, 37 aa, Synthetic construct
AICTGADPPCAACCPCCPGTSCKAESNGVSYCYKDEP

SEQ ID NO: 160 - protein, 37 aa, Synthetic construct
AICTGADRPCAACCPCCPGTSCKAESNGVSYCRVDEP

SEQ ID NO: 161 - protein, 37 aa, Synthetic construct
AICTGADRPCAACCPCCPGTSCKAHSNGVSYCRKDEP

SEQ ID NO: 162 - protein, 37 aa, Synthetic construct
AICTGADRPCAACCPCCPGTSCKAESNGVSYCRKDES

SEQ ID NO: 163 - protein, 37 aa, Synthetic construct
AILTGADRPCAACCPCCPGTSCKAESNGVSYCRKDEP

SEQ ID NO: 164 - protein, 37 aa, Synthetic construct
AVCTGADRPCAACCPCCPGTSCKAESNGVWYCRKDEP

SEQ ID NO: 165 - protein, 37 aa, Synthetic construct
AICTGADRPCACCCPCCPGTSCKAESNGVSYCRKDEP

SEQ ID NO: 166 - protein, 37 aa, Synthetic construct
AICTGADRPCAACCSCCPGTSCKAESNGVSECRKDEP

SEQ ID NO: 167 - protein, 37 aa, Synthetic construct
AICTGADRPCAACCPCCPGTSCKAESNGVYYCRKDEP

SEQ ID NO: 168 - protein, 37 aa, Synthetic construct
AICTGADRPCAACCPCCPGTSCKAESLGVSYCRKDEP

SEQ ID NO: 169 - protein, 37 aa, Synthetic construct
AICTGADRPCAACCPHCPGTSCKAESNGVSYCVKDEP

SEQ ID NO: 170 - protein, 37 aa, Synthetic construct
AICTGADRPCAACCPCCPPTSCKAESNGVSYCRKDEP

SEQ ID NO: 171 - protein, 37 aa, Synthetic construct
AICTGADRPCAQCCPCCPGTSCKAESNGVSYCRKDEP

SEQ ID NO: 172 - protein, 37 aa, Synthetic construct
AICTGADRPCAACCPCCPDTSCKAESNGVSYCRKDEP

SEQ ID NO: 173 - protein, 37 aa, Synthetic construct
AICTGADRPCAACCMCCPGTSCKAESNGVSYCRKDEP

SEQ ID NO: 174 - protein, 37 aa, Synthetic construct
AECTGADRPCDACCPCCPGTSCKAESNGVSYCRKDEP

SEQ ID NO: 175 - protein, 37 aa, Synthetic construct
AICTGADRPCAACCPCNPGTSCKAESNGVSYCRKDEP

SEQ ID NO: 176 - protein, 37 aa, Synthetic construct
AICTGADRPCAACCPCCPGTSCKWESNGVSYCRKDEP

SEQ ID NO: 177 - protein, 37 aa, Synthetic construct
AICTGADRPCAACCPCCPGTSCKAESNGVSSCRKDEP

SEQ ID NO: 178 - protein, 37 aa, Synthetic construct
AICTGADRPCAACCPCCPGTSCKAEGNGVSYCRKDEP

SEQ ID NO: 179 - protein, 37 aa, Synthetic construct
AICTGADRPCAACCPCCPGTSCKAESNGVSRCRKDEP

SEQ ID NO: 180 - protein, 37 aa, Synthetic construct
AICTGADRPCAACCPCCPGTSCKAWSNGVSYCRKDEP

SEQ ID NO: 181 - protein, 37 aa, Synthetic construct
AICTGADRPCAACCPCCPGTSCKAEENGVSYCRKDEP

SEQ ID NO: 182 - protein, 37 aa, Synthetic construct
ATCTGADRPCAACCPCCPGTSCKAESNGVSYCRKDHP

SEQ ID NO: 183 - protein, 37 aa, Synthetic construct
AICTGANRPCAAGCPCCPGTSCKAESNGVSYCRKDEP

SEQ ID NO: 184 - protein, 37 aa, Synthetic construct
AICTGADRPCAACCPCCPGTSCKAESNGVSYCRKDEQ

SEQ ID NO: 185 - protein, 37 aa, Synthetic construct
AICTGADRPCAAFCPCCPGTSCKAESNGVSYCRKDEP

SEQ ID NO: 186 - protein, 37 aa, Synthetic construct
AICTGADRPCAACCPCCPGTSCKAESWGVSYCRKDEP

SEQ ID NO: 187 - protein, 37 aa, Synthetic construct
AICTGAARPCAACCPCCPGTSCKAETNGVSYCRKDEM

SEQ ID NO: 188 - protein, 37 aa, Synthetic construct
AICTGSDRPCAACCPCCPGTSCKAESNGVSYCRKDEP

SEQ ID NO: 189 - protein, 37 aa, Synthetic construct
AICTGADRPCAACCPCCPGTSCKAESNGVSYCQKDEP

SEQ ID NO: 190 - protein, 37 aa, Synthetic construct
AICTGADRACAACCPCCPGTRCKAESNGVSYCRKDEP

SEQ ID NO: 191 - protein, 37 aa, Synthetic construct
AICTGADRPCAACCPCCPGTTCQAESNGVSYCRKDEP

SEQ ID NO: 192 - protein, 37 aa, Synthetic construct
AICTGIDRPCAACCPCCPGTSCKAESNGVSYCRKDKP

SEQ ID NO: 193 - protein, 37 aa, Synthetic construct
AICTGADRPCAACCPRCPGTSCKAESNGVSYCRKDEP

SEQ ID NO: 194 - protein, 37 aa, Synthetic construct
AICTGADRPCAACCPFCPGTSCKAESNGVSYCRKDEP

SEQ ID NO: 195 - protein, 37 aa, Synthetic construct
AICTGAGRPCAACCPCCPGTSCKAESNGVSYCRKDFP

SEQ ID NO: 196 - protein, 37 aa, Synthetic construct
AICTGADRPCAACCPGCPGTSCNAESNHVSYCRKDEP

SEQ ID NO: 197 - protein, 37 aa, Synthetic construct
AICTGADRPCAACCPCCPGTSCKAESNWVSYCRKDEP

SEQ ID NO: 198 - protein, 37 aa, Synthetic construct
AICTGADRPCALCCPCCPGTTCKAESNGVSYCRKDEP

SEQ ID NO: 199 - protein, 37 aa, Synthetic construct
AACTGADRPCAACCPCCPGTSCKAESNGVSYCRKDEP

SEQ ID NO: 200 - protein, 37 aa, Synthetic construct
AICTGADRPCAACCPCCPGTTCKAESNGVTYCRKDEP

SEQ ID NO: 201 - protein, 37 aa, Synthetic construct
AICTGAARPCAACCPCCPGTSCKAESNGVSYCRKDEP

SEQ ID NO: 202 - protein, 37 aa, Synthetic construct
AICTGADRPCAACCPKCPGTSCKAESNGVSYCRKDEP

SEQ ID NO: 203 - protein, 37 aa, Synthetic construct
AICTGADLPCAACCPCCPGTSCKDESNGVSYCRKDEP

SEQ ID NO: 204 - protein, 37 aa, Synthetic construct
AIHTGADRPCAACCPCCPGTSCDAESNGVSYCRKDEP

SEQ ID NO: 205 - protein, 37 aa, Synthetic construct
AICTGADRPCAACCPCCPGTSCAAESNGVSYCRKDEP

SEQ ID NO: 206 - protein, 37 aa, Synthetic construct
AICTGADRPCAACCPCCPGTSCDAESNGVSYCRKDEP

SEQ ID NO: 207 - protein, 37 aa, Synthetic construct
AICTGADRPCAACCPCCPGTSCEAESNGVSYCRKDEP

SEQ ID NO: 208 - protein, 37 aa, Synthetic construct
AICTGADRPCAACCPCCPGTSCGAESNGVSYCRKDEP

SEQ ID NO: 209 - protein, 37 aa, Synthetic construct
AICTGADRPCAACCPCCPGTSCIAESNGVSYCRKDEP

SEQ ID NO: 210 - protein, 37 aa, Synthetic construct
AICTGADRPCAACCPCCPGTSCLAESNGVSYCRKDEP

SEQ ID NO: 211 - protein, 37 aa, Synthetic construct
AICTGADRPCAACCPCCPGTSCMAESNGVSYCRKDEP

SEQ ID NO: 212 - protein, 37 aa, Synthetic construct
AICTGADRPCAACCPCCPGTSCNAESNGVSYCRKDEP

SEQ ID NO: 213 - protein, 37 aa, Synthetic construct
AICTGADRPCAACCPCCPGTSCPAESNGVSYCRKDEP

SEQ ID NO: 214 - protein, 37 aa, Synthetic construct
AICTGADRPCAACCPCCPGTSCQAESNGVSYCRKDEP

SEQ ID NO: 215 - protein, 37 aa, Synthetic construct
AICTGADRPCAACCPCCPGTSCRAESNGVSYCRKDEP

SEQ ID NO: 216 - protein, 37 aa, Synthetic construct
AICTGADRPCAACCPCCPGTSCVAESNGVSYCRKDEP

SEQ ID NO: 217 - protein, 37 aa, Synthetic construct
AICTGADRPCAACCPCCPGTSCYAESNGVSYCRKDEP

SEQ ID NO: 218 - protein, 37 aa, Synthetic construct
AICTGADRPCAACCPCCPGTSCKAESNGVSYCRDDEP

SEQ ID NO: 219 - protein, 37 aa, Synthetic construct
AICTGADRPCAACCPCCPGTSCKAESNGVSYCRFDEP

SEQ ID NO: 220 - protein, 37 aa, Synthetic construct
AICTGADRPCAACCPCCPGTSCKAESNGVSYCRHDEP

SEQ ID NO: 221 - protein, 37 aa, Synthetic construct
AICTGADRPCAACCPCCPGTSCKAESNGVSYCRIDEP

SEQ ID NO: 222 - protein, 37 aa, Synthetic construct
AICTGADRPCAACCPCCPGTSCKAESNGVSYCRPDEP

SEQ ID NO: 223 - protein, 37 aa, Synthetic construct
AICTGADRPCAACCPCCPGTSCKAESNGVSYCRQDEP

SEQ ID NO: 224 - protein, 37 aa, Synthetic construct
AICTGADRPCAACCPCCPGTSCKAESNGVSYCRSDEP

SEQ ID NO: 225 - protein, 37 aa, Synthetic construct
AICTGADRPCAACCPCCPGTSCKAESNGVSYCRTDEP

SEQ ID NO: 226 - protein, 37 aa, Synthetic construct
VICTGADRPCAACCPCCPGTSCKAESNGVSYCRKDEP

SEQ ID NO: 227 - protein, 37 aa, Synthetic construct
LICTGADRPCAACCPCCPGTSCKAESNGVSYCRKDEP

SEQ ID NO: 228 - protein, 35 aa, Synthetic construct
SICTGADRPCAACCPCCPGTSCKAGVNGVAYCRKD

SEQ ID NO: 229 - protein, 35 aa, Synthetic construct
AICTGADRPCAACCPCCPGTSCKAEANGVSRCRKD

SEQ ID NO: 230 - protein, 37 aa, Synthetic construct
AICTGADRPCAACCPCCPGTSCKAEVNGVSYCRKDEP

SEQ ID NO: 231 - protein, 37 aa, Synthetic construct
SICTGADRPCAACCPCCPGTSCKAESNGVSYCRKDEP

SEQ ID NO: 232 - protein, 37 aa, Synthetic construct
DICTGADRPCAACCPCCPGTSCKAESNGVSYCRKDEP

SEQ ID NO: 233 - protein, 37 aa, Synthetic construct
AICTGADRPCAACCPCCPGTSCKAESNGVSLCRKDEP

SEQ ID NO: 234 - protein, 37 aa, Synthetic construct
AICTGADRPCAACCPCCPGTSCKAESNGVSECRKDEP

SEQ ID NO: 235 - protein, 37 aa, Synthetic construct
AICTGADKPCAACCPCCPGTSCKAESNGVSYCRKDEP

SEQ ID NO: 236 - protein, 37 aa, Synthetic construct
AICTGADHPCAACCPCCPGTSCKAESNGVSYCRKDEP

SEQ ID NO: 237 - protein, 37 aa, Synthetic construct
AICTGADYPCAACCPCCPGTSCKAESNGVSYCRKDEP

SEQ ID NO: 238 - protein, 37 aa, Synthetic construct
AICTGADLPCAACCPCCPGTSCKAESNGVSYCRKDEP

SEQ ID NO: 239 - protein, 37 aa, Synthetic construct
AICTGADMPCAACCPCCPGTSCKAESNGVSYCRKDEP

SEQ ID NO: 240 - protein, 37 aa, Synthetic construct
AICTGADSPCAACCPCCPGTSCKAESNGVSYCRKDEP

SEQ ID NO: 241 - protein, 34 aa, Synthetic construct
AICTGADRPCAACCPCCPGTSCKAESNGVSYCHH

SEQ ID NO: 242 - protein, 33 aa, Synthetic construct
AICTGADRPCAACCPCCPGTSCKAESNGVSYCH

SEQ ID NO: 243 - protein, 37 aa, Synthetic construct
AICTGADRPCGGCCPCCPGTSCKAESNGVSYCRKDEP

SEQ ID NO: 244 - protein, 37 aa, Synthetic construct
AICTGADRPCGACCPCCPGTSCKAESNGVSYCRKDEP

SEQ ID NO: 245 - protein, 37 aa, Synthetic construct
AICTGADRPCAGCCPCCPGTSCKAESNGVSYCRKDEP

SEQ ID NO: 246 - protein, 37 aa, Synthetic construct
AICTGADRPCAACCPCCPGTKCKAESNGVSYCRKDEP

SEQ ID NO: 247 - protein, 37 aa, Synthetic construct
AICTGADRPCAACCPCCPGTRCKAESNGVSYCRKDEP

SEQ ID NO: 248 - protein, 37 aa, Synthetic construct
AICTGADGPCAACCPCCPGTSCKAESNGVSYCRKDEP

SEQ ID NO: 249 - protein, 35 aa, Synthetic construct
AICTGADRPCAACCPCCPGTSCKTESNGVSYCRKD

SEQ ID NO: 250 - protein, 37 aa, Synthetic construct
PICTGADRPCAACCPCCPGTSCKAESNGVSYCRKDEP

SEQ ID NO: 251 - protein, 37 aa, Synthetic construct
EAAICTGADRPCAACCPCCPGTSCKAESNGVSYCRKD

SEQ ID NO: 252 - protein, 39 aa, Synthetic construct
NSAICTGADRPCAACCPCCPGTSCKAESNGVSYCRKDEP

SEQ ID NO: 253 - protein, 39 aa, Synthetic construct
GSAICTGADRPCAACCPCCPGTSCKAESNGVSYCRKDEP

SEQ ID NO: 254 - protein, 37 aa, Synthetic construct
GSAICTGADRPCAACCPCCPGTSCKAESNGVSYCRKD

SEQ ID NO: 255 - protein, 39 aa, Synthetic construct
EAAVCTGADRPCAACCPCCPGTSCKAESNGVSYCRKDEP

SEQ ID NO: 256 - protein, 37 aa, Synthetic construct
GSQYCTGADRPCAACCPCCPGTSCKAESNGVSYCRKA

SEQ ID NO: 257 - protein, 37 aa, Synthetic construct
GICTGADRPCAACCPCCPGTSCKAESNGVWYCRKDEP

SEQ ID NO: 258 - protein, 34 aa, Synthetic construct
AICTGADRPCGACCPCCPGTSCKAESNGVSYCRA

SEQ ID NO: 259 - protein, 37 aa, Synthetic construct
GICTGADRPCGACCPCCPGTSCKAESNGVSYCRKDEP

SEQ ID NO: 260 - protein, 37 aa, Synthetic construct
AICTPADRPCAACCPCCPGTSCKAESNGVSYCRKDEP

SEQ ID NO: 261 - protein, 37 aa, Synthetic construct
AICTGADRPCAACCPCCSGASCKAESNGVSYCRKDEP

SEQ ID NO: 262 - protein, 34 aa, Synthetic construct
GICTGADRPCAACCPCCPGASCKAESNGVSYCRA

SEQ ID NO: 263 - protein, 37 aa, Synthetic construct
AICTGADRPCAACCPCCPGTSCSAESNGVSYCRKDEP

SEQ ID NO: 264 - protein, 37 aa, Synthetic construct
AICTGADRPCAACCPCCPGTSCQAASNGVSYCRKDEP

SEQ ID NO: 265 - protein, 38 aa, Synthetic construct
NAICTGADRPCAACCPCCPGTSCYAESNGVSYCRKDEP

SEQ ID NO: 266 - protein, 38 aa, Synthetic construct
QAICTGADRPCAACCPCCPGTSCYAESNGVSYCRKDEP

SEQ ID NO: 267 - protein, 34 aa, Synthetic construct
AICTGADRPCAACCPCCPGTSCVAESNGVSYCRA

SEQ ID NO: 268 - protein, 34 aa, Synthetic construct
GICTGADRPCAACCPCCPGTSCVAESNGVSYCRA

SEQ ID NO: 269 - protein, 37 aa, Synthetic construct
AICTGADRPCAACCPCCPGTSCKAESNGVWYCRKDEP

SEQ ID NO: 270 - protein, 37 aa, Synthetic construct
AICTGADRPCAACCPCCPGTSCKATSNGVSYCRKDEP

SEQ ID NO: 271 - protein, 37 aa, Synthetic construct
AICTGADRPCAACCPCCSGASCTEQSNGVSYCRKDEP

SEQ ID NO: 272 - protein, 34 aa, Synthetic construct
AICTGADRPCAACCPCCSGASCTEQSNGVSYCRA

SEQ ID NO: 273 - protein, 35 aa, Synthetic construct
AICTGADRPCAACCPCCPGTSCKAFSNGVSYCRKD

SEQ ID NO: 274 - protein, 38 aa, Synthetic construct
GAICTGADRPCAACCPCCPGTSCKAESNGVSYCRKDEP

SEQ ID NO: 275 - protein, 36 aa, Synthetic construct
GAICTGADRPCAACCPCCPGTSCKAESNGVSYCRKD

SEQ ID NO: 276 - protein, 38 aa, Synthetic construct
GSICTGADRPCAACCPCCPGTSCKAESNGVSYCRKDEP

SEQ ID NO: 277 - protein, 35 aa, Synthetic construct
SICTGADRPCAACCPCCPGTSCKAFSNGVSYCRKD

SEQ ID NO: 278 - protein, 37 aa, Synthetic construct
SICTGADRPCAACCPCCPGTSCYAESNGVSYCRKDEP

SEQ ID NO: 279 - protein, 35 aa, Synthetic construct
AICTGADRPCAACCPCCPGTSCYAESNGVSYCRKD

SEQ ID NO: 280 - protein, 37 aa, Synthetic construct
SICTGADRPCAACCPCCPGTSCKAESNGVSWCRKDEP

SEQ ID NO: 281 - protein, 37 aa, Synthetic construct
AICTGADRPCAACCPCCPGTSCKARSNGVSYCRKDEP

SEQ ID NO: 282 - protein, 37 aa, Synthetic construct
AICTGADRPCAACCPCCPGTSCKAMSNGVSYCRKDEP

SEQ ID NO: 283 - protein, 37 aa, Synthetic construct
GICTGADRPCAACCPCCPGTSCKAESNGVSYCRKDEP

SEQ ID NO: 284 - protein, 37 aa, Synthetic construct
AICTGADRPCAACCPCCPGTSCKASSNGVSYCRKDEP

SEQ ID NO: 285 - protein, 35 aa, Synthetic construct
LICTGADRPCAACCPCCPGTSCYAESNGVSYCRKD

SEQ ID NO: 286 - protein, 37 aa, Synthetic construct
AICTGADRPCAACCPCCPGTSCKAESNGVSYCRKAEP

SEQ ID NO: 287 - protein, 37 aa, Synthetic construct
AICTGADRPCAACCPCCPGTSCKAESNGVSYCRKEEP

SEQ ID NO: 288 - protein, 37 aa, Synthetic construct
AICTGADRPCAACCPCCPGTSCKAESNGVSYCRKGEP

SEQ ID NO: 289 - protein, 37 aa, Synthetic construct
AICTGADRPCAACCPCCPGTSCKAESNGVSYCRKHEP

SEQ ID NO: 290 - protein, 37 aa, Synthetic construct
AICTGADRPCAACCPCCPGTSCKAESNGVSYCRKIEP

SEQ ID NO: 291 - protein, 37 aa, Synthetic construct
AICTGADRPCAACCPCCPGTSCKAESNGVSYCRKKEP

SEQ ID NO: 292 - protein, 37 aa, Synthetic construct
AICTGADRPCAACCPCCPGTSCKAESNGVSYCRKMEP

SEQ ID NO: 293 - protein, 37 aa, Synthetic construct
AICTGADRPCAACCPCCPGTSCKAESNGVSYCRKPEP

SEQ ID NO: 294 - protein, 37 aa, Synthetic construct
AICTGADRPCAACCPCCPGTSCKAESNGVSYCRKQEP

SEQ ID NO: 295 - protein, 37 aa, Synthetic construct
AICTGADRPCAACCPCCPGTSCKAESNGVSYCRKREP

SEQ ID NO: 296 - protein, 37 aa, Synthetic construct
AICTGADRPCAACCPCCPGTSCKAESNGVSYCRKSEP

SEQ ID NO: 297 - protein, 37 aa, Synthetic construct
AICTGADRPCAACCPCCPGTSCKAESNGVSYCRKVEP

SEQ ID NO: 298 - protein, 37 aa, Synthetic construct
AICTGADRPCAACCPCCPGTSCKAESNGVSYCRKWEP

SEQ ID NO: 299 - protein, 37 aa, Synthetic construct
AICTGADRPCAACCPCCPGTSCKAESNGVSYCRKYEP

SEQ ID NO: 300 - protein, 37 aa, Synthetic construct
AICTGADRPCAACCPCCPGTSCKQEANGVSLCRKDEP

SEQ ID NO: 301 - protein, 37 aa, Synthetic construct
SICTGADRPCAACCPCCPGTSCKIGCNGLHYCRKDEP

SEQ ID NO: 302 - protein, 37 aa, Synthetic construct
SICTGADRPCAACCPCCPGTSCQIWSNGVSYCRKDEP

SEQ ID NO: 303 - protein, 37 aa, Synthetic construct
AICTGADRPCAACCPCCPGTSCKYESLGVSYCRKDEP

SEQ ID NO: 304 - protein, 37 aa, Synthetic construct
AICTGADRPCAACCPCCPGTSCTARQNGVPYCRKDEP

SEQ ID NO: 305 - protein, 37 aa, Synthetic construct
AICTGADRPCAACCPCCPGTSCKSTGNGAGYCRKDEP

SEQ ID NO: 306 - protein, 37 aa, Synthetic construct
AICTGADRPCAACCPCCPGTSCKAGSAGAFYCRKDEP

SEQ ID NO: 307 - protein, 37 aa, Synthetic construct
AICTGADRPCAACCPCCPGTSCKATLNGVSYCRKDEP

SEQ ID NO: 308 - protein, 37 aa, Synthetic construct
AICTGADRPCAACCPCCPGTSCPDESEGVYYCRKDEP

SEQ ID NO: 309 - protein, 37 aa, Synthetic construct
TICGGDDTPCAACCPCCPGTSCKPESNGFSLCRKDEP

SEQ ID NO: 310 - protein, 37 aa, Synthetic construct
SICTGADRPCAACCPCCPGTSCKWESNVESYCRKDEP

SEQ ID NO: 311 - protein, 37 aa, Synthetic construct
AICTGADRPCAACCPCCPGTSCVAESWGVSYCRKDEP

SEQ ID NO: 312 - protein, 37 aa, Synthetic construct
AICTGADRPCAACCPCCPGTSCKADHWGVPYCRKDEP

SEQ ID NO: 313 - protein, 37 aa, Synthetic construct
SICTGADRPCAACCPCCPGTSCKEEIEGPSYCRKDEP

SEQ ID NO: 314 - protein, 37 aa, Synthetic construct
IICTGADRPCAACCPCCPGTSCKKQSNGVSYCRKDEP

SEQ ID NO: 315 - protein, 37 aa, Synthetic construct
AICTGADRPCAACCPCCPGTSCKAEDNGGTYCRKDEP

SEQ ID NO: 316 - protein, 37 aa, Synthetic construct
SICTGADRPCAACCPCCPGTSCSFESGGVSYCRKDEP

SEQ ID NO: 317 - protein, 37 aa, Synthetic construct
AICTGADRPCAACCPCCPGTSCKSESNGSYYCRKDEP

SEQ ID NO: 318 - protein, 37 aa, Synthetic construct
AICTGANRPCAACCPCCPGTSCKAESNGVSYCRKDEP

SEQ ID NO: 319 - protein, 37 aa, Synthetic construct
AICTGANRPCAACCPCCPGTSCVAESNGVSYCRKDEP

SEQ ID NO: 320 - protein, 37 aa, Synthetic construct
AICTGADRPCAACCPCCPGTSCKTESNGVSYCRKDEP

SEQ ID NO: 321 - protein, 34 aa, Synthetic construct
AICTGADRPCAACCPCCPGTSCKAESNGVSYCRA

SEQ ID NO: 322 - protein, 35 aa, Synthetic construct
AICTGADRPCAACCPCCPGTSCKAESNGVSYCRKD

SEQ ID NO: 323 - protein, 37 aa, Synthetic construct
ALCTGADRPCAACCPCCPGTSCKAESNGVSYCRKDEP

SEQ ID NO: 324 - protein, 42 aa, Synthetic construct
AICTGADRPCAACCPCCPGTSWKQRVMVFLIAGRMNRKRPLN

SEQ ID NO: 325 - protein, 38 aa, Synthetic construct
AICTGADRPCAACCPCCPGTSCQGPESNGVSYCRKDEP

SEQ ID NO: 326 - protein, 39 aa, Synthetic construct
TICTGADRPCAACCPCCPGTSCTEPESNGGHTVYYCRND

SEQ ID NO: 327 - protein, 38 aa, Synthetic construct
QACTPADQPCAACCPCCSGASCTEQLNENGHTVYYCRA

SEQ ID NO: 328 - protein, 35 aa, Synthetic construct
AICTGADRPCAACCPCCPGTSCQGPESNGVSYCRA

SEQ ID NO: 329 - protein, 38 aa, Synthetic construct
AICTGADRPCAACCPCCPGTSCAGPESNGVSYCRKDEP

SEQ ID NO: 330 - protein, 38 aa, Synthetic construct
AICTGADRPCAACCPCCPGTSCQGAESNGVSYCRKDEP

SEQ ID NO: 331 - protein, 38 aa, Synthetic construct
AICTGADRPCAACCPCCPGTSCKGPESNGVSYCRKDEP

SEQ ID NO: 332 - protein, 38 aa, Synthetic construct
AICTGADRPCAACCPCCPGTSCNGPESNGVSYCRKDEP

SEQ ID NO: 333 - protein, 39 aa, Synthetic construct
QAICTGADRPCAACCPCCPGTSCQGPESNGVSYCRKDEP

SEQ ID NO: 334 - protein, 39 aa, Synthetic construct
NAICTGADRPCAACCPCCPGTSCQGPESNGVSYCRKDEP

SEQ ID NO: 335 - protein, 41 aa, Synthetic construct
AICTGADRPCAASQCCPCCPGTSCTEPESNGGHTVYYCRND

SEQ ID NO: 336 - protein, 39 aa, Synthetic construct
AICTGADRPCAACCPCCPGTSCTEPESNGGHTVYYCRND

SEQ ID NO: 337 - protein, 39 aa, Synthetic construct
QACTGADRPCAACCPCCPGTSCTEPESNGGHTVYYCRND

SEQ ID NO: 338 - protein, 41 aa, Synthetic construct
QATICTGADRPCAACCPCCPGTSCTEPESNGGHTVYYCRND

SEQ ID NO: 339 - protein, 38 aa, Synthetic construct
AICTGADRPCAACCPCCSGASCGGPESNGVSYCRKDEP

SEQ ID NO: 340 - protein, 35 aa, Synthetic construct
AICTGADRPCAACCPCCSGASCGGPESNGVSYCRA

SEQ ID NO: 341 - protein, 38 aa, Synthetic construct
AICTGADRPCGACCPCCSGASCGGPESNGVSYCRKDEP

SEQ ID NO: 342 - protein, 35 aa, Synthetic construct
AICTGADRPCGACCPCCSGASCGGPESNGVSYCRA

SEQ ID NO: 343 - protein, 36 aa, Synthetic construct
TICTGADRPCAACCPCCPGTSCTEPESNGVSYCRND

SEQ ID NO: 344 - protein, 36 aa, Synthetic construct
AICTGADRPCAACCPCCPGTSCTEPESNGVSYCRND

SEQ ID NO: 345 - protein, 38 aa, Synthetic construct
SICTGADRPCAACCPCCPGTSCQQDEFQNVWNCRKDEP

SEQ ID NO: 346 - protein, 38 aa, Synthetic construct
SICTGADRPCAACCPCCPGTSCHKADSNGLSYCRKDEP

SEQ ID NO: 347 - protein, 38 aa, Synthetic construct
SICTGADRPCAACCPCCPGTSCIKRESNGVSYCRKDEP

SEQ ID NO: 348 - protein, 38 aa, Synthetic construct
AICTGADRPCAACCPCCPGTSCTKRESNGVKYCRKDEP

SEQ ID NO: 349 - protein, 38 aa, Synthetic construct
AICTGADRPCAACCPCCPGTSCQKAESNGVLYCRKDEP

SEQ ID NO: 350 - protein, 38 aa, Synthetic construct
TICSGGDRPRAACCPCCPGTSSGKARVNGVFYCRKDEP

SEQ ID NO: 351 - protein, 38 aa, Synthetic construct
SICTGADRPCAACCPCCPGTSCRKADNGGVDYCRKDEP

SEQ ID NO: 352 - protein, 38 aa, Synthetic construct
AICTGADRPCAACCPCCPGTSCVKARVNGVFYCRKDEP

SEQ ID NO: 353 - protein, 38 aa, Synthetic construct
SICTGADSPCAACCPSCPGTSCKGASWWGVCNCRKDEP

SEQ ID NO: 354 - protein, 38 aa, Synthetic construct
AICTGADRPCAACCPCCPGTSCTKAESNGISYCRKDEP

SEQ ID NO: 355 - protein, 38 aa, Synthetic construct
SICTGADRPCAACCPCCPGTSCQKDDHNGVGYCRKDEP

SEQ ID NO: 356 - protein, 38 aa, Synthetic construct
AICTGADRPCAACCPCCPGTSCYKAPDNGLPYCRKDEP

SEQ ID NO: 357 - protein, 38 aa, Synthetic construct
AICTGADRPCAAYCPCCPGTSCQKALSNGSSYCRKDEP

SEQ ID NO: 358 - protein, 37 aa, Synthetic construct; identical to SEQ ID NO: 160

SEQ ID NO: 359 - protein, 24 aa, Hordeum vulgare
MANKHLSLSLFLVLLGLSASLASG

SEQ ID NO: 360 - protein, 6 aa, Synthetic construct
ALKFLV

SEQ ID NO: 361 - protein, 6 aa, Synthetic construct
ALKLFV

SEQ ID NO: 362 - protein, 6 aa, Synthetic construct
IFVRLR

SEQ ID NO: 363 - protein, 6 aa, Synthetic construct
LFAAPF

SEQ ID NO: 364 - protein, 8 aa, Synthetic construct
ALKFLVGS

SEQ ID NO: 365 - protein, 8 aa, Synthetic construct
ALKLFVGS

SEQ ID NO: 366 - protein, 8 aa, Synthetic construct
IFVRLRGS

SEQ ID NO: 367 - protein, 8 aa, Synthetic construct
LFAAPFGS

SEQ ID NO: 368 - protein, 8 aa, Synthetic construct
LFVRLRGS

SEQ ID NO: 369 - protein, 6 aa, Synthetic construct
LGERGS

SEQ ID NO: 370 - protein, 27 aa, Nicotiana plumbaginifolia
EMGKMASLFASLLVVLVSLSLASESSA

SEQ ID NO: 371 - protein, 26 aa, Nicotiana plumbaginifolia
MGKMASLFATFLVVLVSLSLASESSA

SEQ ID NO: 372 - DNA, 78 bp, Nicotiana plumbaginifolia
  source 1..78: mol_type = other DNA
atgggtaagatggcttctctgtttgcttctctgctggttgttctggtttctctgtctctggcttctgaatcttctgct

SEQ ID NO: 373 - DNA, 78 bp, Nicotiana plumbaginifolia
  source 1..78: mol_type = other DNA
atgggtaagatggcttctctgtttgctacttttctggttgttctggtttctctgtctctggcttctgaatcttctgct

SEQ ID NO: 374 - protein, 238 aa, Aequorea victoria
ASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 375 - protein, 225 aa, Juniperus ashei
MARVSELAFLLAATLAISLHMQEAGVVKFDIKNQCGYTVWAAGLPGGGKRLDQGQTWTVNLAAGTASARFWGRTGCTFDASGKGSCQTGDCGGQLSCTVSGAVPATLAEYTQSDQDYYDVSLVDGFNIPLAINPTNAQCTAPACKADINAVCPSELKVDGGCNSACNVFKTDQYCCRNAYVDNCPATNYSKIFKNQCPQAYSYAKDDTATFACASGTDYSIVFCP

SEQ ID NO: 376 - protein, 224 aa, Juniperus ashei
KFDIKNQCGYTVWAAGLPGGGKRLDQGQTWTVNLAAGTASARFWGRTGCTFDASGKGSCQTGDCGGQLSCTVSGAVPATLAEYTQSDQDYYDVSLVDGFNIPLAINPTNAQCTAPACKADINAVCPSELKVDGGCNSACNVFKTDQYCCRNAYVDNCPATNYSKIFKNQCPQAYSYAKDDTATFACASGTDYSIVFCMARVSELAFLLAATLAISLHMQEAGVV

SEQ ID NO: 377 - protein, 4 aa, Synthetic construct
IGER

SEQ ID NO: 378 - protein, 5 aa, Synthetic construct
EEKKN

SEQ ID NO: 379 - protein, 8 aa, Synthetic construct
ETMFKHGL